(19) United States
(12) Patent Application Publication
(10) Pub. No.: US 2024/0284792 A1
(43) Pub. Date: Aug. 22, 2024

(54) ORGANIC COMPOUND, LIGHT-EMITTING DEVICE, DISPLAY APPARATUS, AND ELECTRONIC DEVICE

(71) Applicant: SEMICONDUCTOR ENERGY LABORATORY CO., LTD., Atsugi-shi (JP)

(72) Inventors: Yuko KUBOTA, Atsugi (JP); Takeyoshi WATABE, Atsugi (JP); Hiromi SEO, Sagamihara (JP); Nobuharu OHSAWA, Zama (JP); Yui YOSHIYASU, Atsugi (JP); Sachiko KAWAKAMI, Atsugi (JP)

(21) Appl. No.: 18/425,324

(22) Filed: Jan. 29, 2024

(30) Foreign Application Priority Data
Feb. 3, 2023 (JP) 2023-015356

(51) Int. Cl. H10K 85/60; C07D 487/04; C09K 11/06; H10K 50/19; H10K 50/17

(52) U.S. Cl. CPC H10K 85/6572; C07D 487/04; C09K 11/06; H10K 50/19; H10K 85/624; H10K 85/654; C09K 2211/1018; H10K 50/171

(57) ABSTRACT

An organic compound with an electron-injection property and low water solubility is provided. An organic compound represented by General Formula (G1) is provided. In General Formula (G1), Ar is an aromatic skeleton represented by General Formula (Ar-1); L represents an alkylene group or an arylene group; n represents an integer of 0 to 3; m represents an integer of 1 to 4; and each of R¹ to R¹² independently represents hydrogen or an alkyl group. In General Formula (Ar-1), each of rings A, B, C, and D independently represents a benzene ring, a naphthalene ring, or a phenanthrene ring; any m carbon atoms on the rings A, B, C, and D each include a bond in General Formula (G1); X¹ represents C, Si, or Ge; and ai represents a single bond, O, S, C including a substituent, Si including a substituent, or Ge including a substituent.

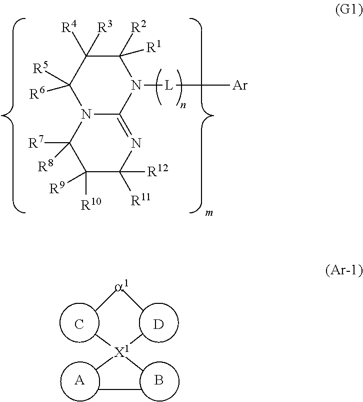

### BACKGROUND OF THE INVENTION

### 1. Field of the Invention

One embodiment of the present invention relates to an organic compound, a light-emitting device, a light-emitting apparatus, a light-emitting and light-receiving apparatus, a display apparatus, an electronic device, and a lighting device. Note that one embodiment of the present invention is not limited to the above technical field. The technical field of one embodiment of the invention disclosed in this specification and the like relates to an object, a method, or a manufacturing method. One embodiment of the present invention relates to a process, a machine, manufacture, or a composition of matter. Specifically, examples of the technical field of one embodiment of the present invention disclosed in this specification include a semiconductor device, a display apparatus, a liquid crystal display apparatus, a light-emitting apparatus, a lighting device, a power storage device, a memory device, an imaging device, a driving method thereof, and a manufacturing method thereof.

### 2. Description of the Related Art

Recent display apparatuses have been expected to be applied to a variety of uses. Usage examples of large-sized display apparatuses include a television device for home use (also referred to as TV or television receiver), digital signage, and a public information display (PID). In addition, a smartphone and a tablet terminal each including a touch panel, for example, are being developed as portable information terminals.

Higher-resolution display apparatuses have been required. For example, devices for virtual reality (VR), augmented reality (AR), substitutional reality (SR), or mixed reality (MR) are given as devices requiring high-resolution display apparatuses and have been actively developed in recent years.

Light-emitting apparatuses including light-emitting devices (also referred to as light-emitting elements) have been developed as display apparatuses, for example. Light-emitting devices utilizing electroluminescence (hereinafter referred to as EL; such devices are also referred to as EL devices or EL elements) have features such as ease of reduction in thickness and weight, high-speed response to input signals, and driving with a constant DC voltage power source, and have been used in display apparatuses.

Patent Document 1 discloses a display apparatus using an organic EL device (also referred to as organic EL element) for VR. Patent Document 2 discloses a light-emitting device that has a low driving voltage and high reliability and includes an electron-injection layer formed using a mixed film of a transition metal and an organic compound including an unshared electron pair.

### REFERENCES

### Patent Documents

[Patent Document 1] International Publication No. WO2018/087625
[Patent Document 2] Japanese Published Patent Application No. 2018-201012

### SUMMARY OF THE INVENTION

As a method for forming an organic semiconductor film in a predetermined shape, a vacuum evaporation method with a metal mask (mask vapor deposition) is widely used. However, in these days of higher density and higher resolution, mask vapor deposition has come close to the limit of increasing the resolution for various reasons such as the alignment accuracy and the distance between the mask and the substrate. By contrast, a finer pattern can be formed by shape processing of an organic semiconductor film by a lithography method. Moreover, because of the easiness of large-area processing in this method, the processing of an organic semiconductor film by a lithography method is being researched.

An organic EL device includes an organic compound layer containing a light-emitting substance (corresponding to the above organic semiconductor film) between electrodes (between a first electrode and a second electrode), and energy generated by recombination of carriers (holes and electrons) injected to the organic compound layer from the electrodes causes light emission.

Carrier injection, especially electron injection into the organic compound layer, through which electricity is difficult to flow, has to overcome a high energy barrier and therefore essentially requires a high voltage. In view of this, currently, an electron-injection layer in contact with the cathode contains a donor substance (also referred to as an electron donor), whereby a reduction in voltage can be achieved. Typical examples of the donor substance include alkali metals such as lithium (Li), which have a low work function, and compounds of the alkali metals.

However, in the case where the aforementioned lithography method is employed to fabricate a light-emitting device including an organic compound layer containing the donor substance, oxygen or water in the air and a chemical solution or water used in the process have caused a significant increase in driving voltage or a marked reduction in current efficiency.

A way of solving the above problem is to perform processing by a lithography method halfway through a process of forming the organic compound layer of a light-emitting device (before forming the layer containing a donor substance). In other words, the organic compound layer is processed by a lithography method prior to the formation of the electron-injection layer, and then the formation of the electron-injection layer and the subsequent steps are performed, whereby degradation of characteristics can be avoided.

However, for a tandem light-emitting device, the above solving way cannot be employed and the processing of the organic compound layer by a lithography method has inevitably caused a significant degradation of characteristics.

This is because a tandem light-emitting device includes an organic compound layer in which a plurality of light-emitting layers are stacked in series with an intermediate layer (also referred to as a charge-generation layer) interposed therebetween, and the intermediate layer contains a donor substance for electron injection into the light-emitting layer on the anode side. Since the intermediate layer is provided between two light-emitting layers, when the organic compound layer including the two light-emitting layers is processed by a lithography method, the intermediate layer is also processed inevitably by a lithography method and is consequently exposed to oxygen or water in the air or a chemical solution or water used in the process.

Like exposure of the electron-injection layer to a processing step by a lithography method, exposure of a layer containing a donor substance in the intermediate layer to oxygen, water, or the like in a processing step by a lithography method have caused a significant increase in driving voltage or a marked reduction in current efficiency of the light-emitting device.

Another way of solving the above problem is using an organic compound having an electron-injection property, instead of a donor substance, for the electron-injection layer or the intermediate layer. Specifically, in the above way, the organic compound layer containing no donor substance is processed by a lithography method, so that it is possible to avoid the degradation of characteristics of the light-emitting device due to a donor substance.

However, if the water solubility of the organic compound is high, the layer including the organic compound is dissolved when exposed to water or a chemical solution containing water as a solvent, which might cause degraded characteristics, a shape defect, or the like.

An object of one embodiment of the present invention is to provide an organic compound having an electron-injection property. Another object of one embodiment of the present invention is to provide an organic compound with low water solubility. Another object of one embodiment of the present invention is to provide an organic compound having a high glass transition temperature (Tg). Another object of one embodiment of the present invention is to provide a light-emitting device with favorable light-emitting characteristics. Another object of one embodiment of the present invention is to provide a novel organic compound, a novel light-emitting device, a novel display apparatus, or a novel electronic device.

Note that the description of these objects does not preclude the existence of other objects. One embodiment of the present invention does not need to achieve all of these objects. Other objects will be apparent from and can be derived from the description of the specification, the drawings, the claims, and the like.

To solve the above problem, one embodiment of the present invention provides an organic compound including a bicyclic guanidine skeleton and an aromatic skeleton. This organic compound has high basicity and an electron-injection property; thus, instead of a donor substance, the organic compound can be used for an electron-injection layer or an intermediate layer in an organic compound layer of a light-emitting device. Thus, as compared to the case of using a donor substance, even when the organic compound layer is exposed to water, oxygen, or the like in a processing step by a lithography method, a significant increase in driving voltage and a marked reduction in current efficiency of the light-emitting device can be prevented, whereby the light-emitting device can have favorable characteristics.

Furthermore, the present inventors have found that increasing the hydrophobic property of the aromatic skeleton in the organic compound can reduce the water solubility of the organic compound.

One embodiment of the present invention is an organic compound represented by General Formula (G1) below.

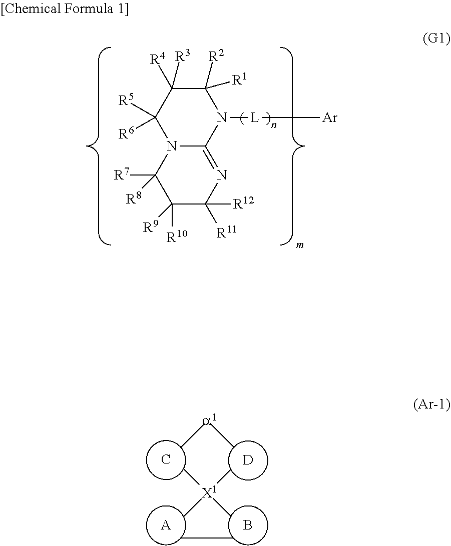

In General Formula (G1), Ar is an aromatic skeleton represented by General Formula (Ar-1); L represents an alkylene group having 1 to 10 carbon atoms, a substituted or unsubstituted arylene group having 6 to 30 carbon atoms, or a substituted or unsubstituted heteroarylene group having 1 to 11 carbon atoms; n represents an integer of 0 to 3, m represents an integer of 1 to 4; and each of R¹ to R¹² independently represents hydrogen (including deuterium) or an alkyl group having 1 to 10 carbon atoms. In the case where n is 2 or more, Ls may be the same or different from each other. In the case where m is 2 or more, Ls may be the same or different from each other, n's may be the same or different from each other, and each of R¹s to R¹²s may be independently the same or different from each other. In General Formula (Ar-1), each of a ring A, a ring B, a ring C, and a ring D independently represents a substituted or unsubstituted benzene ring, a substituted or unsubstituted naphthalene ring, or a substituted or unsubstituted phenanthrene ring; any m carbon atoms on the ring A, the ring B, the ring C, and the ring D each include a bond in General Formula (G1); X¹ represents carbon (C), silicon (Si), or germanium (Ge); and α¹ represents a single bond, oxygen, sulfur, carbon including a substituent, silicon including a substituent, or germanium including a substituent.

Another embodiment of the present invention is the organic compound with the above structure, in which α¹ is represented by any of General Formulae (α-1) to (α-3) below.

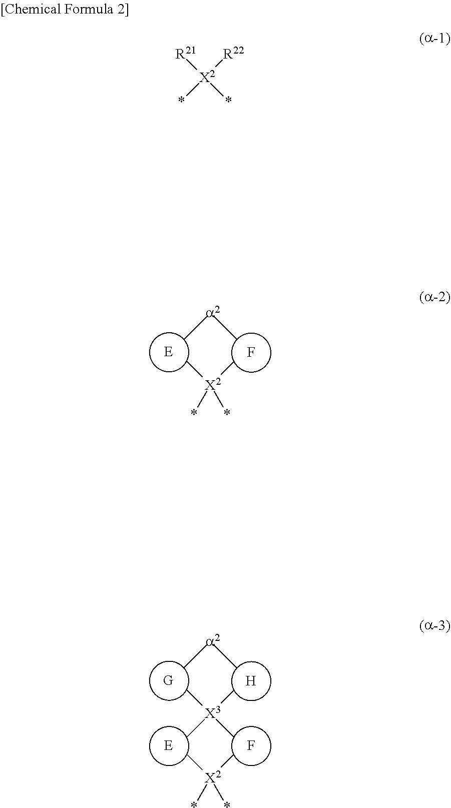

In General Formulae (α-1) to (α-3), each of X² and X³ independently represents carbon, silicon, or germanium; α² represents a single bond, oxygen, sulfur, carbon including a substituent, silicon including a substituent, or germanium including a substituent; each of the ring E, the ring F, the ring G, and the ring H independently represents a substituted or unsubstituted benzene ring, a substituted or unsubstituted naphthalene ring, or a substituted or unsubstituted phenanthrene ring; each of R²¹ and R²² independently represents hydrogen (including deuterium), an alkyl group having 1 to 10 carbon atoms, a cycloalkyl group having 3 to 10 carbon atoms, a substituted or unsubstituted aryl group having 6 to 30 carbon atoms, or a substituted or unsubstituted heteroaryl group having 1 to 30 carbon atoms. Note that * represents bonding positions of the ring C and the ring D in General Formula (Ar-1).

Another embodiment of the present invention is an organic compound represented by General Formula (G1) below.

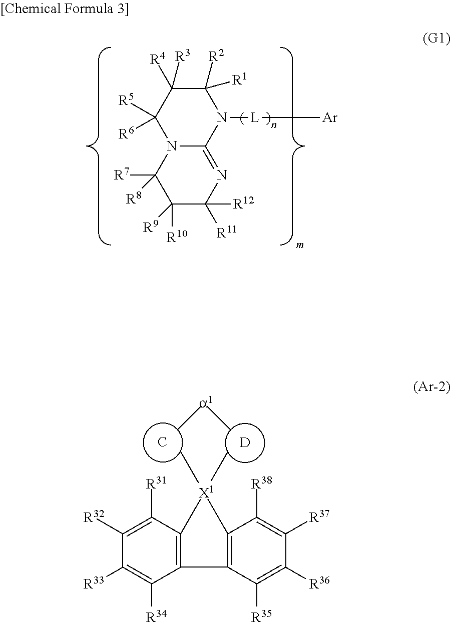

In General Formula (G1), Ar is an aromatic skeleton represented by General Formula (Ar-2); L represents an alkylene group having 1 to 10 carbon atoms, a substituted or unsubstituted arylene group having 6 to 30 carbon atoms, or a substituted or unsubstituted heteroarylene group having 1 to 11 carbon atoms; n represents an integer of 0 to 3; m represents an integer of 1 to 4; and each of R¹ to R¹² independently represents hydrogen (including deuterium) or an alkyl group having 1 to 10 carbon atoms. In the case where n is 2 or more, Ls may be the same or different from each other. In the case where m is 2 or more, Ls may be the same or different from each other, n's may be the same or different from each other, and each of R¹s to R¹²s may be independently the same or different from each other. In General Formula (Ar-2), each of the ring C and the ring D independently represents a substituted or unsubstituted benzene ring, a substituted or unsubstituted naphthalene ring, or a substituted or unsubstituted phenanthrene ring; X¹ represents carbon, silicon, or germanium; α¹ represents a single bond, oxygen, sulfur, carbon including a substituent, silicon including a substituent, or germanium including a substituent; any m groups of R³¹ to R³⁸ represent bonding positions in General Formula (G1); each of the other groups of R³¹ to R³⁸ independently represents hydrogen (including deuterium), an alkyl group having 1 to 10 carbon atoms, a cycloalkyl group having 3 to 10 carbon atoms, a substituted or unsubstituted aryl group having 6 to 30 carbon atoms, or a substituted or unsubstituted heteroaryl group having 1 to 30 carbon atoms.

Another embodiment of the present invention is the organic compound with the above structure, in which α¹ is represented by any of General Formulae (α-4) to (α-6) below.

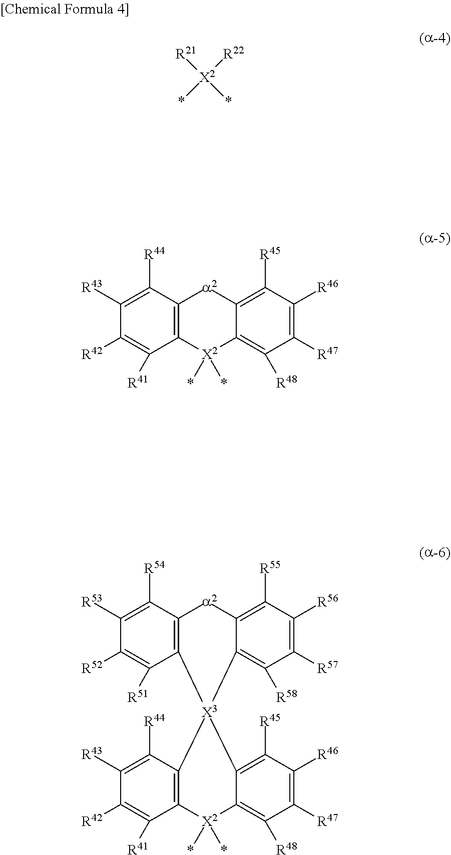

In General Formulae (α-4) to (α-6), each of X² and X³ independently represents carbon, silicon, or germanium; α² represents a single bond, oxygen, sulfur, carbon including a substituent, silicon including a substituent, or germanium including a substituent; each of R²¹, R²², R⁴¹ to R⁴⁸, and R⁵¹ to R⁵⁸ independently represents hydrogen (including deuterium), an alkyl group having 1 to 10 carbon atoms, a cycloalkyl group having 3 to 10 carbon atoms, a substituted or unsubstituted aryl group having 6 to 30 carbon atoms, or a substituted or unsubstituted heteroaryl group having 1 to 30 carbon atoms. Note that * represents bonding positions of the ring C and the ring D in General Formula (Ar-2).

Another embodiment of the present invention is an organic compound represented by General Formula (G1) below.

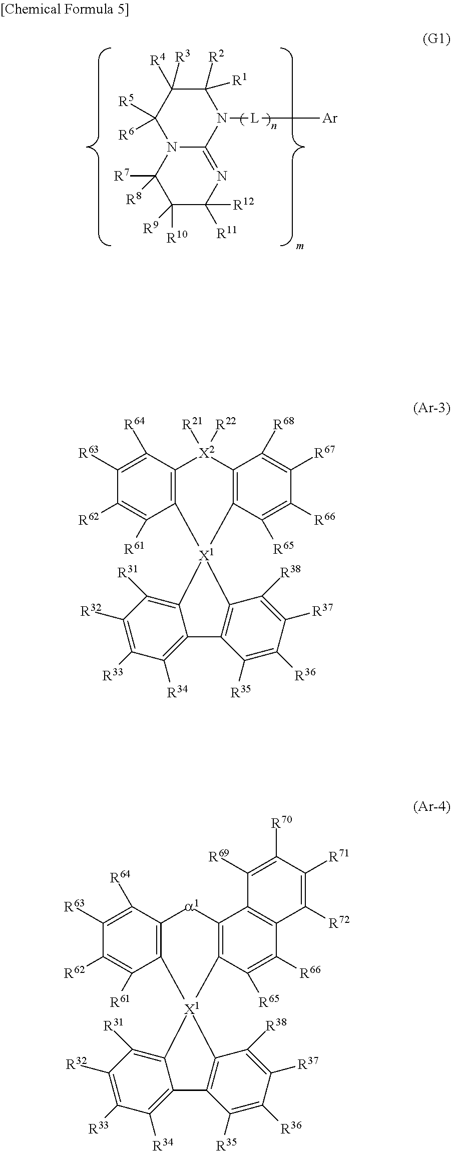

In General Formula (G1), Ar is an aromatic skeleton represented by General Formula (Ar-3) or (Ar-4) above; L represents an alkylene group having 1 to 10 carbon atoms, a substituted or unsubstituted arylene group having 6 to 30 carbon atoms, or a substituted or unsubstituted heteroarylene group having 1 to 11 carbon atoms; n represents an integer of 0 to 3; m represents an integer of 1 to 4; each of R¹ to R¹² independently represents hydrogen (including deuterium) or an alkyl group having 1 to 10 carbon atoms. In the case where n is 2 or more, Ls may be the same or different from each other. In the case where m is 2 or more, Ls may be the same or different from each other, n's may be the same or different from each other, and each of R¹s to R¹²s may be independently the same or different from each other. In General Formulae (Ar-3) and (Ar-4), each of X¹ and X² independently represents carbon, silicon, or germanium; α¹ represents a single bond, oxygen, sulfur, carbon including a substituent, silicon including a substituent, or germanium including a substituent; any m groups of R³¹ to R³⁸ and R⁶¹ to R⁶⁶ represent bonding positions in General Formula (G1); and each of the other groups of R³¹ to R³⁸ and R⁶¹ to R⁶⁶, and R⁶⁷ to R⁷², R²¹, and R²² independently represents hydrogen (including deuterium), an alkyl group having 1 to 10 carbon atoms, a cycloalkyl group having 3 to 10 carbon atoms, a substituted or unsubstituted aryl group having 6 to 30 carbon atoms, or a substituted or unsubstituted heteroaryl group having 1 to 30 carbon atoms.

Another embodiment of the present invention is an organic compound represented by General Formula (G1) below.

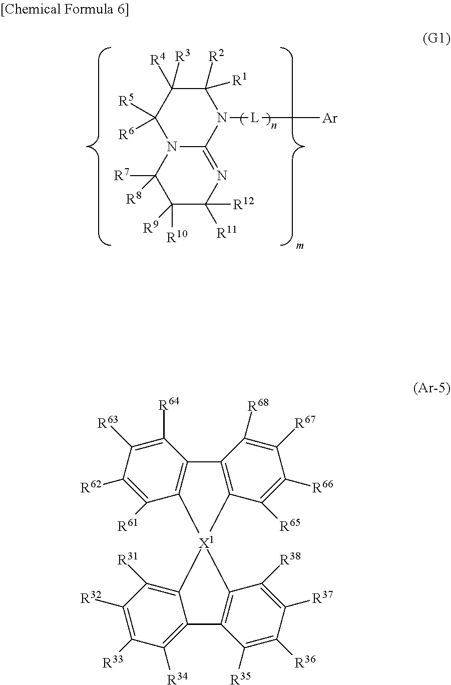

In General Formula (G1), Ar is an aromatic skeleton represented by General Formula (Ar-5); L represents an alkylene group having 1 to 10 carbon atoms, a substituted or unsubstituted arylene group having 6 to 30 carbon atoms, or a substituted or unsubstituted heteroarylene group having 1 to 11 carbon atoms; n represents an integer of 1 to 3; m represents an integer of 1 to 4; and each of R¹ to R¹² independently represents hydrogen (including deuterium) or an alkyl group having 1 to 10 carbon atoms. In the case where n is 2 or more, Ls may be the same or different from each other. In the case where m is 2 or more, Ls may be the same or different from each other, n's may be the same or different from each other, and each of R¹s to R¹²s may be independently the same or different from each other. In General Formula (Ar-5), X¹ represents carbon, silicon, or germanium; any m groups of R³¹ to R³⁸ and R⁶¹ to R⁶⁸ represent bonding positions in General Formula (G1); each of the other groups independently represents hydrogen (including deuterium), an alkyl group having 1 to 10 carbon atoms, a cycloalkyl group having 3 to 10 carbon atoms, a substituted or unsubstituted aryl group having 6 to 30 carbon atoms, or a substituted or unsubstituted heteroaryl group having 1 to 30 carbon atoms.

Another embodiment of the present invention is an organic compound represented by any one of General Formulae (G2-1) to (G2-3) below.

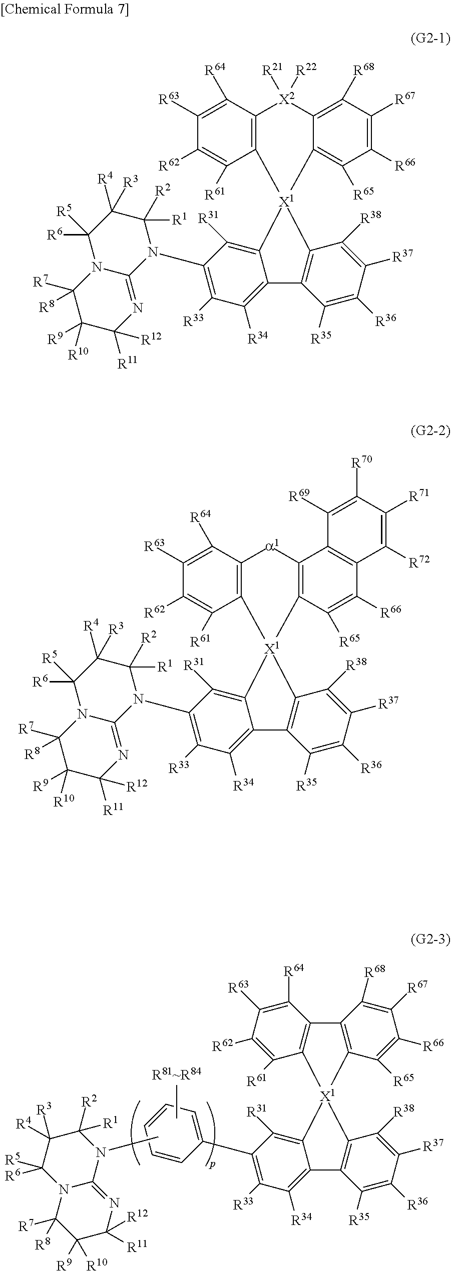

In General Formulae (G2-1) to (G2-3), each of R¹ to R¹² independently represents hydrogen (including deuterium) or an alkyl group having 1 to 10 carbon atoms. Each of X¹ and X² independently represents carbon, silicon, or germanium; α¹ represents a single bond, oxygen, sulfur, carbon including a substituent, silicon including a substituent, or germanium including a substituent; each of R³¹, R³³ to R³⁸, R⁶¹ to R⁷², R⁸¹ to R⁸⁴, R²¹, and R²² independently represents hydrogen (including deuterium), an alkyl group having 1 to 10 carbon atoms, a cycloalkyl group having 3 to 10 carbon atoms, a substituted or unsubstituted aryl group having 6 to 30 carbon atoms, or a substituted or unsubstituted heteroaryl group having 1 to 30 carbon atoms; and p represents an integer of 1 to 3.

Another embodiment of the present invention is an organic compound represented by any one of Structural Formulae (100), (115), (133), (137), and (153) below.

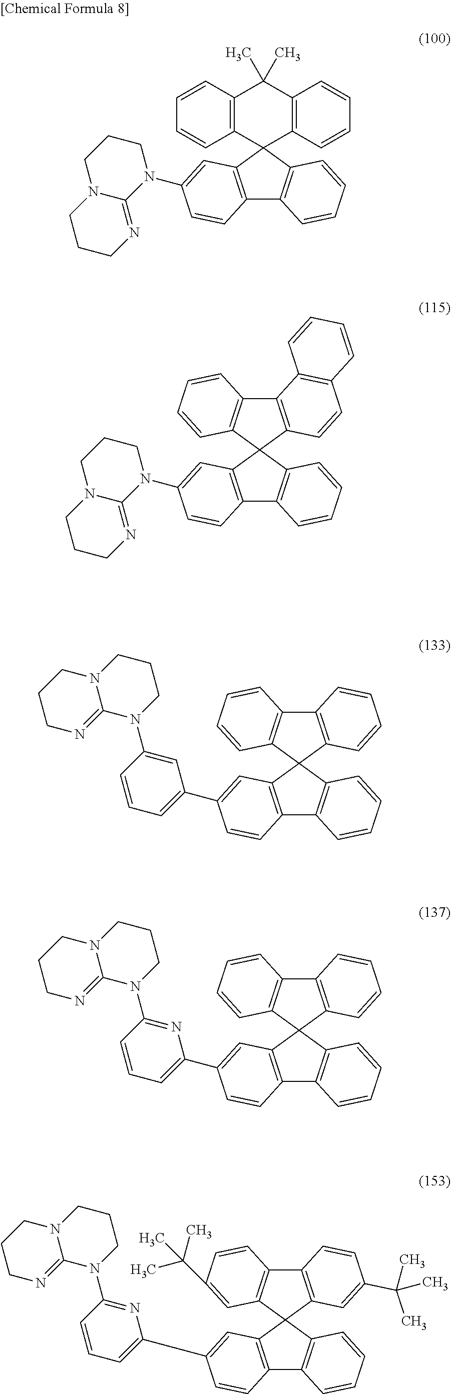 [SMILES: CC1(C)c2ccccc2C2(c3ccccc3-c3ccc(N4CCCN5CCCN=C54)cc32)c2ccccc21 ; c1ccc2c(c1)-c1ccc(N3CCCN4CCCN=C43)cc1C21c2ccccc2-c2c1ccc1ccccc21 ; c1cc(-c2ccc3c(c2)C2(c4ccccc4-c4ccccc42)c2ccccc2-3)cc(N2CCCN3CCCN=C32)c1 ; c1cc(-c2ccc3c(c2)C2(c4ccccc4-c4ccccc42)c2ccccc2-3)nc(N2CCCN3CCCN=C32)c1 ; CC(C)(C)c1ccc2c(c1)C1(c3ccccc3-c3ccc(-c4cccc(N5CCCN6CCCN=C65)n4)cc31)c1cc(C(C)(C)C)ccc1-2]

Another embodiment of the present invention is a light-emitting device including the organic compound having any of the above structures.

Another embodiment of the present invention is a display apparatus including the above light-emitting device, and at least one of a transistor and a substrate.

Another embodiment of the present invention is an electronic device including the above display apparatus; and a sensor unit, an input unit, or a communication unit.

Note that the display apparatus in this specification includes, in its category, an image display device that uses a light-emitting device. The display apparatus may also include a module in which a light-emitting device over a substrate is provided with a connector such as an anisotropic conductive film or a tape carrier package (TCP), a module in which a printed wiring board is provided at the end of a TCP, and a module in which an integrated circuit (IC) is directly mounted on a light-emitting device by a chip on glass (COG) method. Furthermore, a lighting device or the like may include the display apparatus.

One embodiment of the present invention can provide an organic compound having an electron-injection property. Another embodiment can provide an organic compound with low water solubility. Another embodiment of the present invention can provide an organic compound having a high glass transition temperature (Tg). Another embodiment can provide a light-emitting device with favorable characteristics. Another embodiment can provide a novel organic compound, a novel light-emitting device, a novel display apparatus, or a novel electronic device.

Note that the description of these effects does not preclude the existence of other effects. One embodiment of the present invention does not necessarily have all of these effects. Other effects can be derived from the description of the specification, the drawings, and the claims.

### BRIEF DESCRIPTION OF THE DRAWINGS

In the accompanying drawings:

FIGS. 1A to 1C each illustrate a light-emitting device;

FIG. 2 illustrates a light-emitting device;

FIGS. 3A and 3B are, respectively, a top view and a cross-sectional view of a display apparatus;

FIGS. 4A to 4D each illustrate a light-emitting device;

FIGS. 5A to 5E are cross-sectional views illustrating an example of a method for manufacturing a display apparatus;

FIGS. 6A to 6D are cross-sectional views illustrating the example of a method for manufacturing a display apparatus;

FIGS. 7A to 7D are cross-sectional views illustrating the example of a method for manufacturing a display apparatus;

FIGS. 8A to 8C are cross-sectional views illustrating the example of a method for manufacturing a display apparatus;

FIGS. 9A to 9C are cross-sectional views illustrating the example of a method for manufacturing a display apparatus;

FIGS. 10A to 10C are cross-sectional views illustrating the example of a method for manufacturing a display apparatus;

FIGS. 11A to 11C illustrate a conventional structure;

FIGS. 12A to 12E illustrate a film processing method;

FIGS. 13A to 13E illustrate a film processing method;

FIGS. 14A and 14B are perspective views illustrating a structure example of a display module;

FIGS. 15A and 15B are cross-sectional views illustrating structure examples of a display apparatus;

FIGS. 16A to 16D illustrate examples of electronic devices;

FIGS. 17A to 17F illustrate examples of electronic devices;

FIG. 18 is a ¹H NMR spectrum of Fha-hpp;

FIG. 19 is a ¹H NMR spectrum of SBF-hpp;

FIG. 20 is a ¹H NMR spectrum of mSFP-hpp;

FIG. 21 shows the luminance-current density characteristics of a light-emitting device 1;

FIG. 22 shows the luminance-voltage characteristics of the light-emitting device 1;

FIG. 23 shows the current efficiency-luminance characteristics of the light-emitting device 1;

FIG. 24 shows the current density-voltage characteristics of the light-emitting device 1;

FIG. 25 shows the electroluminescence spectrum of the light-emitting device 1;

FIG. 26 shows a luminance change over driving time of the light-emitting device 1;

FIG. 27 shows the luminance-current density characteristics of a light-emitting device 2;

FIG. 28 shows the luminance-voltage characteristics of the light-emitting device 2;

FIG. 29 shows the current efficiency-luminance characteristics of the light-emitting device 2;

FIG. 30 shows the current density-voltage characteristics of the light-emitting device 2;

FIG. 31 shows the electroluminescence spectrum of the light-emitting device 2;

FIG. 32 shows a luminance change over driving time of the light-emitting device 2;

FIG. 33 shows the luminance-current density characteristics of a light-emitting device 3;

FIG. 34 shows the luminance-voltage characteristics of the light-emitting device 3;

FIG. 35 shows the current efficiency-luminance characteristics of the light-emitting device 3;

FIG. 36 shows the current density-voltage characteristics of the light-emitting device 3;

FIG. 37 shows the electroluminescence spectrum of the light-emitting device 3;

FIG. 38 shows a luminance change over driving time of the light-emitting device 3;

FIG. 39 shows a ¹H NMR spectrum of SFPy-hpp;

FIG. 40 shows a ¹H NMR spectrum of tBuSFPy-hpp;

FIG. 41 shows the luminance-current density characteristics of a light-emitting device 4 and a light-emitting device 5;

FIG. 42 shows the luminance-voltage characteristics of the light-emitting device 4 and the light-emitting device 5;

FIG. 43 shows the current efficiency-luminance characteristics of the light-emitting device 4 and the light-emitting device 5;

FIG. 44 shows the current density-voltage characteristics of the light-emitting device 4 and the light-emitting device 5;

FIG. 45 shows the electroluminescence spectra of the light-emitting device 4 and the light-emitting device 5; and

FIG. 46 shows luminance changes over driving time of the light-emitting device 4 and the light-emitting device 5.

### DETAILED DESCRIPTION OF THE INVENTION

Embodiments will be described in detail with reference to the drawings. Note that the embodiments of the present invention are not limited to the following description, and it will be readily appreciated by those skilled in the art that modes and details of the present invention can be modified in various ways without departing from the spirit and scope of the present invention. Therefore, the present invention should not be construed as being limited to the description in the following embodiments.

Note that in structures of the invention described below, the same portions or portions having similar functions are denoted by the same reference numerals in different drawings, and the description thereof is not repeated. The same hatching pattern is used for portions having similar functions, and the portions are not denoted by specific reference numerals in some cases.

The position, size, range, or the like of each component illustrated in drawings does not represent the actual position, size, range, or the like in some cases for easy understanding. Therefore, the disclosed invention is not necessarily limited to the position, size, range, or the like disclosed in the drawings.

Note that the terms “film” and “layer” can be used interchangeably depending on the case or the circumstances. For example, the term “conductive layer” can be replaced with the term “conductive film”. As another example, the term “insulating film” can be replaced with the term “insulating layer”.

In this specification and the like, a device formed using a metal mask or a fine metal mask (FMM) is sometimes referred to as a device having a metal mask (MM) structure. In this specification and the like, a device formed without using a metal mask or an FMM is sometimes referred to as a device having a metal maskless (MML) structure.

In this specification and the like, a light-emitting device (also referred to as a light-emitting element) includes an EL layer between a pair of electrodes. The EL layer includes at least a light-emitting layer.

In this specification and the like, a tapered shape indicates a shape in which at least part of a side surface of a component is inclined to a substrate surface. For example, a tapered shape preferably includes a region where the angle formed by the inclined side surface and the substrate surface (such an angle is also referred to as a taper angle) is less than 90°. Note that the side surface of the component and the substrate surface is not necessarily completely flat, and may have a substantially planar shape with a small curvature or slight unevenness.

### Embodiment 1

In this embodiment, an organic compound of one embodiment of the present invention will be described.

One embodiment of the present invention provides an organic compound including a bicyclic guanidine skeleton and an aromatic skeleton. This organic compound has high basicity and an electron-injection property; thus, instead of a donor substance, the organic compound can be used for an electron-injection layer or an intermediate layer in an organic compound layer of a light-emitting device. Thus, as compared to the case of using a donor substance, even when the organic compound layer is exposed to water, oxygen, or the like in a processing step by a lithography method, a significant increase in driving voltage and a marked reduction in current efficiency of the light-emitting device can be prevented, whereby the light-emitting device can have favorable characteristics.

However, the dipole moment of an organic compound having high basicity is large and accordingly the water solubility of the organic compound is high. This allows, for example, dissolution of a layer including the organic compound or permeation of a chemical solution into the layer including the organic compound in a processing step by a lithography method involving exposure to water or the chemical solution containing water as a solvent, which might cause degraded characteristics, a shape defect, or the like, of a light-emitting device. Thus, the water solubility needs to be reduced while the electron-injection property sufficient for use in a light-emitting device is maintained. One possible way to reduce the water solubility is increasing the hydrophobic property of the aromatic skeleton.

Since a bicyclic guanidine skeleton has high planarity, an organic compound including the skeleton is likely to be crystallized. Thus, the use of the organic compound including the bicyclic guanidine skeleton for a light-emitting device might decrease the reliability of the light-emitting device. One possible solution is making the aromatic skeleton bulky.

When the aromatic skeleton is bulky, the organic compound including the aromatic skeleton sometimes easily absorbs light in a visible-light region with expansion of a π-conjugated system. It is undesirable that a material used for an intermediate layer easily absorb light in a visible-light region because the element efficiency might decrease. One embodiment of the present invention uses an aromatic skeleton containing a spiro atom in its center. This can cut the π-conjugated system and accordingly can inhibit absorption in a visible-light region.

Thus, the organic compound of one embodiment of the present invention is represented by General Formula (G1).

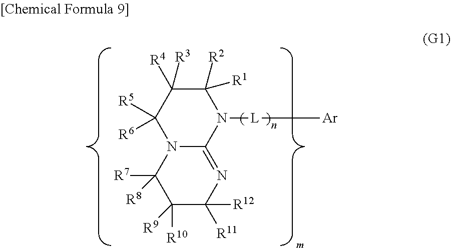

In General Formula (G1), Ar is an aromatic skeleton containing a spiro atom; L represents an alkylene group having 1 to 10 carbon atoms, a substituted or unsubstituted arylene group having 6 to 30 carbon atoms, or a substituted or unsubstituted heteroarylene group having 1 to 11 carbon atoms; n represents an integer of 0 to 3; m represents an integer of 1 to 4; and each of R¹ to R¹² independently represents hydrogen (including deuterium) or an alkyl group having 1 to 10 carbon atoms. In the case where n is 2 or more, Ls may be the same or different from each other. In the case where m is 2 or more, Ls may be the same or different from each other, n's may be the same or different from each other, and each of R¹s to R¹²s may be independently the same or different from each other.

An aromatic skeleton containing a spiro atom has a high molecular weight and thus can reduce the water solubility of the organic compound. This accordingly can avoid dissolution of the layer including the organic compound in a processing step by a lithography method.

In addition, an aromatic skeleton containing a spiro atom is bulky and thus can decrease the planarity of the whole organic compound and the crystallinity of the layer including the organic compound. Accordingly, the glass transition temperature (T_{g}) of the organic compound can be increased.

In some cases, a film including an organic compound with a low glass transition temperature has unstable film quality and might cause decrease in reliability of a light-emitting device. However, the organic compound of one embodiment of the present invention, which uses an aromatic skeleton containing a spiro atom, can have a high glass transition temperature. Thus, the use of the organic compound can improve the reliability of the light-emitting device.

As the spiro atom in Ar, carbon, silicon, or germanium can be used. Carbon is particularly preferable in terms of easy synthesis and low cost.

In General Formula (G1), in the case where n is 0, the organic compound can be easily synthesized. In the case where n is an integer of 1 to 3, as compared to the case where n is 0, the number of hydrophobic substituents (an alkylene group having 1 to 10 carbon atoms, a substituted or unsubstituted arylene group having 6 to 30 carbon atoms, or a substituted or unsubstituted heteroarylene group having 1 to 11 carbon atoms) increases and the molecular weight of the organic compound increases, whereby the water solubility can be further reduced.

In General Formula (G1), in the case where m is an integer of 2 to 4, the basicity of the organic compound can be increased. In the case where m is 1, the water solubility of the organic compound can be reduced.

As the aromatic skeleton containing a spiro atom in General Formula (G1), an aromatic skeleton represented by General Formula (Ar-1) below can be used, for example.

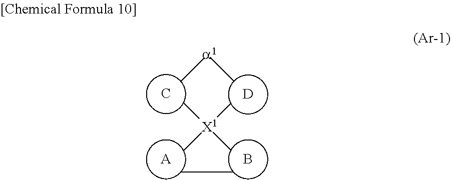

In General Formula (Ar-1), each of the ring A, the ring B, the ring C, and the ring D independently represents a substituted or unsubstituted benzene ring, a substituted or unsubstituted naphthalene ring, or a substituted or unsubstituted phenanthrene ring; any m carbon atoms on the ring A, the ring B, the ring C, and the ring D each include a bond in General Formula (G1); X¹ represents carbon, silicon, or germanium; and α¹ represents a single bond, oxygen, sulfur, carbon including a substituent, silicon including a substituent, or germanium including a substituent.

In General Formula (Ar-1), in the case where the ring A to the ring D each include a substituent, specific examples of the substituent include an alkyl group having 1 to 10 carbon atoms, a cycloalkyl group having 3 to 10 carbon atoms, a substituted or unsubstituted aryl group having 6 to 30 carbon atoms, and a substituted or unsubstituted heteroaryl group having 1 to 30 carbon atoms.

In the aromatic skeleton represented by General Formula (Ar-1), the ring A, the ring B, the ring C, and the ring D are bonded to X¹ that is a spiro atom, the ring A and the ring B are bonded to each other by a single bond, and the ring C and the ring D are bonded to each other through α¹, so that a spiro cyclic skeleton is formed. Such an aromatic skeleton has a hydrophobic property and a high molecular weight, and thus can reduce the water solubility of an organic compound when used for the organic compound. This accordingly can avoid dissolution of the layer including the organic compound in a processing step by a lithography method. In addition, the aromatic skeleton is bulky and thus can reduce the planarity of the whole organic compound and the crystallinity of the layer including the organic compound. Accordingly, the glass transition temperature of the organic compound can be increased, thereby preventing abnormality in film quality of the layer including the organic compound in a heating step.

As described above, a film including an organic compound with a low glass transition temperature sometimes has unstable film quality and might cause decrease in reliability of a light-emitting device. However, the use of the aromatic skeleton represented by General Formula (Ar-1) can increase the glass transition temperature of the organic compound. Thus, the use of the organic compound can prevent abnormality in film quality of the layer including the organic compound in a heating step and can improve the reliability of the light-emitting device.

When an aromatic ring with high visible-light-absorption intensity (e.g., an acetylene ring or a tetracene ring) is used as the ring A, the ring B, the ring C, and the ring D, the organic compound absorbs a large amount of visible light and decreases the emission efficiency of the light-emitting device. In view of this, as the ring A, the ring B, the ring C, and the ring D, it is preferable to use a benzene ring, a naphthalene ring, or a phenanthrene ring whose visible-light-absorption intensity is not high. In that case, the organic compound can be inhibited from having high visible-light-absorption intensity, so that a decrease in emission efficiency of the light-emitting device can be prevented.

In the case where the ring A, the ring B, the ring C, and the ring D are all benzene rings, α¹ is preferably oxygen, sulfur, carbon including a substituent, silicon including a substituent, or germanium including a substituent. In that case, the organic compound can have a higher molecular weight and lower water solubility. Furthermore, the organic compound becomes bulky and accordingly can have a higher glass transition temperature. Thus, the use of the organic compound can prevent abnormality in film quality of the layer including the organic compound in a heating step, and can improve the reliability of the light-emitting device. In the case where the ring A, the ring B, the ring C, and the ring D are all benzene rings and α¹ is a single bond, the number of hydrophobic substituents or the molecular weight of the organic compound can be increased and accordingly the water solubility can be reduced by providing a substituent for one or more of the benzene rings or by setting n in General Formula (G1) to an integer of 1 to 3, for example.

Specific examples of carbon including a substituent, silicon including a substituent, and germanium including a substituent which can be used in General Formula (Ar-1) include skeletons represented by General Formulae (α-1) to (α-3) below.

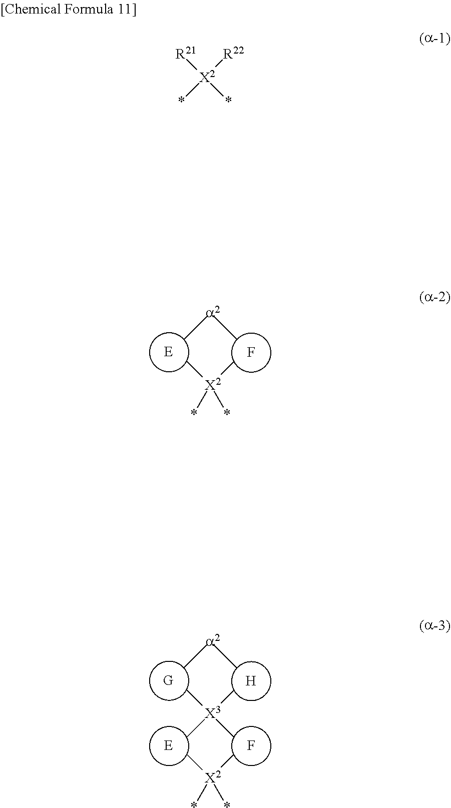

In General Formulae (α-1) to (α-3), each of X² and X³ independently represents carbon, silicon, or germanium; α² represents a single bond, oxygen, sulfur, carbon including a substituent, silicon including a substituent, or germanium including a substituent; each of the ring E, the ring F, the ring G, and the ring H independently represents a substituted or unsubstituted benzene ring, a substituted or unsubstituted naphthalene ring, or a substituted or unsubstituted phenanthrene ring; each of R²¹ and R²² independently represents hydrogen (including deuterium), an alkyl group having 1 to 10 carbon atoms, a substituted or unsubstituted cycloalkyl group having 3 to 10 carbon atoms, a substituted or unsubstituted aryl group having 6 to 30 carbon atoms, or a substituted or unsubstituted heteroaryl group having 1 to 30 carbon atoms. Note that * represents bonding positions of the ring C and the ring D in General Formula (Ar-1).

In the above formulae, in the case where a ring E to a ring H each include a substituent, specific examples of the substituent include an alkyl group having 1 to 10 carbon atoms, a cycloalkyl group having 3 to 10 carbon atoms, a substituted or unsubstituted aryl group having 6 to 30 carbon atoms, and a substituted or unsubstituted heteroaryl group having 1 to 30 carbon atoms.

When a skeleton represented by General Formula (α-1) is used as α¹ of the aromatic skeleton represented by General Formula (Ar-1), the number of hydrophobic substituents and the molecular weight of the organic compound can be increased. This can reduce the water solubility of the organic compound. Furthermore, the aromatic skeleton contains two or more spiro atoms and accordingly becomes bulky, thereby enabling the organic compound to have lower crystallinity. Thus, the organic compound can have a higher glass transition temperature.

Note that the organic compound of one embodiment of the present invention preferably has a glass transition temperature higher than or equal to 100° C., for example, in which case water, an atmospheric component, or the like adsorbed onto the organic compound in a processing step by a lithography method can be removed in a heating step without causing abnormality in film quality and the reliability of the light-emitting device including the organic compound can be improved.

Alternatively, as the aromatic skeleton containing a spiro atom in General Formula (G1), an aromatic skeleton represented by General Formula (Ar-2) below can be used, for example. Note that General Formula (Ar-2) shows a structure where the ring A and the ring B in General Formula (Ar-1) are limited to substituted or unsubstituted benzene rings. Thus, description of the structure, effect, and the like of General Formula (Ar-1) can be applied to General Formula (Ar-2).

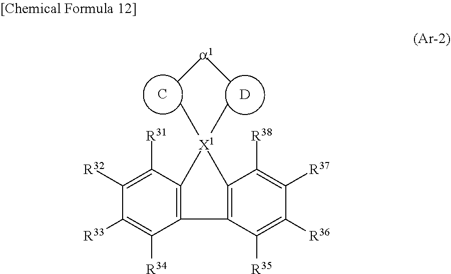

In General Formula (Ar-2), each of the ring C and the ring D independently represents a substituted or unsubstituted benzene ring, a substituted or unsubstituted naphthalene ring, or a substituted or unsubstituted phenanthrene ring; X¹ represents carbon, silicon, or germanium; α¹ represents a single bond, oxygen, sulfur, carbon including a substituent, silicon including a substituent, or germanium including a substituent; any m groups of R³¹ to R³⁸ represent bonding positions in General Formula (G1); and each of the other groups of R³¹ to R³⁸ independently represents hydrogen (including deuterium), an alkyl group having 1 to 10 carbon atoms, a cycloalkyl group having 3 to 10 carbon atoms, a substituted or unsubstituted aryl group having 6 to 30 carbon atoms, or a substituted or unsubstituted heteroaryl group having 1 to 30 carbon atoms.

In the above general formula, in the case where the ring C and the ring D each include a substituent, specific examples of the substituent include an alkyl group having 1 to 10 carbon atoms, a cycloalkyl group having 3 to 10 carbon atoms, a substituted or unsubstituted aryl group having 6 to 30 carbon atoms, and a substituted or unsubstituted heteroaryl group having 1 to 30 carbon atoms.

As described above, the aromatic skeleton represented by General Formula (Ar-2) has a structure where the ring A and the ring B in the aromatic skeleton represented by General Formula (Ar-1) are limited to substituted or unsubstituted benzene rings. Among a benzene ring, a naphthalene ring, and a phenanthrene ring, the benzene ring is an aromatic compound that absorbs the least amount of visible light and is stable. Thus, when the ring A and the ring B are limited to substituted or unsubstituted benzene rings, an increase in visible-light-absorption intensity of the organic compound can be suppressed and a decrease in emission efficiency of the light-emitting device can be prevented.

In the case where both the ring C and the ring D are benzene rings, α¹ is preferably oxygen, sulfur, carbon including a substituent, silicon including a substituent, or germanium including a substituent. In this case, the organic compound can have a higher molecular weight and lower water solubility. In the case where both the ring C and the ring D are benzene rings and α¹ is a single bond, the number of hydrophobic substituents or the molecular weight of the organic compound can be increased and accordingly the water solubility can be reduced by providing a substituent for one or more of the benzene rings or by setting n in General Formula (G1) to an integer of 1 to 3, for example.

Specific examples of carbon including a substituent, silicon including a substituent, and germanium including a substituent which can be used in General Formula (Ar-2) include skeletons represented by General Formulae (α-4) to (α-6) below. Note that General Formula (α-4) is the same as General Formula (α-1). General Formula (α-5) shows a structure where the ring E and the ring F in General Formula (α-2) are limited to substituted or unsubstituted benzene rings. General Formula (α-6) shows a structure where the ring E, the ring F, the ring G, and the ring H in General Formula (α-3) are limited to substituted or unsubstituted benzene rings. Thus, description of the structure, effect, and the like of General Formulae (α-1) to (α-3) can be applied to General Formulae (α-4) to (α-6).

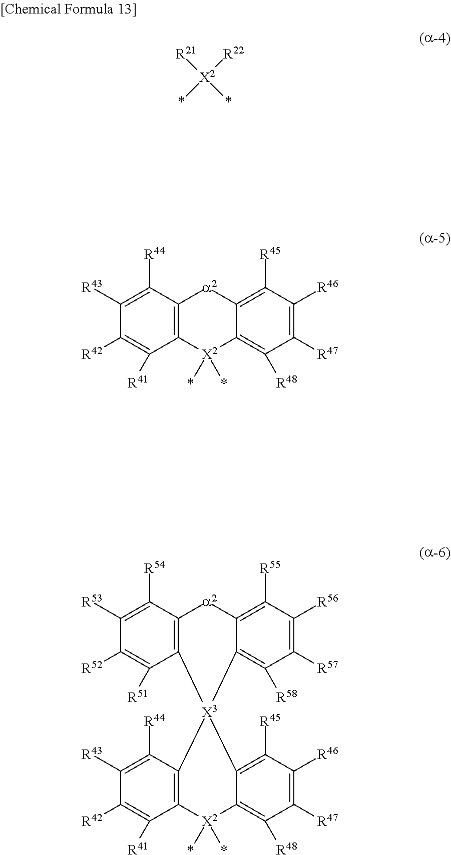

In General Formulae (α-4) to (α-6), each of X² and X³ independently represents carbon, silicon, or germanium; α² represents a single bond, oxygen, sulfur, carbon including a substituent, silicon including a substituent, or germanium including a substituent; and each of R²¹, R²², R⁴¹ to R⁴⁸, and R⁵¹ to R⁵⁸ independently represents hydrogen (including deuterium), an alkyl group having 1 to 10 carbon atoms, a substituted or unsubstituted cycloalkyl group having 3 to 10 carbon atoms, a substituted or unsubstituted aryl group having 6 to 30 carbon atoms, or a substituted or unsubstituted heteroaryl group having 1 to 30 carbon atoms. Note that * represents bonding positions of the ring C and the ring D in General Formula (Ar-2).

As described above, General Formula (α-5) shows the structure where the ring E and the ring F in General Formula (α-2) are limited to substituted or unsubstituted benzene rings. General Formula (α-6) shows the structure where the ring E, the ring F, the ring G, and the ring H in General Formula (α-3) are limited to substituted or unsubstituted benzene rings. Thus, when the ring E, the ring F, the ring G, and the ring H are limited to substituted or unsubstituted benzene rings, an increase in visible-light-absorption intensity of the organic compound can be suppressed and a decrease in emission efficiency of the light-emitting device can be prevented.

Alternatively, as the aromatic skeleton containing a spiro atom in General Formula (G1), an aromatic skeleton represented by General Formula (Ar-3) or (Ar-4) below can be used, for example. Note that General Formula (Ar-3) shows a structure where the ring A, the ring B, the ring C, and the ring D in General Formula (Ar-1) are limited to substituted or unsubstituted benzene rings and α¹ is limited to carbon including a substituent, silicon including a substituent, or germanium including a substituent. General Formula (Ar-4) shows a structure where the ring A, the ring B, and the ring C in General Formula (Ar-1) are limited to substituted or unsubstituted benzene rings and the ring D is limited to a naphthalene ring. Thus, description of the structure, effect, and the like of General Formula (Ar-1) can be applied to General Formulae (Ar-3) and (Ar-4).

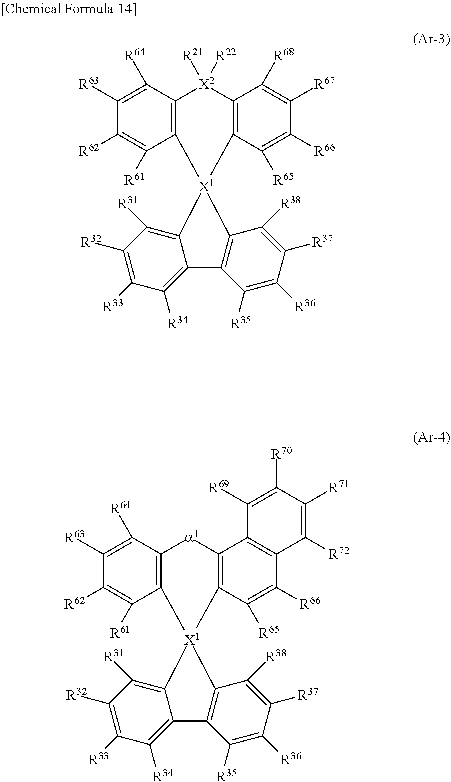

In General Formulae (Ar-3) and (Ar-4), each of X¹ and X² independently represents carbon, silicon, or germanium; α¹ represents a single bond, oxygen, sulfur, carbon including a substituent, silicon including a substituent, or germanium including a substituent; any m groups of R³¹ to R³⁸ and R⁶¹ to R⁶⁶ represent bonding positions in General Formula (G1); each of the other groups of R³¹ to R³⁸ and R⁶¹ to R⁶⁶ and R⁶⁷ to R⁷², R²¹, and R²² independently represents hydrogen (including deuterium), an alkyl group having 1 to 10 carbon atoms, a cycloalkyl group having 3 to 10 carbon atoms, a substituted or unsubstituted aryl group having 6 to 30 carbon atoms, or a substituted or unsubstituted heteroaryl group having 1 to 30 carbon atoms.

As described above, General Formula (Ar-3) shows the structure where the ring A, the ring B, the ring C, and the ring D in General Formula (Ar-1) are limited to substituted or unsubstituted benzene rings and α¹ is limited to carbon including a substituent, silicon including a substituent, or germanium including a substituent. Thus, when the ring A, the ring B, the ring C, and the ring D are limited to substituted or unsubstituted benzene rings, an increase in visible-light-absorption intensity of the organic compound can be suppressed and a decrease in emission efficiency of the light-emitting device can be prevented. When α¹ is limited to carbon including a substituent, silicon including a substituent, or germanium including a substituent, the molecular weight of the organic compound can be increased. Accordingly, the water solubility of the organic compound can be reduced.

As described above, General Formula (Ar-4) shows the structure where the ring A, the ring B, and the ring C in General Formula (Ar-1) are limited to substituted or unsubstituted benzene rings and the ring D is limited to a naphthalene ring. When the ring A, the ring B, and the ring C are limited to substituted or unsubstituted benzene rings, an increase in visible-light-absorption intensity of the organic compound can be suppressed and a decrease in emission efficiency of the light-emitting device can be prevented.

Alternatively, as the aromatic skeleton containing a spiro atom in General Formula (G1), an aromatic skeleton represented by General Formula (Ar-5) below can be used, for example. Note that General Formula (Ar-5) shows a structure where the ring A, the ring B, the ring C, and the ring D in General Formula (Ar-1) are limited to substituted or unsubstituted benzene rings, and α¹ is limited to a single bond. Thus, description of the structure, effect, and the like of General Formula (Ar-1) can be applied to General Formula (Ar-5).

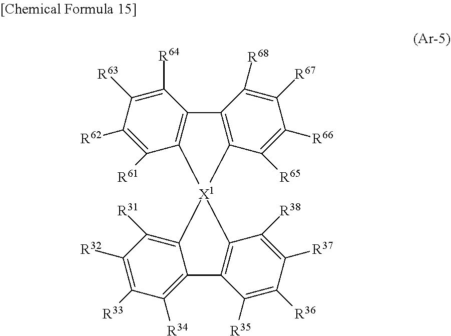

In General Formula (Ar-5), X¹ represents carbon, silicon, or germanium; any m groups of R³¹ to R³⁸ and R⁶¹ to R⁶⁸ represent bonding positions in General Formula (G1); each of the other groups independently represents hydrogen (including deuterium), an alkyl group having 1 to 10 carbon atoms, a cycloalkyl group having 3 to 10 carbon atoms, a substituted or unsubstituted aryl group having 6 to 30 carbon atoms, or a substituted or unsubstituted heteroaryl group having 1 to 30 carbon atoms.

As described above, General Formula (Ar-5) is different from General Formula (Ar-1) in that the ring A, the ring B, the ring C, and the ring D are limited to substituted or unsubstituted benzene rings. Thus, an increase in visible-light-absorption intensity of the organic compound can be suppressed and a decrease in emission efficiency of the light-emitting device can be prevented.

As described above, General Formula (Ar-5) is different from General Formula (Ar-1) in that α¹ is limited to a single bond. Thus, the sublimation temperature of the organic compound can be made low.

While the above-described effects are obtained, the organic compound employing General Formula (Ar-5) is less likely to be increased in molecular weight and thus has high water solubility in some cases. Thus, in the case where General Formula (Ar-5) is employed for the organic compound represented by General Formula (G1), n in General Formula (G1) is preferably limited to an integer of 1 to 3. In other words, a structure is preferable where a skeleton containing a spiro atom and a bicyclic guanidine skeleton are bonded to each other through an alkylene group having 1 to 10 carbon atoms, a substituted or unsubstituted arylene group having 6 to 30 carbon atoms, or a substituted or unsubstituted heteroarylene group having 1 to 11 carbon atoms. This can increase the number of hydrophobic substituents and the molecular weight of the organic compound, thereby reducing the water solubility.

Another embodiment of the present invention is an organic compound represented by any one of General Formulae (G2-1) to (G2-3) below. Note that an organic compound represented by General Formula (G2-1) has a structure where the aromatic skeleton represented by General Formula (Ar-3) is used in the organic compound represented by General Formula (G1), n is limited to 0, m is limited to 1, and the bonding position is limited to R³². An organic compound represented by General Formula (G2-2) has a structure where the aromatic skeleton represented by General Formula (Ar-4) is used in the organic compound represented by General Formula (G1), n is limited to 0, m is limited to 1, and the bonding position is limited to R³². An organic compound represented by General Formula (G2-3) has a structure where the aromatic skeleton represented by General Formula (Ar-5) is used in the organic compound represented by General Formula (G1), n is limited to 1 to 3, m is limited to 1, the bonding position is limited to R³², and L is limited to a substituted or unsubstituted phenylene group. Thus, description of the structure, effect, and the like of General Formulae (G1) and (Ar-3) to (Ar-5) can be applied to General Formulae (G2-1) to (G2-3).

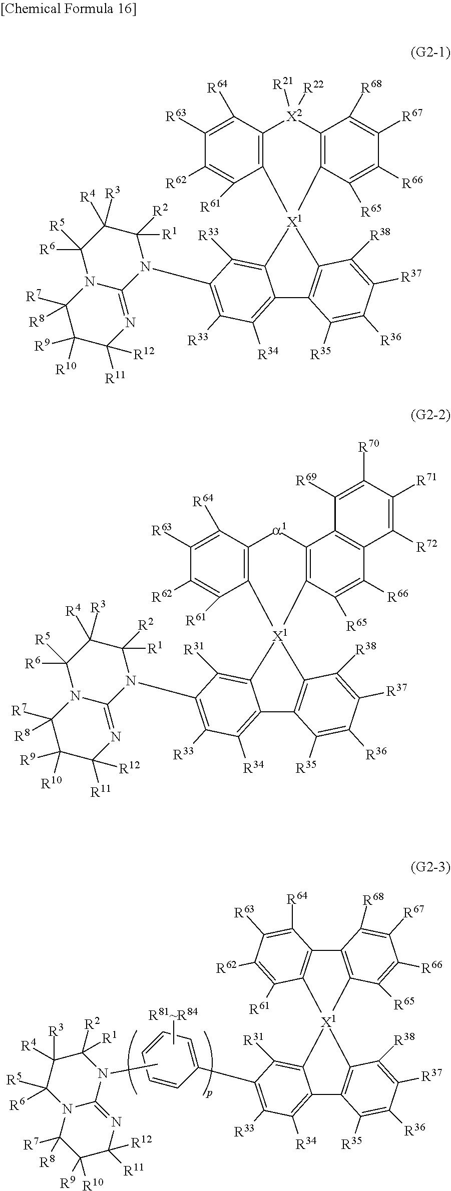

In General Formulae (G2-1) to (G2-3), each of R¹ to R¹² independently represents hydrogen (including deuterium) or an alkyl group having 1 to 10 carbon atoms. Each of X¹ and X² independently represents carbon, silicon, or germanium; α¹ represents a single bond, oxygen, sulfur, carbon including a substituent, silicon including a substituent, or germanium including a substituent; each of R³¹, R³³ to R³⁸, R⁶¹ to R⁷², R⁸¹ to R⁸⁴, R²¹, and R²² independently represents hydrogen (including deuterium), an alkyl group having 1 to 10 carbon atoms, a cycloalkyl group having 3 to 10 carbon atoms, a substituted or unsubstituted aryl group having 6 to 30 carbon atoms, or a substituted or unsubstituted heteroaryl group having 1 to 30 carbon atoms; and p represents an integer of 1 to 3.

The organic compounds represented by General Formulae (G2-1) to (G2-3) each include only one bicyclic guanidine skeleton in a molecule (corresponding to the case where m in General Formula (G1) is 0). Thus, the water solubility of the organic compound can be reduced.

In the organic compounds represented by General Formulae (G2-1) and (G2-2), a bicyclic guanidine skeleton is directly bonded to an aromatic skeleton (corresponding to the case where n in General Formula (G1) is 0). In this case, the organic compound is easily synthesized.

In the organic compound represented by General Formula (G2-3), a bicyclic guanidine skeleton and an aromatic skeleton are directly bonded to each other through a phenylene group (corresponding to the case where n in General Formula (G1) is 1). The organic compound thus can have a higher molecular weight and lower water solubility than an organic compound where the bicyclic guanidine skeleton is directly bonded to the aromatic skeleton. Moreover, the glass transition temperature can be further increased, whereby a film can be kept stable without abnormality in film quality in a heating step.

The organic compounds represented by General Formula (G2-1) to (G2-3) each include the bicyclic guanidine skeleton at a position of R³² in General Formulae (Ar-3) to (Ar-5). This can reduce the water solubility and increase heat resistance without hindering carrier transport.

Next, specific examples of an alkyl group having 1 to 10 carbon atoms, an alkylene group having 1 to 10 carbon atoms, a cycloalkyl group having 3 to 10 carbon atoms, an aryl group having 6 to 30 carbon atoms, an arylene group having 6 to 30 carbon atoms, and a heteroaryl group having 1 to 30 carbon atoms that can be used in General Formulae (G1), (Ar-1) to (Ar-5), and (G2-1) to (G2-3) will be described. Note that in the specific examples described below, some or all of hydrogen atoms may be deuterium. The groups that can be used in the above general formulae are not limited to the following specific examples.

### Specific Examples of Alkyl Group Having 1 to 10 Carbon Atoms

An alkyl group having 1 to 10 carbon atoms is a monovalent group with a structure where one hydrogen atom is removed from an alkane having 1 to 10 carbon atoms. Specific examples include a methyl group, an ethyl group, a propyl group, an isopropyl group, a butyl group, a sec-butyl group, an isobutyl group, a tert-butyl group, a pentyl group, an isopentyl group, a sec-pentyl group, a tert-pentyl group, a neo-pentyl group, a hexyl group, an isohexyl group, a sec-hexyl group, a tert-hexyl group, a neo-hexyl group, a 3-methylpentyl group, a 2-methylpentyl group, a 2-ethylbutyl group, a 1,2-dimethylbutyl group, a 2,3-dimethylbutyl group, and a 1-ethylhexyl group. In particular, the use of a branched alkyl group such as a tert-butyl group can further reduce the water solubility of the organic compound.

### Specific Examples of Alkylene Group Having 1 to 10 Carbon Atoms

An alkylene group having 1 to 10 carbon atoms is a bivalent group with a structure where two hydrogen atoms are removed from an alkane having 1 to 19 carbon atoms. Specific examples include a bivalent group with a structure where one more hydrogen atom is removed from the above specific example of the alkyl group having 1 to 10 carbon atoms.

### Specific Examples of Cycloalkyl Group Having 3 to 10 Carbon Atoms

Specific examples of a cycloalkyl group having 3 to 10 carbon atoms include a cyclopropyl group, a cyclobutyl group, a methylcyclobutyl group, a cyclopentyl group, a methylcyclopentyl group, an isopropylcyclopentyl group, a tert-butylcyclopropyl group, a cyclohexyl group, a methylcyclohexyl group, an isopropylcyclohexyl group, a tert-butylcyclohexyl group, a cycloheptyl group, a methylcycloheptyl group, an isopropylcycloheptyl group, a cyclooctyl group, a methylcyclooctyl group, an isopropylcycloheptyl group, a cyclononyl group, a methylcyclononyl group, a cyclodecyl group, and an adamantyl group.

### Specific Examples of Aryl Group Having 6 to 30 Carbon Atoms

An aryl group having 6 to 30 carbon atoms is a monovalent group with a structure where one hydrogen atom is removed from an aromatic hydrocarbon having 6 to 30 carbon atoms. Specific examples include a phenyl group, an o-tolyl group, an m-tolyl group, a p-tolyl group, a mesityl group, an o-biphenyl group, an m-biphenyl group, a p-biphenyl group, a 1-naphthyl group, a 2-naphthyl group, a fluorenyl group, a spirobifluorenyl group, a phenanthrenyl group, an anthracenyl group, and a fluoranthenyl group. In the case where the aryl group having 6 to 30 carbon atoms includes a substituent, examples of the substituent include an alkyl group having 1 to 10 carbon atoms and a phenyl group.

### Specific Examples of Arylene Group Having 6 to 30 Carbon Atoms

An arylene group having 6 to 30 carbon atoms is a bivalent group with a structure where two hydrogen atoms are removed from an aromatic hydrocarbon having 6 to 30 carbon atoms. Specific examples include a bivalent group with a structure where one more hydrogen atom is removed from the above specific example of the aryl group having 6 to 30 carbon atoms. In the case where the arylene group having 6 to 30 carbon atoms includes a substituent, examples of the substituent include an alkyl group having 1 to 10 carbon atoms and a phenyl group.

### Specific Examples of Heteroaryl Group Having 1 to 30 Carbon Atoms

Specific examples of the heteroaryl group having 1 to 30 carbon atoms include carbazole, benzocarbazole, dibenzocarbazole, indolocarbazole, benzindolocarbazole, dibenzindolocarbazole, benzindolobenzocarbazole, dibenzothiophene, benzonaphthothiophene, dibenzofuran, and benzonaphthofuran. In the case where the heteroaryl group having 1 to 30 carbon atoms includes a substituent, examples of the substituent include an alkyl group having 1 to 10 carbon atoms and a phenyl group.

### Specific Examples of Heteroarylene Group Having 1 to 11 Carbon Atoms

Specific examples of a heteroarylene group having 1 to 11 carbon atoms include a pyridinediyl group, a pyrazinediyl group, a pyrimidinediyl group, a triazinediyl group, a triazolediyl group, an oxadiazolediyl group, a thiadiazolediyl group, an oxazolediyl group, a thiazolediyl group, a thiophenediyl group, a pyrrolediyl group, a furandiyl group, a selenophenediyl group, a benzothiophenediyl group, a benzopyrrolediyl group, a benzofurandiyl group, a quinolinediyl group, an isoquinolinediyl group, a dibenzothiophenediyl group, a carbazolediyl group, and a dibenzofurandiyl group. In the case where the heteroarylene group having 1 to 11 carbon atoms includes a substituent, examples of the substituent include an alkyl group having 1 to 10 carbon atoms and a phenyl group.

The above examples of the substituent can be employed in the above general formulae.

Specific examples of the organic compound of one embodiment of the present invention represented by the above general formulae include organic compounds represented by Structural Formulae (100) to (153) below.

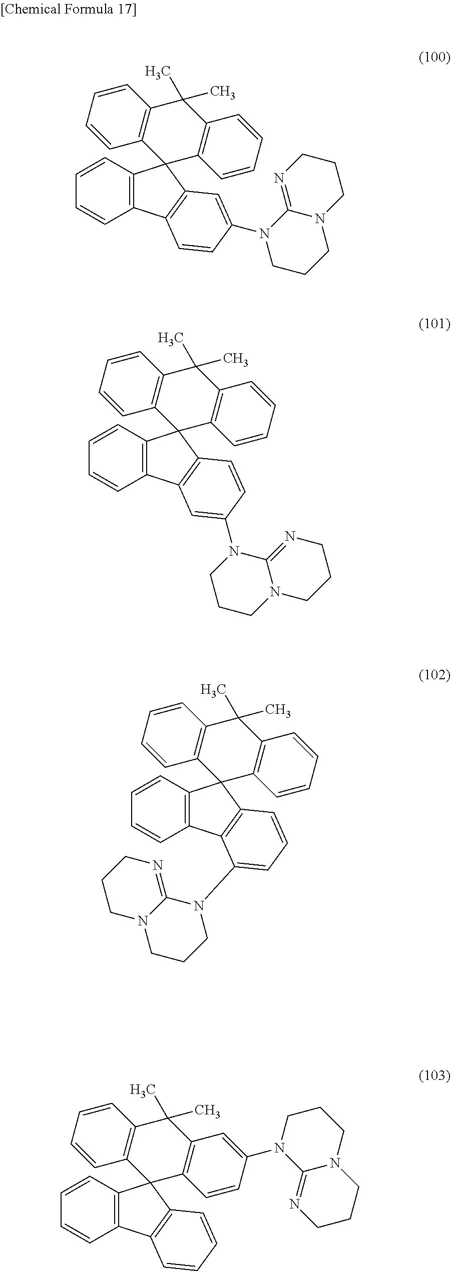

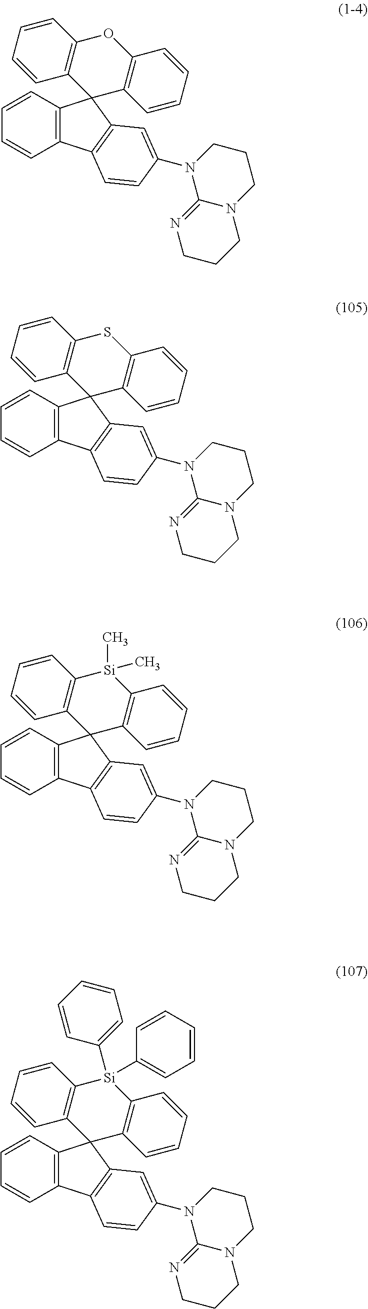

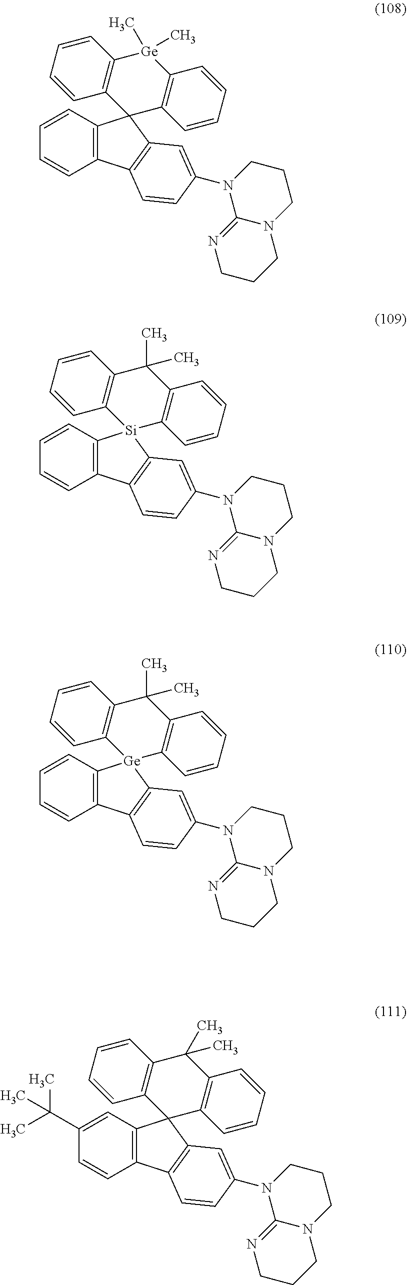

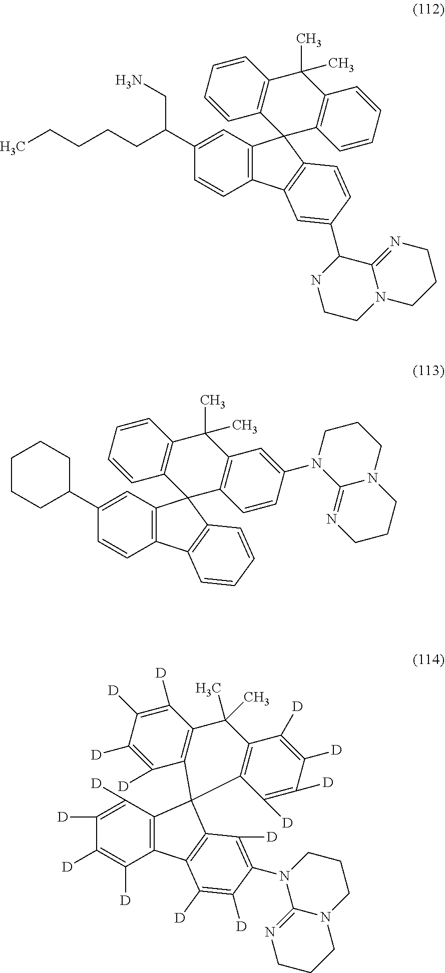

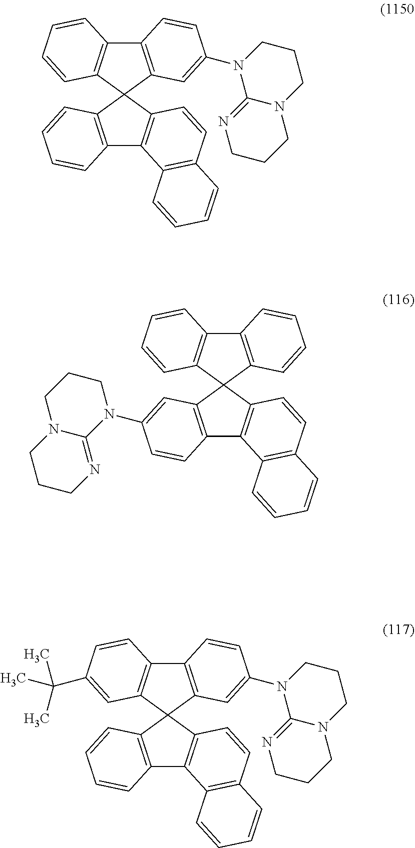

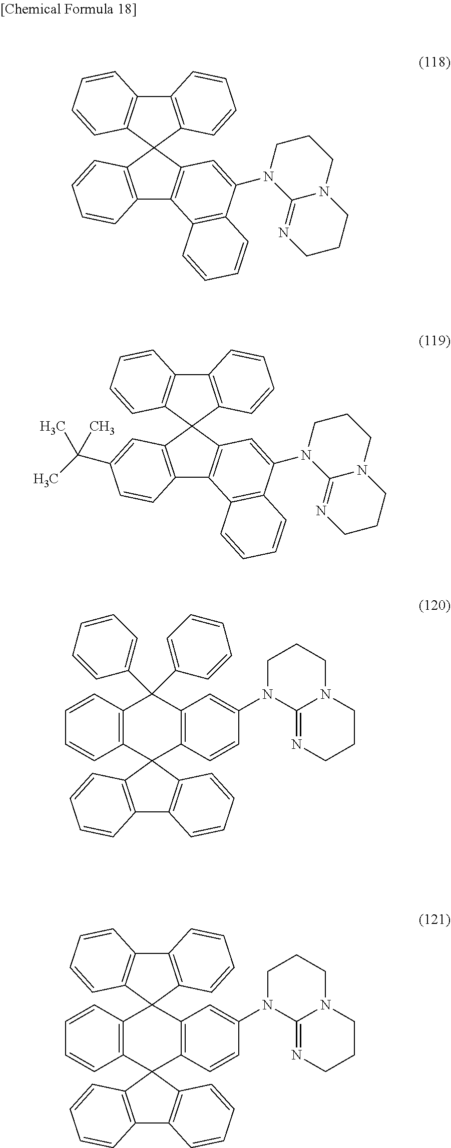

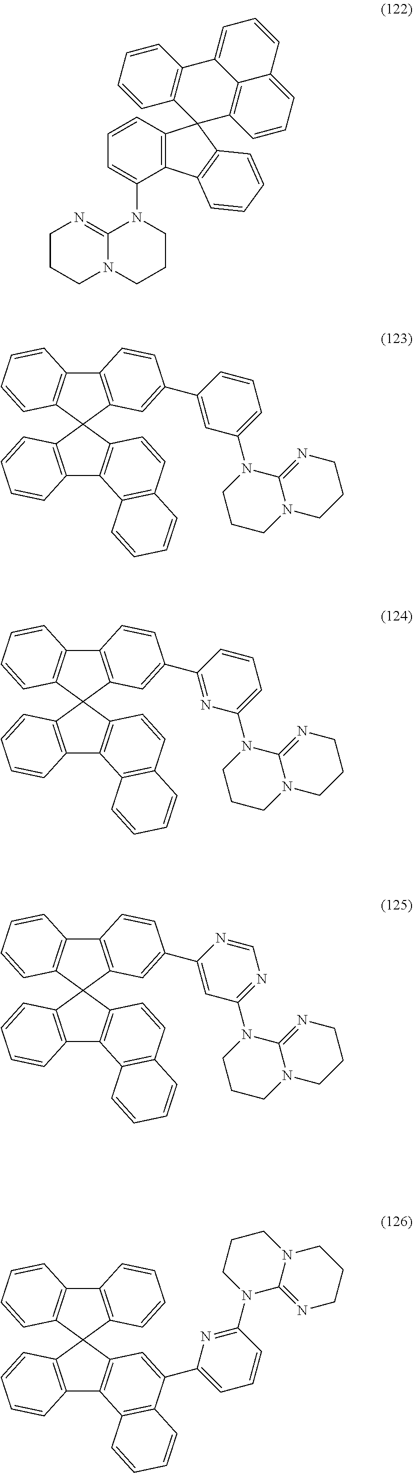

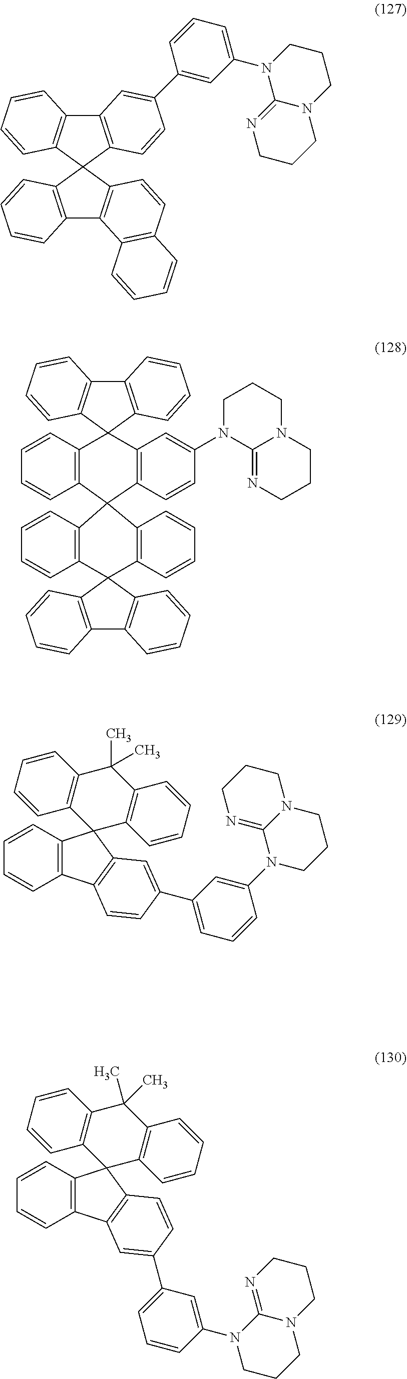

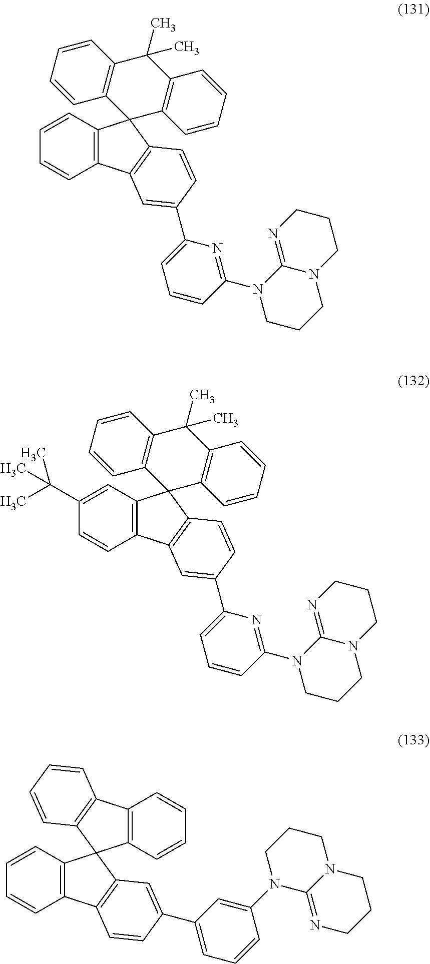

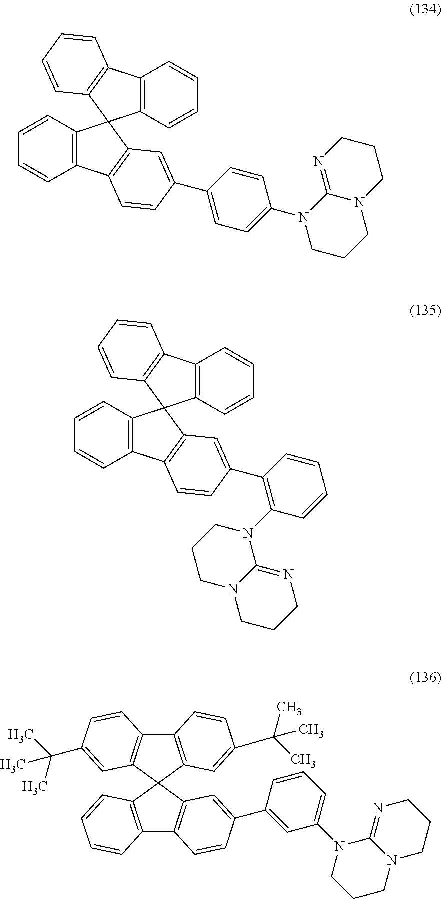

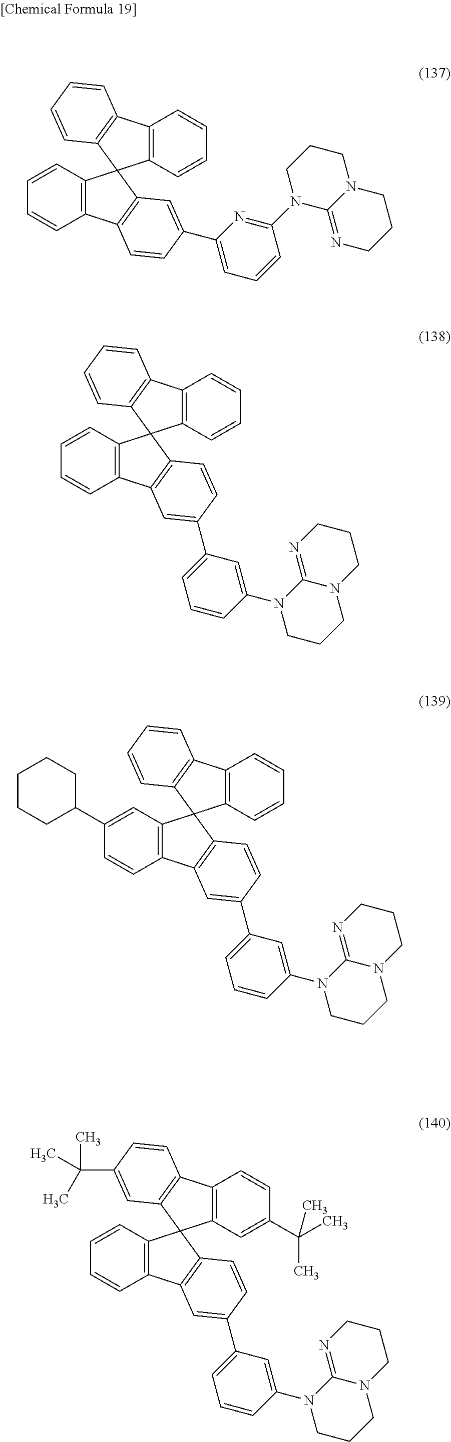

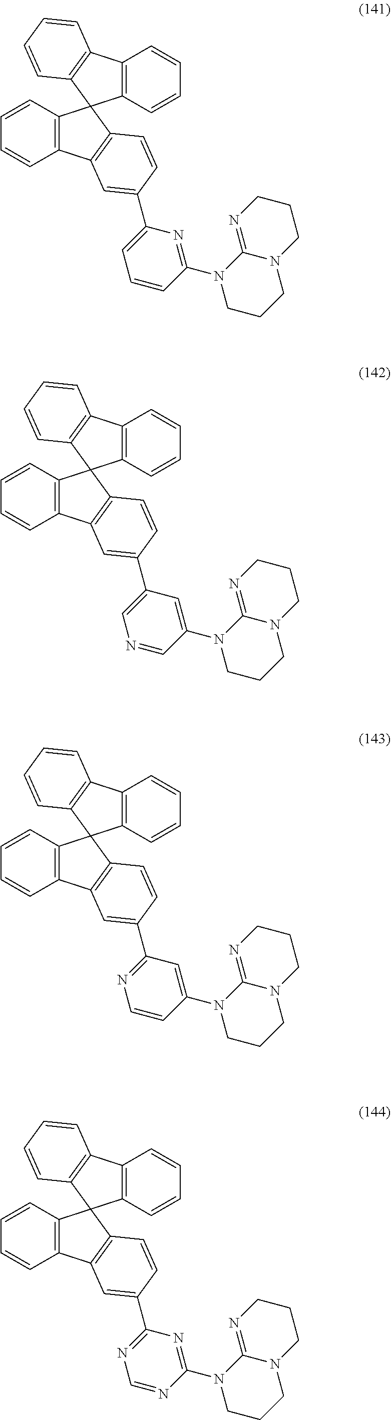

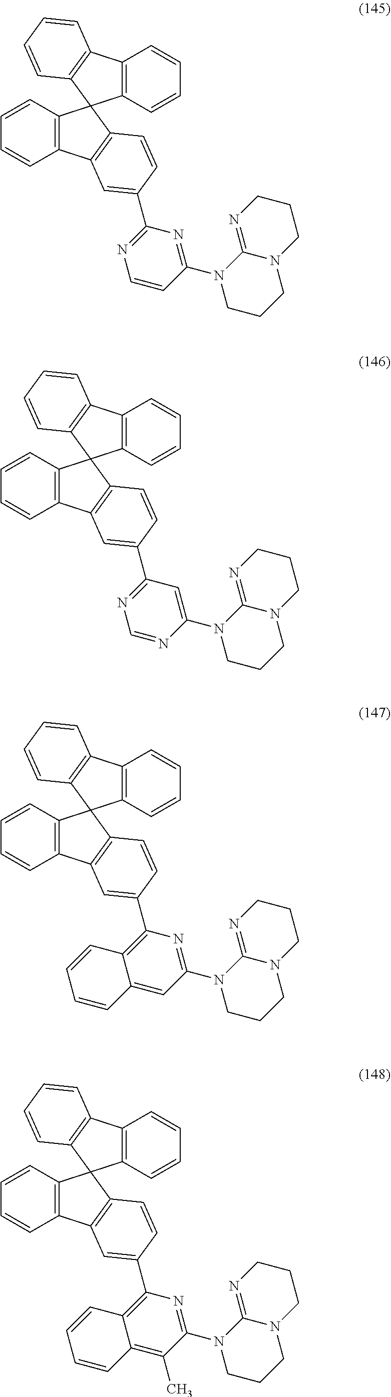

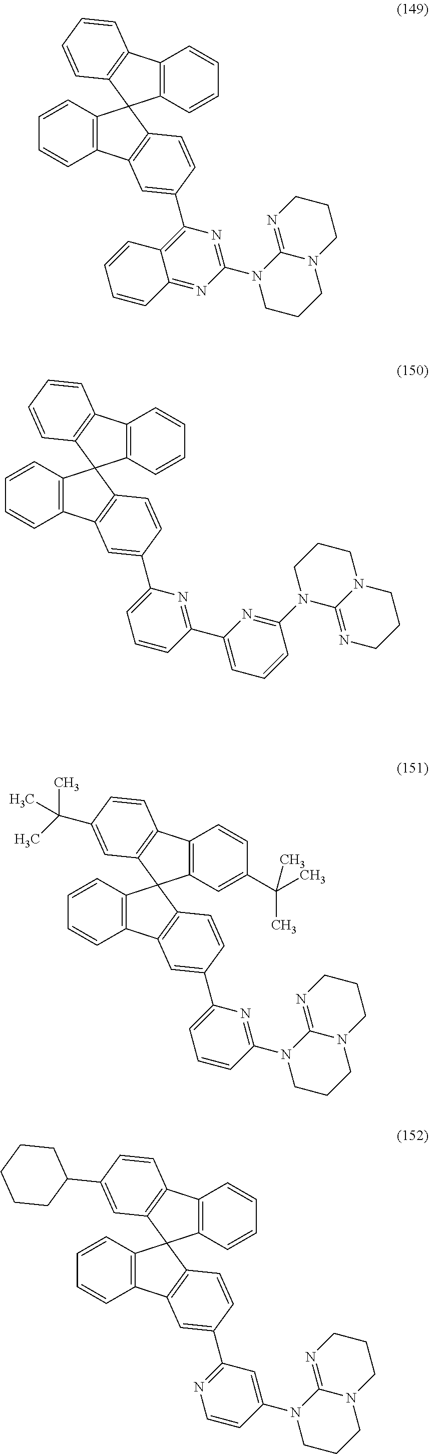

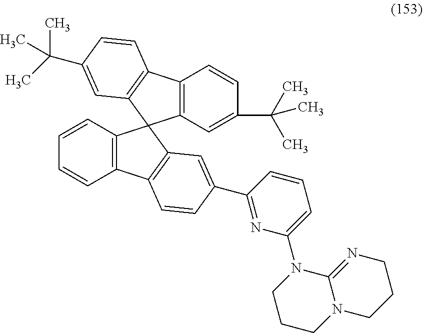

The organic compounds represented by Structural Formulae (100) to (153) are examples of the organic compound of one embodiment of the present invention; however, one embodiment of the present invention is not limited thereto.

Next, as an example of a synthesis method of the organic compound of one embodiment of the present invention, a synthesis method of the organic compound represented by General Formula (G1) below is described. Note that the synthesis method of the organic compound represented by General Formula (G1) can employ a variety of reactions and is not limited to the following synthesis methods.

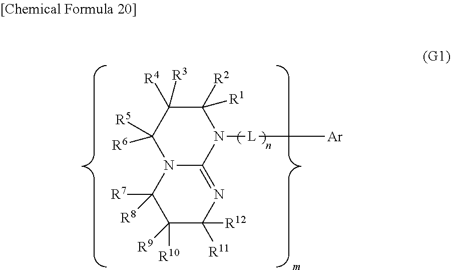

In General Formula (G1), Ar is an aromatic skeleton represented by any of General Formulae (Ar-1) to (Ar-5) described above; L represents an alkylene group having 1 to 10 carbon atoms, a substituted or unsubstituted arylene group having 6 to 30 carbon atoms, or a substituted or unsubstituted heteroarylene group having 1 to 11 carbon atoms; n represents an integer of 0 to 3; m represents an integer of 1 to 4; each of R¹ to R¹² independently represents hydrogen (including deuterium) or an alkyl group having 1 to 10 carbon atoms. In the case where n is 2 or more, Ls may be the same or different from each other. In the case where m is 2 or more, Ls may be the same or different from each other, n's may be the same or different from each other, and each of R's to R¹²s may be independently the same or different from each other.

As shown in the following synthesis scheme, the organic compound represented by General Formula (G1) can be obtained by coupling a halogen compound having an aromatic skeleton or a compound having an aromatic skeleton and a triflate group (a1) with a bicyclic guanidine derivative (a2) by Buchwald-Hartwig reaction.

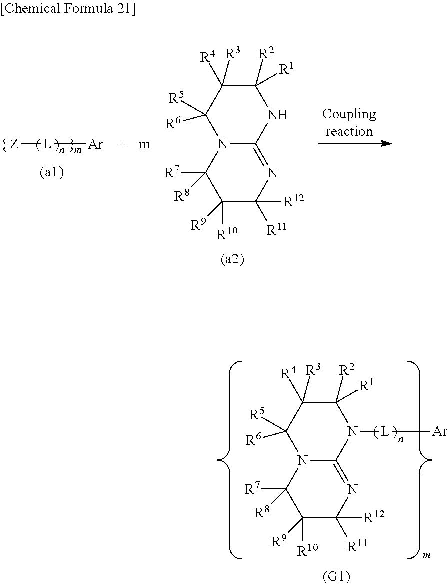

In General Formula (a1), Z represents halogen or a triflate group; Ar is an aromatic skeleton represented by any of General Formulae (Ar-1) to (Ar-5) described above; L represents an alkylene group having 1 to 10 carbon atoms, a substituted or unsubstituted arylene group having 6 to 30 carbon atoms, or a substituted or unsubstituted heteroarylene group having 1 to 11 carbon atoms; n represents an integer of 0 to 3; and m represents an integer of 1 to 4. In the case where n is 2 or more, Ls may be the same or different from each other. In the case where m is 2 or more, Ls may be the same or different from each other, n's may be the same or different from each other, and each of R¹s to R¹²s may be independently the same or different from each other.

In General Formula (a2), each of R¹ to R¹² independently represents hydrogen (including deuterium) or an alkyl group having 1 to 10 carbon atoms. In actual synthesis reaction, the organic compound represented by General Formula (a2) is preferably added excessively.

Examples of a palladium catalyst that can be used in the coupling reaction represented by the above synthesis scheme include palladium(II) acetate, tetrakis(triphenylphosphine)palladium(0), and bis(triphenylphosphine)palladium(II) dichloride.

Examples of a ligand in the above palladium catalyst include (±)-2,2′-bis(diphenylphosphino)-1,1′-binaphthyl, tri(ortho-tolyl)phosphine, triphenylphosphine, and tricyclohexylphosphine.

Examples of a base that can be used in the coupling reaction represented by the above synthesis scheme include an organic base such as potassium tert-butoxide and an inorganic base such as potassium carbonate or sodium carbonate.

Examples of a solvent that can be used in the coupling reaction represented by the above synthesis scheme include toluene, xylene, mesitylene, benzene, tetrahydrofuran, and dioxane. However, the solvent that can be used is not limited to these solvents.

The reaction in the above synthesis scheme is not limited to the Buchwald-Hartwig reaction. A Migita-Kosugi-Stille coupling reaction using an organotin compound, a coupling reaction using a Grignard reagent, an Ullmann reaction using copper or a copper compound, a nucleophilic substitution reaction, or the like can be used.

A variety of kinds of the above compounds (a1) and (a2) are commercially available or can be synthesized.

The organic compound of one embodiment of the present invention can be synthesized in the above manner, but the present invention is not limited to this and other synthesis methods may be employed.

Among the above organic compounds, an organic compound with low water solubility is preferable because of its high resistance to processing by a lithography method. For example, the organic compound preferably has water solubility lower than 3.9×10⁻⁴ g/mL, further preferably lower than 6.1×10⁻⁵ g/mL, still further preferably lower than 2.3×10⁻⁶ g/mL at a pressure of one atmosphere at room temperature (RT). Note that the solubility in this specification refers to a value obtained by dividing the weight of a solute by the weight of a solution.

The structures described in this embodiment can be used in combination with any of the structures described in the other embodiments as appropriate.

### Embodiment 2

In this embodiment, a structure of a light-emitting device using the organic compound of one embodiment of the present invention is described. Since the organic compound of one embodiment of the present invention has low water solubility, even in the manufacture of a light-emitting device using the organic compound by a method including a process using water, it is possible to prevent problems such as dissolution of a layer including the organic compound and permeation of a chemical solution into the layer including the organic compound. Consequently, the light-emitting device can have favorable characteristics.

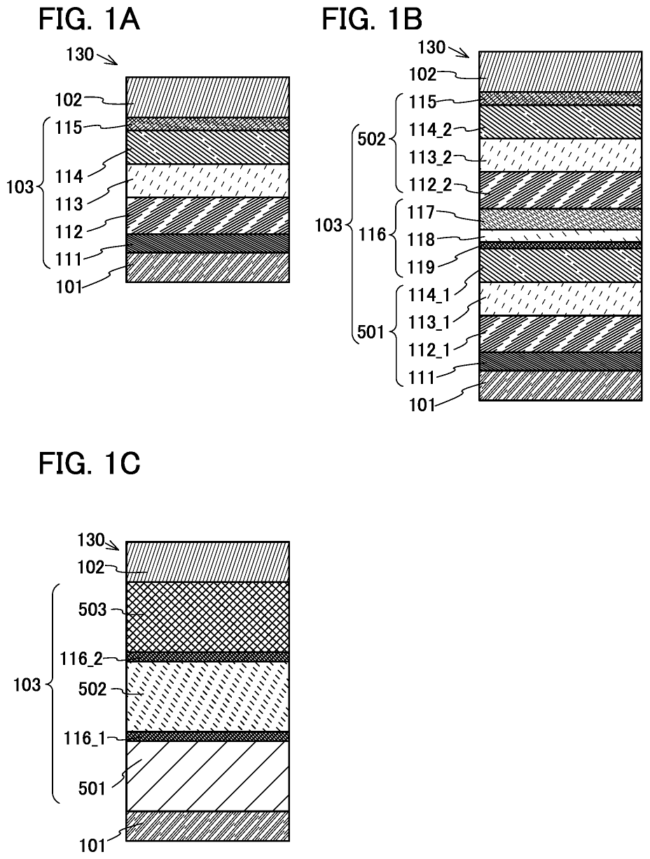

FIG. 1A illustrates a light-emitting device 130, which is an example of the light-emitting device of one embodiment of the present invention. The light-emitting device 130 includes an organic compound layer 103 including a light-emitting layer 113 between a first electrode 101 including an anode and a second electrode 102 including a cathode.

FIG. 1B illustrates the light-emitting device 130 that is another example of the light-emitting device of one embodiment of the present invention. The light-emitting device 130 is a tandem light-emitting device. The light-emitting device 130 includes a first light-emitting unit 501 including a first light-emitting layer 113_1, a second light-emitting unit 502 including a second light-emitting layer 113_2, and an intermediate layer 116, as the organic compound layer 103.

Although a light-emitting device including one intermediate layer 116 and two light-emitting units is described as an example in this embodiment, a light-emitting device including n intermediate layer(s) (n is an integer greater than or equal to 1) and n+1 light-emitting units may be employed.

For example, the light-emitting device 130 illustrated in FIG. 1C is an example of the tandem light-emitting device where n is 2, including the first light-emitting unit 501, a first intermediate layer 116_1, the second light-emitting unit 502, a second intermediate layer 116_2, and a third light-emitting unit 503, as the organic compound layer 103. Note that the color gamut of light emitted by the light-emitting layers in the light-emitting units may be the same or different. In addition, the light-emitting layer may have a single-layer structure or a stacked-layer structure. For example, the light-emitting layers in the first and third light-emitting units emit light in a blue region while the stacked light-emitting layers in the second light-emitting unit emit light in a red region and light in a green region, whereby white light emission can be obtained.

The light-emitting device 130 may be fabricated by a lithography method, for example. In the case of the light-emitting device fabricated by a lithography method, at least the light-emitting layer 113 (or the second light-emitting layer 113_2) and the layer(s) in the organic compound layer that is/are closer to the first electrode 101 than the light-emitting layer are processed at the same time; consequently, their end portions are substantially aligned in the perpendicular direction.

The organic compound layer 103 may include another functional layer in addition to the light-emitting layer. FIG. 1A illustrates a structure where, in addition to the light-emitting layer 113, a hole-injection layer 111, a hole-transport layer 112, an electron-transport layer 114, and an electron-injection layer 115 are provided in the organic compound layer 103. Furthermore, the first light-emitting unit 501 and the second light-emitting unit 502 may each include another functional layer in addition to the light-emitting layer. FIG. 1B illustrates a structure where the hole-injection layer 111, a first hole-transport layer 112_1, and a first electron-transport layer 114_1, in addition to the first light-emitting layer 113_1, are provided in the first light-emitting unit 501 and a second hole-transport layer 112_2, a second electron-transport layer 114_2, and the electron-injection layer 115, in addition to the second light-emitting layer 113_2, are provided in the second light-emitting unit 502. The structure of the organic compound layer 103 in the present invention is not limited to these structures; any of the layers may be absent or another layer maybe added. A carrier-blocking layer (a hole-blocking layer or an electron-blocking layer), an exciton-blocking layer, or the like may be typically added.

### <<Structure of Intermediate Layer>>

First, a material that can be used for the intermediate layer 116 is described. The organic compound of one embodiment of the present invention described in Embodiment 1 can be used for the intermediate layer 116. The intermediate layer 116 preferably has a stacked-layer structure including a first layer 119 and a second layer 117, and instead of the donor substance, the organic compound of one embodiment of the present invention described in Embodiment 1 is preferably used for the first layer 119. Since the organic compound of one embodiment of the present invention has low water solubility, even in the manufacture of a light-emitting device using the organic compound by a method including a process using water, it is possible to prevent problems such as dissolution of a layer including the organic compound and permeation of a chemical solution into the layer including the organic compound. Consequently, the light-emitting device can have favorable characteristics.

The second layer 117 is positioned closer to the second electrode 102 than the first layer 119 is. Between the first layer 119 and the second layer 117, a third layer 118 for smoothing electron transfer between the two layers may be provided.

Since the first layer 119 is included in the intermediate layer 116, the first layer 119 serves as an electron-injection layer in the light-emitting unit closer to the anode. Thus, an electron-injection layer is not necessarily provided in the light-emitting unit on the anode side (the first light-emitting unit 501 in FIG. 1). Similarly, since the second layer 117 is included in the intermediate layer 116, the second layer 117 serves as a hole-injection layer in the light-emitting unit closer to the cathode. Thus, a hole-injection layer is not necessarily provided in the light-emitting unit on the cathode side (the second light-emitting unit 502 in FIG. 1).

The first layer 119 may include an organic compound having an electron-transport property in addition to the organic compound of one embodiment of the present invention. For example, as the first layer 119, a mixed layer of the organic compound of one embodiment of the present invention and an organic compound having an electron-transport property can be used. When the organic compound of one embodiment of the present invention and an organic compound having an electron-transport property are used for the first layer 119, the light-emitting device can have higher emission efficiency.

The organic compound having an electron-transport property that can be used for the first layer 119 is preferably a substance with an electron mobility higher than or equal to 1×10⁻⁷ cm²/Vs, preferably higher than or equal to 1×10⁻⁶ cm²/Vs, when the square root of electric field strength [V/cm] is 600. Note that any other substance can be used as long as the substance has an electron-transport property higher than a hole-transport property.

An organic compound including a π-electron deficient heteroaromatic ring is preferable as the above organic compound. The organic compound including a π-electron deficient heteroaromatic ring is preferably one or more of an organic compound including a heteroaromatic ring having a polyazole skeleton, an organic compound including a heteroaromatic ring having a pyridine skeleton, an organic compound including a heteroaromatic ring having a diazine skeleton, and an organic compound including a heteroaromatic ring having a triazine skeleton.

Specific examples of the organic compound having an electron-transport property that can be used for the first layer 119 include the following compounds: organic compounds having an azole skeleton, such as 2-(4-biphenylyl)-5-(4-tert-butylphenyl)-1,3,4-oxadiazole (abbreviation: PBD), 3-(4-biphenylyl)-4-phenyl-5-(4-tert-butylphenyl)-1,2,4-triazole (abbreviation: TAZ), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: CO11), 2,2′,2″-(1,3,5-benzenetriyl)tris(1-phenyl-1H-benzimidazole) (abbreviation: TPBI), 2-[3-(dibenzothiophen-4-yl)phenyl]-1-phenyl-1H-benzimidazole (abbreviation: mDBTBIm-II), and 4,4′-bis(5-methylbenzoxazol-2-yl)stilbene (abbreviation: BzOs); organic compounds having a heteroaromatic ring having a pyridine skeleton, such as 3,5-bis[3-(9H-carbazol-9-yl)phenyl]pyridine (abbreviation: 35DCzPPy), 1,3,5-tri[3-(3-pyridyl)phenyl]benzene (abbreviation: TmPyPB), bathophenanthroline (abbreviation: BPhen), bathocuproine (abbreviation: BCP), 2,9-di(naphthalen-2-yl)-4,7-diphenyl-1,10-phenanthroline (abbreviation: NBPhen), and 2,2′-(1,3-phenylene)bis(9-phenyl-1,10-phenanthroline) (abbreviation: mPPhen2P); organic compounds having a diazine skeleton, such as 2-[3-(dibenzothiophen-4-yl)phenyl]dibenzo[f,h]quinoxaline (abbreviation: 2mDBTPDBq-II), 2-[3-(3′-(dibenzothiophen-4-yl)biphenyl-3-yl]dibenzo[f,h]quinoxaline (abbreviation: 2mDBTBPDBq-II), 2-[3′-(9H-carbazol-9-yl)biphenyl-3-yl]dibenzo[f,h]quinoxaline (abbreviation: 2mCzBPDBq), 2-[4′-(9-phenyl-9H-carbazol-3-yl)-3,1′-biphenyl-1-yl]dibenzo[f,h]quinoxaline (abbreviation: 2mpPCBPDBq), 2-[4-(3,6-diphenyl-9H-carbazol-9-yl)phenyl]dibenzo[f,h]quinoxaline (abbreviation: 2CzPDBq-III), 7-[3-(dibenzothiophen-4-yl)phenyl]dibenzo[f,h]quinoxaline (abbreviation: 7mDBTPDBq-II), 6-[3-(dibenzothiophen-4-yl)phenyl]dibenzo[f,h]quinoxaline (abbreviation: 6mDBTPDBq-II), 9-[(3′-dibenzothiophen-4-yl)biphenyl-3-yl]naphtho[1′,2′:4,5]furo[2,3-b]pyrazine (abbreviation: 9mDBtBPNfpr), 9-[3′-dibenzothiophen-4-yl)biphenyl-4-yl]naphtho[1′,2′:4,5]furo[2,3-b]pyrazine (abbreviation: 9pmDBtBPNfpr), 4,6-bis[3-(phenanthren-9-yl)phenyl]pyrimidine (abbreviation: 4,6mPnP2Pm), 4,6-bis[3-(4-dibenzothiophen-4-yl)phenyl]pyrimidine (abbreviation: 4,6mDBTP2Pm-II), 4,6-bis[3-(9H-carbazol-9-yl)phenyl]pyrimidine (abbreviation: 4,6mCzP2Pm), 9,9′-[pyrimidine-4,6-diylbis(biphenyl-3,3′-diyl)]bis(9H-carbazole) (abbreviation: 4,6mCzBP2Pm), 8-(biphenyl-4-yl)-4-[3-(dibenzothiophen-4-yl)phenyl]-[1]benzofuro[3,2-d]pyrimidine (abbreviation: 8BP-4mDBtPBfpm), 3,8-bis[3-(dibenzothiophen-4-yl)phenyl]benzofuro[2,3-b]pyrazine (abbreviation: 3,8mDBtP2Bfpr), 4,8-bis[3-(dibenzothiophen-4-yl)phenyl]-[1]benzofuro[3,2-d]pyrimidine (abbreviation: 4,8mDBtP2Bfpm), 8-[3′-(dibenzothiophen-4-yl) (1,1′-biphenyl-3-yl)]naphtho[1′,2′:4,5]furo[3,2-d]pyrimidine (abbreviation: 8mDBtBPNfpm), 8-[(2,2′-binaphthalen)-6-yl)]-4-[3-(dibenzothiophen-4-yl)phenyl]-[1]benzofuro[3,2-d]pyrimidine (abbreviation: 8(βN2)-4mDBtPBfpm), 2,2′-(pyridine-2,6-diyl)bis(4-phenylbenzo[h]quinazoline) (abbreviation: 2,6(P-Bqn)2Py), 2,2′-(pyridine-2,6-diyl)bis{4-[4-(2-naphthyl)phenyl]-6-phenylpyrimidine} (abbreviation: 2,6(NP-PPm)2Py), 6-(1,1′-biphenyl-3-yl)-4-[3,5-bis(9H-carbazol-9-yl)phenyl]-2-phenylpyrimidine (abbreviation: 6mBP-4Cz2PPm), 2,6-bis(4-naphthalen-1-ylphenyl)-4-[4-(3-pyridyl)phenyl]pyrimidine (abbreviation: 2,4NP-6PyPPm), 4-[3,5-bis(9H-carbazol-9-yl)phenyl]-2-phenyl-6-(biphenyl-4-yl)pyrimidine (abbreviation: 6BP-4Cz2PPm), and 7-[4-(9-phenyl-9H-carbazol-2-yl)quinazolin-2-yl]-7H-dibenzo[c,g]carbazol (abbreviation: PC-cgDBCzQz); and organic compounds having a triazine skeleton, such as 2-(biphenyl-4-yl)-4-phenyl-6-(9,9′-spirobi[9H-fluoren]-2-yl)-1,3,5-triazine (abbreviation: BP-SFTzn), 2-{3-[3-(benzo[b]naphtho[1,2-d]furan-8-yl)phenyl]phenyl}-4,6-diphenyl-1,3,5-triazine (abbreviation: mBnfBPTzn), 2-{3-[3-(benzo[b]naphtho[1,2-d]furan-6-yl)phenyl]phenyl}-4,6-diphenyl-1,3,5-triazine (abbreviation: mBnfBPTzn-02), 2-{4-[3-(N-phenyl-9H-carbazol-3-yl)-9H-carbazol-9-yl]phenyl}-4,6-diphenyl-1,3,5-triazine (abbreviation: PCCzPTzn), 9-[3-(4,6-diphenyl-1,3,5-triazin-2-yl)phenyl]-9′-phenyl-2,3′-bi-9H-carbazole (abbreviation: mPCCzPTzn-02), 2-[3′-(9,9-dimethyl-9H-fluoren-2-yl)-1,1′-biphenyl-3-yl]-4,6-diphenyl-1,3,5-triazine (abbreviation: mFBPTzn), 5-[3-(4,6-diphenyl-1,3,5-triazin-2-yl)phenyl]-7,7-dimethyl-5H,7H-indeno[2,1-b]carbazole (abbreviation: mINc(II)PTzn), 2-{3-[3-(dibenzothiophen-4-yl)phenyl]phenyl}-4,6-diphenyl-1,3,5-triazine (abbreviation: mDBtBPTzn), 2,4,6-tris[3′-(pyridin-3-yl)biphenyl-3-yl]-1,3,5-triazine (abbreviation: TmPPPyTz), 2-[3-(2,6-dimethyl-3-pyridinyl)-5-(9-phenanthrenyl)phenyl]-4,6-diphenyl-1,3,5-triazine (abbreviation: mPn-mDMePyPTzn), 11-[4-(biphenyl-4-yl)-6-phenyl-1,3,5-triazin-2-yl]-11,12-dihydro-12-phenylindolo[2,3-a]carbazole (abbreviation: BP-Icz(II)Tzn), 2-[3′-(triphenylen-2-yl)-1,1′-biphenyl-3-yl]-4,6-diphenyl-1,3,5-triazine (abbreviation: mTpBPTzn), 3-[9-(4,6-diphenyl-1,3,5-triazin-2-yl)-2-dibenzofuranyl]-9-phenyl-9H-carbazole (abbreviation: PCDBfTzn), and 2-(biphenyl-3-yl)-4-phenyl-6-{8-[(1,1′:4′,1″-terphenyl)-4-yl]-1-dibenzofuranyl}-1,3,5-triazine (abbreviation: mBP-TPDBfTzn). In particular, organic compounds having a phenanthroline skeleton, such as BPhen, BCP, NBphen, and mPPhen2P, are preferred, and an organic compound having a phenanthroline dimeric structure, such as mPPhen2P, is further preferred because of its excellent stability.

The second layer 117 is preferably formed using a composite material of a material having an acceptor property and an organic compound having a hole-transport property. As the organic compound having a hole-transport property used in the composite material, any of a variety of organic compounds such as aromatic amine compounds, heteroaromatic compounds, aromatic hydrocarbons, and high molecular compounds (e.g., oligomers, dendrimers, and polymers) can be used. Note that the organic compound having a hole-transport property preferably has a hole mobility higher than or equal to 1×10⁻⁶ cm²/Vs. The organic compound having a hole-transport property used in the composite material is preferably a compound having a condensed aromatic hydrocarbon ring or a π-electron rich heteroaromatic ring. As the condensed aromatic hydrocarbon ring, an anthracene ring, a naphthalene ring, or the like is preferable. As the π-electron rich heteroaromatic ring, a condensed aromatic ring having at least one of a pyrrole skeleton, a furan skeleton, and a thiophene skeleton is preferable; specifically, a carbazole ring, a dibenzothiophene ring, or a ring in which an aromatic ring or a heteroaromatic ring is further fused to the carbazole ring or the dibenzothiophene ring is preferable.

Such an organic compound having a hole-transport property further preferably has any of a carbazole skeleton, a dibenzofuran skeleton, a dibenzothiophene skeleton, and an anthracene skeleton. In particular, an aromatic amine including a substituent having a dibenzofuran ring or a dibenzothiophene ring, an aromatic monoamine having a naphthalene ring, or an aromatic monoamine in which a 9-fluorenyl group is bonded to the nitrogen of the amine through an arylene group may be used. Note that the organic compound having a hole-transport property preferably has an N,N-bis(4-biphenyl)amino group to enable fabrication of a light-emitting device having a long lifetime.

Specific examples of the organic compound having a hole-transport property include N-(4-biphenyl)-6,N-diphenylbenzo[b]naphtho[1,2-d]furan-8-amine (abbreviation: BnfABP), N,N-bis(4-biphenyl)-6-phenylbenzo[b]naphtho[1,2-d]furan-8-amine (abbreviation: BBABnf), 4,4′-bis(6-phenylbenzo[b]naphtho[1,2-d]furan-8-yl)-4″-phenyltriphenylamine (abbreviation: BnfBB1BP), N,N-bis(4-biphenyl)benzo[b]naphtho[1,2-d]furan-6-amine (abbreviation: BBABnf(6)), N,N-bis(4-biphenyl)benzo[b]naphtho[1,2-d]furan-8-amine (abbreviation: BBABnf(8)), N,N-bis(4-biphenyl)benzo[b]naphtho[2,3-d]furan-4-amine (abbreviation: BBABnf(II)(4)), N,N-bis[4-(dibenzofuran-4-yl)phenyl]-4-amino-p-terphenyl (abbreviation: DBfBB1TP), N-[4-(dibenzothiophen-4-yl)phenyl]-N-phenyl-4-biphenylamine (abbreviation: ThBA1BP), 4-(2-naphthyl)-4′,4″-diphenyltriphenylamine (abbreviation: BBAβNB), 4-[4-(2-naphthyl)phenyl]-4′,4″-diphenyltriphenylamine (abbreviation: BBAβNBi), 4,4′-diphenyl-4″-(6;1′-binaphthyl-2-yl)triphenylamine (abbreviation: BBAαNβNB), 4,4′-diphenyl-4″-(7;1′-binaphthyl-2-yl)triphenylamine (abbreviation: BBAαNβNB-03), 4,4′-diphenyl-4″-(7-phenyl)naphthyl-2-yltriphenylamine (abbreviation: BBAPβNB-03), 4,4′-diphenyl-4″-(6;2′-binaphthyl-2-yl)triphenylamine (abbreviation: BBA(βN2)B), 4,4′-diphenyl-4″-(7;2′-binaphthyl-2-yl)triphenylamine (abbreviation: BBA(βN2)B-03), 4,4′-diphenyl-4″-(4;2′-binaphthyl-1-yl)triphenylamine (abbreviation: BBAβNαNB), 4,4′-diphenyl-4″-(5;2′-binaphthyl-1-yl)triphenylamine (abbreviation: BBAβNαNB-02), 4-(4-biphenylyl)-4′-(2-naphthyl)-4″-phenyltriphenylamine (abbreviation: TPBiAβNB), 4-(3-biphenylyl)-4′-[4-(2-naphthyl)phenyl]-4″-phenyltriphenylamine (abbreviation: mTPBiAβNBi), 4-(4-biphenylyl)-4′-[4-(2-naphthyl)phenyl]-4″-phenyltriphenylamine (abbreviation: TPBiAβNBi), 4-phenyl-4′-(1-naphthyl)triphenylamine (abbreviation: αNBA1BP), 4,4′-bis(1-naphthyl)triphenylamine (abbreviation: αNBB1BP), 4,4′-diphenyl-4″-[4′-(carbazol-9-yl)biphenyl-4-yl]triphenylamine (abbreviation: YGTBi1BP), 4′-[4-(3-phenyl-9H-carbazol-9-yl)phenyl]tris(biphenyl-4-yl)amine (abbreviation: YGTBi1BP-02), 4-[4′-(carbazol-9-yl)biphenyl-4-yl]-4′-(2-naphthyl)-4″-phenyltriphenylamine (abbreviation: YGTBiβNB), N-[4-(9-phenyl-9H-carbazol-3-yl)phenyl]-N-[4-(1-naphthyl)phenyl]-9,9′-spirobi[9H-fluoren]-2-amine (abbreviation: PCBNBSF), N,N-bis(biphenyl-4-yl)-9,9′-spirobi[9H-fluoren]-2-amine (abbreviation: BBASF), N,N-bis(biphenyl-4-yl)-9,9′-spirobi[9H-fluoren]-4-amine (abbreviation: BBASF(4)), N-(biphenyl-2-yl)-N-(9,9-dimethyl-9H-fluoren-2-yl)-9,9′-spirobi[9H-fluoren]-4-amine (abbreviation: oFBiSF), N-(biphenyl-4-yl)-N-(9,9-dimethyl-9H-fluoren-2-yl)dibenzofuran-4-amine (abbreviation: FrBiF), N-[4-(1-naphthyl)phenyl]-N-[3-(6-phenyldibenzofuran-4-yl)phenyl]-1-naphthylamine (abbreviation: mPDBfBNBN), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-[4-(9-phenylfluoren-9-yl)phenyl]triphenylamine (abbreviation: BPAFLBi), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), N-phenyl-N-[4-(9-phenyl-9H-carbazol-3-yl)phenyl]-9,9′-spirobi[9H-fluoren]-2-amine (abbreviation: PCBASF), N-(biphenyl-4-yl)-N-[4-(9-phenyl-9H-carbazol-3-yl)phenyl]-9,9-dimethyl-9H-fluoren-2-amine (abbreviation: PCBBiF), N,N-bis(9,9-dimethyl-9H-fluoren-2-yl)-9,9′-spirobi-9H-fluoren-4-amine, N,N-bis(9,9-dimethyl-9H-fluoren-2-yl)-9,9′-spirobi-9H-fluoren-3-amine, N,N-bis(9,9-dimethyl-9H-fluoren-2-yl)-9,9′-spirobi-9H-fluoren-2-amine, and N,N-bis(9,9-dimethyl-9H-fluoren-2-yl)-9,9′-spirobi-9H-fluoren-1-amine.

Examples of the aromatic amine compounds that can be used as the material having a hole-transport property include N,N-di(p-tolyl)-N,N-diphenyl-p-phenylenediamine (abbreviation: DTDPPA), 4,4′-bis[N-(4-diphenylaminophenyl)-N-phenylamino]biphenyl (abbreviation: DPAB), 4,4′-bis(N-{4-[N-(3-methylphenyl)-N-phenylamino]phenyl}-N-phenylamino)biphenyl (abbreviation: DNTPD), and 1,3,5-tris[N-(4-diphenylaminophenyl)-N-phenylamino]benzene (abbreviation: DPA3B).

As the substance having an acceptor property included in the second layer 117, it is possible to use an organic compound having an electron-withdrawing group (e.g., a halogen group or a cyano group); for example, 7,7,8,8-tetracyano-2,3,5,6-tetrafluoroquinodimethane (abbreviation: F₄-TCNQ), chloranil, 2,3,6,7,10,11-hexacyano-1,4,5,8,9,12-hexaazatriphenylene (abbreviation: HAT-CN), 1,3,4,5,7,8-hexafluorotetracyano-naphthoquinodimethane (abbreviation: F₆-TCNNQ), or 2-(7-dicyanomethylene-1,3,4,5,6,8,9,10-octafluoro-7H-pyren-2-ylidene)malononitrile can be used. A compound in which electron-withdrawing groups are bonded to a condensed aromatic ring having a plurality of heteroatoms, such as HAT-CN, is particularly preferable because it is thermally stable. A [3]radialene derivative having an electron-withdrawing group (in particular, a cyano group, a halogen group such as a fluoro group, or the like) has a very high electron-accepting property and thus is preferable. Specific examples include α,α′,α″-1,2,3-cyclopropanetriylidenetris[4-cyano-2,3,5,6-tetrafluorobenzeneacetonitrile], α,α′,α″-1,2,3-cyclopropanetriylidenetris[2,6-dichloro-3,5-difluoro-4-(trifluoromethyl)benzeneacetonitrile], and α,α,α″-1,2,3-cyclopropanetriylidenetris[2,3,4,5,6-pentafluorobenzeneacetonitrile]. As the substance having an acceptor property, a transition metal oxide such as molybdenum oxide, vanadium oxide, ruthenium oxide, tungsten oxide, or manganese oxide can be used, other than the above-described organic compounds.

The third layer 118 includes a substance having an electron-transport property and has a function of preventing an interaction between the first layer 119 and the second layer 117 and transferring electrons smoothly. The LUMO level of the substance having an electron-transport property included in the third layer 118 is preferably between the LUMO level of the acceptor substance in the second layer 117 and the LUMO level of the organic compound included in a layer (the first electron-transport layer 1141 in the first light-emitting unit 501 in FIG. 1B) which is included in the light-emitting unit on the first electrode 101 side and is in contact with the intermediate layer 116. As a specific value of the energy level, the LUMO level of the substance having an electron-transport property in the third layer 118 is preferably higher than or equal to −5.0 eV, further preferably higher than or equal to −5.0 eV and lower than or equal to −3.0 eV, still further preferably higher than or equal to −4.30 eV and lower than or equal to −3.00 eV, yet still further preferably higher than or equal to −4.30 eV and lower than or equal to −3.30 eV, in which case electrons generated in the second layer 117 can be easily injected to the first layer 119 and accordingly an increase in the driving voltage of the light-emitting device can be inhibited. Note that as the substance having an electron-transport property in the third layer 118, a phthalocyanine-based material or a metal complex having a metal-oxygen bond and an aromatic ligand is preferably used.

Specifically, it is possible to use a perylenetetracarboxylic acid derivative such as diquinoxalino[2,3-a:2′,3′-c]phenazine (abbreviation: HATNA), 2,3,8,9,14,15-hexafluorodiquinoxalino[2,3-a:2′,3′-c]phenazine (abbreviation: HATNA-F6), 3,4,9,10-perylenetetracarboxylic diimide (abbreviation: PTCDI), or 3,4,9,10-perylenetetracarboxyl-bis-benzimidazole (abbreviation: PTCBI), (C₆₀-Iₕ)[5,6]fullerene (abbreviation: C₆₀), (C₇₀-D₅ₕ)[5,6]fullerene (abbreviation: C₇₀), or phthalocyanine (abbreviation: H₂Pc). Alternatively, it is possible to use a metal phthalocyanine containing copper, zinc, cobalt, iron, chromium, nickel, or the like or a derivative thereof, such as copper phthalocyanine (abbreviation: CuPc), zinc phthalocyanine (abbreviation: ZnPc), cobalt phthalocyanine (abbreviation: CoPc), iron phthalocyanine (abbreviation: FePc), tin phthalocyanine (abbreviation: SnPc), tin oxide phthalocyanine (abbreviation: SnOPc), titanium oxide phthalocyanine (abbreviation: TiOPc), or vanadium oxide phthalocyanine (abbreviation: VOPc). It is particularly preferable to use a phthalocyanine-based metal complex such as copper phthalocyanine or zinc phthalocyanine or 2,3,8,9,14,15-hexafluorodiquinoxalino[2,3-a:2′,3′-c]phenazine.

The thickness of the third layer 118 is greater than or equal to 1 nm and less than or equal to 10 nm, preferably greater than or equal to 2 nm and less than or equal to 5 nm.

Then, components of the above light-emitting device 130, other than the intermediate layer 116, are described.

### <<Structure of First Electrode>>

The first electrode 101 is the electrode including an anode. The first electrode 101 may have a stacked structure where the layer in contact with the organic compound layer 103 functions as the anode. The anode is preferably formed using any of metals, alloys, and conductive compounds with a high work function (specifically, higher than or equal to 4.0 eV), mixtures thereof, and the like. Specific examples include indium oxide-tin oxide (ITO: indium tin oxide), indium oxide-tin oxide containing silicon or silicon oxide, indium oxide-zinc oxide, and indium oxide containing tungsten oxide and zinc oxide (IWZO). Such conductive metal oxide films are usually formed by a sputtering method, but may be formed by application of a sol-gel method or the like. In an example of the formation method, a film of indium oxide-zinc oxide is formed by a sputtering method using a target obtained by adding 1 wt % to 20 wt % of zinc oxide to indium oxide. Furthermore, a film of indium oxide containing tungsten oxide and zinc oxide (IWZO) can be formed by a sputtering method using a target in which 0.5 wt % to 5 wt % tungsten oxide and 0.1 wt % to 1 wt % zinc oxide are added to indium oxide. Alternatively, gold (Au), platinum (Pt), nickel (Ni), tungsten (W), chromium (Cr), molybdenum (Mo), iron (Fe), cobalt (Co), copper (Cu), palladium (Pd), nitride of a metal material (e.g., titanium nitride), or the like can be used for the anode. Graphene can also be used for the anode. Note that an electrode material can be selected regardless of the work function when the composite material forming the second layer 117 in the above intermediate layer 116 is used for the layer (typically the hole-injection layer) in contact with the anode.

### <<Structure of Hole-Injection Layer>>

The hole-injection layer 111 is provided in contact with the anode and has a function of facilitating injection of holes into the organic compound layer 103 (the first light-emitting unit 501). The hole-injection layer 111 can be formed using a phthalocyanine-based compound or complex compound such as phthalocyanine (abbreviation: H₂Pc) and copper phthalocyanine (abbreviation: CuPc), an aromatic amine compound such as 4,4′-bis[N-(4-diphenylaminophenyl)-N-phenylamino]biphenyl (abbreviation: DPAB) and 4,4′-bis(N-{4-[N′-(3-methylphenyl)-N′-phenylamino]phenyl}-N-phenylamino)biphenyl (abbreviation: DNTPD), or a high molecular compound such as poly(3,4-ethylenedioxythiophene)/poly(styrenesulfonic acid) (abbreviation: PEDOT/PSS).

The hole-injection layer 111 may be formed using a substance having an electron-accepting property. As the substance having an acceptor property, any of the substances described as the acceptor substance used for the composite material forming the second layer 117 in the above intermediate layer 116 can be used similarly.

The composite material forming the second layer 117 in the above intermediate layer 116 may be similarly used to form the hole-injection layer 111.

In the hole-injection layer 111, it is further preferable that the organic compound having a hole-transport property used in the composite material have a relatively deep HOMO level higher than or equal to −5.7 eV and lower than or equal to −5.4 eV. Using the organic compound having a hole-transport property which has a relatively deep HOMO level in the composite material makes it easy to inject holes into the hole-transport layer and to obtain a light-emitting device having a long lifetime. In addition, when the organic compound having a hole-transport property used in the composite material has a relatively deep HOMO level, induction of holes can be inhibited properly so that the light-emitting device can have a longer lifetime.

The formation of the hole-injection layer 111 can improve the hole-injection property, which allows the light-emitting device to be driven at a low voltage.

Among substances having an acceptor property, the organic compound having an acceptor property is easy to use because it is easily deposited by evaporation.

The second light-emitting unit 502 includes no hole-injection layer because the second layer 117 in the intermediate layer 116 functions as a hole-injection layer; however, the second light-emitting unit 502 may include a hole-injection layer.

The hole-transport layer (the first hole-transport layer 112_1 or the second hole-transport layer 112_2) includes an organic compound having a hole-transport property. The organic compound having a hole-transport property preferably has a hole mobility higher than or equal to 1×10⁻⁶ cm²/Vs.

Examples of the aforementioned organic compound having a hole-transport property include the following compounds: compounds having an aromatic amine skeleton, such as 4,4′-bis[N-(1-naphthyl)-N-phenylamino]biphenyl (abbreviation: NPB), N,N-diphenyl-N,N-bis(3-methylphenyl)-4,4′-diaminobiphenyl (abbreviation: TPD), N,N-bis(9,9′-spirobi[9H-fluoren]-2-yl)-N,N-diphenyl-4,4′-diaminobiphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 9,9-dimethyl-N-phenyl-N-[4-(9-phenyl-9H-carbazol-3-yl)phenyl]fluoren-2-amine (abbreviation: PCBAF), and N-phenyl-N-[4-(9-phenyl-9H-carbazol-3-yl)phenyl]-9,9′-spirobi[9H-fluoren]-2-amine (abbreviation: PCBASF); compounds having a carbazole skeleton, such as 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), 3,3′-bis(9-phenyl-9H-carbazole) (abbreviation: PCCP); 9,9′-bis(biphenyl-4-yl)-3,3′-bi-9H-carbazole (abbreviation: BisBPCz), 9,9′-bis(biphenyl-3-yl)-3,3′-bi-9H-carbazole (abbreviation: BismBPCz), 9-(biphenyl-3-yl)-9′-(biphenyl-4-yl)-9H,9′H-3,3′-bicarbazole (abbreviation: mBPCCBP), 9-(2-naphthyl)-9′-phenyl-9H,9′H-3,3′-bicarbazole (abbreviation: βNCCP), 9-(3-biphenyl)-9′(2-naphthyl)-3,3′-bi-9H-carbazole (abbreviation: βNCCmBP), 9-(4-binaphthyl)-9′-(2-naphthyl)-3,3′-bi-9H-carbazole (abbreviation: βNCCBP), 9,9′-di-2-naphthyl-3,3′-9H,9′H-bicarbazole (abbreviation: BisβNCz), 9-(2-naphthyl)-9′-[1,1′:4′,1″-terphenyl]-3-yl-3,3′-9H,9′H-bicarbazole, 9-(2-naphthyl)-9′-[1,1′:3′,1″-terphenyl]-3-yl-3,3′-9H,9′H-bicarbazole, 9-(2-naphthyl)-9′-[1,1′:3′,1″-terphenyl]-5′-yl-3,3′-9H,9′H-bicarbazole, 9-(2-naphthyl)-9′-[1,1′:4′,1″-terphenyl]-4-yl-3,3′-9H,9′H-bicarbazole, 9-(2-naphthyl)-9′-[1,1′:3′,1″-terphenyl]-4-yl-3,3′-9H,9′H-bicarbazole, 9-(2-naphthyl)-9′-(triphenylen-2-yl)-3,3′-9H,9′H-bicarbazole, 9-phenyl-9′-(triphenylen-2-yl)-3,3′-9H,9′H-bicarbazole (abbreviation: PCCzTp), 9,9′-bis(triphenylen-2-yl)-3,3′-9H,9′H-bicarbazole, 9-(4-biphenyl)-9′-(triphenylen-2-yl)-3,3′-9H,9′H-bicarbazole, and 9-(triphenylen-2-yl)-9′-[1,1′:3′,1″-terphenyl]-4-yl-3,3′-9H,9′H-bicarbazole; compounds having a thiophene skeleton, such as 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), and 4-[4-(9-phenyl-9H-fluoren-9-yl)phenyl]-6-phenyldibenzothiophene (abbreviation: DBTFLP-IV); and compounds having a furan skeleton, such as 4,4′,4″-(benzene-1,3,5-triyl)tri(dibenzofuran) (abbreviation: DBF3P-II) and 4-{3-[3-(9-phenyl-9H-fluoren-9-yl)phenyl]phenyl}dibenzofuran (abbreviation: mmDBFFLBi-II). Among the above materials, the compound having an aromatic amine skeleton and the compound having a carbazole skeleton are preferable because these compounds are highly reliable and have high hole-transport properties to contribute to a reduction in driving voltage. Note that any of the substances given as examples of the organic compound having a hole-transport property that is used for the composite material in the hole-injection layer 111 can also be suitably used as the material included in the hole-transport layer.

### <<Structure of Light-Emitting Layer>>

The light-emitting layer (the light-emitting layer 113, the first light-emitting layer 1131, or the second light-emitting layer 1132) preferably includes alight-emitting substance and a host material. The light-emitting layer may additionally include other materials. Alternatively, the light-emitting layer may be a stack of two layers with different compositions.

As the light-emitting substance, fluorescent substances, phosphorescent substances, substances exhibiting thermally activated delayed fluorescence (TADF), or other light-emitting substances may be used.

Examples of the material that can be used as a fluorescent substance in the light-emitting layer are as follows. Other fluorescent substances can also be used.

The examples include 5,6-bis[4-(10-phenyl-9-anthryl)phenyl]-2,2′-bipyridine (abbreviation: PAP2BPy), 5,6-bis[4′-(10-phenyl-9-anthryl)biphenyl-4-yl]-2,2′-bipyridine (abbreviation: PAPP2BPy), N,N-diphenyl-N,N-bis[4-(9-phenyl-9H-fluoren-9-yl)phenyl]pyrene-1,6-diamine (abbreviation: 1,6FLPAPrn), N,N-bis(3-methylphenyl)-N,N-bis[3-(9-phenyl-9H-fluoren-9-yl)phenyl]pyrene-1,6-diamine (abbreviation: 1,6mMemFLPAPrn), N,N-bis[4-(9H-carbazol-9-yl)phenyl]-N,N-diphenylstilbene-4,4′-diamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA), N,9-diphenyl-N-[4-(10-phenyl-9-anthryl)phenyl]-9H-carbazol-3-amine (abbreviation: PCAPA), perylene, 2,5,8,11-tetra(tert-butyl)perylene (abbreviation: TBP), 4-(10-phenyl-9-anthryl)-4′-(9-phenyl-9H-carbazol-3-yl)triphenylamine (abbreviation: PCBAPA), N,N″-(2-tert-butylanthracene-9,10-diyldi-4,1-phenylene)bis(N,N,N-triphenyl-1,4-phenylenediamine) (abbreviation: DPABPA), N,9-diphenyl-N-[4-(9,10-diphenyl-2-anthryl)phenyl]-9H-carbazol-3-amine (abbreviation: 2PCAPPA), N-[4-(9,10-diphenyl-2-anthryl)phenyl]-N,N,N-triphenyl-1,4-phenylenediamine (abbreviation: 2DPAPPA), N,N,N,N,N″ N″ N″,N″-octaphenyldibenzo[g,p]chrysene-2,7,10,15-tetraamine (abbreviation: DBC1), coumarin 30, N-(9,10-diphenyl-2-anthryl)-N,9-diphenyl-9H-carbazol-3-amine (abbreviation: 2PCAPA), N-[9,10-bis(biphenyl-2-yl)-2-anthryl]-N,9-diphenyl-9H-carbazol-3-amine (abbreviation: 2PCABPhA), N-(9,10-diphenyl-2-anthryl)-N,N,N-triphenyl-1,4-phenylenediamine (abbreviation: 2DPAPA), 9,10-bis(2-biphenyl)-2-(N,N,N-triphenyl-1,4-phenylenediamin-N-yl)anthracene (abbreviation: 2DPABPhA), 9,10-bis(biphenyl-2-yl)-N-[4-(9H-carbazol-9-yl)phenyl]-N-phenylanthracen-2-amine (abbreviation: 2YGABPhA), N,N,9-triphenylanthracen-9-amine (abbreviation: DPhAPhA), coumarin 545T, N,N-diphenylquinacridone (abbreviation: DPQd), rubrene, 5,12-bis(biphenyl-4-yl)-6,11-diphenyltetracene (abbreviation: BPT), 2-(2-{2-[4-(dimethylamino)phenyl]ethenyl}-6-methyl-4H-pyran-4-ylidene)propanedinitrile (abbreviation: DCM1), 2-{2-methyl-6-[2-(2,3,6,7-tetrahydro-1H,5H-benzo[ij]quinolizin-9-yl)ethenyl]-4H-pyran-4-ylidene}propanedinitrile (abbreviation: DCM2), N,N,N,N-tetrakis(4-methylphenyl)tetracene-5,11-diamine (abbreviation: p-mPhTD), 7,14-diphenyl-N,N,N,N-tetrakis(4-methylphenyl)acenaphtho[1,2-a]fluoranthene-3,10-diamine (abbreviation: p-mPhAFD), 2-{2-isopropyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTI), 2-{2-tert-butyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTB), 2-(2,6-bis{2-[4-(dimethylamino)phenyl]ethenyl}-4H-pyran-4-ylidene)propanedinitrile (abbreviation: BisDCM), 2-{2,6-bis[2-(8-methoxy-1,1,7,7-tetramethyl-2,3,6,7-tetrahydro-1H,5H-benzo[ij]quinolizin-9-yl)ethenyl]-4H-pyran-4-ylidene}propanedinitrile (abbreviation: BisDCJTM), N,N-diphenyl-N,N′-(1,6-pyrene-diyl)bis[(6-phenylbenzo[b]naphtho[1,2-d]furan)-8-amine] (abbreviation: 1,6BnfAPrn-03), 3,10-bis[N-(9-phenyl-9H-carbazol-2-yl)-N-phenylamino]naphtho[2,3-b;6,7-b′]bisbenzofuran (abbreviation: 3,10PCA2Nbf(IV)-02), and 3,10-bis[N-(dibenzofuran-3-yl)-N-phenylamino]naphtho[2,3-b;6,7-b′]bisbenzofuran (abbreviation: 3,10FrA2Nbf(IV)-02). Condensed aromatic diamine compounds typified by pyrenediamine compounds such as 1,6FLPAPrn, 1,6mMemFLPAPrn, and 1,6BnfAPrn-03 are particularly preferable because of their high hole-trapping properties and high emission efficiency or high reliability.

Examples of the material that can be used when a phosphorescent substance is used as the light-emitting substance in the light-emitting layer are as follows.

The examples include an organometallic iridium complex having a 4H-triazole skeleton, such as tris{2-[5-(2-methylphenyl)-4-(2,6-dimethylphenyl)-4H-1,2,4-triazol-3-yl-κN²]phenyl-κC}iridium(III) (abbreviation: [Ir(mpptz-dmp)₃]), tris(5-methyl-3,4-diphenyl-4H-1,2,4-triazolato)iridium(III) (abbreviation: [Jr(Mptz)₃]), and tris[4-(3-biphenyl)-5-isopropyl-3-phenyl-4H-1,2,4-triazolato]iridium(III) (abbreviation: [Ir(iPrptz-3b)₃]); an organometallic iridium complex having a 1H-triazole skeleton, such as tris[3-methyl-1-(2-methylphenyl)-5-phenyl-1H-1,2,4-triazolato]iridium(III) (abbreviation: [Ir(Mptz1-mp)₃]) and tris(1-methyl-5-phenyl-3-propyl-1H-1,2,4-triazolato)iridium(III) (abbreviation: [Ir(Prptz1-Me)₃]); an organometallic iridium complex having an imidazole skeleton, such as fac-tris[1-(2,6-diisopropylphenyl)-2-phenyl-1H-imidazole]iridium(III) (abbreviation: [Ir(iPrpim)₃]) and tris[3-(2,6-dimethylphenyl)-7-methylimidazo[1,2-f]phenanthridinato]iridium(III) (abbreviation: [Ir(dmpimpt-Me)₃]); and an organometallic iridium complex in which a phenylpyridine derivative having an electron-withdrawing group is a ligand, such as bis[2-(4′,6′-difluorophenyl)pyridinato-N,C^{2′}]iridium(III) tetrakis(1-pyrazolyl)borate (abbreviation: FIr6), bis[2-(4′,6′-difluorophenyl)pyridinato-N,C^{2′}]iridium(III) picolinate (abbreviation: FIrpic), bis{2-[3′,5′-bis(trifluoromethyl)phenyl]pyridinato-N,C^{2′}}iridium(III) picolinate (abbreviation: [Ir(CF₃ppy)₂(pic)]), and bis[2-(4′,6′-difluorophenyl)pyridinato-N,C^{2′}]iridium(III) acetylacetonate (abbreviation: FIracac). These compounds emit blue phosphorescent light and have an emission peak in the wavelength range from 450 nm to 520 nm.

Other examples include organometallic iridium complexes having a pyrimidine skeleton, such as tris(4-methyl-6-phenylpyrimidinato)iridium(III) (abbreviation: [Ir(mppm)₃]), tris(4-t-butyl-6-phenylpyrimidinato)iridium(III) (abbreviation: [Ir(tBuppm)₃]), (acetylacetonato)bis(6-methyl-4-phenylpyrimidinato)iridium(III) (abbreviation: [Ir(mppm)2(acac)]), (acetylacetonato)bis(6-tert-butyl-4-phenylpyrimidinato)iridium(III) (abbreviation: [Ir(tBuppm)₂(acac)]), (acetylacetonato)bis[6-(2-norbornyl)-4-phenylpyrimidinato]iridium(III) (abbreviation: [Ir(nbppm)₂(acac)]), (acetylacetonato)bis[5-methyl-6-(2-methylphenyl)-4-phenylpyrimidinato]iridium(III) (abbreviation: [Ir(mpmppm)₂(acac)]), and (acetylacetonato)bis(4,6-diphenylpyrimidinato)iridium(III) (abbreviation: [Ir(dppm)₂(acac)]); organometallic iridium complexes having a pyrazine skeleton, such as (acetylacetonato)bis(3,5-dimethyl-2-phenylpyrazinato)iridium(III) (abbreviation: [Ir(mppr-Me)₂(acac)]) and (acetylacetonato)bis(5-isopropyl-3-methyl-2-phenylpyrazinato)iridium(III) (abbreviation: [Jr(mppr-iPr)₂(acac)]); organometallic iridium complexes having a pyridine skeleton, such as tris(2-phenylpyridinato-N,C^{2′})iridium(III) (abbreviation: [Ir(ppy)₃]), bis(2-phenylpyridinato-N,CT)iridium(III) acetylacetonate (abbreviation: [Ir(ppy)₂(acac)]), bis(benzo[h]quinolinato)iridium(III) acetylacetonate (abbreviation: [Ir(bzq)₂(acac)]), tris(benzo[h]quinolinato)iridium(III) (abbreviation: [Ir(bzq)₃]), tris(2-phenylquinolinato-N,C^{2′})iridium(III) (abbreviation: [Ir(pq)₃]), bis(2-phenylquinolinato-N,C^{2′})iridium(III) acetylacetonate (abbreviation: [Ir(pq)₂(acac)]); [2-d₃-methyl-8-(2-pyridinyl-κN)benzofuro[2,3-b]pyridine-κC]bis[2-(5-d₃-methyl-2-pyridinyl-κN²)phenyl-κC]iridium(III) (abbreviation: Ir(5mppy-d₃)₂(mbfpypy-d₃)), {[2-(methyl-d₃)-8-[4-(1-methylethyl-1-d)-2-pyridinyl-κN]benzofuro[2,3-b]pyridin-7-yl-κC}bis{5-(methyl-d₃)-2-[5-(methyl-d₃)-2-pyridinyl-κN]phenyl-κC}iridium(III) (abbreviation: Ir(5mtpy-d₆)₂(mbfpypy-iPr-d4)), [2-d₃-methyl-(2-pyridinyl-κN)benzofuro[2,3-b]pyridine-κC]bis[2-(2-pyridinyl-κN)phenyl-κC]iridium(III) (abbreviation: Ir(ppy)₂(mbfpypy-d₃)), and [2-(4-methyl-5-phenyl-2-pyridinyl-κN)phenyl-κC]bis[2-(2-pyridinyl-κN)phenyl-κC]iridium(III) (abbreviation: Ir(ppy)₂(mdppy)); and a rare earth metal complex such as tris(acetylacetonato) (monophenanthroline)terbium(III) (abbreviation: [Tb(acac)₃(Phen)]). These are mainly compounds that emit green phosphorescent light and have an emission peak in the wavelength range from 500 nm to 600 nm. Note that organometallic iridium complexes including a pyrimidine skeleton have distinctively high reliability or emission efficiency and thus are particularly preferable.

Other examples include organometallic iridium complexes having a pyrimidine skeleton, such as (diisobutyrylmethanato)bis[4,6-bis(3-methylphenyl)pyrimidinato]iridium(III) (abbreviation: [Ir(5mdppm)₂(dibm)]), bis[4,6-bis(3-methylphenyl)pyrimidinato](dipivaloylmethanato)iridium(III) (abbreviation: [Ir(5mdppm)₂(dpm)]), and bis[4,6-di(naphthalen-1-yl)pyrimidinato](dipivaloylmethanato)iridium(III) (abbreviation: [Ir(d1npm)₂(dpm)]); organometallic iridium complexes having a pyrazine skeleton, such as (acetylacetonato)bis(2,3,5-triphenylpyrazinato)iridium(III) (abbreviation: [Ir(tppr)₂(acac)]), bis(2,3,5-triphenylpyrazinato)(dipivaloylmethanato)iridium(III) (abbreviation: [Ir(tppr)₂(dpm)]), and (acetylacetonato)bis[2,3-bis(4-fluorophenyl)quinoxalinato]iridium(III) (abbreviation: [Ir(Fdpq)₂(acac)]); organometallic iridium complexes having a pyridine skeleton, such as tris(1-phenylisoquinolinato-N,C^{2′})iridium(III) (abbreviation: [Ir(piq)₃]) and bis(1-phenylisoquinolinato-N,C^{2′})iridium(III) acetylacetonate (abbreviation: [Ir(piq)₂(acac)]); platinum complexes such as 2,3,7,8,12,13,17,18-octaethyl-21H,23H-porphyrinplatinum(II) (abbreviation: PtOEP); and rare earth metal complexes such as tris(1,3-diphenyl-1,3-propanedionato)(monophenanthroline)europium(III) (abbreviation: [Eu(DBM)₃(Phen)]) and tris[1-(2-thenoyl)-3,3,3-trifluoroacetonato](monophenanthroline)europium(III) (abbreviation: [Eu(TTA)₃(Phen)]). These compounds emit red phosphorescent light and have an emission peak in the wavelength range from 600 nm to 700 nm. Furthermore, the organometallic iridium complexes having a pyrazine skeleton can provide red light emission with favorable chromaticity.

Besides the above phosphorescent compounds, known phosphorescent compounds may be selected and used.

Examples of the TADF material include a fullerene, a derivative thereof, an acridine, a derivative thereof, and an eosin derivative. Furthermore, a metal-containing porphyrin, such as a porphyrin containing magnesium (Mg), zinc (Zn), cadmium (Cd), tin (Sn), platinum (Pt), indium (In), or palladium (Pd), can be given. Examples of the metal-containing porphyrin include a protoporphyrin-tin fluoride complex (SnF₂(Proto IX)), a mesoporphyrin-tin fluoride complex (SnF₂(Meso IX)), a hematoporphyrin-tin fluoride complex (SnF₂(Hemato IX)), a coproporphyrin tetramethyl ester-tin fluoride complex (SnF₂(Copro III-4Me)), an octaethylporphyrin-tin fluoride complex (SnF₂(OEP)), an etioporphyrin-tin fluoride complex (SnF₂(Etio I)), and an octaethylporphyrin-platinum chloride complex (PtCl₂OEP), which are represented by the following structure formulae.

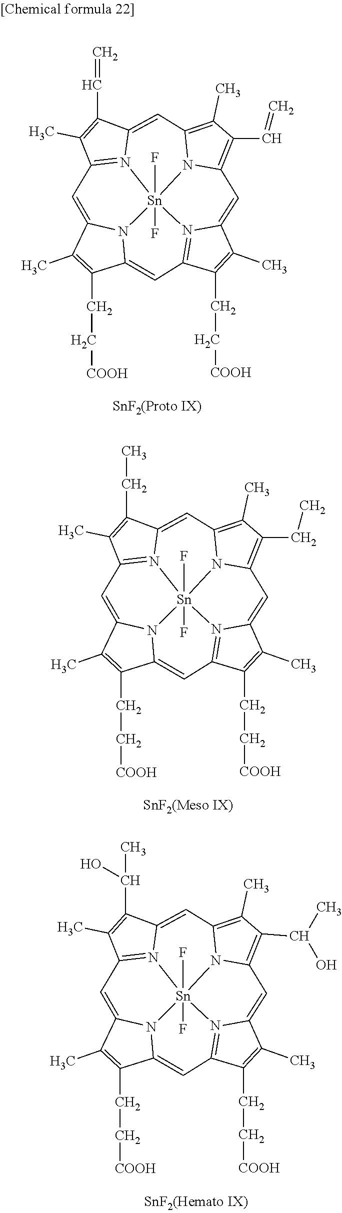

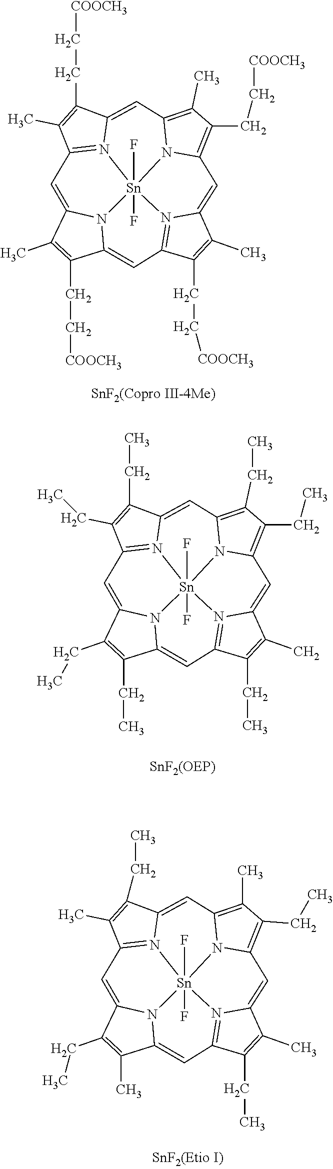

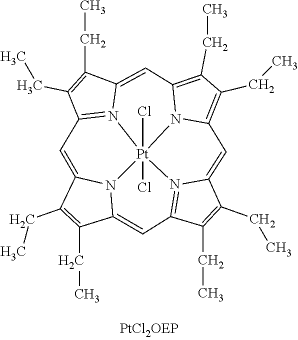

Alternatively, a heterocyclic compound having one or both of a π-electron rich heteroaromatic ring and a π-electron deficient heteroaromatic ring that is represented by the following structure formulae, such as 2-(biphenyl-4-yl)-4,6-bis(12-phenylindolo[2,3-a]carbazol-11-yl)-1,3,5-triazine (abbreviation: PIC-TRZ), 9-(4,6-diphenyl-1,3,5-triazin-2-yl)-9′-phenyl-9H,9′H-3,3′-bicarbazole (abbreviation: PCCzTzn), 2-{4-[3-(N-phenyl-9H-carbazol-3-yl)-9H-carbazol-9-yl]phenyl}-4,6-diphenyl-1,3,5-triazine (abbreviation: PCCzPTzn), 2-[4-(10H-phenoxazin-10-yl)phenyl]-4,6-diphenyl-1,3,5-triazine (abbreviation: PXZ-TRZ), 3-[4-(5-phenyl-5,10-dihydrophenazin-10-yl)phenyl]-4,5-diphenyl-1,2,4-triazole (abbreviation: PPZ-3TPT), 3-(9,9-dimethyl-9H-acridin-10-yl)-9H-xanthen-9-one (abbreviation: ACRXTN), bis[4-(9,9-dimethyl-9,10-dihydroacridine)phenyl]sulfone (abbreviation: DMAC-DPS), or 10-phenyl-10H,10′H-spiro[acridin-9,9′-anthracen]-10′-one (abbreviation: ACRSA) can be used. Such a heterocyclic compound is preferable because of having high electron-transport and hole-transport properties owing to a π-electron rich heteroaromatic ring and a π-electron deficient heteroaromatic ring. Among skeletons having the π-electron deficient heteroaromatic ring, a pyridine skeleton, a diazine skeleton (a pyrimidine skeleton, a pyrazine skeleton, and a pyridazine skeleton), and a triazine skeleton are preferred because of their high stability and reliability. In particular, a benzofuropyrimidine skeleton, a benzothienopyrimidine skeleton, a benzofuropyrazine skeleton, and a benzothienopyrazine skeleton are preferred because of their high acceptor properties and high reliability. Among skeletons having the π-electron rich heteroaromatic ring, an acridine skeleton, a phenoxazine skeleton, a phenothiazine skeleton, a furan skeleton, a thiophene skeleton, and a pyrrole skeleton have high stability and reliability; thus, at least one of these skeletons is preferably included. A dibenzofuran skeleton is preferable as a furan skeleton, and a dibenzothiophene skeleton is preferable as a thiophene skeleton. As a pyrrole skeleton, an indole skeleton, a carbazole skeleton, an indolocarbazole skeleton, a bicarbazole skeleton, and a 3-(9-phenyl-9H-carbazol-3-yl)-9H-carbazole skeleton are particularly preferable. Note that a substance in which the π-electron rich heteroaromatic ring is directly bonded to the π-electron deficient heteroaromatic ring is particularly preferred because the electron-donating property of the π-electron rich heteroaromatic ring and the electron-accepting property of the π-electron deficient heteroaromatic ring are both improved, the energy difference between the S₁ level and the T₁ level becomes small, and thus thermally activated delayed fluorescence can be obtained with high efficiency. Note that an aromatic ring to which an electron-withdrawing group such as a cyano group is bonded may be used instead of the π-electron deficient heteroaromatic ring. As a π-electron rich skeleton, an aromatic amine skeleton, a phenazine skeleton, or the like can be used. As a π-electron deficient skeleton, a xanthene skeleton, a thioxanthene dioxide skeleton, an oxadiazole skeleton, a triazole skeleton, an imidazole skeleton, an anthraquinone skeleton, a skeleton containing boron such as phenylborane or boranthrene, an aromatic ring or a heteroaromatic ring having a cyano group or a nitrile group such as benzonitrile or cyanobenzene, a carbonyl skeleton such as benzophenone, a phosphine oxide skeleton, a sulfone skeleton, or the like can be used. As described above, a π-electron deficient skeleton and a π-electron rich skeleton can be used instead of at least one of the π-electron deficient heteroaromatic ring and the π-electron rich heteroaromatic ring.

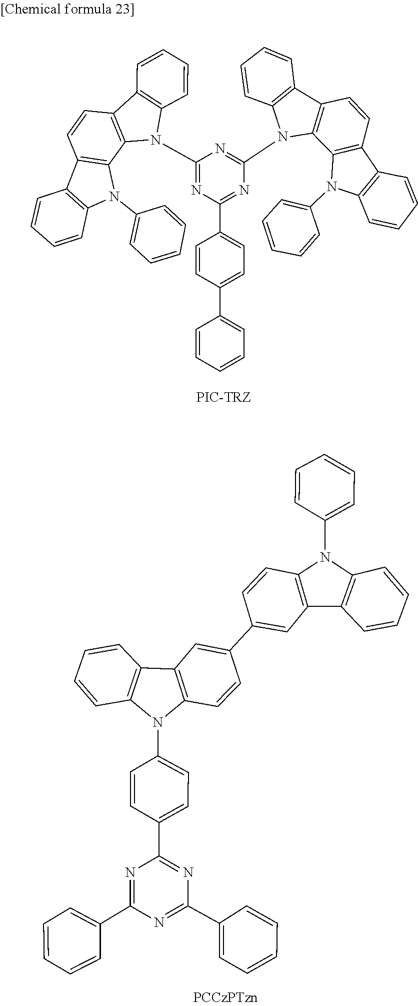

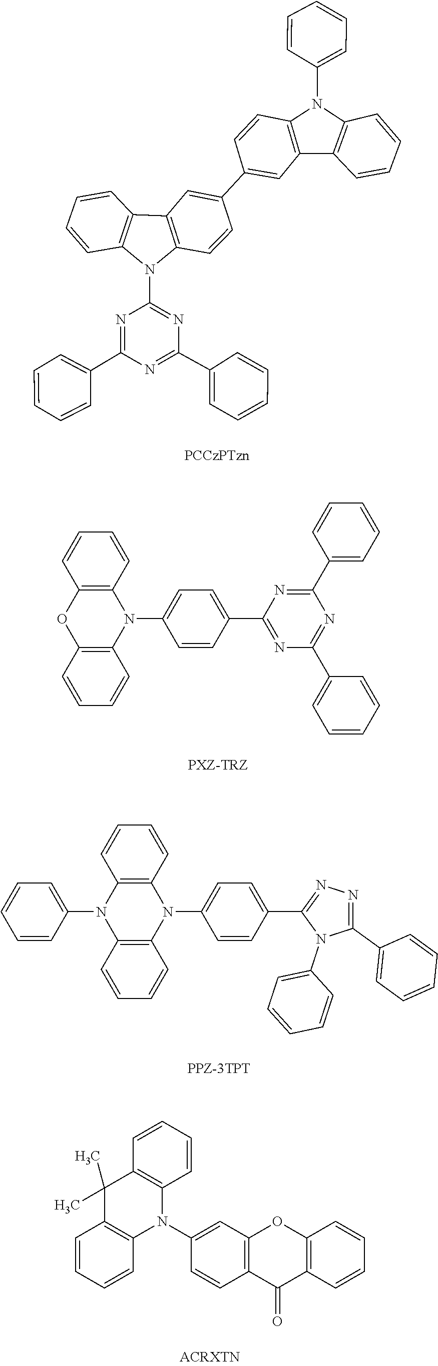 [SMILES: c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2nc(-c3ccccc3)nc(-c3ccc(N4c5ccccc5Oc5ccccc54)cc3)n2)cc1 ; c1ccc(-c2nnc(-c3ccc(N4c5ccccc5N(c5ccccc5)c5ccccc54)cc3)n2-c2ccccc2)cc1 ; CC1(C)c2ccccc2N(c2ccc3c(=O)c4ccccc4oc3c2)c2ccccc21]

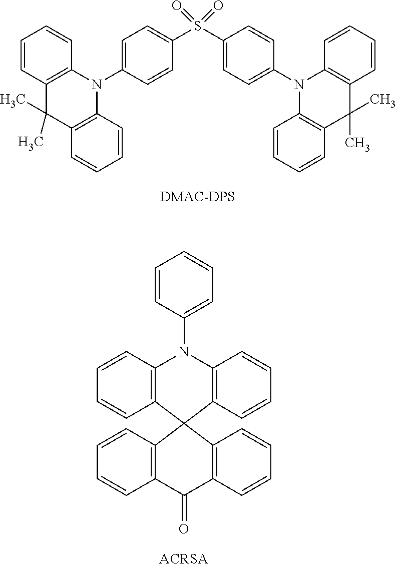 [SMILES: CC1(C)c2ccccc2N(c2ccc(S(=O)(=O)c3ccc(N4c5ccccc5C(C)(C)c5ccccc54)cc3)cc2)c2ccccc21 ; O=C1c2ccccc2C2(c3ccccc31)c1ccccc1N(c1ccccc1)c1ccccc12]

Alternatively, a TADF material whose singlet excited state and triplet excited state are in a thermal equilibrium state may be used. Since such a TADF material enables a short emission lifetime (excitation lifetime), an efficiency decrease of a light-emitting device in a high-luminance region can be inhibited. Specifically, a material having the following molecular structure can be used.

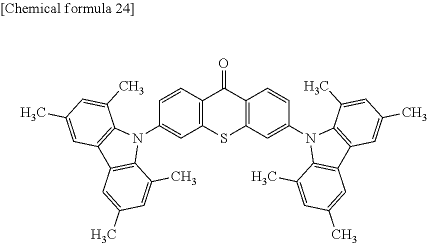

Note that a TADF material is a material having a small difference between the S₁ level and the T₁ level and a function of converting triplet excitation energy into singlet excitation energy by reverse intersystem crossing. Thus, a TADF material enables upconversion of triplet excitation energy into singlet excitation energy (i.e., reverse intersystem crossing) using a small amount of thermal energy and can efficiently generate a singlet excited state. In addition, the triplet excitation energy can be converted into light emission.

An exciplex whose excited state is formed of two kinds of substances has an extremely small difference between the S₁ level and the T₁ level and functions as a TADF material capable of converting triplet excitation energy into singlet excitation energy.

A phosphorescent spectrum observed at a low temperature (e.g., 77 K to 10 K) is used for an index of the T₁ level. When the level of energy with a wavelength of the line obtained by extrapolating a tangent to the fluorescent spectrum at a tail on the short wavelength side is the Si level and the level of energy with a wavelength of the line obtained by extrapolating a tangent to the phosphorescent spectrum at a tail on the short wavelength side is the T₁ level, the difference between the S₁ level and the T₁ level of the TADF material is preferably smaller than or equal to 0.3 eV, further preferably smaller than or equal to 0.2 eV.

When a TADF material is used as the light-emitting substance, the S₁ level of the host material is preferably higher than that of the TADF material. In addition, the T₁ level of the host material is preferably higher than that of the TADF material.

As the host material in the light-emitting layer, various carrier-transport materials such as materials having an electron-transport property and/or materials having a hole-transport property, and the TADF materials can be used.

As the material having a hole-transport property, the aforementioned material given as the material having a hole-transport property can be favorably used similarly.

As the material having an electron-transport property, the aforementioned material given as the material having an electron-transport property can be favorably used similarly.

As the TADF material that can be used as the host material, the aforementioned materials given as the TADF material can also be used. When the TADF material is used as the host material, triplet excitation energy generated in the TADF material is converted into singlet excitation energy by reverse intersystem crossing and transferred to the light-emitting substance, whereby the emission efficiency of the light-emitting device can be increased. Here, the TADF material functions as an energy donor, and the light-emitting substance functions as an energy acceptor.

This is very effective in the case where the light-emitting substance is a fluorescent substance. In that case, the S₁ level of the TADF material is preferably higher than that of the fluorescent substance in order that high emission efficiency can be achieved. Furthermore, the T₁ level of the TADF material is preferably higher than the S₁ level of the fluorescent substance. Therefore, the T₁ level of the TADF material is preferably higher than that of the fluorescent substance.

It is also preferable to use a TADF material emitting light whose wavelength overlaps with the wavelength on a lowest-energy-side absorption band of the fluorescent substance, in which case excitation energy is transferred smoothly from the TADF material to the fluorescent substance and light emission can be obtained efficiently.

In addition, in order to efficiently generate singlet excitation energy from the triplet excitation energy by reverse intersystem crossing, carrier recombination preferably occurs in the TADF material. It is also preferable that the triplet excitation energy generated in the TADF material not be transferred to the triplet excitation energy of the fluorescent substance. For that reason, the fluorescent substance preferably has a protective group around a luminophore (a skeleton which causes light emission) of the fluorescent substance. As the protective group, a substituent having no 7G bond and a saturated hydrocarbon are preferably used. Specific examples include an alkyl group having 3 to 10 carbon atoms, a substituted or unsubstituted cycloalkyl group having 3 to 10 carbon atoms, and a trialkylsilyl group having 3 to 10 carbon atoms. It is further preferable that the fluorescent substance have a plurality of protective groups. The substituents having no π bond are poor in carrier transport performance, whereby the TADF material and the luminophore of the fluorescent substance can be made away from each other with little influence on carrier transportation or carrier recombination. Here, the luminophore refers to an atomic group (skeleton) that causes light emission in a fluorescent substance. The luminophore is preferably a skeleton having a π bond, further preferably includes an aromatic ring, and still further preferably includes a condensed aromatic ring or a condensed heteroaromatic ring. Examples of such a luminophore include a phenanthrene skeleton, a stilbene skeleton, an acridone skeleton, a phenoxazine skeleton, a phenothiazine skeleton, a naphthalene skeleton, an anthracene skeleton, a fluorene skeleton, a chrysene skeleton, a triphenylene skeleton, a tetracene skeleton, a pyrene skeleton, a perylene skeleton, a coumarin skeleton, a quinacridone skeleton, and a naphthobisbenzofuran skeleton. Specifically, a fluorescent substance having any of a naphthalene skeleton, an anthracene skeleton, a fluorene skeleton, a chrysene skeleton, a triphenylene skeleton, a tetracene skeleton, a pyrene skeleton, a perylene skeleton, a coumarin skeleton, a quinacridone skeleton, and a naphthobisbenzofuran skeleton is preferable because of its high fluorescence quantum yield.

In the case where a fluorescent substance is used as the light-emitting substance, a material having an anthracene skeleton is suitably used as the host material. The use of a substance having an anthracene skeleton as the host material for the fluorescent substance makes it possible to obtain a light-emitting layer with high emission efficiency and high durability. Among the substances having an anthracene skeleton, a substance having a diphenylanthracene skeleton, in particular, a substance having a 9,10-diphenylanthracene skeleton, is chemically stable and thus is preferably used as the host material. The host material preferably has a carbazole skeleton because the hole-injection and hole-transport properties are improved; further preferably, the host material has a benzocarbazole skeleton in which a benzene ring is further fused to the carbazole skeleton because the HOMO level thereof is shallower than that of the host material having the carbazole skeleton by approximately 0.1 eV and thus holes enter the host material easily. In particular, the host material preferably has a dibenzocarbazole skeleton because the HOMO level thereof is shallower than that of the host material having the carbazole skeleton by approximately 0.1 eV and thus holes enter the host material easily, the hole-transport property is improved, and the heat resistance is increased. Accordingly, a substance having both a 9,10-diphenylanthracene skeleton and a carbazole skeleton (or a benzocarbazole or dibenzocarbazole skeleton) is further preferable as the host material. Note that in terms of the hole-injection and hole-transport properties described above, instead of a carbazole skeleton, a benzofluorene skeleton or a dibenzofluorene skeleton may be used. Examples of such a substance include 9-phenyl-3-[4-(10-phenyl-9-anthryl)phenyl]-9H-carbazole (abbreviation: PCzPA), 3-[4-(1-naphthyl)phenyl]-9-phenyl-9H-carbazole (abbreviation: PCPN), 9-[4-(10-phenyl-9-anthracenyl)phenyl]-9H-carbazole (abbreviation: CzPA), 7-[4-(10-phenyl-9-anthryl)phenyl]-7H-dibenzo[c,g]carbazole (abbreviation: cgDBCzPA), 6-[3-(9,10-diphenyl-2-anthryl)phenyl]benzo[b]naphtho[1,2-d]furan (abbreviation: 2mBnfPPA), 9-phenyl-10-[4-(9-phenyl-9H-fluoren-9-yl)biphenyl-4′-yl]anthracene (abbreviation: FLPPA), 9-(1-naphthyl)-10-[4-(2-naphthyl)phenyl]anthracene (abbreviation: αN-βNPAnth), 9-(1-naphthyl)-10-(2-naphthyl)anthracene (abbreviation: α,β-ADN), 2-(10-phenylanthracen-9-yl)dibenzofuran, 2-(10-phenyl-9-anthracenyl)-benzo[b]naphtho[2,3-d]furan (abbreviation: Bnf(II)PhA), 9-(2-naphthyl)-10-[3-(2-naphthyl)phenyl]anthracene (abbreviation: βN-mβNPAnth), and 1-{4-[10-(biphenyl-4-yl)-9-anthracenyl]phenyl}-2-ethyl-1H-benzimidazole (abbreviation: EtBImPBPhA). In particular, CzPA, cgDBCzPA, 2mBnfPPA, and PCzPA have excellent characteristics and thus are preferably selected.

Note that the host material may be a mixture of a plurality of kinds of substances; in the case of using a mixed host material, it is preferable to mix a material having an electron-transport property with a material having a hole-transport property. By mixing the material having an electron-transport property with the material having a hole-transport property, the transport property of the light-emitting layer 113 can be easily adjusted and a recombination region can be easily controlled. The weight ratio of the content of the material having a hole-transport property to the content of the material having an electron-transport property may be 1:19 to 19:1.

Note that a phosphorescent substance can be used as part of the mixed material. When a fluorescent substance is used as the light-emitting substance, a phosphorescent substance can be used as an energy donor supplying excitation energy to the fluorescent substance.

An exciplex may be formed of these mixed materials. These mixed materials are preferably selected so as to form an exciplex that exhibits light emission whose wavelength overlaps with the wavelength on a lowest-energy-side absorption band of the light-emitting substance, in which case energy can be transferred smoothly and light emission can be obtained efficiently. The use of such a structure is preferable because the driving voltage can also be reduced.

Note that at least one of the materials forming an exciplex may be a phosphorescent substance. In this case, triplet excitation energy can be efficiently converted into singlet excitation energy by reverse intersystem crossing.

Combination of a material having an electron-transport property and a material having a hole-transport property whose HOMO level is higher than or equal to that of the material having an electron-transport property is preferable for forming an exciplex efficiently. In addition, the LUMO level of the material having a hole-transport property is preferably higher than or equal to that of the material having an electron-transport property. Note that the LUMO levels and the HOMO levels of the materials can be derived from the electrochemical characteristics (the reduction potentials and the oxidation potentials) of the materials that are measured by cyclic voltammetry (CV).

The formation of an exciplex can be confirmed by a phenomenon in which the emission spectrum of the mixed film in which the material having a hole-transport property and the material having an electron-transport property are mixed is shifted to the longer wavelength than the emission spectrum of each of the materials (or has another peak on the longer wavelength side) observed by comparison of the emission spectra of the material having a hole-transport property, the material having an electron-transport property, and the mixed film of these materials, for example. Alternatively, the formation of an exciplex can be confirmed by a difference in transient response, such as a phenomenon in which the transient PL lifetime of the mixed film has longer lifetime components or has a larger proportion of delayed components than that of each of the materials, observed by comparison of transient photoluminescence (PL) of the material having a hole-transport property, the material having an electron-transport property, and the mixed film of these materials. The transient PL can be rephrased as transient electroluminescence (EL). That is, the formation of an exciplex can also be confirmed by a difference in transient response observed by comparison of the transient EL of the material having a hole-transport property, the material having an electron-transport property, and the mixed film of these materials.

### <<Structure of Electron-Transport Layer>>

The electron-transport layer (the electron-transport layer 114, the first electron-transport layer 114_1, or the second electron-transport layer 114_2) contains a substance having an electron-transport property. The material having an electron-transport property is preferably a substance with an electron mobility higher than or equal to 1×10⁻⁷ cm²/Vs, preferably higher than or equal to 1×10⁻⁶ cm²/Vs, when the square root of electric field strength [V/cm] is 600. Note that any other substance can be used as long as the substance has an electron-transport property higher than a hole-transport property. An organic compound including a π-electron deficient heteroaromatic ring is preferable as the above organic compound. The organic compound including a π-electron deficient heteroaromatic ring is preferably one or more of an organic compound including a heteroaromatic ring having a polyazole skeleton, an organic compound including a heteroaromatic ring having a pyridine skeleton, an organic compound including a heteroaromatic ring having a diazine skeleton, and an organic compound including a heteroaromatic ring having a triazine skeleton.

As the organic compound having an electron-transport property which can be used for the electron-transport layer, the organic compound that can be used as the organic compound having an electron-transport property in the first layer of the intermediate layer 116 can be used similarly. Among the above organic compounds, the organic compound including a heteroaromatic ring having a diazine skeleton, the organic compound including a heteroaromatic ring having a pyridine skeleton, and the organic compound including a heteroaromatic ring having a triazine skeleton have high reliability and thus are preferable. In particular, the organic compound including a heteroaromatic ring having a diazine (pyrimidine or pyrazine) skeleton and the organic compound including a heteroaromatic ring having a triazine skeleton have a good electron-transport property to contribute to a reduction in driving voltage.

The electron mobility of the electron-transport layer is preferably higher than or equal to 1×10⁻⁷ cm²/Vs and lower than or equal to 5×10⁻⁵ cm²/Vs, when the square root of the electric field strength [V/cm] is 600. The amount of electrons injected into the light-emitting layer can be controlled by the decrease in the electron-transport property of the electron-transport layer, whereby the light-emitting layer can be prevented from having excess electrons. It is particularly preferable to employ this structure when the hole-injection layer is formed using a composite material that includes a material having a hole-transport property with a relatively deep HOMO level higher than or equal to −5.7 eV or and lower than or equal to −5.4 eV, in which case a long lifetime can be achieved. In this case, the material having an electron-transport property preferably has a HOMO level higher than or equal to −6.0 eV.

### <<Structure of Electron-Injection Layer>>

As the electron-injection layer 115, a layer containing an alkali metal, an alkaline earth metal, a rare earth metal, or a compound thereof, or a complex thereof, such as lithium fluoride (LiF), cesium fluoride (CsF), calcium fluoride (CaF₂), 8-quinolinolato lithium (abbreviation: Liq), or ytterbium (Yb) may be provided. An electrode or a layer that is formed using a substance having an electron-transport property and that includes an alkali metal, an alkaline earth metal, or a compound thereof can be used as the electron-injection layer 115. Examples of the electrode include a substance in which electrons are added at a high concentration to calcium oxide-aluminum oxide.

Note that as the electron-injection layer 115, it is possible to use a layer containing a substance that has an electron-transport property (preferably an organic compound having a bipyridine skeleton) and contains a fluoride of the alkali metal or the alkaline earth metal at a concentration higher than or equal to that at which the electron-injection layer 115 becomes in a microcrystalline state (50 wt % or higher). Since the layer has a low refractive index, a light-emitting device including the layer can have high external quantum efficiency.

The organic compound of one embodiment of the present invention described in Embodiment 1 can be used for the electron-injection layer 115. The electron-injection layer 115 may contain a substance having an electron-transport property in addition to the organic compound of one embodiment of the present invention described in Embodiment 1.

### <<Structure of Second Electrode>>

The second electrode 102 is the electrode including a cathode. The second electrode 102 may have a stacked structure where the layer in contact with the organic compound layer 103 functions as the cathode. For the cathode, a metal, an alloy, an electrically conductive compound, or a mixture thereof each having a low work function (specifically, lower than or equal to 3.8 eV) or the like can be used. Specific examples of such a cathode material are elements belonging to Group 1 or 2 of the periodic table, such as alkali metals (e.g., lithium (Li) or cesium (Cs)), magnesium (Mg), calcium (Ca), and strontium (Sr), alloys containing these elements (e.g., MgAg and AlLi), rare earth metals such as europium (Eu) and ytterbium (Yb), and alloys containing these rare earth metals. However, when the electron-injection layer is provided between the second electrode 102 and the electron-transport layer, a variety of conductive materials such as Al, Ag, ITO, or indium oxide-tin oxide containing silicon or silicon oxide can be used for the cathode regardless of the work function.

When the second electrode 102 is formed using a material transmitting visible light, the light-emitting device can emit light from the second electrode 102 side.

Films of these conductive materials can be formed by a dry process such as a vacuum evaporation method or a sputtering method, an ink-jet method, a spin coating method, or the like. Alternatively, a wet process using a sol-gel method or a wet process using a paste of a metal material may be employed.

Furthermore, any of a variety of methods can be used to form the organic compound layer 103, regardless of a dry method or a wet method. For example, a vacuum evaporation method, a gravure printing method, an offset printing method, a screen printing method, an ink-jet method, a spin coating method, or the like may be used.

Different methods may be used to form the electrodes or the layers described above.

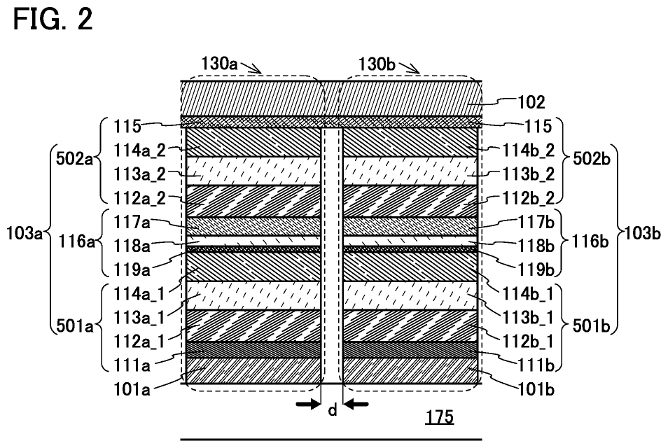

FIG. 2 illustrates two light-emitting devices (a light-emitting device 130a and a light-emitting device 130b) which are adjacent to each other in a display apparatus of one embodiment of the present invention.

The light-emitting device 130a includes an organic compound layer 103a between a first electrode 101a and the second electrode 102 over an insulating layer 175. In the organic compound layer 103α, a first light-emitting unit 501a and a second light-emitting unit 502a are stacked with an intermediate layer 116a interposed therebetween. Although two light-emitting units are stacked in the example illustrated in FIG. 2, three or more light-emitting units may be stacked. The first light-emitting unit 501a includes a hole-injection layer 111a, a first hole-transport layer 112a_1, a first light-emitting layer 113a_1, and a first electron-transport layer 114a_1. The intermediate layer 116a includes a second layer 117α, a third layer 118α, and a first layer 119a. The third layer 118a may be present or absent. The second light-emitting unit 502a includes a second hole-transport layer 112a_2, a second light-emitting layer 113a_2, a second electron-transport layer 114a_2, and the electron-injection layer 115.

The light-emitting device 130b includes an organic compound layer 103b between a first electrode 101b and the second electrode 102 over the insulating layer 175. In the organic compound layer 103b, a first light-emitting unit 501b and a second light-emitting unit 502b are stacked with an intermediate layer 116b interposed therebetween. Although two light-emitting units are stacked in the example illustrated in FIG. 2, three or more light-emitting units may be stacked. The first light-emitting unit 501b includes a hole-injection layer 111b, a first hole-transport layer 112b_1, a first light-emitting layer 113b_1, and a first electron-transport layer 114b_1. The intermediate layer 116b includes a second layer 117b, a third layer 118b, and a first layer 119b. The third layer 118b may be present or absent. The second light-emitting unit 502b includes a second hole-transport layer 112b_2, a second light-emitting layer 113b_2, a second electron-transport layer 114b_2, and the electron-injection layer 115.

The electron-injection layer 115 and the second electrode 102 are preferably a continuous layer shared by the light-emitting devices 130a and 130b. Except for the electron-injection layer 115, the organic compound layers 103a and 103b are isolated from each other because they are processed by a lithography method after the formation of the second electron-transport layer 114a_2 and after the formation of the second electron-transport layer 114b_2. The end portions (outlines) of the layers in the organic compound layer 103a except the electron-injection layer 115 are substantially aligned in the direction perpendicular to the substrate due to the processing by a lithography method. The end portions (outlines) of the layers in the organic compound layer 103b except the electron-injection layer 115 are substantially aligned in the direction perpendicular to the substrate due to the processing by a lithography method.

Since the organic compound layers are processed by a lithography method, a distance d between the first electrodes 101a and 101b can be shorter than that in the case of employing mask vapor deposition; the distance d can be longer than or equal to 2 μm and shorter than or equal to 5 μm.

This embodiment can be combined as appropriate with any of the other embodiments and examples. In this specification, in the case where a plurality of structure examples are shown in one embodiment, the structure examples can be combined as appropriate.

### Embodiment 3

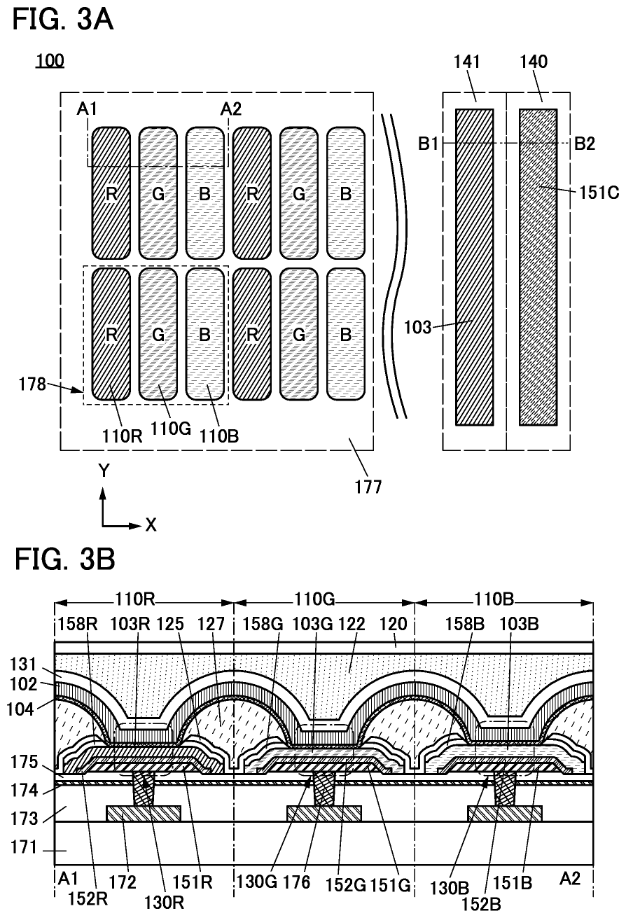

As illustrated in FIGS. 3A and 3B, a plurality of the light-emitting devices 130 are formed over the insulating layer 175 to constitute a display apparatus. In this embodiment, the display apparatus of one embodiment of the present invention will be described in detail.

A display apparatus 100 includes a pixel portion 177 in which a plurality of pixels 178 are arranged in matrix. The pixel 178 includes a subpixel 110R, a subpixel 110G, and a subpixel 110B.

In this specification and the like, for example, description common to the subpixels 110R, 110G, and 110B is sometimes made using the collective term “subpixel 110”. As for other components that are distinguished from each other using letters of the alphabet, matters common to the components are sometimes described using reference numerals excluding the letters of the alphabet.

The subpixel 110R emits red light, the subpixel 110G emits green light, and the subpixel 110B emits blue light. Thus, an image can be displayed on the pixel portion 177. Note that in this embodiment, three colors of red (R), green (G), and blue (B) are given as examples of colors of light emitted by the subpixels; however, subpixels of a different combination of colors may be employed. The number of subpixels is not limited to three, and may be four or more. Examples of four subpixels include subpixels emitting light of four colors of R, G, B, and white (W), subpixels emitting light of four colors of R, G, B, and Y, and four subpixels emitting R, G, B, and infrared (IR) light.

In this specification and the like, the row direction and the column direction are sometimes referred to as the X direction and the Y direction, respectively. The X direction and the Y direction intersect with each other and are perpendicular to each other, for example.

FIG. 3A illustrates an example where subpixels of different colors are arranged in the X direction and subpixels of the same color are arranged in the Y direction. Note that subpixels of different colors may be arranged in the Y direction, and subpixels of the same color may be arranged in the X direction.

A connection portion 140 and a region 141 may be provided outside the pixel portion 177, and the region 141 is provided between the pixel portion 177 and the connection portion 140. The organic compound layer 103 is provided in the region 141. A conductive layer 151C is provided in the connection portion 140.

Although FIG. 3A illustrates an example where the region 141 and the connection portion 140 are positioned on the right side of the pixel portion 177, the positions of the region 141 and the connection portion 140 are not particularly limited. The number of regions 141 and the number of connection portions 140 can each be one or more.

FIG. 3B is a cross-sectional view along the dashed-dotted line A1-A2 in FIG. 3A. As illustrated in FIG. 3B, the display apparatus 100 includes an insulating layer 171, a conductive layer 172 over the insulating layer 171, an insulating layer 173 over the insulating layer 171 and the conductive layer 172, an insulating layer 174 over the insulating layer 173, and the insulating layer 175 over the insulating layer 174. The insulating layer 171 is provided over a substrate (not illustrated). An opening reaching the conductive layer 172 is provided in the insulating layers 175, 174, and 173, and a plug 176 is provided to fill the opening.

In the pixel portion 177, the light-emitting device 130 is provided over the insulating layer 175 and the plug 176. A protective layer 131 is provided to cover the light-emitting device 130. A substrate 120 is attached to the protective layer 131 with a resin layer 122. An inorganic insulating layer 125 and an insulating layer 127 over the inorganic insulating layer 125 are preferably provided between the adjacent light-emitting devices 130.

Although each of the inorganic insulating layer 125 and the insulating layer 127 looks like a plurality of layers in the cross-sectional view in FIG. 3B, each of the inorganic insulating layer 125 and the insulating layer 127 is preferably one continuous layer when the display apparatus 100 is seen from above. In other words, the inorganic insulating layer 125 and the inorganic insulating layer 127 preferably include opening portions over the first electrode.

In FIG. 3B, a light-emitting device 130R, a light-emitting device 130G, and a light-emitting device 130B are illustrated as the light-emitting device 130. The light-emitting devices 130R, 130G, and 130B emit light of different colors. For example, the light-emitting device 130R can emit red light, the light-emitting device 130G can emit green light, and the light-emitting device 130B can emit blue light. Alternatively, the light-emitting device 130R, 130G, or 130B may emit visible light of another color or infrared light.

The display apparatus of one embodiment of the present invention can be, for example, a top-emission display apparatus where light is emitted in the direction opposite to a substrate over which light-emitting devices are formed. Note that the display apparatus of one embodiment of the present invention may be of a bottom-emission type.

Examples of a light-emitting substance contained in the light-emitting device 130 include organic compounds or organometallic complexes such as a substance emitting fluorescent light (a fluorescent material), a substance emitting phosphorescent light (a phosphorescent material), and a substance exhibiting thermally activated delayed fluorescence (a thermally activated delayed fluorescent (TADF) material). Other examples include inorganic compounds (e.g., a quantum dot material).

The light-emitting device 130R has a structure as described in Embodiment 2. The light-emitting device 130R includes a first electrode (pixel electrode) including a conductive layer 151R and a conductive layer 152R, an organic compound layer 103R over the first electrode, a common layer 104 over the organic compound layer 103R, and a second electrode (common electrode) 102 over the common layer 104. Although the common layer 104 is not necessarily provided, it is preferable to provide the common layer 104 to reduce damage to the organic compound layer 103R during processing. In the case where the common layer 104 is provided, the common layer 104 is preferably an electron-injection layer. Furthermore, in the case where the common layer 104 is provided, a stack of the organic compound layer 103R and the common layer 104 corresponds to the organic compound layer 103 described in Embodiment 2.

The light-emitting device 130G has a structure as described in Embodiment 2. The light-emitting device 130G includes the first electrode (pixel electrode) including a conductive layer 151G and a conductive layer 152G, an organic compound layer 103G over the first electrode, the common layer 104 over the organic compound layer 103G, and the second electrode (common electrode) 102 over the common layer 104. Although the common layer 104 is not necessarily provided, it is preferable to provide the common layer 104 to reduce damage to the organic compound layer 103G during processing. In the case where the common layer 104 is provided, the common layer 104 is preferably an electron-injection layer. Furthermore, in the case where the common layer 104 is provided, a stack of the organic compound layer 103G and the common layer 104 corresponds to the organic compound layer 103 described in Embodiment 2.

The light-emitting device 130B has a structure as described in Embodiment 2. The light-emitting device 130B includes the first electrode (pixel electrode) including a conductive layer 151B and a conductive layer 152B, an organic compound layer 103B over the first electrode, the common layer 104 over the organic compound layer 103B, and the second electrode (common electrode) 102 over the common layer 104. Although the common layer 104 is not necessarily provided, it is preferable to provide the common layer 104 to reduce damage to the organic compound layer 103B during processing. In the case where the common layer 104 is provided, the common layer 104 is preferably an electron-injection layer. Furthermore, in the case where the common layer 104 is provided, a stack of the organic compound layer 103B and the common layer 104 corresponds to the organic compound layer 103 described in Embodiment 2.

In the light-emitting device, one of the pixel electrode and the common electrode functions as an anode and the other functions as a cathode. Hereinafter, description is made on the assumption that the pixel electrode functions as the anode and the common electrode functions as the cathode unless otherwise specified.

The organic compound layers 103R, the organic compound layers 103G, and the organic compound layers 103B are island-shaped layers and are isolated on a light-emitting device basis or on an emission color basis. Providing the island-shaped organic compound layer 103 in each of the light-emitting devices 130 can inhibit leakage current between the adjacent light-emitting devices 130 even in a high-resolution display apparatus. This can prevent crosstalk, so that a display apparatus with extremely high contrast can be obtained. Specifically, a display apparatus having high current efficiency at low luminance can be obtained.

The island-shaped organic compound layer 103 is formed by forming an EL film and processing the EL film by a lithography method.

The organic compound layer 103 is preferably provided to cover the top surface and the side surface of the first electrode (pixel electrode) of the light-emitting device 130. In this case, the aperture ratio of the display apparatus 100 can be easily increased as compared to the structure where an end portion of the organic compound layer 103 is positioned inward from an end portion of the pixel electrode. Covering the side surface of the pixel electrode of the light-emitting device 130 with the organic compound layer 103 can inhibit the pixel electrode from being in contact with the second electrode 102; hence, a short circuit of the light-emitting device 130 can be inhibited. Furthermore, the distance between the light-emitting region (i.e., the region overlapping with the pixel electrode) of the organic compound layer 103 and the end portion of the organic compound layer 103 can be increased. Since the end portion of the organic compound layer 103 might be damaged by processing, using a region that is away from the end portion of the organic compound layer 103 as the light-emitting region can increase the reliability of the light-emitting device 130.

In the display apparatus of one embodiment of the present invention, the first electrode (pixel electrode) of the light-emitting device preferably has a stacked-layer structure. For example, in the example illustrated in FIG. 3B, the first electrode of the light-emitting device 130 is a stack of the conductive layer 151 and the conductive layer 152. In the case where the display apparatus 100 is of a top-emission type and the pixel electrode of the light-emitting device 130 functions as the anode, for example, the conductive layer 151 preferably has high visible light reflectance, and the conductive layer 152 preferably has a visible-light-transmitting property and a work function higher than that of the conductive layer 151. In the case where the display apparatus 100 is of a top-emission type, the higher the visible light reflectance of the pixel electrode is, the higher the light extraction efficiency of the organic compound layer 103 is. In the case where the pixel electrode functions as the anode, the higher the work function of the pixel electrode is, the easier hole injection into the organic compound layer 103 is. Accordingly, when the pixel electrode of the light-emitting device 130 is a stack of the conductive layer 151 with high visible light reflectance and the conductive layer 152 with a high work function, the light-emitting device 130 can have high light extraction efficiency and a low driving voltage.

In the case where the conductive layer 151 has high visible light reflectance, the visible light reflectance of the conductive layer 151 is preferably higher than or equal to 40% and lower than or equal to 100%, further preferably higher than or equal to 70% and lower than or equal to 100%, for example. When used as an electrode having a visible-light-transmitting property, the conductive layer 152 preferably has a visible light transmittance higher than or equal to 40%, for example.

Here, such a pixel electrode being a stack composed of a plurality of layers might change in quality as a result of, for example, a reaction between the plurality of layers. For example, when a film formed after the formation of the pixel electrode is removed by a wet etching method, contact of a chemical solution with the pixel electrode might cause galvanic corrosion.

In view of the above, the conductive layer 152 is formed to cover the top surface and the side surface of the conductive layer 151 in the display apparatus 100 of this embodiment. This can inhibit a chemical solution from coming into contact with the conductive layer 151 when a film that is formed after formation of the pixel electrode including the conductive layer 151 and the conductive layer 152 is removed by a wet etching method, for example. Accordingly, occurrence of galvanic corrosion in the pixel electrode can be inhibited, for example. This allows the display apparatus 100 to be manufactured by a high-yield method and to be accordingly inexpensive. In addition, generation of a defect in the display apparatus 100 can be inhibited, which makes the display apparatus 100 highly reliable.

A metal material can be used for the conductive layer 151, for example. Specifically, it is possible to use a metal such as aluminum (Al), titanium (Ti), chromium (Cr), manganese (Mn), iron (Fe), cobalt (Co), nickel (Ni), copper (Cu), gallium (Ga), zinc (Zn), indium (In), tin (Sn), molybdenum (Mo), tantalum (Ta), tungsten (W), palladium (Pd), gold (Au), platinum (Pt), silver (Ag), yttrium (Y), or neodymium (Nd) or an alloy containing an appropriate combination of any of these metals, for example.

For the conductive layer 152, an oxide containing one or more selected from indium, tin, zinc, gallium, titanium, aluminum, and silicon can be used. For example, it is preferable to use a conductive oxide containing one or more of indium oxide, indium tin oxide, indium zinc oxide, zinc oxide, zinc oxide containing gallium, titanium oxide, indium zinc oxide containing gallium, indium zinc oxide containing aluminum, indium tin oxide containing silicon, indium zinc oxide containing silicon, and the like. In particular, indium tin oxide containing silicon can be suitably used for the conductive layer 152 because of having a work function higher than or equal to 4.0 eV, for example.

The conductive layer 151 and the conductive layer 152 may each be a stack of a plurality of layers containing different materials. In this case, the conductive layer 151 may include a layer formed using a material that can be used for the conductive layer 152, such as a conductive oxide. Furthermore, the conductive layer 152 may include a layer formed using a material that can be used for the conductive layer 151, such as a metal material. In the case where the conductive layer 151 is a stack of two or more layers, for example, a layer in contact with the conductive layer 152 can be formed using a material that can be used for the conductive layer 152.

The conductive layer 151 preferably has a side surface with a tapered shape. Specifically, the end portion of the conductive layer 151 preferably has a tapered shape with a taper angle of less than 90°. In that case, the conductive layer 152 provided along the end portion of the conductive layer 151 also has a tapered shape. When the side surface of the conductive layer 152 has a tapered shape, coverage with the organic compound layer 103 provided along the side surface of the conductive layer 152 can be improved.

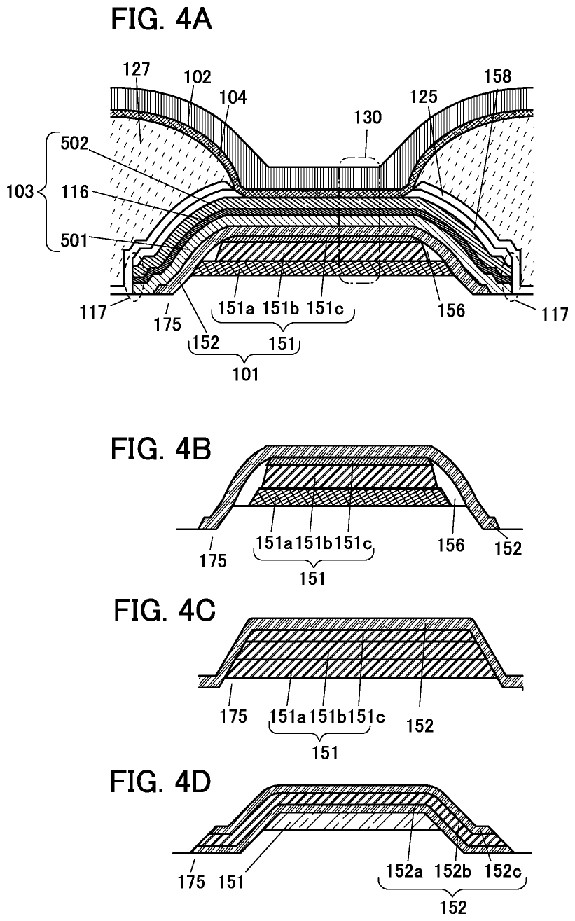

FIG. 4A illustrates the cases where the conductive layer 151 has a stacked-layer structure of a plurality of layers containing different materials. As illustrated in FIG. 4A, the conductive layer 151 includes a conductive layer 151a, a conductive layer 151b over the conductive layer 151a, and a conductive layer 151c over the conductive layer 151b. In other words, the conductive layer 151 illustrated in FIG. 4A has a three-layer structure. In the case where the conductive layer 151 has a stacked-layer structure of a plurality of layers as described above, the visible light reflectance of at least one of the layers included in the conductive layer 151 is made higher than that of the conductive layer 152.

In the example illustrated in FIG. 4A, the conductive layer 151b is interposed between the conductive layers 151a and 151c. A material that is less likely to change in quality than the conductive layer 151b is preferably used for the conductive layers 151a and 151c. The conductive layer 151a can be formed using, for example, a material that is less likely to migrate owing to contact with the insulating layer 175 than the material for the conductive layer 151b. The conductive layer 151c can be formed using a material an oxide of which has lower electrical resistivity than an oxide of the material for the conductive layer 151b and which is less likely to be oxidized than the material for the conductive layer 151b.

In this manner, the structure where the conductive layer 151b is interposed between the conductive layers 151a and 151c can expand the range of choices for the material for the conductive layer 151b. The conductive layer 151b, for example, can thus have higher visible light reflectance than at least one of the conductive layers 151a and 151c. For example, aluminum can be used for the conductive layer 151b. The conductive layer 151b may be formed using an alloy containing aluminum. The conductive layer 151a can be formed using titanium; titanium has lower visible light reflectance than aluminum but is less likely to migrate owing to contact with the insulating layer 175 than aluminum. Furthermore, the conductive layer 151c can be formed using titanium; titanium is less likely to be oxidized than aluminum and an oxide of titanium has lower electrical resistivity than aluminum oxide, although titanium has lower visible light reflectance than aluminum.

The conductive layer 151c may be formed using silver or an alloy containing silver. Silver is characterized by its visible light reflectance higher than that of titanium. In addition, silver is characterized by being less likely to be oxidized than aluminum, and silver oxide is characterized by having electrical resistivity lower than that of aluminum oxide. Thus, the conductive layer 151c formed using silver or an alloy containing silver can suitably increase the visible light reflectance of the conductive layer 151 and inhibit an increase in the electrical resistance of the pixel electrode due to oxidation of the conductive layer 151b. Here, as the alloy containing silver, an alloy of silver, palladium, and copper (also referred to as Ag—Pd—Cu or APC) can be used, for example. When the conductive layer 151c is formed using silver or an alloy containing silver and the conductive layer 151b is formed using aluminum, the visible light reflectance of the conductive layer 151c can be higher than that of the conductive layer 151b. Here, the conductive layer 151b may be formed using silver or an alloy containing silver. The conductive layer 151a may be formed using silver or an alloy containing silver.

Meanwhile, a film formed using titanium has better processability in etching than a film formed using silver. Thus, use of titanium for the conductive layer 151c can facilitate formation of the conductive layer 151c. Note that a film formed using aluminum also has better processability in etching than a film formed using silver.

The conductive layer 151 having a stacked-layer structure of a plurality of layers as described above can improve the characteristics of the display apparatus. For example, the display apparatus 100 can have high light extraction efficiency and high reliability.

Here, in the case where the light-emitting device 130 has a microcavity structure, use of silver or an alloy containing silver, i.e., a material with high visible light reflectance, for the conductive layer 151c can favorably increase the light extraction efficiency of the display apparatus 100.

As already described above, the conductive layer 151 preferably has a side surface with a tapered shape. Specifically, the side surface of the conductive layer 151 preferably has a tapered shape with a taper angle less than 90°. For example, in the conductive layer 151 illustrated in FIG. 4A, the side surface of at least one of the conductive layer 151a, the conductive layer 151b, and the conductive layer 151c preferably has a tapered shape.

The conductive layer 151 illustrated in FIG. 4A can be formed by a lithography method. Specifically, first, a conductive film to be the conductive layer 151α, a conductive film to be the conductive layer 151b, and a conductive film to be the conductive layer 151c are sequentially formed. Next, a resist mask is formed over the conductive film to be the conductive layer 151c. Then, the conductive films in the region not overlapping with the resist mask are removed by etching. Here, when the conductive films are processed under conditions where the resist mask is easily recessed (reduced in size) as compared to the case where the conductive layer 151 is formed such that the side surface does not have a tapered shape (i.e., the conductive layer 151 is formed to have a perpendicular side surface), the side surface of the conductive layer 151 can have a tapered shape.

Here, when the conductive films are processed under conditions where the resist mask is easily recessed (reduced in size), the conductive films might be easily processed in the horizontal direction. That is, the etching sometimes might become isotropic as compared to the case where the conductive layer 151 is formed to have a perpendicular side surface.

In the case where the conductive layer 151 is a stack of a plurality of layers formed of different materials, the plurality of layers sometimes differ in processability in the horizontal direction. For example, the conductive layer 151α, the conductive layer 151b, and the conductive layer 151c sometimes differ in processability in the horizontal direction.

In that case, after the processing of the conductive film, as illustrated in FIG. 4A, the side surface of the conductive layer 151b is sometimes positioned inward from the side surfaces of the conductive layers 151a and 151c and results in the formation of a protruding portion. This might impair coverage of the conductive layer 151 with the conductive layer 152 to cause step disconnection of the conductive layer 152.

In view of this, an insulating layer 156 is preferably provided as illustrated in FIG. 4A. FIG. 4A illustrates an example where the insulating layer 156 is provided over the conductive layer 151a to include a region overlapping with the side surface of the conductive layer 151b. In this structure, occurrence of step disconnection or thinning of the conductive layer 152 due to the protruding portion can be inhibited, so that poor connection or an increase in driving voltage can be inhibited.

Although FIG. 4A illustrates the structure where the side surface of the conductive layer 151b is entirely covered with the insulating layer 156, part of the side surface of the conductive layer 151b is not necessarily covered with the insulating layer 156. Also in a pixel electrode with a later-described structure, part of the side surface of the conductive layer 15lb is not necessarily covered with the insulating layer 156.

In the case where the conductive layer 151 has the structure illustrated in FIG. 4A, the conductive layer 152 is provided to cover the conductive layers 151a, 151b, and 151c and the insulating layer 156 and to be electrically connected to the conductive layers 151a, 151b, and 151c. This can prevent a chemical solution from coming into contact with the conductive layers 151a, 151b, and 151c when a film formed after formation of the conductive layer 152 is removed by a wet etching method, for example. It is thus possible to inhibit occurrence of corrosion in the conductive layers 151a, 151b, and 151c. Hence, the display apparatus 100 can be manufactured by a high-yield method. Moreover, the display apparatus 100 can have high reliability since generation of defects is inhibited therein.

Here, the insulating layer 156 preferably has a curved surface as illustrated in FIG. 4A. In this case, step disconnection in the conductive layer 152 covering the insulating layer 156 is less likely to occur than in the case where the insulating layer 156 has a perpendicular side surface (a side surface parallel to the Z direction), for example. In addition, step disconnection in the conductive layer 152 covering the insulating layer 156 is less likely to occur also in the case where the side surface of the insulating layer 156 has a tapered shape, specifically, a tapered shape with a taper angle of less than 90°, than in the case where the insulating layer 156 has a perpendicular side surface, for example. As described above, the display apparatus 100 can be manufactured by a high-yield method. Moreover, the display apparatus 100 can have high reliability since generation of defects is inhibited therein.

FIG. 4A illustrates the structure where the side surface of the conductive layer 151b is positioned inward from that of the conductive layer 151a and that of the conductive layer 151c; however, one embodiment of the present invention is not limited thereto. For example, the side surface of the conductive layer 151b may be positioned outward from that of the conductive layer 151a. The side surface of the conductive layer 151b may be positioned outward from that of the conductive layer 151c.

FIGS. 4B to 4D illustrate other structures of the first electrode 101. FIG. 4B illustrates a variation structure of the first electrode 101 in FIG. 4A, in which the insulating layer 156 covers the side surfaces of the conductive layers 151a, 151b, and 151c instead of covering only the side surface of the conductive layer 151b.

FIG. 4C illustrates a variation structure of the first electrode 101 in FIG. 4A, in which the insulating layer 156 is not provided.

FIG. 4D illustrates a variation structure of the first electrode 101 in FIG. 4A, in which the conductive layer 151 does not have a stacked-layer structure but the conductive layer 152 has a stacked-layer structure.

A conductive layer 152a has higher adhesion to a conductive layer 152b than the insulating layer 175 does, for example. For the conductive layer 152a, an oxide containing one or more selected from indium, tin, zinc, gallium, titanium, aluminum, and silicon, for example, can be used. For example, it is preferable to use a conductive oxide containing one or more of indium oxide, indium tin oxide, indium zinc oxide, zinc oxide, zinc oxide containing gallium, titanium oxide, indium titanium oxide, zinc titanate, aluminum zinc oxide, indium zinc oxide containing gallium, indium zinc oxide containing aluminum, indium tin oxide containing silicon, indium zinc oxide containing silicon, and the like. Accordingly, peeling of the conductive layer 152b can be inhibited. The conductive layer 152b is not in contact with the insulating layer 175.

The conductive layer 152b is a layer whose visible light reflectance (e.g., reflectance with respect to light with a predetermined wavelength longer than or equal to 400 nm and shorter than 750 nm) is higher than that of the conductive layers 151, 152a, and 152c. The visible light reflectance of the conductive layer 152b can be, for example, higher than or equal to 70% and lower than or equal to 100%, and is preferably higher than or equal to 80% and lower than or equal to 100%, further preferably higher than or equal to 90% and lower than or equal to 100%. For the conductive layer 152b, a material having higher visible light reflectance than aluminum can be used, for example. Specifically, for the conductive layer 152b, silver or an alloy containing silver can be used, for example. An example of the alloy containing silver is an alloy of silver, palladium, and copper (APC). In the above manner, the display apparatus 100 can have high light extraction efficiency. Note that a metal other than silver may be used for the conductive layer 152b.

When the conductive layers 151 and 152 serve as the anode, a layer having a high work function is preferably used as the conductive layer 152c. The conductive layer 152c has a higher work function than the conductive layer 152b, for example. For the conductive layer 152c, a material similar to the material usable for the conductive layer 152a can be used, for example. For example, the conductive layers 152a and 152c can be formed using the same kind of material. For example, in the case where indium tin oxide is used for the conductive layer 152a, indium tin oxide can also be used for the conductive layer 152c.

When the conductive layers 151 and 152 serve as the cathode, the conductive layer 152c is preferably a layer having a low work function. The conductive layer 152c has a lower work function than the conductive layer 152b, for example.

The conductive layer 152c is preferably a layer having high visible light transmittance (e.g., transmittance with respect to light with a predetermined wavelength longer than or equal to 400 nm and shorter than 750 nm). For example, the visible light transmittance of the conductive layer 152c is preferably higher than that of the conductive layers 151 and 152b. The visible light transmittance of the conductive layer 152c can be, for example, higher than or equal to 60% and lower than or equal to 100%, and is preferably higher than or equal to 70% and lower than or equal to 100%, further preferably higher than or equal to 80% and lower than or equal to 100%. Accordingly, the amount of light absorbed by the conductive layer 152c among light emitted from the organic compound layer 103 can be reduced. As described above, the conductive layer 152b under the conductive layer 152c can be a layer having high visible light reflectance. Thus, the display apparatus 100 can have high light extraction efficiency.

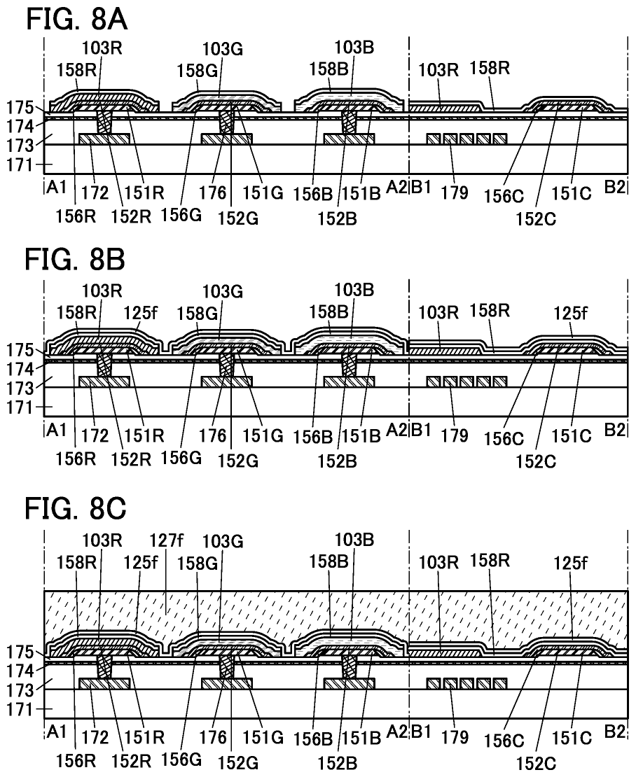

Next, a method for manufacturing the display apparatus 100 having the structure illustrated in FIG. 3A is described with reference to FIGS. 8A to 8C, FIGS. 9A to 9C, FIGS. 10A to 10C, FIGS. 11A to 11C, FIGS. 12A to 12E, FIGS. 13A to 13E, FIGS. 14A and 14B, FIGS. 15A and 15B, and FIGS. 16A to 16D. An organic layer of the light-emitting device included in the display apparatus 100 is formed by manufacturing steps including a process using water. The use of the organic compound of one embodiment of the present invention for the organic layer of the light-emitting device included in the display apparatus of one embodiment of the present invention prevents problems such as dissolution of a layer containing the organic compound and permeation of a chemical solution into the layer even in the manufacture by a manufacturing method including a process using water; consequently, the light-emitting device can have favorable characteristics.

### Manufacturing Method Example

Thin films included in the display apparatus (e.g., insulating films, semiconductor films, and conductive films) can be formed by a sputtering method, a chemical vapor deposition (CVD) method, a vacuum evaporation method, a pulsed laser deposition (PLD) method, an ALD method, or the like. Examples of a CVD method include a plasma-enhanced CVD (PECVD) method and a thermal CVD method. An example of a thermal CVD method is a metal organic CVD (MOCVD) method.

Thin films included in the display apparatus (e.g., insulating films, semiconductor films, and conductive films) can also be formed by a wet process such as spin coating, dipping, spray coating, ink-jetting, dispensing, screen printing, offset printing, doctor blade coating, slit coating, roll coating, curtain coating, or knife coating.

Specifically, for fabrication of the light-emitting device, a vacuum process such as an evaporation method and a solution process such as a spin coating method or an ink-jet method can be used. Examples of an evaporation method include physical vapor deposition methods (PVD methods) such as a sputtering method, an ion plating method, an ion beam evaporation method, a molecular beam evaporation method, and a vacuum evaporation method, and a chemical vapor deposition method (CVD method). Specifically, the functional layers (e.g., the hole-injection layer, the hole-transport layer, the hole-blocking layer, the light-emitting layer, the electron-blocking layer, the electron-transport layer, and the electron-injection layer) included in the organic compound layer can be formed by an evaporation method (e.g., a vacuum evaporation method), a coating method (e.g., a dip coating method, a die coating method, a bar coating method, a spin coating method, or a spray coating method), a printing method (e.g., ink-jetting, screen printing (stencil), offset printing (planography), flexography (relief printing), gravure printing, or micro-contact printing), or the like.

Thin films included in the display apparatus can be processed by a lithography method, for example. Alternatively, a nanoimprinting method, a sandblasting method, a lift-off method, or the like may be used to process thin films. Alternatively, island-shaped thin films may be directly formed by a film formation method using a shielding mask such as a metal mask.

As a lithography method, for example, a photolithography method can be used. There are two typical examples of photolithography methods. In one of the methods, a resist mask is formed over a thin film that is to be processed, the thin film is processed by etching, for example, and then the resist mask is removed. In the other method, a photosensitive thin film is formed and then processed into a desired shape by light exposure and development.

As light used for exposure in the photolithography method, for example, light with an i-line (wavelength: 365 nm), light with a g-line (wavelength: 436 nm), light with an h-line (wavelength: 405 nm), or light in which the i-line, the g-line, and the h-line are mixed can be used. Alternatively, ultraviolet rays, KrF laser light, ArF laser light, or the like can be used. Exposure may be performed by liquid immersion exposure technique. As the light for exposure, extreme ultraviolet (EUV) light or X-rays may also be used. Furthermore, instead of the light used for exposure, an electron beam can be used. It is preferable to use EUV light, X-rays, or an electron beam to perform extremely minute processing. Note that when exposure is performed by scanning of a beam such as an electron beam, a photomask is not needed.

For etching of thin films, a dry etching method, a wet etching method, a sandblasting method, or the like can be used.

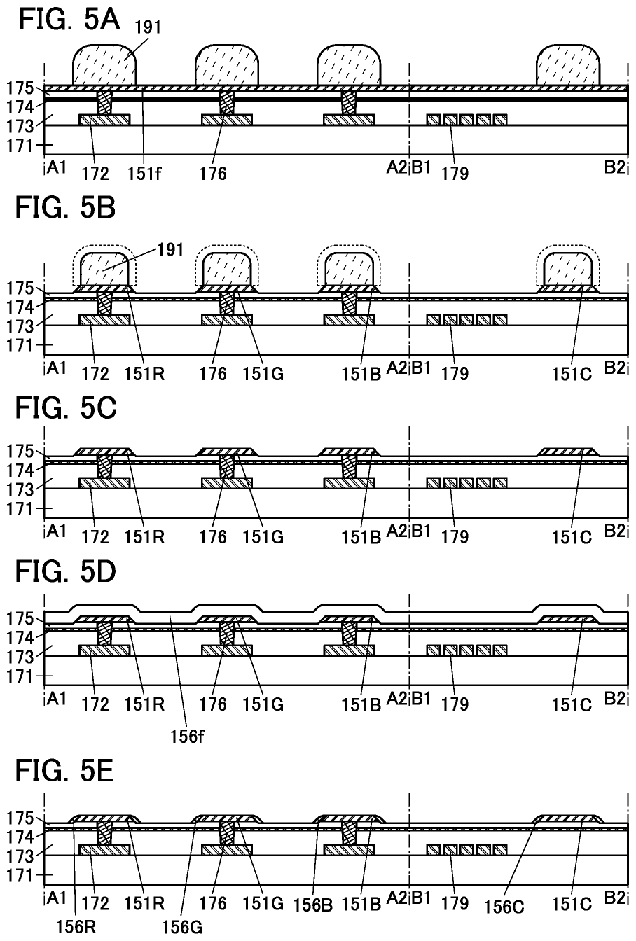

First, as illustrated in FIG. 5A, the insulating layer 171 is formed over a substrate (not illustrated). Next, the conductive layer 172 and a conductive layer 179 are formed over the insulating layer 171, and the insulating layer 173 is formed over the insulating layer 171 so as to cover the conductive layer 172 and the conductive layer 179. Then, the insulating layer 174 is formed over the insulating layer 173, and the insulating layer 175 is formed over the insulating layer 174.

As the substrate, a substrate having heat resistance high enough to withstand at least heat treatment performed later can be used. When an insulating substrate is used, it is possible to use a glass substrate, a quartz substrate, a sapphire substrate, a ceramic substrate, an organic resin substrate, or the like. Alternatively, it is possible to use a semiconductor substrate such as a single crystal semiconductor substrate or a polycrystalline semiconductor substrate of silicon, silicon carbide, or the like; a compound semiconductor substrate of silicon germanium or the like; or an SOI substrate.

Next, as illustrated in FIG. 5A, openings reaching the conductive layer 172 are formed in the insulating layers 175, 174, and 173. Then, the plugs 176 are formed to fill the openings.

Next, as illustrated in FIG. 5A, a conductive film 151f to be the conductive layers 151R, 151G, 151B, and 151C is formed over the plugs 176 and the insulating layer 175. The conductive film 151f can be formed by a sputtering method or a vacuum evaporation method, for example. A metal material can be used for the conductive film 151f, for example.

Subsequently, a resist mask 191 is formed over the conductive film 151f as illustrated in FIG. 5A. The resist mask 191 can be formed by application of a photosensitive material (photoresist), light exposure, and development.

Subsequently, as illustrated in FIG. 5B, the conductive film 151f in a region not overlapping with the resist mask 191, for example, is removed by an etching method, specifically, a dry etching method, for instance. Note that in the case where the conductive film 151f includes a layer formed using a conductive oxide such as indium tin oxide, for example, the layer may be removed by a wet etching method. In this manner, the conductive layer 151 is formed. In the case where part of the conductive film 151f is removed by a dry etching method, for example, a recessed portion may be formed in a region of the insulating layer 175 not overlapping with the conductive layer 151.

Next, the resist mask 191 is removed as illustrated in FIG. 5C. The resist mask 191 can be removed by ashing using oxygen plasma, for example. Alternatively, an oxygen gas and any of CF₄, C₄F₈, SF₆, CHF₃, Cl₂, H₂O, BCl₃, and a Group 18 element such as He may be used. Alternatively, the resist mask 191 may be removed by wet etching.

Then, as illustrated in FIG. 5D, an insulating film 156f to be an insulating layer 156R, an insulating layer 156G, an insulating layer 156B, and an insulating layer 156C is formed over the conductive layer 151R, the conductive layer 151G, the conductive layer 151B, the conductive layer 151C, and the insulating layer 175. The insulating film 156f can be formed by a CVD method, an ALD method, a sputtering method, or a vacuum evaporation method, for example.

For the insulating film 156f, an inorganic material can be used. As the insulating film 156f, an inorganic insulating film such as an oxide insulating film, a nitride insulating film, an oxynitride insulating film, or a nitride oxide insulating film can be used, for example. For example, an oxide insulating film containing silicon, a nitride insulating film containing silicon, an oxynitride insulating film containing silicon, a nitride oxide insulating film containing silicon, or the like can be used as the insulating film 156f. For the insulating film 156f, silicon oxynitride can be used, for example.

Subsequently, as illustrated in FIG. 5E, the insulating film 156f is processed to form the insulating layers 156R, 156G, 156B, and 156C. The insulating layer 156 can be formed by performing etching substantially uniformly on the top surface of the insulating film 156f, for example. Such uniform etching for planarization is also referred to as etch back treatment. Note that the insulating layer 156 may be formed by a lithography method.

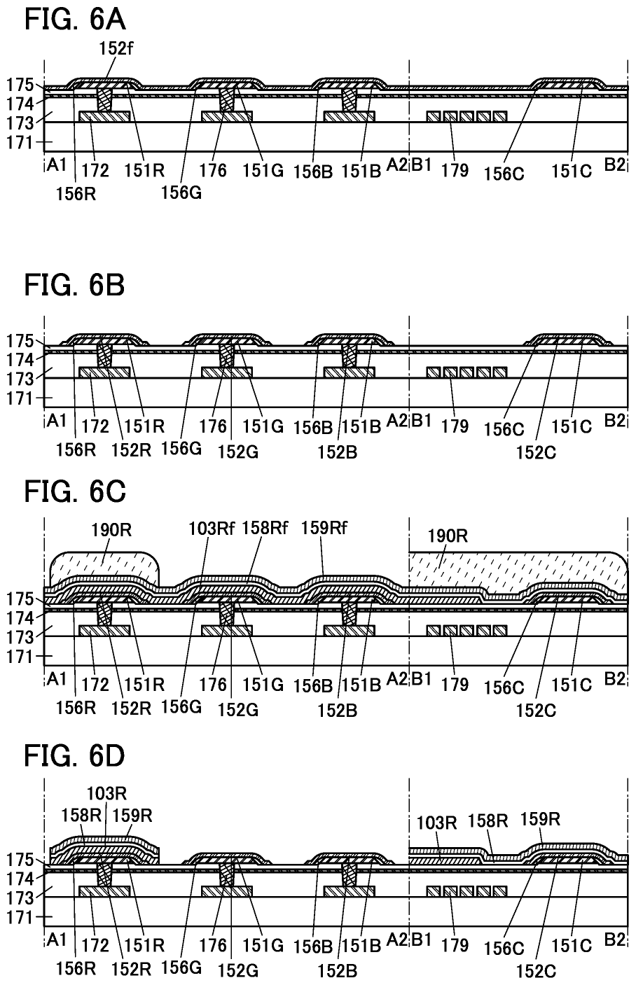

Then, as illustrated in FIG. 6A, a conductive film 152f to be the conductive layers 152R, 152G, and 152B and a conductive layer 152C is formed over the conductive layers 151R, 151G, 151B, and 151C and the insulating layers 156R, 156G, 156B, 156C, and 175. Specifically, the conductive film 152f is formed to cover the conductive layers 151R, 151G, 151B, and 151C and the insulating layers 156R, 156G, 156B, and 156C, for example.

The conductive film 152f can be formed by a sputtering method or a vacuum evaporation method, for example. A conductive oxide can be used for the conductive film 152f, for example. The conductive film 152f can be a stack of a film formed using a metal material and a film formed thereover using a conductive oxide. For example, the conductive film 152f can be a stack of a film formed using titanium, silver, or an alloy containing silver and a film formed thereover using a conductive oxide.

The conductive film 152f can be formed by an ALD method. In this case, for the conductive film 152f, an oxide containing one or more selected from indium, tin, zinc, gallium, titanium, aluminum, and silicon can be used. In this case, the conductive film 152f can be formed by repeating a cycle of introduction of a precursor (generally referred to as a metal precursor or the like in some cases), purge of the precursor, introduction of an oxidizer (generally referred to as a reactant, a non-metal precursor, or the like in some cases), and purge of the oxidizer. Here, in the case where an oxide film containing a plurality of kinds of metals (e.g., an indium tin oxide film) is formed as the conductive film 152f, the composition of the metals can be controlled by varying the number of cycles for different kinds of precursors.

For example, in the case where an indium tin oxide film is formed as the conductive film 152f, after a precursor containing indium is introduced, the precursor is purged, and an oxidizer is introduced to form an In—O film, and then a precursor containing tin is introduced, the precursor is purged, and an oxidizer is introduced to form a Sn—O film. Here, when the number of cycles of forming an In—O film is larger than the number of cycles of forming a Sn—O film, the number of In atoms contained in the conductive film 152f can be larger than the number of Sn atoms contained therein.

For example, to form a zinc oxide film as the conductive film 152f, a Zn—O film is formed in the above procedure. As another example, to form an aluminum zinc oxide film as the conductive film 152f, a Zn—O film and an Al—O film are formed in the above procedure. As another example, to form a titanium oxide film as the conductive film 152f, a Ti—O film is formed in the above procedure. As another example, to form an indium tin oxide film containing silicon as the conductive film 152f, an In—O film, a Sn—O film, and a Si—O film are formed in the above procedure. As another example, to form a zinc oxide film containing gallium, a Ga—O film and a Zn—O film are formed in the above procedure.

As a precursor containing indium, it is possible to use, for example, triethylindium, trimethylindium, or [1,1,1-trimethyl-N-(trimethylsilyl)amide]-indium. As a precursor containing tin, it is possible to use, for example, tin chloride or tetrakis(dimethylamido)tin. As a precursor containing zinc, it is possible to use, for example, diethylzinc or dimethylzinc. As a precursor containing gallium, it is possible to use, for example, triethylgallium. As a precursor containing titanium, it is possible to use, for example, titanium chloride, tetrakis(dimethylamido)titanium, or tetraisopropyl titanate. As a precursor containing aluminum, it is possible to use, for example, aluminum chloride or trimethylaluminum. As a precursor containing silicon, it is possible to use, for example, trisilylamine, bis(diethylamino)silane, tris(dimethylamino)silane, bis(tert-butylamino)silane, or bis(ethylmethylamino)silane. As the oxidizer, water vapor, oxygen plasma, or an ozone gas can be used.

Then, as illustrated in FIG. 6B, the conductive film 152f is processed by a lithography method, for example, whereby the conductive layers 152R, 152G, 152B, and 152C are formed. Specifically, after a resist mask is formed, part of the conductive film 152f is removed by an etching method, for example. The conductive film 152f can be removed by a wet etching method, for example. The conductive film 152f may be removed by a dry etching method. Through the above steps, the pixel electrode including the conductive layer 151 and the conductive layer 152 is formed.

Next, hydrophobization treatment is preferably performed on the conductive layer 152. The hydrophobization treatment can change the hydrophilic properties of the subject surface to hydrophobic properties or increase the hydrophobic properties of the subject surface. The hydrophobization treatment for the conductive layer 152 can increase the adhesion between the conductive layer 152 and the organic compound layer 103 formed in a later step and inhibit film peeling. Note that the hydrophobization treatment is not necessarily performed.

Next, as illustrated in FIG. 6C, an organic compound film 103Rf to be an organic compound layer 103R is formed over the conductive layers 152R, 152G, and 152B and the insulating layer 175.

As illustrated in FIG. 6C, the organic compound film 103Rf is not formed over the conductive layer 152C. For example, a mask for specifying a film formation area (also referred to as an area mask, a rough metal mask, or the like to be distinguished from a fine metal mask) is used, so that the organic compound film 103Rf can be formed only in a desired region. Employing a film formation step using an area mask and a processing step using a resist mask enables a light-emitting device to be manufactured by a relatively easy process.

The organic compound film 103Rf can be formed by an evaporation method, specifically a vacuum evaporation method, for example. The organic compound film 103Rf may be formed by a transfer method, a printing method, an ink-jet method, a coating method, or the like.

Next, as illustrated in FIG. 6C, a sacrificial film 158Rf to be a sacrificial layer 158R and a mask film 159Rf to be a mask layer 159R are sequentially formed over the organic compound film 103Rf, the conductive layer 152C, and the insulating layer 175.

Although this embodiment shows an example where a mask film having a two-layer structure of the sacrificial film 158Rf and the mask film 159Rf is formed, a mask film may have a single-layer structure or a stacked-layer structure of three or more layers. In this specification and the like, a mask layer may be referred to as a sacrificial layer.

Providing the sacrificial layer over the organic compound film 103Rf can reduce damage to the organic compound film 103Rf in the manufacturing process of the display apparatus, resulting in an increase in reliability of the light-emitting device.

As the sacrificial film 158Rf, a film that is highly resistant to the process conditions for the organic compound film 103Rf, specifically, a film having high etching selectivity with respect to the organic compound film 103Rf is used. For the mask film 159Rf, a film having high etching selectivity with respect to the sacrificial film 158Rf is used.

The sacrificial film 158Rf and the mask film 159Rf are formed at a temperature lower than the upper temperature limit of the organic compound film 103Rf. The typical substrate temperatures in formation of the sacrificial film 158Rf and the mask film 159Rf are each lower than or equal to 200° C., preferably lower than or equal to 150° C., further preferably lower than or equal to 120° C., still further preferably lower than or equal to 100° C., yet still further preferably lower than or equal to 80° C.

The sacrificial film 158Rf and the mask film 159Rf are preferably films that can be removed by a wet etching method. The use of a wet etching method can reduce damage to the organic compound film 103Rf in processing of the sacrificial film 158Rf and the mask film 159Rf, as compared to the case of using a dry etching method.

The sacrificial film 158Rf and the mask film 159Rf can be formed by a sputtering method, an ALD method (including a thermal ALD method or a PEALD method), a CVD method, or a vacuum evaporation method, for example. Alternatively, the sacrificial film 158Rf and the mask film 159Rf may be formed by the above-described wet process.

Note that the sacrificial film 158Rf that is formed over and in contact with the organic compound film 103Rf is preferably formed by a formation method that is less likely to damage the organic compound film 103Rf than a formation method of the mask film 159Rf. For example, the sacrificial film 158Rf is preferably formed by an ALD method or a vacuum evaporation method rather than a sputtering method.

As each of the sacrificial film 158Rf and the mask film 159Rf, one or more of a metal film, an alloy film, a metal oxide film, a semiconductor film, an organic insulating film, and an inorganic insulating film, for example, can be used.

For each of the sacrificial film 158Rf and the mask film 159Rf, it is preferable to use a metal material such as gold, silver, platinum, magnesium, nickel, tungsten, chromium, molybdenum, iron, cobalt, copper, palladium, titanium, aluminum, yttrium, zirconium, or tantalum or an alloy material containing any of the metal materials, for example. It is particularly preferable to use a low-melting-point material such as aluminum or silver. It is preferable to use a metal material that can block ultraviolet rays for one or both of the sacrificial film 158Rf and the mask film 159Rf, in which case the organic compound film 103Rf can be inhibited from being irradiated with ultraviolet rays and deteriorating.

The sacrificial film 158Rf and the mask film 159Rf can each be formed using a metal oxide such as In—Ga—Zn oxide, indium oxide, In—Zn oxide, In—Sn oxide, indium titanium oxide (In—Ti oxide), indium tin zinc oxide (In—Sn—Zn oxide), indium titanium zinc oxide (In—Ti—Zn oxide), indium gallium tin zinc oxide (In—Ga—Sn—Zn oxide), or indium tin oxide containing silicon.

In addition, in place of gallium described above, an element M (M is one or more of aluminum, silicon, boron, yttrium, copper, vanadium, beryllium, titanium, iron, nickel, germanium, zirconium, molybdenum, lanthanum, cerium, neodymium, hafnium, tantalum, tungsten, and magnesium) may be used.

As each of the sacrificial film and the mask film, a film containing a material having a light-blocking property, particularly with respect to ultraviolet rays, is preferably used. Although a variety of materials such as a metal, an insulator, a semiconductor, and a metalloid that have a property of blocking ultraviolet rays can be used as a light-blocking material, each of the sacrificial film and the mask film is preferably a film capable of being processed by etching and is particularly preferably a film having good processability because part or the whole of each of the sacrificial film and the mask film is removed in a later step.

For example, a semiconductor material with excellent compatibility with a semiconductor manufacturing process, such as silicon or germanium, is preferably used for the sacrificial film and the mask film. An oxide or a nitride of the semiconductor material can be used. A non-metallic material such as carbon or a compound thereof can be used. A metal such as titanium, tantalum, tungsten, chromium, or aluminum or an alloy containing at least one of these metals can be used. Alternatively, an oxide containing the above-described metal, such as titanium oxide or chromium oxide, or a nitride such as titanium nitride, chromium nitride, or tantalum nitride can be used.

When a film containing a material having a property of blocking ultraviolet rays is used as each of the sacrificial film and the mask film, the organic compound layer can be inhibited from being irradiated with ultraviolet rays in a light exposure step, for example. The organic compound layer is inhibited from being damaged by ultraviolet rays, so that the reliability of the light-emitting device can be improved.

Note that the same effect is obtained when a film containing a material having a property of blocking ultraviolet rays is used for an after-mentioned inorganic insulating film 125f.

As each of the sacrificial film 158Rf and the mask film 159Rf, any of a variety of inorganic insulating films can be used. In particular, an oxide insulating film is preferable because its adhesion to the organic compound film 103Rf is higher than that of a nitride insulating film. For example, an inorganic insulating material such as aluminum oxide, hafnium oxide, or silicon oxide can be used for the sacrificial film 158Rf and the mask film 159Rf. As the sacrificial film 158Rf and the mask film 159Rf, aluminum oxide films can be formed by an ALD method, for example. An ALD method is preferably used, in which case damage to a base (in particular, the organic compound layer) can be reduced.

For example, an inorganic insulating film (e.g., an aluminum oxide film) formed by an ALD method can be used as the sacrificial film 158Rf, and an inorganic film (e.g., an In—Ga—Zn oxide film, an aluminum film, or a tungsten film) formed by a sputtering method can be used as the mask film 159Rf.

Note that the same inorganic insulating film can be used for both the sacrificial film 158Rf and the inorganic insulating layer 125 that is to be formed later. For example, an aluminum oxide film formed by an ALD method can be used for both the sacrificial film 158Rf and the inorganic insulating layer 125. For the sacrificial film 158Rf and the inorganic insulating layer 125, the same deposition conditions may be used or different deposition conditions may be used. For example, when the sacrificial film 158Rf is formed under conditions similar to those of the inorganic insulating layer 125, the sacrificial film 158Rf can be an insulating layer having a high barrier property against at least one of water and oxygen. Meanwhile, since the sacrificial film 158Rf is a layer a large part or the whole of which is to be removed in a later step, it is preferable that the processing of the sacrificial film 158Rf be easy. Therefore, the sacrificial film 158Rf is preferably formed with a substrate temperature lower than that for formation of the inorganic insulating layer 125.

One or both of the sacrificial film 158Rf and the mask film 159Rf may be formed using an organic material. For example, as the organic material, a material that can be dissolved in a solvent chemically stable with respect to at least the uppermost film of the organic compound film 103Rf may be used. Specifically, a material that will be dissolved in water or an alcohol can be suitably used. In forming a film of such a material, it is preferable to apply the material dissolved in a solvent such as water or an alcohol by a wet process and then perform heat treatment for evaporating the solvent. At this time, the heat treatment is preferably performed in a reduced-pressure atmosphere, in which case the solvent can be removed at a low temperature in a short time and thermal damage to the organic compound film 103Rf can be reduced accordingly.

The sacrificial film 158Rf and the mask film 159Rf may be formed using an organic resin such as polyvinyl alcohol (PVA), polyvinyl butyral, polyvinylpyrrolidone, polyethylene glycol, polyglycerin, pullulan, water-soluble cellulose, an alcohol-soluble polyamide resin, or a fluorine resin like perfluoropolymer.

For example, an organic film (e.g., a PVA film) formed by an evaporation method or any of the above wet processes can be used as the sacrificial film 158Rf, and an inorganic film (e.g., a silicon nitride film) formed by a sputtering method can be used as the mask film 159Rf.

Subsequently, a resist mask 190R is formed over the mask film 159Rf as illustrated in FIG. 6C. The resist mask 190R can be formed by application of a photosensitive material (photoresist), light exposure, and development.

The resist mask 190R may be formed using either a positive resist material or a negative resist material.

The resist mask 190R is provided at a position overlapping with the conductive layer 152R. The resist mask 190R is preferably provided also at a position overlapping with the conductive layer 152C. This can inhibit the conductive layer 152C from being damaged during the process of manufacturing the display apparatus. Note that the resist mask 190R is not necessarily provided over the conductive layer 152C. The resist mask 190R is preferably provided to cover the area from the end portion of the organic compound film 103Rf to the end portion of the conductive layer 152C (the end portion closer to the organic compound film 103Rf), as illustrated in the cross-sectional view along the line B1-B2 in FIG. 6C.

Next, as illustrated in FIG. 6D, part of the mask film 159Rf is removed using the resist mask 190R, whereby the mask layer 159R is formed. The mask layer 159R remains over the conductive layers 152R and 152C. After that, the resist mask 190R is removed. Then, part of the sacrificial film 158Rf is removed using the mask layer 159R as a mask (also referred to as a hard mask), whereby the sacrificial layer 158R is formed.

Each of the sacrificial film 158Rf and the mask film 159Rf can be processed by a wet etching method or a dry etching method. The sacrificial film 158Rf and the mask film 159Rf are preferably processed by isotropic etching.

The use of a wet etching method can reduce damage to the organic compound film 103Rf in processing of the sacrificial film 158Rf and the mask film 159Rf, as compared to the case of using a dry etching method. In the case of using a wet etching method, it is preferable to use a developer, an aqueous solution of tetramethylammonium hydroxide (TMAH), dilute hydrofluoric acid, oxalic acid, phosphoric acid, acetic acid, nitric acid, or a chemical solution containing a mixed solution of any of these acids, for example.

Since the organic compound film 103Rf is not exposed in the processing of the mask film 159Rf, the range of choice for a processing method for the mask film 159Rf is wider than that for the sacrificial film 158Rf. Specifically, even in the case where a gas containing oxygen is used as the etching gas in the processing of the mask film 159Rf, deterioration of the organic compound film 103Rf can be inhibited.

In the case of using a dry etching method to process the sacrificial film 158Rf, deterioration of the organic compound film 103Rf can be inhibited by not using a gas containing oxygen as the etching gas. In the case of using a dry etching method, it is preferable to use a gas containing CF₄, C₄F₈, SF₆, CHF₃, Cl₂, H₂O, BCl₃, or a Group 18 element such as He, for example, as the etching gas.

For example, in the case where an aluminum oxide film formed by an ALD method is used as the sacrificial film 158Rf, part of the sacrificial film 158Rf can be removed by a dry etching method using CHF₃ and He or a combination of CHF₃, He, and CH₄. In the case where an In—Ga—Zn oxide film formed by a sputtering method is used as the mask film 159Rf, part of the mask film 159Rf can be removed by a wet etching method using diluted phosphoric acid. Alternatively, part of the mask film 159Rf may be removed by a dry etching method using CH₄ and Ar. Alternatively, part of the mask film 159Rf can be removed by a wet etching method using diluted phosphoric acid. In the case where a tungsten film formed by a sputtering method is used as the mask film 159Rf, part of the mask film 159Rf can be removed by a dry etching method using a combination of SF₆, CF₄, and O₂ or a combination of CF₄, Cl₂, and O₂.

The resist mask 190R can be removed by a method similar to that for the resist mask 191. For example, the resist mask 190R can be removed by ashing using oxygen plasma. Alternatively, an oxygen gas and any of CF₄, C₄F₈, SF₆, CHF₃, Cl₂, H₂O, BCl₃, and a Group 18 element such as He may be used. Alternatively, the resist mask 190R may be removed by wet etching. At this time, the sacrificial film 158Rf is positioned on the outermost surface, and the organic compound film 103Rf is not exposed; thus, the organic compound film 103Rf can be inhibited from being damaged in the step of removing the resist mask 190R. In addition, the range of choice for the method for removing the resist mask 190R can be widened.

Next, as illustrated in FIG. 6D, the organic compound film 103Rf is processed, so that the organic compound layer 103R is formed. For example, part of the organic compound film 103Rf is removed using the mask layer 159R and the sacrificial layer 158R as a hard mask, whereby the organic compound layer 103R is formed.

Accordingly, as illustrated in FIG. 6D, the stacked-layer structure of the organic compound layer 103R, the sacrificial layer 158R, and the mask layer 159R remains over the conductive layer 152R. The conductive layers 152G and 152B are exposed.

In the example illustrated in FIG. 6D, the end portion of the organic compound layer 103R is positioned outward from the end portion of the conductive layer 152R. Such a structure can increase the aperture ratio of the pixel. Although not illustrated in FIG. 6D, by the above etching treatment, a recessed portion may be formed in the insulating layer 175 in a region not overlapping with the organic compound layer 103R.

Since the organic compound layer 103R covers the top surface and the side surface of the conductive layer 152R, the subsequent steps can be performed without exposure of the conductive layer 152R. If the end portion of the conductive layer 152R is exposed, there is a possibility that corrosion occurs in an etching step, for example. A product generated by corrosion of the conductive layer 152R may be unstable, and for example, might be dissolved in a solution when wet etching is performed and might be scattered in an atmosphere when dry etching is performed. By dissolution of the product in a solution or scattering of the product in the atmosphere, the product might be attached to a subject surface and the side surface of the organic compound layer 103R, for example, which might adversely affect the characteristics of the light-emitting device or form a leak path between a plurality of light-emitting devices. In a region where the end portion of the conductive layer 152R is exposed, adhesion between layers in contact with each other might be lowered, which might be likely to cause peeling of the organic compound layer 103R or the conductive layer 152R.

Accordingly, the structure where the organic compound layer 103R covers the top surface and the side surface of the conductive layer 152R can improve the yield and characteristics of the light-emitting device, for example.

As described above, the resist mask 190R is preferably provided to cover the area from the end portion of the organic compound layer 103R to the end portion of the conductive layer 152C (the end portion closer to the organic compound layer 103R) in the cross section B1-B2. Thus, as illustrated in FIG. 6D, the sacrificial layer 158R and the mask layer 159R are provided to cover the area from the end portion of the organic compound layer 103R to the end portion of the conductive layer 152C (the end portion closer to the organic compound layer 103R) in the cross section B1-B2. Hence, the insulating layer 175 can be inhibited from being exposed in the cross section B1-B2, for example. This can prevent the insulating layers 175, 174, and 173 from being partly removed by etching and thus prevent the conductive layer 179 from being exposed. Accordingly, the conductive layer 179 can be inhibited from being unintentionally electrically connected to another conductive layer. For example, a short circuit between the conductive layer 179 and a common electrode 155 formed in a later step can be inhibited.

The organic compound film 103Rf is preferably processed by anisotropic etching. Anisotropic dry etching is particularly preferable. Alternatively, wet etching may be used.

In the case of using a dry etching method, deterioration of the organic compound film 103Rf can be inhibited by not using a gas containing oxygen as the etching gas.

A gas containing oxygen may be used as the etching gas. When the etching gas contains oxygen, the etching rate can be increased. Therefore, the etching can be performed under a low-power condition while an adequately high etching rate is maintained. Accordingly, damage to the organic compound film 103Rf can be reduced. Furthermore, a defect such as attachment of a reaction product generated in the etching can be inhibited.

In the case of using a dry etching method, it is preferable to use a gas containing at least one of H₂, CF₄, C₄F₈, SF₆, CHF₃, Cl₂, H₂O, BCl₃, and a Group 18 element such as He and Ar as the etching gas, for example. Alternatively, a gas containing oxygen and at least one of the above is preferably used as the etching gas. Alternatively, an oxygen gas may be used as the etching gas. Specifically, for example, a gas containing H₂ and Ar or a gas containing CF₄ and He can be used as the etching gas. As another example, a gas containing CF₄, He, and oxygen can be used as the etching gas. As another example, a gas containing H₂ and Ar and a gas containing oxygen can be used as the etching gas.

As described above, in one embodiment of the present invention, the mask layer 159R is formed in the following manner: the resist mask 190R is formed over the mask film 159Rf and part of the mask film 159Rf is removed using the resist mask 190R. After that, part of the organic compound film 103Rf is removed using the mask layer 159R as a hard mask, so that the organic compound layer 103R is formed. In other words, the organic compound layer 103R is formed by processing the organic compound film 103Rf by a lithography method. Note that part of the organic compound film 103Rf may be removed using the resist mask 190R. Then, the resist mask 190R may be removed.

Next, hydrophobization treatment for the conductive layer 152G, for example, is preferably performed. At the time of processing the organic compound film 103Rf, a surface of the conductive layer 152G changes to have hydrophilic properties in some cases, for example. The hydrophobization treatment for the conductive layer 152G, for example, can increase the adhesion between the conductive layer 152G and a layer to be formed in a later step (which is the organic compound layer 103G here) and inhibit film peeling. Note that the hydrophobization treatment is not necessarily performed.

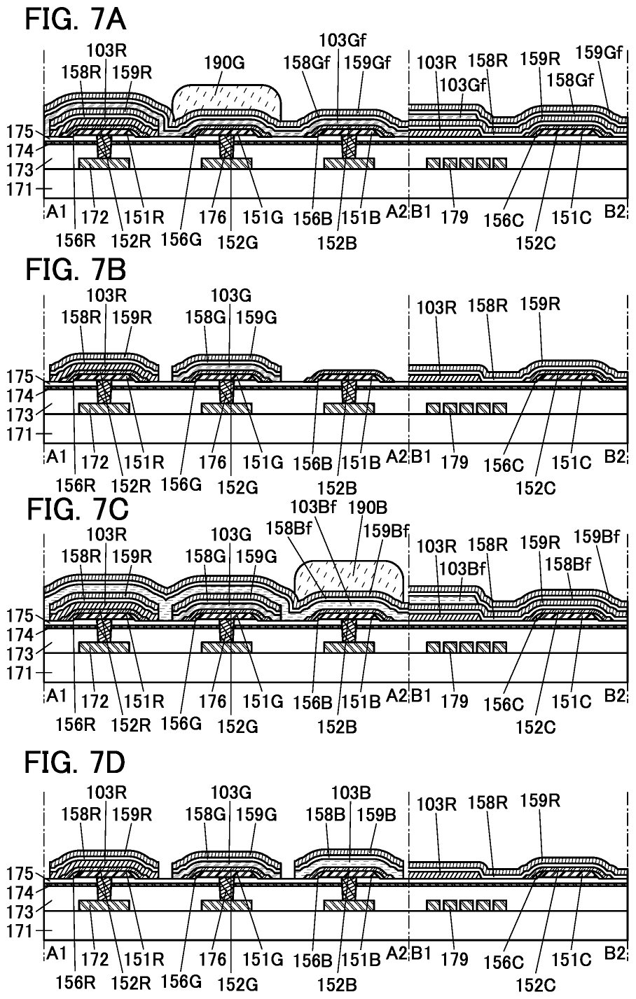

Next, as illustrated in FIG. 7A, an organic compound film 103Gf to be the organic compound layer 103G is formed over the conductive layer 152G, the conductive layer 152B, the mask layer 159R, and the insulating layer 175.

The organic compound film 103Gf can be formed by a method similar to that for forming the organic compound film 103Rf. The organic compound film 103Gf can have a structure similar to that of the organic compound film 103Rf.

Then, as illustrated in FIG. 7A, a sacrificial film 158Gf to be a sacrificial layer 158G and a mask film 159Gf to be a mask layer 159G are sequentially formed over the organic compound film 103Gf and the mask layer 159R. After that, a resist mask 190G is formed. The materials and the formation methods of the sacrificial film 158Gf and the mask film 159Gf are similar to those of the sacrificial film 158Rf and the mask film 159Rf. The material and the formation method of the resist mask 190G are similar to those of the resist mask 190R.

The resist mask 190G is provided at a position overlapping with the conductive layer 152G.

Subsequently, as illustrated in FIG. 7B, part of the mask film 159Gf is removed using the resist mask 190G, whereby the mask layer 159G is formed. The mask layer 159G remains over the conductive layer 152G. After that, the resist mask 190G is removed. Then, part of the sacrificial film 158Gf is removed using the mask layer 159G as a mask, whereby the sacrificial layer 158G is formed. Next, the organic compound film 103Gf is processed to form the organic compound layer 103G. For example, part of the organic compound film 103Gf is removed using the mask layer 159G and the sacrificial layer 158G as a hard mask to form the organic compound layer 103G.

Accordingly, as illustrated in FIG. 7B, the stacked-layer structure of the organic compound layer 103G, the sacrificial layer 158G, and the mask layer 159G remains over the conductive layer 152G. The mask layer 159R and the conductive layer 152B are exposed.

Next, hydrophobization treatment for the conductive layer 152B, for example, is preferably performed. At the time of processing the organic compound film 103Gf, a surface of the conductive layer 152B changes to have hydrophilic properties in some cases, for example. The hydrophobization treatment for the conductive layer 152B, for example, can increase the adhesion between the conductive layer 152B and a layer to be formed in a later step (which is the organic compound layer 103B here) and inhibit film peeling. Note that the hydrophobization treatment is not necessarily performed.

Next, as illustrated in FIG. 7C, an organic compound film 103Bf to be the organic compound layer 103B is formed over the conductive layer 152B, the mask layer 159R, the mask layer 159G, and the insulating layer 175.

The organic compound film 103Bf can be formed by a method similar to that for forming the organic compound film 103Rf. The organic compound film 103Bf can have a structure similar to that of the organic compound film 103Rf.

Then, as illustrated in FIG. 7C, a sacrificial film 158Bf to be a sacrificial layer 158B and a mask film 159Bf to be a mask layer 159B are sequentially formed over the organic compound film 103Bf and the mask layer 159R. After that, a resist mask 190B is formed. The materials and the formation methods of the sacrificial film 158Bf and the mask film 159Bf are similar to those of the sacrificial film 158Rf and the mask film 159Rf. The material and the formation method of the resist mask 190B are similar to those of the resist mask 190R.

The resist mask 190B is provided at a position overlapping with the conductive layer 152B.

Subsequently, as illustrated in FIG. 7D, part of the mask film 159Bf is removed using the resist mask 190B, whereby the mask layer 159B is formed. The mask layer 159B remains over the conductive layer 152B. After that, the resist mask 190B is removed. Then, part of the sacrificial film 158Bf is removed using the mask layer 159B as a mask, whereby the sacrificial layer 158B is formed. Next, the organic compound film 103Bf is processed to form the organic compound layer 103B. For example, part of the organic compound film 103Bf is removed using the mask layer 159B and the sacrificial layer 158B as a hard mask to form the organic compound layer 103B.

Accordingly, as illustrated in FIG. 7D, the stacked-layer structure of the organic compound layer 103B, the sacrificial layer 158B, and the mask layer 159B remains over the conductive layer 152B. The mask layers 159R and 159G are exposed.

Note that the side surfaces of the organic compound layers 103R, 103G, and 103B are preferably perpendicular or substantially perpendicular to their formation surfaces. For example, the angle between the formation surfaces and these side surfaces is preferably greater than or equal to 60° and less than or equal to 90°.

The distance between two adjacent layers among the organic compound layers 103R, 103G, and 103B, which are formed by a lithography method as described above, can be shortened to less than or equal to 8 μm, less than or equal to 5 μm, less than or equal to 3 μm, less than or equal to 2 μm, or less than or equal to 1 μm. Here, the distance can be specified, for example, by a distance between opposite end portions of two adjacent layers among the organic compound layers 103R, 103G, and 103B. Shortening the distance between the island-shaped organic compound layers can provide a display apparatus having high resolution and a high aperture ratio. In addition, the distance between the first electrodes of adjacent light-emitting devices can also be shortened to be, for example, less than or equal to 10 μm, less than or equal to 8 μm, less than or equal to 5 μm, less than or equal to 3 μm, or less than or equal to 2 μm. Note that the distance between the first electrodes of adjacent light-emitting devices is preferably greater than or equal to 2 μm and less than or equal to 5 μm.

Next, as illustrated in FIG. 8A, the mask layers 159R, 159G, and 159B are preferably removed. The sacrificial layers 158R, 158G, and 158B and the mask layers 159R, 159G, and 159B remain in the display apparatus in some cases depending on the subsequent steps. Removing the mask layers 159R, 159G, and 159B at this stage can inhibit the mask layers 159R, 159G, and 159B from being left in the display apparatus. For example, in the case where a conductive material is used for the mask layers 159R, 159G, and 159B, removing the mask layers 159R, 159G, and 159B in advance can inhibit generation of a leakage current, formation of a capacitor, and the like due to the remaining mask layers 159R, 159G, and 159B.

This embodiment shows an example where the mask layers 159R, 159G, and 159B are removed; however, it is possible that the mask layers 159R, 159G, and 159B are not removed. For example, in the case where the mask layers 159R, 159G, and 159B contain the above-described material having a property of blocking ultraviolet rays, the procedure preferably proceeds to the next step without removing the mask layers 159R, 159G, and 159B, in which case the organic compound layer can be protected from ultraviolet rays.

The step of removing the mask layers can be performed by a method similar to that for the step of processing the mask layers. Specifically, by using a wet etching method, damage applied to the organic compound layers 103R, 103G, and 103B at the time of removing the mask layers can be reduced as compared to the case of using a dry etching method.

The mask layers may be removed by being dissolved in a solvent such as water or an alcohol. Examples of an alcohol include ethyl alcohol, methyl alcohol, isopropyl alcohol (IPA), and glycerin.

After the mask layers are removed, drying treatment may be performed in order to remove water included in the organic compound layers 103R, 103G, and 103B and water adsorbed onto the surfaces of the organic compound layers 103R, 103G, and 103B. For example, heat treatment in an inert gas atmosphere or a reduced-pressure atmosphere can be performed. The heat treatment can be performed at a substrate temperature higher than or equal to 50° C. and lower than or equal to 200° C., preferably higher than or equal to 60° C. and lower than or equal to 150° C., further preferably higher than or equal to 70° C. and lower than or equal to 120° C. The heat treatment is preferably performed in a reduced-pressure atmosphere, in which case drying at a lower temperature is possible.

Next, as illustrated in FIG. 8B, the inorganic insulating film 125f to be the inorganic insulating layer 125 is formed to cover the organic compound layers 103R, 103G, and 103B and the sacrificial layers 158R, 158G, and 158B.

As described later, an insulating film to be the insulating layer 127 is formed in contact with the top surface of the inorganic insulating film 125f. Therefore, the top surface of the inorganic insulating film 125f preferably has a high affinity for the material used for the insulating film (e.g., a photosensitive resin composition containing an acrylic resin). To improve the affinity, surface treatment is preferably performed so that the top surface of the inorganic insulating film 125f is made hydrophobic or its hydrophobic properties are improved. For example, it is preferable to perform the treatment using a silylation agent such as hexamethyldisilazane (HMIDS). By making the top surface of the inorganic insulating film 125f hydrophobic in such a manner, the above insulating film 127f can be formed with favorable adhesion. Note that the above-described hydrophobization treatment may be performed as the surface treatment.

Then, as illustrated in FIG. 8C, an insulating film 127f to be the insulating layer 127 is formed over the inorganic insulating film 125f.

The inorganic insulating film 125f and the insulating film 127f are preferably formed by a formation method by which the organic compound layers 103R, 103G, and 103B are less damaged. The inorganic insulating film 125f, which is formed in contact with the side surfaces of the organic compound layers 103R, 103G, and 103B, is particularly preferably formed by a formation method that causes less damage to the organic compound layers 103R, 103G, and 103B than the formation method of the insulating film 127f.

Each of the inorganic insulating film 125f and the insulating film 127f is formed at a temperature lower than the upper temperature limits of the organic compound layers 103R, 103G, and 103B. When the inorganic insulating film 125f is formed at a high substrate temperature, the formed inorganic insulating film 125f, even with a small thickness, can have a low impurity concentration and a high barrier property against at least one of water and oxygen.

The substrate temperature at the time of forming the inorganic insulating film 125f and the insulating film 127f is preferably higher than or equal to 60° C., higher than or equal to 80° C., higher than or equal to 100° C., or higher than or equal to 120° C. and lower than or equal to 200° C., lower than or equal to 180° C., lower than or equal to 160° C., lower than or equal to 150° C., or lower than or equal to 140° C.

As the inorganic insulating film 125f, an insulating film having a thickness greater than or equal to 3 nm, greater than or equal to 5 nm, or greater than or equal to 10 nm and less than or equal to 200 nm, less than or equal to 150 nm, less than or equal to 100 nm, or less than or equal to 50 nm is preferably formed in the above-described range of the substrate temperature.

The inorganic insulating film 125f is preferably formed by an ALD method, for example. An ALD method is preferably used, in which case deposition damage is reduced and a film with good coverage can be formed. As the inorganic insulating film 125f, an aluminum oxide film is preferably formed by an ALD method, for example.

Alternatively, the inorganic insulating film 125f may be formed by a sputtering method, a CVD method, or a PECVD method, each of which has a higher deposition rate than an ALD method. In that case, a highly reliable display apparatus can be manufactured with high productivity.

The insulating film 127f is preferably formed by the aforementioned wet process. The insulating film 127f is preferably formed by spin coating using a photosensitive material, for example, and specifically preferably formed using a photosensitive resin composition containing an acrylic resin.

The insulating film 127f is preferably formed using a resin composition containing a polymer, an acid-generating agent, and a solvent, for example. The polymer is formed using one or more kinds of monomers and has a structure where one or more kinds of structural units (also referred to as building blocks) are repeated regularly or irregularly. As the acid-generating agent, one or both of a compound that generates an acid by light irradiation and a compound that generates an acid by heating can be used. The resin composition may also include one or more of a photosensitizing agent, a sensitizer, a catalyst, an adhesive aid, a surface-active agent, and an antioxidant.

Heat treatment (also referred to as prebaking) is preferably performed after the insulating film 127f is formed. The heat treatment is performed at a temperature lower than the upper temperature limits of the organic compound layers 103R, 103G, and 103B. The substrate temperature in the heat treatment is preferably higher than or equal to 50° C. and lower than or equal to 200° C., further preferably higher than or equal to 60° C. and lower than or equal to 150° C., still further preferably higher than or equal to 70° C. and lower than or equal to 120° C. Accordingly, the solvent contained in the insulating film 127f can be removed.

Then, part of the insulating film 127f is exposed to visible light or ultraviolet rays. Here, when a positive photosensitive resin composition containing an acrylic resin is used for the insulating film 127f, a region where the insulating layer 127 is not formed in a later step is irradiated with visible light or ultraviolet rays. The insulating layer 127 is formed in regions that are interposed between any two of the conductive layers 152R, 152G, and 152B and around the conductive layer 152C. Thus, the top surfaces of the conductive layers 152R, 152G, 152B, and 152C are irradiated with visible light or ultraviolet rays. Note that when a negative photosensitive material is used for the insulating film 127f, the region where the insulating layer 127 is to be formed is irradiated with visible light or ultraviolet rays.

The width of the insulating layer 127 that is to be formed later can be controlled in accordance with the exposed region of the insulating film 127f. In this embodiment, processing is performed such that the insulating layer 127 includes a portion overlapping with the top surface of the conductive layer 151.

Light used for exposure preferably includes the i-line (wavelength: 365 nm). Furthermore, light used for exposure may include at least one of the g-line (wavelength: 436 nm) and the h-line (wavelength: 405 nm).

Here, when a barrier insulating layer against oxygen (e.g., an aluminum oxide film) is provided as one or both of the sacrificial layer 158 (the sacrificial layers 158R, 158G, and 158B) and the inorganic insulating film 125f, diffusion of oxygen into the organic compound layers 103R, 103G, and 103B can be inhibited. When the organic compound layer is irradiated with light (visible light or ultraviolet rays), the organic compound contained in the organic compound layer is brought into an excited state and a reaction between the organic compound and oxygen in the atmosphere is promoted in some cases. Specifically, when the organic compound layer is irradiated with light (visible light or ultraviolet rays) in an atmosphere containing oxygen, oxygen might be bonded to the organic compound contained in the organic compound layer. By providing the sacrificial layer 158 and the inorganic insulating film 125f over the island-shaped organic compound layer, bonding of oxygen in the atmosphere to the organic compound contained in the organic compound layer can be inhibited.

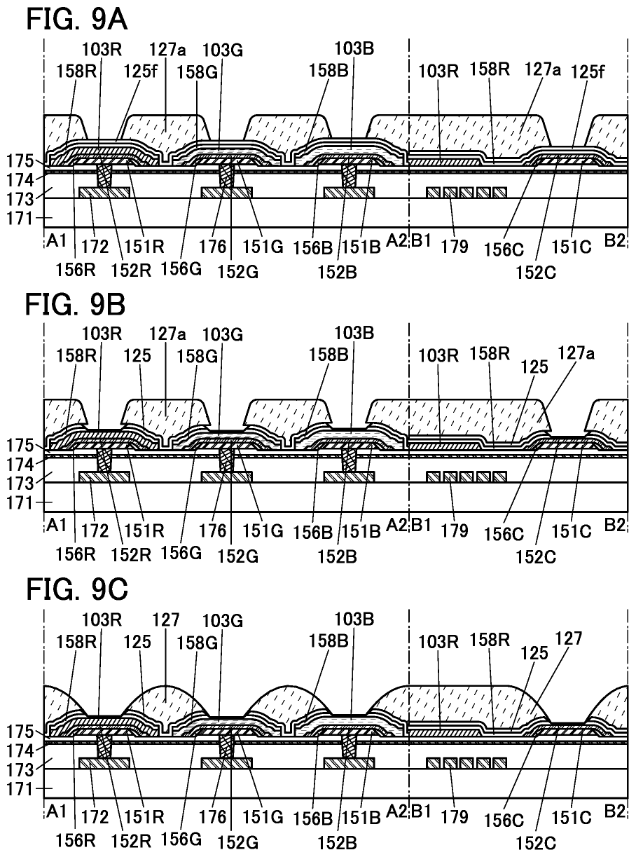

Next, as illustrated in FIG. 9A, development is performed to remove the exposed region of the insulating film 127f, whereby an insulating layer 127a is formed. The insulating layer 127a is formed in regions that are interposed between any two of the conductive layers 152R, 152G, and 152B and a region surrounding the conductive layer 152C. Here, when an acrylic resin is used for the insulating film 127f, an alkaline solution, such as TMAH, can be used as a developer.

Then, a residue (scum) due to the development may be removed. For example, the residue can be removed by ashing using oxygen plasma.

Etching may be performed so that the surface level of the insulating layer 127a is adjusted. The insulating layer 127a maybe processed by ashing using oxygen plasma, for example. In the case where a non-photosensitive material is used for the insulating film 127f, the surface level of the insulating film 127f can be adjusted by the ashing, for example.

Next, as illustrated in FIG. 9B, etching treatment is performed using the insulating layer 127a as a mask to remove part of the inorganic insulating film 125f and reduce the thickness of part of the sacrificial layers 158R, 158G, and 158B. Thus, the inorganic insulating layer 125 is formed under the insulating layer 127a. Moreover, the surfaces of the thin portions in the sacrificial layers 158R, 158G, and 158B are exposed. Note that the etching treatment using the insulating layer 127a as a mask may be hereinafter referred to as first etching treatment.

The first etching treatment can be performed by dry etching or wet etching. Note that the inorganic insulating film 125f is preferably formed using a material similar to that of the sacrificial layers 158R, 158G, and 158B, in which case the first etching treatment can be performed concurrently.

By etching using the insulating layer 127a with a tapered side surface as a mask, the side surface of the inorganic insulating layer 125 and upper end portions of the side surfaces of the sacrificial layers 158R, 158G, and 158B can be made to have a tapered shape relatively easily.

In the case of performing dry etching, a chlorine-based gas is preferably used. As the chlorine-based gas, one of Cl₂, BCl₃, SiCl₄, CCl₄, and the like or a mixture of two or more of them can be used. Moreover, one of an oxygen gas, a hydrogen gas, a helium gas, an argon gas, and the like or a mixture of two or more of them can be added as appropriate to the chlorine-based gas. By the dry etching, the thin regions of the sacrificial layers 158R, 158G, and 158B can be formed with favorable in-plane uniformity.

As a dry etching apparatus, a dry etching apparatus including a high-density plasma source can be used. As the dry etching apparatus including a high-density plasma source, an inductively coupled plasma (ICP) etching apparatus can be used, for example. Alternatively, a capacitively coupled plasma (CCP) etching apparatus including parallel plate electrodes can be used. The capacitively coupled plasma etching apparatus including parallel plate electrodes may have a structure where a high-frequency voltage is applied to one of the parallel plate electrodes. Alternatively, the capacitively coupled plasma etching apparatus may have a structure where different high-frequency voltages are applied to one of the parallel-plate electrodes. Alternatively, the capacitively coupled plasma etching apparatus may have a structure where high-frequency voltages with the same frequency are applied to the parallel-plate electrodes. Alternatively, the capacitively coupled plasma etching apparatus may have a structure where high-frequency voltages with different frequencies are applied to the parallel-plate electrodes.

In the case of performing dry etching, a by-product or the like generated by the dry etching might be deposited on the top surface and the side surface of the insulating layer 127a, for example. Accordingly, a component of the etching gas, a component of the inorganic insulating film 125f, a component of the sacrificial layers 158R, 158G, and 158B, and the like might be included in the insulating layer 127 in the completed display apparatus.

The first etching treatment is preferably performed by wet etching. The use of a wet etching method can reduce damage to the organic compound layers 103R, 103G, and 103B, as compared to the case of using a dry etching method. For example, the wet etching can be performed using an alkaline solution. For instance, TMAH, which is an alkaline solution, can be used for the wet etching of an aluminum oxide film. In this case, puddle wet etching can be performed. Note that the inorganic insulating film 125f is preferably formed using a material similar to that of the sacrificial layers 158R, 158G, and 158B, in which case the above etching treatment can be performed concurrently.

The sacrificial layers 158R, 158G, and 158B are not removed completely by the first etching treatment, and the etching treatment is stopped when the thicknesses of the sacrificial layers 158R, 158G, and 158B are reduced. The corresponding sacrificial layers 158R, 158G, and 158B remain over the organic compound layers 103R, 103G, and 103B in this manner, whereby the organic compound layers 103R, 103G, and 103B can be prevented from being damaged by treatment in a later step.

Next, light exposure is preferably performed on the entire substrate so that the insulating layer 127a is irradiated with visible light or ultraviolet rays. The energy density for the light exposure is preferably greater than 0 mJ/cm² and less than or equal to 800 mJ/cm², further preferably greater than 0 mJ/cm² and less than or equal to 500 mJ/cm². Performing such light exposure after the development can sometimes increase the degree of transparency of the insulating layer 127a. In addition, it is sometimes possible to lower the substrate temperature required for subsequent heat treatment for changing the shape of the insulating layer 127a to a tapered shape.

Here, when a barrier insulating layer against oxygen (e.g., an aluminum oxide film) exists as each of the sacrificial layers 158R, 158G, and 158B, diffusion of oxygen into the organic compound layers 103R, 103G, and 103B can be inhibited. When the organic compound layer is irradiated with light (visible light or ultraviolet rays), the organic compound contained in the organic compound layer is brought into an excited state and a reaction between the organic compound and oxygen in the atmosphere is promoted in some cases. Specifically, when the organic compound layer is irradiated with light (visible light or ultraviolet rays) in an atmosphere containing oxygen, oxygen might be bonded to the organic compound contained in the organic compound layer. By providing the sacrificial layers 158R, 158G, and 158B over the island-shaped organic compound layer, bonding of oxygen in the atmosphere to the organic compound contained in the organic compound layer can be inhibited.

Then, heat treatment (also referred to as post-baking) is performed. The heat treatment can change the insulating layer 127a into the insulating layer 127 having a tapered side surface (FIG. 9C). The heat treatment is performed at a temperature lower than the upper temperature limit of the organic compound layer. The heat treatment can be performed at a substrate temperature higher than or equal to 50° C. and lower than or equal to 200° C., preferably higher than or equal to 60° C. and lower than or equal to 150° C., further preferably higher than or equal to 70° C. and lower than or equal to 130° C. The heating atmosphere maybe an air atmosphere or an inert gas atmosphere. Moreover, the heating atmosphere may be an atmospheric-pressure atmosphere or a reduced-pressure atmosphere. The substrate temperature in the heat treatment of this step is preferably higher than that in the heat treatment (prebaking) after the formation of the insulating film 127f. Accordingly, adhesion between the insulating layer 127 and the inorganic insulating layer 125 can be improved, and corrosion resistance of the insulating layer 127 can be increased.

When the sacrificial layers 158R, 158G, and 158B are not completely removed by the first etching treatment and the thinned sacrificial layers 158R, 158G, and 158B are left, the organic compound layers 103R, 103G, and 103B can be prevented from being damaged and deteriorating in the heat treatment. This increases the reliability of the light-emitting device.

Note that the side surface of the insulating layer 127 may have a concave shape depending on the material of the insulating layer 127 and the temperature, time, and atmosphere of the post-baking. For example, when the temperature of the post-baking is higher or the duration of the post-baking is longer, the insulating layer 127 is more likely to change in shape and thus is more likely to have a concave shape.

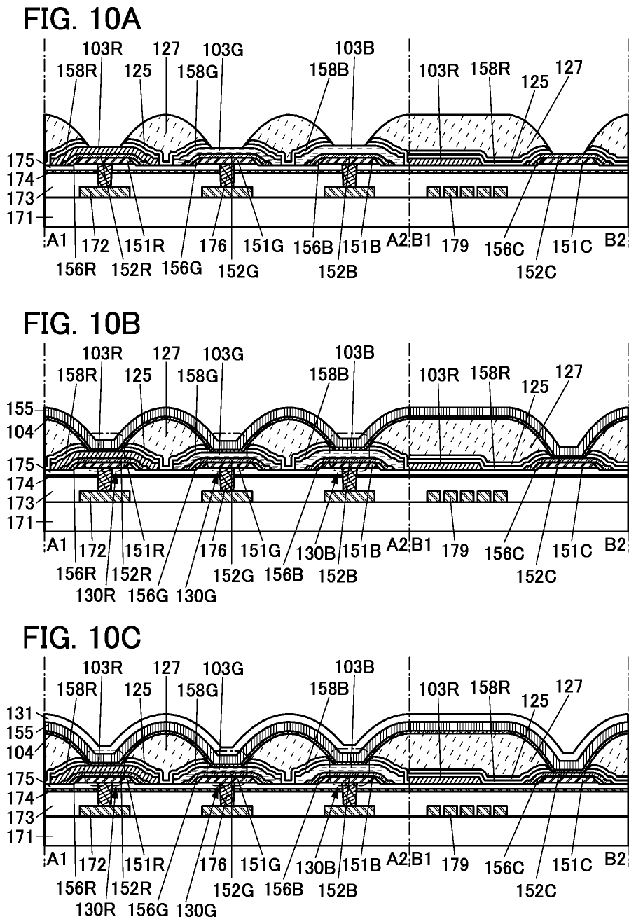

Next, as illustrated in FIG. 10A, etching treatment is performed using the insulating layer 127 as a mask to remove part of the sacrificial layers 158R, 158G, and 158B. Note that part of the inorganic insulating layer 125 is also removed in some cases. Thus, openings are formed in the sacrificial layers 158R, 158G, and 158B, and the top surfaces of the organic compound layers 103R, 103G, and 103B and the conductive layer 152C are exposed. Note that the etching treatment using the insulating layer 127 as a mask may be hereinafter referred to as second etching treatment.

The end portion of the inorganic insulating layer 125 is covered with the insulating layer 127. FIG. 10A illustrates an example where part of the end portion of the sacrificial layer 158G (specifically a tapered portion formed by the first etching treatment) is covered with the insulating layer 127 and a tapered portion formed by the second etching treatment is exposed.

If the first etching treatment is not performed and the inorganic insulating layer 125 and the mask layer are collectively etched after the post-baking, the inorganic insulating layer 125 and the mask layer under the end portion of the insulating layer 127 may disappear because of side etching and a void may be formed. The void causes unevenness on the formation surface of the common electrode 155, so that step disconnection is more likely to be caused in the common electrode 155. Even when a void is formed owing to side etching of the inorganic insulating layer 125 and the mask layer by the first etching treatment, the post-baking performed subsequently can make the insulating layer 127 fill the void. After that, the thinned mask layer is etched by the second etching treatment; thus, the amount of side etching decreases, a void is less likely to be formed, and even if a void is formed, it can be extremely small. Consequently, the formation surface of the common electrode 155 can be made flatter.

Note that the insulating layer 127 may cover the entire end portion of the sacrificial layer 158G. For example, the end portion of the insulating layer 127 may droop to cover the end portion of the sacrificial layer 158G. As another example, the end portion of the insulating layer 127 may be in contact with the top surface of at least one of the organic compound layers 103R, 103G, and 103B. As described above, when light exposure is not performed on the insulating layer 127a after the development, the shape of the insulating layer 127 may be likely to change.

The second etching treatment is performed by wet etching. The use of a wet etching method can reduce damage to the organic compound layers 103R, 103G, and 103B, as compared to the case of using a dry etching method. The wet etching can be performed using an alkaline solution such as TMAH, for example.

Meanwhile, in the case where the second etching treatment is performed by a wet etching method and gaps due to, for example, poor adhesion between the organic compound layer 103 and another layer exist at the interface between the organic compound layer 103 and the sacrificial layer 158, the interface between the organic compound layer 103 and the inorganic insulating layer 125, and the interface between the organic compound layer 103 and the insulating layer 175, the chemical solution used in the second etching treatment sometimes enters the gaps to come into contact with the pixel electrode. Here, when the chemical solution comes into contact with both the conductive layer 151 and the conductive layer 152, one of the conductive layers 151 and 152 that has a lower spontaneous potential than the other suffers from galvanic corrosion in some cases. For example, when the conductive layer 151 is formed using aluminum and the conductive layer 152 is formed using indium tin oxide, the conductive layer 152 sometimes corrodes. As a result, the yield of the display apparatus decreases in some cases. Moreover, the reliability of the display apparatus decreases in some cases.

The conductive layer 152, which covers the top and side surfaces of the conductive layer 151 as described above, can prevent the chemical solution from coming into contact with the conductive layer 151 in the second etching treatment even when gaps exist at the interface between the organic compound layer 103 and the sacrificial layer 158, the interface between the organic compound layer 103 and the inorganic insulating layer 125, and the interface between the organic compound layer 103 and the insulating layer 175. Thus, corrosion of the pixel electrode, e.g., the conductive layer 152, can be prevented.

Furthermore, when the insulating layer 156 is formed to include a region overlapping with the side surface of the conductive layer 151 and the conductive layer 152 is formed to cover the conductive layer 151 and the insulating layer 156, the step disconnection can be prevented, whereby the chemical solution can be prevented from coming into contact with the conductive layer 151 in the second etching treatment, for example. Thus, corrosion of the pixel electrode, e.g., the conductive layer 152, can be prevented.

As described above, by providing the insulating layer 127, the inorganic insulating layer 125, and the sacrificial layers 158R, 158G, and 158B, poor connection due to a disconnected portion and an increase in electrical resistance due to a locally thinned portion can be inhibited from occurring in the common electrode 155 between the light-emitting devices. Thus, the display apparatus of one embodiment of the present invention can have improved display quality.

Heat treatment is performed after the organic compound layers 103R, 103G, and 103B are partly exposed. By the heat treatment, water included in the organic compound layers and water adsorbed onto the surfaces of the organic compound layers, for example, can be removed. The shape of the insulating layer 127 may be changed by the heat treatment. Specifically, the insulating layer 127 may be widened to cover at least one of the end portion of the inorganic insulating layer 125, the end portions of the sacrificial layers 158R, 158G, and 158B, and the top surfaces of the organic compound layers 103R, 103G, and 103B.

If the temperature of the heat treatment is too low, water included in the organic compound layers and water adsorbed onto the surface of the organic compound layers, for example, cannot be sufficiently removed. If the temperature of the heat treatment is too high, the organic compound layer 103 might deteriorate and the insulating layer 127 might change in shape excessively. Therefore, the temperature of the heat treatment is preferably higher than the temperature at which water is released from the organic compound layer 103 and lower than the glass transition temperature of an organic compound included in the organic compound layer 103, further preferably lower than the glass transition temperature of an organic compound included in the upper surface of the organic compound layer 103. Specifically, the substrate temperature is higher than or equal to 80° C. and lower than or equal to 130° C., preferably higher than or equal to 90° C. and lower than or equal to 120° C., further preferably higher than or equal to 100° C. and lower than or equal to 120° C., further preferably higher than or equal to 100° C. and lower than or equal to 110° C. The heating atmosphere may be an air atmosphere or an inert gas atmosphere. Although the heating atmosphere maybe an atmospheric-pressure atmosphere or a reduced-pressure atmosphere, a reduced-pressure atmosphere is preferred to prevent re-adsorption of water released from the organic compound layer 103.

By the heat treatment, water included in the organic compound layers and water adsorbed onto the surface of the organic compound layers, for example, can be sufficiently removed without deterioration of the organic compound layers 103R, 103G, and 103B and an excessive change in the shape of the insulating layer 127. Thus, degradation of the characteristics of the light-emitting device can be prevented.

Next, as illustrated in FIG. 10B, the common layer 104 and the common electrode 155 are formed over the organic compound layers 103R, 103G, and 103B, the conductive layer 152C, and the insulating layer 127. The common layer 104 and common electrode 155 can be formed by a sputtering method, a vacuum evaporation method, or the like. The common layer 104 may be formed by an evaporation method while the common electrode 155 may be formed by a sputtering method.

Next, as illustrated in FIG. 10C, the protective layer 131 is formed over the common electrode 155. The protective layer 131 can be formed by a vacuum evaporation method, a sputtering method, a CVD method, an ALD method, or the like.

Then, the substrate 120 is attached to the protective layer 131 using the resin layer 122, whereby the display apparatus can be manufactured. In the method for manufacturing the display apparatus of one embodiment of the present invention, the insulating layer 156 is formed to include a region overlapping with the side surface of the conductive layer 151 and the conductive layer 152 is formed to cover the conductive layer 151 and the insulating layer 156 as described above. This can increase the yield of the display apparatus and inhibit generation of defects.

As described above, in the method for manufacturing the display apparatus of one embodiment of the present invention, the island-shaped organic compound layers 103R, 103G, and 103B are formed not by using a fine metal mask but by processing a film formed on the entire surface; thus, the island-shaped layers can be formed to have a uniform thickness. Consequently, a high-resolution display apparatus or a display apparatus with a high aperture ratio can be obtained. Furthermore, even when the resolution or the aperture ratio is high and the distance between the subpixels is extremely short, the organic compound layers 103R, 103G, and 103B can be inhibited from being in contact with each other in the adjacent subpixels. As a result, generation of a leakage current between the subpixels can be inhibited. This can prevent crosstalk, so that a display apparatus with extremely high contrast can be obtained. Moreover, even a display apparatus including tandem light-emitting devices formed by a lithography method can have favorable characteristics.

### Embodiment 4

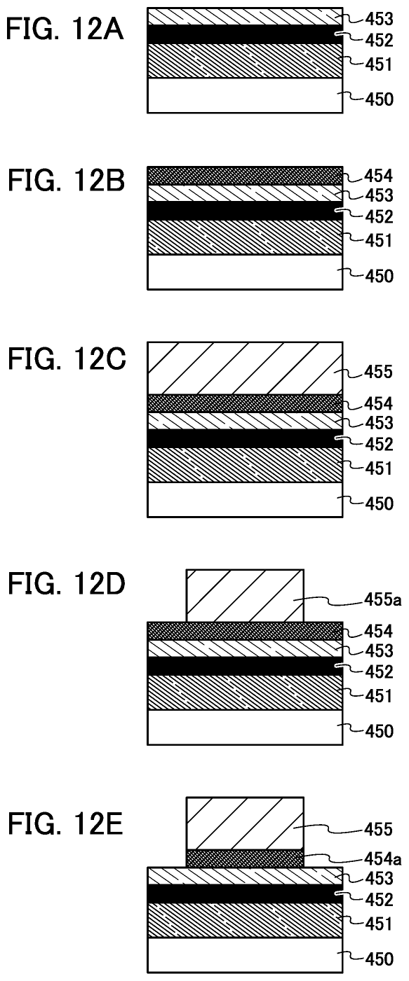

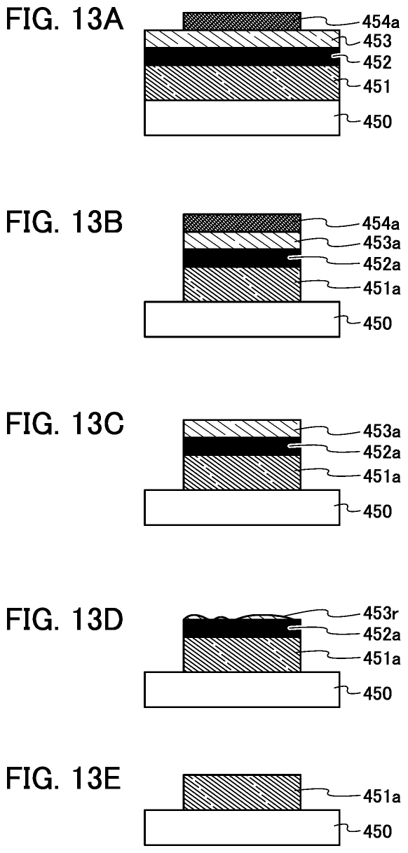

In this embodiment, a processing method of an organic compound film with the use of the organic compound of one embodiment of the present invention for an organic mask film is described with reference to FIGS. 12A to 12E and FIGS. 13A to 13E.

It is necessary to solve many problems in order that the shape of the organic compound film can be processed by a lithography method. Examples of the problems include an effect of exposure of the organic compound film to the air, an effect of light irradiation when a photosensitive resin is exposed to light, an effect of developer when the exposed photosensitive resin is developed, and an effect of formation of a metal film that is sometimes formed to reduce the effect of the developer.

These effects are regarded as problems because an organic compound film itself is removed or damage to a surface of the organic compound film significantly degrades the characteristics of a device manufactured later, for example.

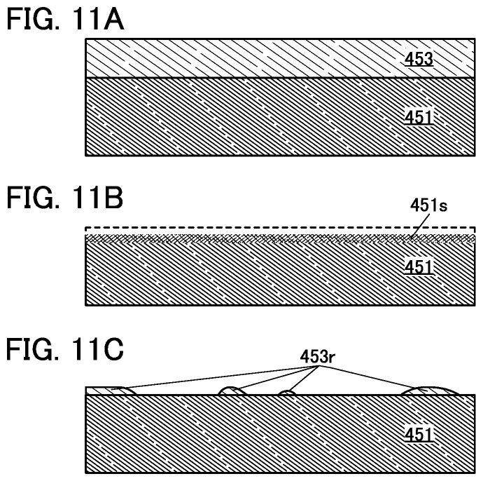

Here, as one means to solve the above problems, an inorganic mask film 453 is provided as a protective film over and in contact with an organic compound film 451 as illustrated in FIG. 11A, and then a step that would cause the above-described problems is performed. As the inorganic mask film, for example, an aluminum oxide film is used. Since an aluminum oxide film can be formed to be dense and is highly capable of blocking liquid and gas, the adverse effects caused by the step can be inhibited. Furthermore, the aluminum oxide film can be formed and removed by a method that causes less damage to the organic compound film, and thus is extremely suitable as the inorganic mask film 453 protecting the organic compound film 451.

Note that an atomic layer deposition (ALD) method that is capable of forming a denser film and causes less damage to the organic compound film is preferably employed as a formation method of the aluminum oxide film.

In this manner, the aluminum oxide film causes relatively little damage to the organic compound film when being formed and removed, and thus is suitably used as a protective film in processing the organic compound film by a lithography method. However, when the surface of the organic compound film is excessively exposed in a removal step of the aluminum oxide film, a surface 451s of the organic compound film 451 is damaged as illustrated in FIG. 11B, whereby the characteristics of the organic semiconductor might be degraded. Thus, time for removing the aluminum oxide is preferably as short as possible.

In order to complete the removal step in the shortest period of time, the step is preferably ended once the inorganic mask film 453 is removed from an upper surface of the organic compound film. However, it is extremely difficult to determine whether the inorganic mask film 453 is removed from the upper surface of the organic compound film. In addition, in-plane variation of the film quality of the inorganic mask film 453 may cause in-plane variation of the etching rate in etching that is the removal step of the inorganic mask film 453. As illustrated in FIG. 11C, even when the inorganic mask film can be removed partially, inorganic mask residues 453r might remain in some portions. In particular, in the case where an aluminum oxide film is provided as the inorganic mask film 453 over an organic compound film 451 by an ALD method, the film formation cannot be performed at high temperature and the above-described in-plane variation is easily caused. Accordingly, the inorganic mask residues 453r, which remain due to the in-plane variation, may be generated in some portions. When the inorganic mask film remains over the organic compound film, the driving voltage of a device to be manufactured later might increase. Note that excessive etching for removing all of the remaining inorganic mask residues 453r is extremely undesirable because such etching might cause side etching in a lateral direction of the inorganic mask film that is provided in an adjacent pixel direction and should remain in the process (unremoved inorganic mask film).

Thus, in one embodiment of the present invention, an organic mask film 452 is provided between the organic compound film 451 and the inorganic mask film 453 to facilitate the removal of the aluminum oxide film. For the organic mask film 452, the organic compound of one embodiment of the present invention can be used. Note that for the organic mask film 452, an organic compound with high water solubility is preferably used. As described above, the organic compound of one embodiment of the present invention sometimes has increased water solubility depending on the number of ring members of the bicyclic guanidine skeleton. The organic compound of one embodiment of the present invention having such increased water solubility is preferably used for the organic mask film 452.

First, the organic compound film 451 is formed over a base film 450 (FIG. 12A). The base film may be either an insulating film or a conductive film depending on a device manufactured later. The organic compound film 451 may be formed by a dry method such as an evaporation method or a wet method such as a spin coating method.

Next, the organic mask film 452 including the organic compound of one embodiment of the present invention described in Embodiment 1 is formed over the organic compound film 451 (FIG. 12A). The organic mask film 452 is preferably formed by a vacuum evaporation method.

Next, the inorganic mask film 453 is formed over the organic mask film 452 (FIG. 12A). The inorganic mask film 453 is preferably formed by a method that causes less damage to the film in contact with the organic compound film 451.

A film 454 to be a hard mask formed of a metal film or a metal compound film is preferably formed over the inorganic mask film 453 (FIG. 12B). Since damage to the organic compound film 451 can be reduced by the inorganic mask film 453, the film 454 to be a hard mask can be formed by a formation method that causes relatively great damage to a formation surface, such as a sputtering method.

Examples of a material for the film 454 to be a hard mask include silicon, silicon nitride, silicon oxide, tungsten, titanium, molybdenum, tantalum, tantalum nitride, an alloy containing molybdenum and niobium, an alloy metal oxide containing molybdenum and tungsten, and a metal oxide such as an indium gallium zinc oxide (also denoted as In—Ga—Zn oxide or IGZO). It is also possible to use indium oxide, indium zinc oxide (In—Zn oxide), indium tin oxide (In—Sn oxide), indium titanium oxide (In—Ti oxide), indium tin zinc oxide (In—Sn—Zn oxide), indium titanium zinc oxide (In—Ti—Zn oxide), indium gallium tin zinc oxide (In—Ga—Sn—Zn oxide), or the like. Alternatively, indium tin oxide containing silicon can also be used, for example.

Then, a photosensitive resin is applied to the film 454 to be a hard mask, so that a resin film 455 is formed. The photosensitive resin may be either a positive type resist or a negative type resist.

Next, a photomask layer 455a is formed by performing light exposure in accordance with the photosensitivity of the resin and performing development (FIG. 12D), and the film 454 to be a hard mask is etched with the use of the photomask layer 455α, whereby a hard mask layer 454a is formed (FIG. 12E).

The film 454 to be a hard mask may be etched by either wet etching or dry etching. This etching is preferably performed under the condition where the film 454 to be a hard mask has a high selectivity with respect to the inorganic mask film 453.

After the hard mask layer 454a is formed, the photomask layer 455a is removed (FIG. 13A). Owing to the film 454 to be a hard mask and the inorganic mask film 453, the organic compound film 451 is prevented from being adversely affected, for example, being eliminated or being damaged, by treatment in forming and removing the photomask layer 455a, and thus, an organic device having favorable characteristics can be manufactured.

After that, etching is performed with the use of the film 454 to be a hard mask as a mask, whereby an organic compound layer 451α, an organic mask layer 452α, and an inorganic mask layer 453a are formed (FIG. 13B). This etching may be either wet etching or dry etching, but dry etching is preferable.

After processing the organic compound layer 451a is completed, the hard mask layer 454a is removed (FIG. 13C). The hard mask layer 454a is removed by etching. Either wet etching or dry etching can be performed, but dry etching is preferable. This etching is preferably performed under the condition where the hard mask layer 454a has a high selectivity with respect to the inorganic mask layer 453a.

Lastly, the inorganic mask layer 453a and the organic mask layer 452a are treated with water or a liquid including water as a solvent to be removed at once (FIG. 13E).

As the removing method, the inorganic mask layer 453a and the organic mask layer 452a are immersed in water or the liquid including water as a solvent for a predetermined time, and then showered with pure water. The hard mask layer 454a and the organic mask layer 452a can be removed by just performing this step when the organic mask film 452 contains an organic compound with high water solubility. The liquid used for removing is preferably water because of less damage to the organic compound layer 451a.

Note that after the hard mask layer 454a is removed, the inorganic mask layer 453a may be removed to some extent before the organic mask layer 452a is treated with water or the liquid including water as a solvent (FIG. 13D). The organic mask layer 453a can be removed by etching. Although either wet etching or dry etching can be performed, wet etching using an alkaline solution or an acid solution is preferable, and wet etching using an alkaline solution is further preferable. The surface of the organic compound layer 451a is prevented from being exposed to the alkaline solution or the acid solution owing to the organic mask layer 452a thereover, whereby degradation of characteristics can be prevented. Furthermore, treatment is performed such that the inorganic mask residues 453r remain over the organic mask layer 452a to some extent, whereby the next step of removing the organic mask layer 452a can be performed more smoothly.

Since the organic mask layer 452a employs the organic compound film of one embodiment of the present invention having an electron-injection property, the organic mask film 452 is not necessarily removed completely and may remain partially or remain on the entire surface of the organic compound film 451.

The organic compound layer 451a processed through the above steps has less processing damage, so that an organic device can have favorable characteristics. Furthermore, the inorganic mask residues 453r are inhibited from remaining on the surface of the organic compound layer 451α, whereby an increase in voltage of an organic device to be manufactured later can be prevented.

The structure described in this embodiment can be used in combination with any of the structures described in other embodiments as appropriate.

### Embodiment 5

In this embodiment, a display apparatus of one embodiment of the present invention will be described.

The display apparatus in this embodiment can be a high-resolution display apparatus. Thus, the display apparatus in this embodiment can be used for display portions of information terminals (wearable devices) such as watch-type and bracelet-type information terminals and display portions of wearable devices that can be worn on a head, such as a VR device like a head mounted display (HMD) and a glasses-type AR device.

The display apparatus in this embodiment can be a high-definition display apparatus or a large-sized display apparatus. Accordingly, the display apparatus in this embodiment can be used for display portions of a digital camera, a digital video camera, a digital photo frame, a mobile phone, a portable game console, a portable information terminal, and an audio reproducing device, in addition to display portions of electronic devices with a relatively large screen, such as a television device, desktop and notebook personal computers, a monitor of a computer and the like, digital signage, and a large game machine such as a pachinko machine.

### [Display Module]

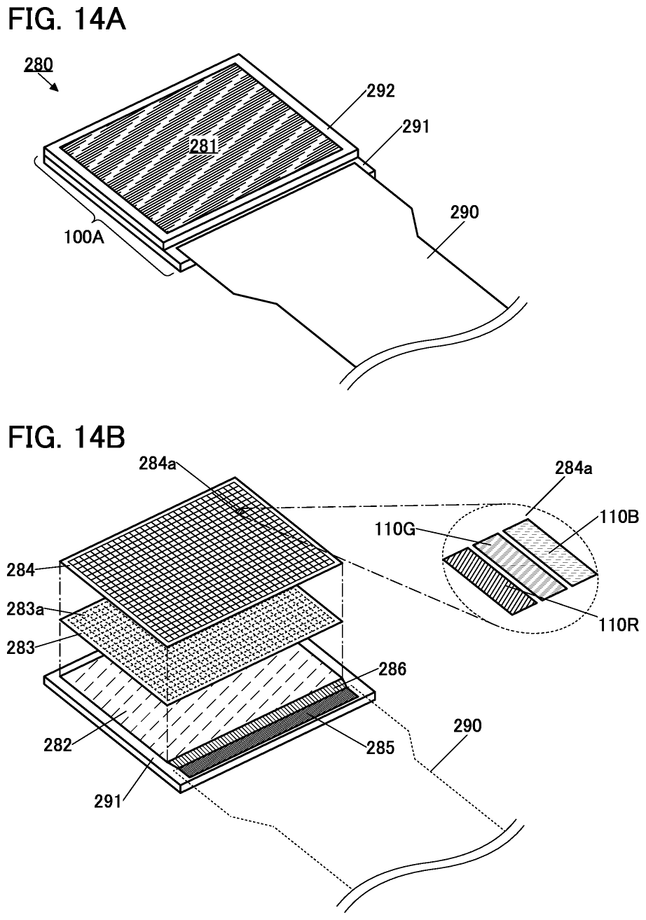

FIG. 14A is a perspective view of a display module 280. The display module 280 includes a display apparatus 100A and an FPC 290.

The display module 280 includes a substrate 291 and a substrate 292. The display module 280 includes a display portion 281. The display portion 281 is a region of the display module 280 where an image is displayed, and is a region where light emitted from pixels provided in a pixel portion 284 described later can be seen.

FIG. 14B is a perspective view schematically illustrating the structure on the substrate 291 side. Over the substrate 291, a circuit portion 282, a pixel circuit portion 283 over the circuit portion 282, and the pixel portion 284 over the pixel circuit portion 283 are stacked. In addition, a terminal portion 285 for connection to the FPC 290 is included in a portion not overlapping with the pixel portion 284 over the substrate 291. The terminal portion 285 and the circuit portion 282 are electrically connected to each other through a wiring portion 286 formed of a plurality of wirings.

The pixel portion 284 includes a plurality of pixels 284a arranged periodically. An enlarged view of one pixel 284a is illustrated on the right side in FIG. 14B. The pixels 284a can employ any of the structures described in the above embodiments. FIG. 14B illustrates an example where the pixel 284a has a structure similar to that of the pixel 178 illustrated in FIGS. 3A and 3B.

The pixel circuit portion 283 includes a plurality of pixel circuits 283a arranged periodically.

One pixel circuit 283a is a circuit that controls driving of a plurality of elements included in one pixel 284a. One pixel circuit 283a can be provided with three circuits each of which controls light emission of one light-emitting device. For example, the pixel circuit 283a can include at least one selection transistor, one current control transistor (driving transistor), and a capacitor for one light-emitting device. A gate signal is input to a gate of the selection transistor, and a video signal is input to a source or a drain of the selection transistor. With such a structure, an active-matrix display apparatus is achieved.

The circuit portion 282 includes a circuit for driving the pixel circuits 283a in the pixel circuit portion 283. For example, the circuit portion 282 preferably includes one or both of a gate line driver circuit and a source line driver circuit. The circuit portion 282 may also include at least one of an arithmetic circuit, a memory circuit, a power supply circuit, and the like.

The FPC 290 functions as a wiring for supplying a video signal, a power supply potential, or the like to the circuit portion 282 from the outside. An IC may be mounted on the FPC 290.

The display module 280 can have a structure where one or both of the pixel circuit portion 283 and the circuit portion 282 are stacked below the pixel portion 284; hence, the aperture ratio (effective display area ratio) of the display portion 281 can be significantly high. For example, the aperture ratio of the display portion 281 can be higher than or equal to 40% and lower than 100%, preferably higher than or equal to 50% and lower than or equal to 95%, further preferably higher than or equal to 60% and lower than or equal to 95%. Furthermore, the pixels 284a can be arranged extremely densely and thus the display portion 281 can have significantly high resolution. For example, the pixels 284a are preferably arranged in the display portion 281 with a resolution higher than or equal to 2000 ppi, further preferably higher than or equal to 3000 ppi, still further preferably higher than or equal to 5000 ppi, yet still further preferably higher than or equal to 6000 ppi, and lower than or equal to 20000 ppi or lower than or equal to 30000 ppi.

Such a display module 280 has extremely high resolution, and thus can be suitably used for a VR device such as an HMD or a glasses-type AR device. For example, even in the case of a structure where the display portion of the display module 280 is seen through a lens, pixels of the extremely-high-resolution display portion 281 included in the display module 280 are prevented from being recognized when the display portion is enlarged by the lens, so that display providing a high sense of immersion can be performed. Without being limited thereto, the display module 280 can be suitably used for electronic devices including a relatively small display portion. For example, the display module 280 can be favorably used in a display portion of a wearable electronic device, such as a wrist watch.

### [Display Apparatus 100A]

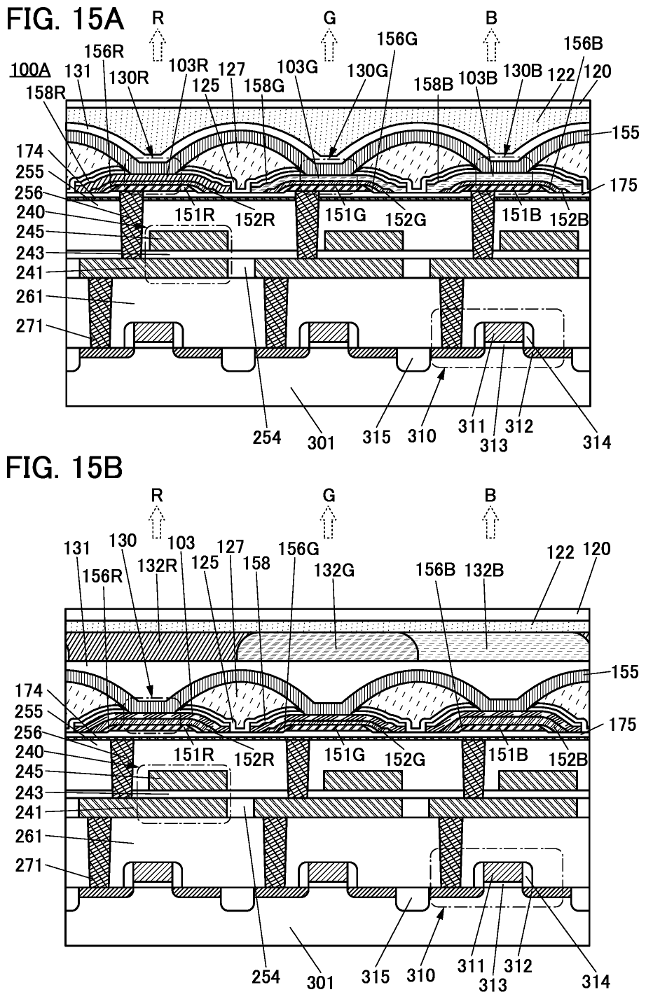

The display apparatus 100A illustrated in FIG. 15A includes a substrate 301, the light-emitting devices 130R, 130G, and 130B, a capacitor 240, and a transistor 310.

The substrate 301 corresponds to the substrate 291 in FIGS. 14A and 14B. The transistor 310 includes a channel formation region in the substrate 301. As the substrate 301, a semiconductor substrate such as a single crystal silicon substrate can be used, for example. The transistor 310 includes part of the substrate 301, a conductive layer 311, a low-resistance region 312, an insulating layer 313, and an insulating layer 314. The conductive layer 311 functions as a gate electrode. The insulating layer 313 is positioned between the substrate 301 and the conductive layer 311 and functions as a gate insulating layer. The low-resistance region 312 is a region where the substrate 301 is doped with an impurity, and functions as a source or a drain. The insulating layer 314 is provided to cover the side surface of the conductive layer 311.

An element isolation layer 315 is provided between two adjacent transistors 310 to be embedded in the substrate 301.

An insulating layer 261 is provided to cover the transistor 310, and the capacitor 240 is provided over the insulating layer 261.

The capacitor 240 includes a conductive layer 241, a conductive layer 245, and an insulating layer 243 between the conductive layers 241 and 245. The conductive layer 241 functions as one electrode of the capacitor 240, the conductive layer 245 functions as the other electrode of the capacitor 240, and the insulating layer 243 functions as a dielectric of the capacitor 240.

The conductive layer 241 is provided over the insulating layer 261 and is embedded in an insulating layer 254. The conductive layer 241 is electrically connected to one of the source and the drain of the transistor 310 through a plug 271 embedded in the insulating layer 261. The insulating layer 243 is provided to cover the conductive layer 241. The conductive layer 245 is provided in a region overlapping with the conductive layer 241 with the insulating layer 243 therebetween.

An insulating layer 255 is provided to cover the capacitor 240. The insulating layer 174 is provided over the insulating layer 255. The insulating layer 175 is provided over the insulating layer 174. The light-emitting devices 130R, 130G, and 130B are provided over the insulating layer 175. FIG. 15A illustrates an example where the light-emitting devices 130R, 130G, and 130B each have the stacked-layer structure illustrated in FIG. 6A. An insulator is provided in regions between adjacent light-emitting devices. For example, in FIG. 15A, the inorganic insulating layer 125 and the insulating layer 127 over the inorganic insulating layer 125 are provided in those regions.

The insulating layer 156R is provided to include a region overlapping with the side surface of the conductive layer 151R of the light-emitting device 130R. The insulating layer 156G is provided to include a region overlapping with the side surface of the conductive layer 151G of the light-emitting device 130G. The insulating layer 156B is provided to include a region overlapping with the side surface of the conductive layer 151B of the light-emitting device 130B. The conductive layer 152R is provided to cover the conductive layer 151R and the insulating layer 156R. The conductive layer 152G is provided to cover the conductive layer 151G and the insulating layer 156G. The conductive layer 152B is provided to cover the conductive layer 151B and the insulating layer 156B. The sacrificial layer 158R is positioned over the organic compound layer 103R of the light-emitting device 130R. The sacrificial layer 158G is positioned over the organic compound layer 103G of the light-emitting device 130G. The sacrificial layer 158B is positioned over the organic compound layer 103B of the light-emitting device 130B.

Each of the conductive layers 151R, 151G, and 151B is electrically connected to one of the source and the drain of the corresponding transistor 310 through a plug 256 embedded in the insulating layers 243, 255, 174, and 175, the conductive layer 241 embedded in the insulating layer 254, and the plug 271 embedded in the insulating layer 261. The top surface of the insulating layer 175 and the top surface of the plug 256 are level with or substantially level with each other. Any of a variety of conductive materials can be used for the plugs.

The protective layer 131 is provided over the light-emitting devices 130R, 130G, and 130B. The substrate 120 is attached to the protective layer 131 with the resin layer 122. Embodiment 3 can be referred to for the details of the light-emitting device 130 and the components thereover up to the substrate 120. The substrate 120 corresponds to the substrate 292 in FIG. 14A.

FIG. 15B illustrates a variation example of the display apparatus 100A illustrated in FIG. 15A. The display apparatus illustrated in FIG. 15B includes the coloring layers 132R, 132G, and 132B, and each of the light-emitting devices 130 includes a region overlapping with one of the coloring layers 132R, 132G, and 132B. In the display apparatus illustrated in FIG. 15B, the light-emitting device 130 can emit white light, for example. For example, the coloring layer 132R, the coloring layer 132G, and the coloring layer 132B can transmit red light, green light, and blue light, respectively.

This embodiment can be combined as appropriate with the other embodiments or the examples. In this specification, in the case where a plurality of structure examples are shown in one embodiment, the structure examples can be combined as appropriate.

### Embodiment 6

In this embodiment, electronic devices of embodiments of the present invention will be described.

Electronic devices of this embodiment include the display apparatus of one embodiment of the present invention in their display portions. The display apparatus of one embodiment of the present invention is highly reliable and can be easily increased in resolution and definition. Thus, the display apparatus of one embodiment of the present invention can be used for display portions of a variety of electronic devices.

Examples of the electronic devices include a digital camera, a digital video camera, a digital photo frame, a mobile phone, a portable game console, a portable information terminal, and an audio reproducing device, in addition to electronic devices with a relatively large screen, such as a television device, desktop and notebook personal computers, a monitor of a computer and the like, digital signage, and a large game machine such as a pachinko machine.

In particular, the display apparatus of one embodiment of the present invention can have high resolution, and thus can be favorably used for an electronic device having a relatively small display portion. Examples of such an electronic device include watch-type and bracelet-type information terminal devices (wearable devices) and wearable devices that can be worn on a head, such as a VR device like a head-mounted display, a glasses-type AR device, and an MR device.

The definition of the display apparatus of one embodiment of the present invention is preferably as high as HD (number of pixels: 1280×720), FHD (number of pixels: 1920×1080), WQHD (number of pixels: 2560×1440), WQXGA (number of pixels: 2560×1600), 4K (number of pixels: 3840×2160), or 8K (number of pixels: 7680×4320). In particular, definition of 4K, 8K, or higher is preferable. The pixel density (resolution) of the display apparatus of one embodiment of the present invention is preferably higher than or equal to 100 ppi, further preferably higher than or equal to 300 ppi, further preferably higher than or equal to 500 ppi, further preferably higher than or equal to 1000 ppi, still further preferably higher than or equal to 2000 ppi, still further preferably higher than or equal to 3000 ppi, still further preferably higher than or equal to 5000 ppi, yet further preferably higher than or equal to 7000 ppi. With such a display apparatus having one or both of high definition and high resolution, the electronic device can provide higher realistic sensation, sense of depth, and the like in personal use such as portable use or home use. There is no particular limitation on the screen ratio (aspect ratio) of the display apparatus of one embodiment of the present invention. For example, the display apparatus is compatible with a variety of screen ratios such as 1:1 (a square), 4:3, 16:9, and 16:10.

The electronic device in this embodiment may include a sensor (a sensor having a function of measuring force, displacement, position, speed, acceleration, angular velocity, rotational frequency, distance, light, liquid, magnetism, temperature, a chemical substance, sound, time, hardness, electric field, current, voltage, electric power, radiation, flow rate, humidity, gradient, oscillation, odor, or infrared rays).

The electronic device in this embodiment can have a variety of functions. For example, the electronic device in this embodiment can have a function of displaying a variety of data (e.g., a still image, a moving image, and a text image) on the display portion, a touch panel function, a function of displaying a calendar, date, time, and the like, a function of executing a variety of software (programs), a wireless communication function, and a function of reading out a program or data stored in a recording medium.

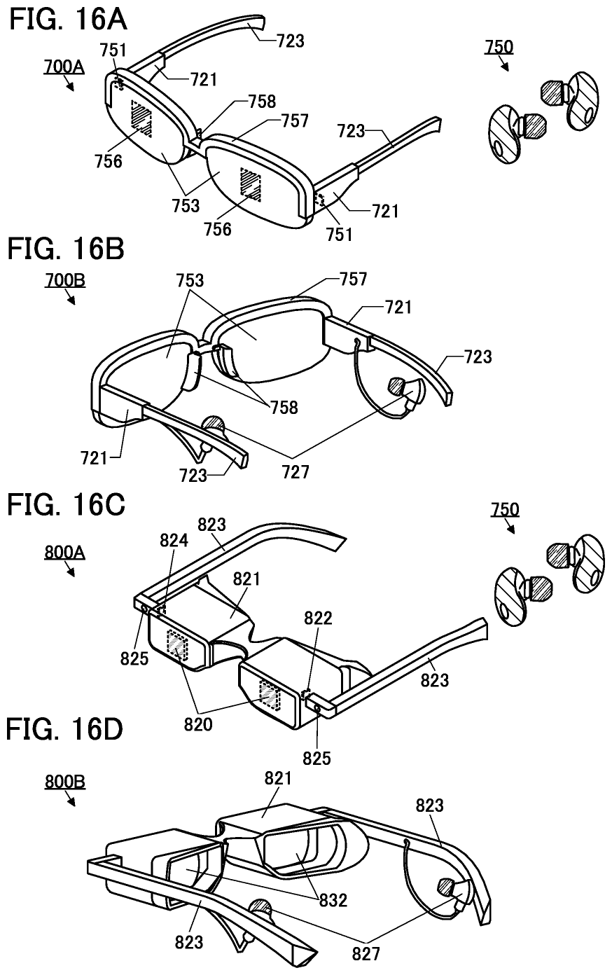

Examples of wearable devices that can be worn on a head are described with reference to FIGS. 16A to 16D. These wearable devices have at least one of a function of displaying AR contents, a function of displaying VR contents, a function of displaying SR contents, and a function of displaying MR contents. The electronic device having a function of displaying contents of at least one of AR, VR, SR, MR, and the like enables the user to feel a higher level of immersion.

An electronic device 700A illustrated in FIG. 16A and an electronic device 700B illustrated in FIG. 16B each include a pair of display panels 751, a pair of housings 721, a communication portion (not illustrated), a pair of wearing portions 723, a control portion (not illustrated), an image capturing portion (not illustrated), a pair of optical members 753, a frame 757, and a pair of nose pads 758.

The display apparatus of one embodiment of the present invention can be used for the display panels 751. Thus, a highly reliable electronic device is obtained.

The electronic devices 700A and 700B can each project images displayed on the display panels 751 onto display regions 756 of the optical members 753. Since the optical members 753 have a light-transmitting property, the user can see images displayed on the display regions, which are superimposed on transmission images seen through the optical members 753. Accordingly, the electronic devices 700A and 700B are electronic devices capable of AR display.

In the electronic devices 700A and 700B, a camera capable of capturing images of the front side may be provided as the image capturing portion. Furthermore, when the electronic devices 700A and 700B are provided with an acceleration sensor such as a gyroscope sensor, the orientation of the user's head can be sensed and an image corresponding to the orientation can be displayed on the display regions 756.

The communication portion includes a wireless communication device, and a video signal, for example, can be supplied by the wireless communication device. Instead of or in addition to the wireless communication device, a connector that can be connected to a cable for supplying a video signal and a power supply potential may be provided.

The electronic devices 700A and 700B are provided with a battery, so that they can be charged wirelessly and/or by wire.

A touch sensor module may be provided in the housing 721. The touch sensor module has a function of detecting a touch on the outer surface of the housing 721. Detecting a tap operation, a slide operation, or the like by the user with the touch sensor module enables various types of processing. For example, a video can be paused or restarted by a tap operation, and can be fast-forwarded or fast-reversed by a slide operation. When the touch sensor module is provided in each of the two housings 721, the range of the operation can be increased.

Various touch sensors can be applied to the touch sensor module. For example, any of touch sensors of the following types can be used: a capacitive type, a resistive type, an infrared type, an electromagnetic induction type, a surface acoustic wave type, and an optical type. In particular, a capacitive sensor or an optical sensor is preferably used for the touch sensor module.

In the case of using an optical touch sensor, a photoelectric conversion device (also referred to as a photoelectric conversion element) can be used as a light-receiving element. One or both of an inorganic semiconductor and an organic semiconductor can be used for an active layer of the photoelectric conversion device.

An electronic device 800A illustrated in FIG. 16C and an electronic device 800B illustrated in FIG. 16D each include a pair of display portions 820, a housing 821, a communication portion 822, a pair of wearing portions 823, a control portion 824, a pair of image capturing portions 825, and a pair of lenses 832.

The display apparatus of one embodiment of the present invention can be used for the display portions 820. Thus, a highly reliable electronic device is obtained.

The display portions 820 are positioned inside the housing 821 so as to be seen through the lenses 832. When the pair of display portions 820 display different images, three-dimensional display using parallax can be performed.

The electronic devices 800A and 800B can be regarded as electronic devices for VR. The user who wears the electronic device 800A or the electronic device 800B can see images displayed on the display portions 820 through the lenses 832.

The electronic devices 800A and 800B preferably include a mechanism for adjusting the lateral positions of the lenses 832 and the display portions 820 so that the lenses 832 and the display portions 820 are positioned optimally in accordance with the positions of the user's eyes. Moreover, the electronic devices 800A and 800B preferably include a mechanism for adjusting focus by changing the distance between the lenses 832 and the display portions 820.

The electronic device 800A or the electronic device 800B can be mounted on the user's head with the wearing portions 823. FIG. 16C, for instance, illustrates an example where the wearing portion 823 has a shape like a temple (also referred to as a joint or the like) of glasses; however, one embodiment of the present invention is not limited thereto. The wearing portion 823 can have any shape with which the user can wear the electronic device, for example, a shape of a helmet or a band.

The image capturing portion 825 has a function of obtaining information on the external environment. Data obtained by the image capturing portion 825 can be output to the display portion 820. An image sensor can be used for the image capturing portion 825. Moreover, a plurality of cameras may be provided so as to cover a plurality of fields of view, such as a telescope field of view and a wide field of view.

Although an example where the image capturing portions 825 are provided is shown here, a range sensor (hereinafter also referred to as a sensing portion) capable of measuring a distance between the user and an object just needs to be provided. In other words, the image capturing portion 825 is one embodiment of the sensing portion. As the sensing portion, an image sensor or a range image sensor such as a light detection and ranging (LIDAR) sensor can be used, for example. By using images obtained by the camera and images obtained by the range image sensor, more information can be obtained and a gesture operation with higher accuracy is possible.

The electronic device 800A may include a vibration mechanism that functions as bone-conduction earphones. For example, at least one of the display portion 820, the housing 821, and the wearing portion 823 can include the vibration mechanism. Thus, without additionally requiring an audio device such as headphones, earphones, or a speaker, the user can enjoy video and sound only by wearing the electronic device 800A. The electronic devices 800A and 800B may each include an input terminal. To the input terminal, a cable for supplying a video signal from a video output device or the like, power for charging a battery provided in the electronic device, and the like can be connected.

The electronic device of one embodiment of the present invention may have a function of performing wireless communication with earphones 750. The earphones 750 include a communication portion (not illustrated) and has a wireless communication function. The earphones 750 can receive information (e.g., audio data) from the electronic device with the wireless communication function. For example, the electronic device 700A in FIG. 16A has a function of transmitting information to the earphones 750 with the wireless communication function. As another example, the electronic device 800A in FIG. 16C has a function of transmitting information to the earphones 750 with the wireless communication function.

The electronic device may include an earphone portion. The electronic device 700B in FIG. 16B includes earphone portions 727. For example, the earphone portion 727 can be connected to the control portion by wire. Part of a wiring that connects the earphone portion 727 and the control portion may be positioned inside the housing 721 or the wearing portion 723.

Similarly, the electronic device 800B in FIG. 16D includes earphone portions 827. For example, the earphone portion 827 can be connected to the control portion 824 by wire. Part of a wiring that connects the earphone portion 827 and the control portion 824 maybe positioned inside the housing 821 or the wearing portion 823. Alternatively, the earphone portions 827 and the wearing portions 823 may include magnets. This is preferred because the earphone portions 827 can be fixed to the wearing portions 823 with magnetic force and thus can be easily housed.

The electronic device may include an audio output terminal to which earphones, headphones, or the like can be connected. The electronic device may include one or both of an audio input terminal and an audio input mechanism. As the audio input mechanism, a sound collecting device such as a microphone can be used, for example. The electronic device may have a function of a headset by including the audio input mechanism.

As described above, both the glasses-type device (e.g., the electronic devices 700A and 700B) and the goggles-type device (e.g., the electronic devices 800A and 800B) are preferable as the electronic device of one embodiment of the present invention.

The electronic device of one embodiment of the present invention can transmit information to earphones by wire or wirelessly.

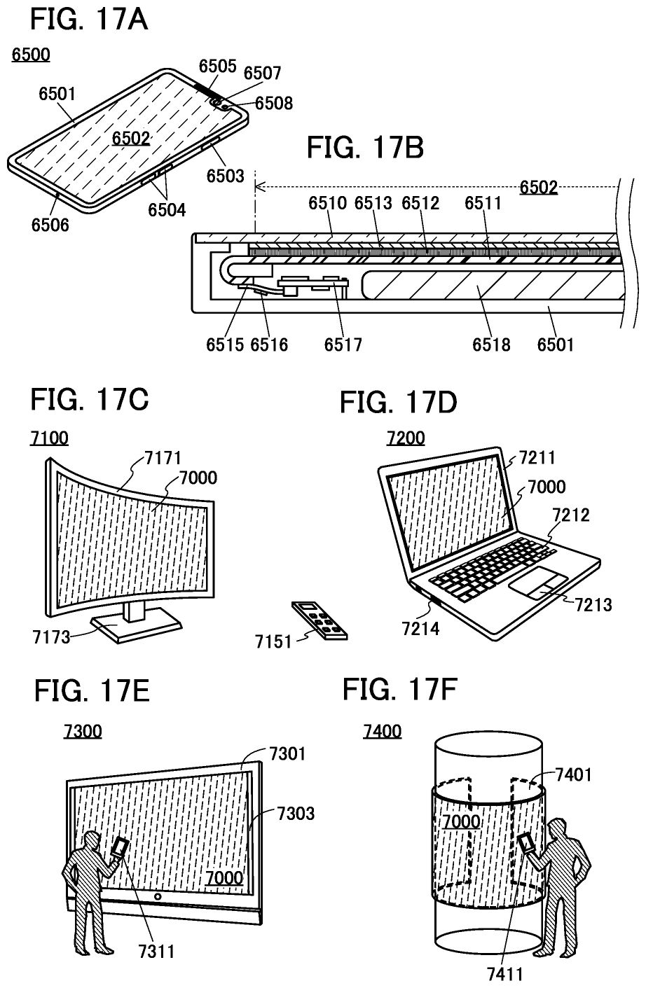

An electronic device 6500 illustrated in FIG. 17A is a portable information terminal that can be used as a smartphone.

The electronic device 6500 includes a housing 6501, a display portion 6502, a power button 6503, buttons 6504, a speaker 6505, a microphone 6506, a camera 6507, a light source 6508, and the like. The display portion 6502 has a touch panel function.

The display apparatus of one embodiment of the present invention can be used for the display portion 6502. Thus, a highly reliable electronic device is obtained.

FIG. 17B is a schematic cross-sectional view including an end portion of the housing 6501 on the microphone 6506 side.

A protection member 6510 having a light-transmitting property is provided on the display surface side of the housing 6501. A display panel 6511, an optical member 6512, a touch sensor panel 6513, a printed circuit board 6517, a battery 6518, and the like are provided in a space surrounded by the housing 6501 and the protection member 6510.

The display panel 6511, the optical member 6512, and the touch sensor panel 6513 are fixed to the protection member 6510 with an adhesive layer (not illustrated). Part of the display panel 6511 is folded back in a region outside the display portion 6502, and an FPC 6515 is connected to the part that is folded back. An IC 6516 is mounted on the FPC 6515. The FPC 6515 is connected to a terminal provided on the printed circuit board 6517.

A flexible display of one embodiment of the present invention can be used for the display panel 6511. Thus, an extremely lightweight electronic device can be achieved. Since the display panel 6511 is extremely thin, the battery 6518 with high capacity can be mounted without an increase in the thickness of the electronic device. Moreover, part of the display panel 6511 is folded back so that a connection portion with the FPC 6515 is provided on the back side of the pixel portion, whereby an electronic device with a narrow bezel can be achieved.

FIG. 17C illustrates an example of a television device. In a television device 7100, a display portion 7000 is incorporated in a housing 7171. Here, the housing 7171 is supported by a stand 7173.

The display apparatus of one embodiment of the present invention can be used for the display portion 7000. Thus, a highly reliable electronic device is obtained.

Operation of the television device 7100 illustrated in FIG. 17C can be performed with an operation switch provided in the housing 7171 and a separate remote controller 7151. Alternatively, the display portion 7000 may include a touch sensor, and the television device 7100 may be operated by touch on the display portion 7000 with a finger or the like. The remote controller 7151 may be provided with a display portion for displaying information output from the remote controller 7151. With operation keys or a touch panel of the remote controller 7151, channels and volume can be controlled and images displayed on the display portion 7000 can be controlled.

Note that the television device 7100 includes a receiver, a modem, and the like. A general television broadcast can be received with the receiver. When the television device is connected to a communication network by wire or wirelessly via the modem, one-way (from a transmitter to a receiver) or two-way (e.g., between a transmitter and a receiver or between receivers) information communication can be performed.

FIG. 17D illustrates an example of a notebook personal computer. A notebook personal computer 7200 includes a housing 7211, a keyboard 7212, a pointing device 7213, an external connection port 7214, and the like. The display portion 7000 is incorporated in the housing 7211.

The display apparatus of one embodiment of the present invention can be used for the display portion 7000. Thus, a highly reliable electronic device is obtained.

FIGS. 17E and 17F illustrate examples of digital signage.

Digital signage 7300 illustrated in FIG. 17E includes a housing 7301, the display portion 7000, a speaker 7303, and the like. The digital signage 7300 can also include an LED lamp, operation keys (including a power switch or an operation switch), a connection terminal, a variety of sensors, a microphone, and the like.

FIG. 17F illustrates digital signage 7400 attached to a cylindrical pillar 7401. The digital signage 7400 includes the display portion 7000 provided along a curved surface of the pillar 7401.

In FIGS. 17E and 17F, the display apparatus of one embodiment of the present invention can be used for the display portion 7000. Thus, a highly reliable electronic device is obtained.

A larger area of the display portion 7000 can increase the amount of information that can be provided at a time. The display portion 7000 having a larger area attracts more attention, so that the effectiveness of the advertisement can be increased, for example.

The touch panel is preferably used in the display portion 7000, in which case in addition to display of still or moving images on the display portion 7000, intuitive operation by a user is possible. Moreover, in the case of an application for providing information such as route information or traffic information, usability can be enhanced by intuitive operation.

As illustrated in FIGS. 17E and 17F, it is preferable that the digital signage 7300 or the digital signage 7400 can work with an information terminal 7311 or an information terminal 7411, such as a smartphone that a user has, through wireless communication. For example, information of an advertisement displayed on the display portion 7000 can be displayed on a screen of the information terminal 7311 or the information terminal 7411. By operation of the information terminal 7311 or the information terminal 7411, a displayed image on the display portion 7000 can be switched.

It is possible to make the digital signage 7300 or the digital signage 7400 execute a game with the use of the screen of the information terminal 7311 or the information terminal 7411 as an operation means (controller). Thus, an unspecified number of users can join in and enjoy the game concurrently.

This embodiment can be combined as appropriate with the other embodiments or the examples. In this specification, in the case where a plurality of structure examples are shown in one embodiment, the structure examples can be combined as appropriate.

### Example 1

### Synthesis Example 1

This example describes a synthesis method of 1-(10,10-dimethyl-10H-spiro[anthracene-9,9′-fluoren]-2′-yl)-1,3,4,6,7,8-hexahydro-2H-pyrimido[1,2-a]pyrimidine (abbreviation: Fha-hpp) (Structural Formula (100)), which is an organic compound of one embodiment of the present invention. The structure of Fha-hpp is shown below.

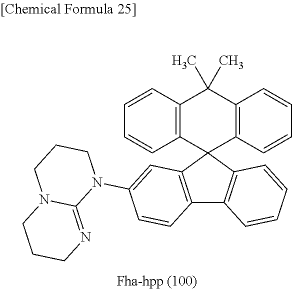 [SMILES: CC1(C)c2ccccc2C2(c3ccccc3-c3ccc(N4CCCN5CCCN=C54)cc32)c2ccccc21]

### <Synthesis of Fha-hpp>

In a 300-mL three-neck flask were put 4.4 g (10 mmol) of 2′-bromo-10,10-dimethyl-10H-spiro[anthracen-9,9′-fluorene], 1.8 g (13 mmol) of 1,3,4,6,7,8-hexahydro-2H-pyrimido[1,2-a]pyrimidine, 0.38 mg (0.61 mmol) of (±)-2,2′-bis(diphenylphosphino)-1,1′-binaphthyl (abbreviation: rac-BINAP), 2.8 g (25 mmol) of potassium tert-butoxide, and 100 mL of dehydrated toluene, and the mixture was degassed by stirring under reduced pressure. To this mixture was added 91 mg (0.41 mmol) of palladium(II) acetate and stirring was performed at 90° C. under a nitrogen stream for 4 hours. After the stirring, the mixture was cooled down to room temperature. An insoluble matter was separated from the mixture by suction filtration, and the obtained filtrate was subjected to extraction with toluene. The extracted solution was concentrated to give an oily substance. The oily substance was recrystallized with a mixed solvent of toluene and hexane. A precipitated solid was collected by suction filtration to give 1.2 g of a target yellow solid in a yield of 24%. The synthesis scheme of Fha-hpp is shown in Formula (α-1) below.

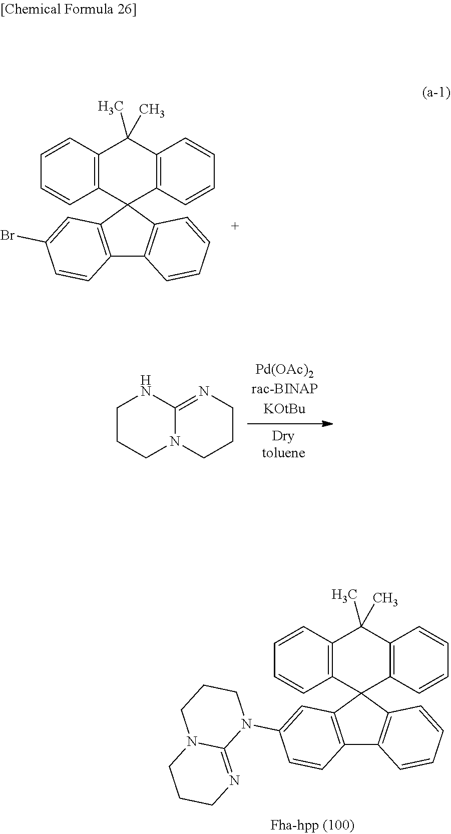 [SMILES: CC1(C)c2ccccc2C2(c3ccccc3-c3ccc(Br)cc32)c2ccccc21 ; C1CN=C2NCCCN2C1 ; CC1(C)c2ccccc2C2(c3ccccc3-c3ccc(N4CCCN5CCCN=C54)cc32)c2ccccc21]

By a train sublimation method, 1.2 g of the obtained yellow solid was purified by sublimation. In the purification by sublimation, heating was performed for 24 hours at an argon flow rate of 5 mL/min, a pressure of 3.1 Pa, and a heating temperature of 195° C. As a result, 0.92 g of a target yellow solid was obtained at a collection rate of 77%.

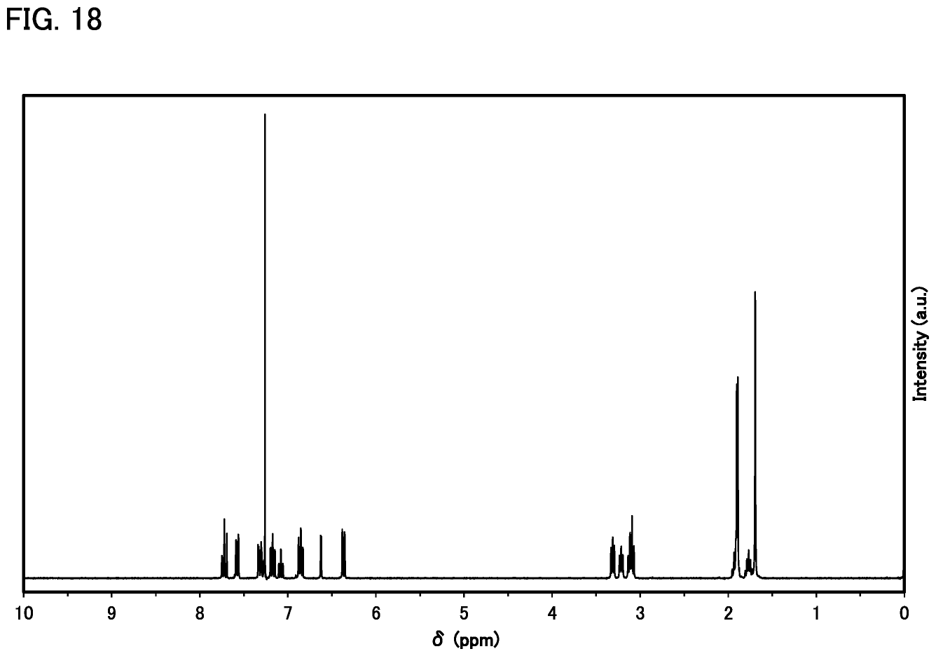

FIG. 18 shows a ¹H NMR spectrum of Fha-hpp after the purification by sublimation. Results of ¹H NMR measurement are shown below. The results show that Fha-hpp was obtained.

¹H NMR (CDCl₃, 300 MHz): δ=7.75-7.69 (m, 2H), 7.58 (dd, J=8.1 Hz, 1.2 Hz, 2H), 7.34-7.27 (m, 2H), 7.20-7.15 (m, 2H), 7.08 (td, J=7.4 Hz, 0.90 Hz, 1H), 6.88-6.83 (m, 3H), 6.62 (sd, J=1.8 Hz, 1H), 6.37 (dd, J=8.1 Hz, 1.5 Hz, 2H), 3.31 (t, J=5.9 Hz, 2H), 3.22 (t, J=5.7 Hz, 2H), 3.14-3.07 (m, 4H), 1.96-1.88 (m, 8H), 1.81-1.73 (m, 2H).

The glass transition temperature (Tg) of Fha-hpp was measured. Note that Tg was measured with a differential scanning calorimeter (DSC8500, manufactured by PerkinElmer Japan Co., Ltd.) in a state where a powder was put on an aluminum cell and the temperature was increased at a rate of 40° C./min. As a result, Tg of Fha-hpp was 103° C.

### Example 2

### Synthesis Example 2

This example describes a synthesis method of 1-(spiro[7H-benzo[c]fluorene-7,9′-9H-fluoren]-2′-yl)-1,3,4,6,7,8-hexahydro-2H-pyrimido[1,2-a]pyrimidine (abbreviation: SBF-hpp) (Structural Formula (115)), which is an organic compound of one embodiment of the present invention. The structure of SBF-hpp is shown below.

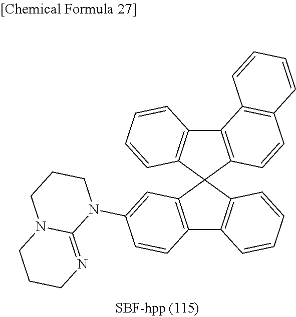 [SMILES: c1ccc2c(c1)-c1ccc(N3CCCN4CCCN=C43)cc1C21c2ccccc2-c2c1ccc1ccccc21]

### <Synthesis of SBF-hpp>

In a 300-mL three-neck flask were put 4.4 g (9.9 mmol) of 2′-bromospiro[7H-benzo[c]fluorene-7,9′-9H-fluorene], 1.7 g (12 mmol) of 1,3,4,6,7,8-hexahydro-2H-pyrimido[1,2-a]pyrimidine, 0.37 g (0.59 mmol) of (±)-2,2′-bis(diphenylphosphino)-1,1′-binaphthyl (abbreviation: rac-BINAP), 2.8 g (25 mmol) of potassium tert-butoxide, and 99 mL of dehydrated toluene, and the mixture was degassed by stirring under reduced pressure. To this mixture was added 89 mg (0.40 mmol) of palladium(II) acetate, and the mixture was stirred at 90° C. under a nitrogen stream for 9 hours. After the stirring, the mixture was cooled down to room temperature. An insoluble matter was separated from the mixture by suction filtration, and the obtained filtrate was subjected to extraction with toluene. The extracted solution was concentrated to give an oily substance. A small amount of toluene and saturated saline were added to the oily substance, irradiation with ultrasonic waves was performed, and an organic layer was subjected to extraction with toluene. The extracted solution was concentrated to give a solid. A small amount of toluene was added to the solid, irradiation with ultrasonic waves was performed, and the solid was collected by suction filtration. A sodium hydroxide solution and toluene were added to the solid, stirring was performed at room temperature overnight, and extraction with toluene was performed. The extracted solution was concentrated to give an oily substance. A small amount of toluene and hexane were added to the oily substance, irradiation with ultrasonic waves was performed, and a precipitated solid was collected by suction filtration, whereby 1.9 g of a target pale yellow solid was obtained in a yield of 38%. The synthesis scheme of SBF-hpp is shown in Formula (b-1) below.

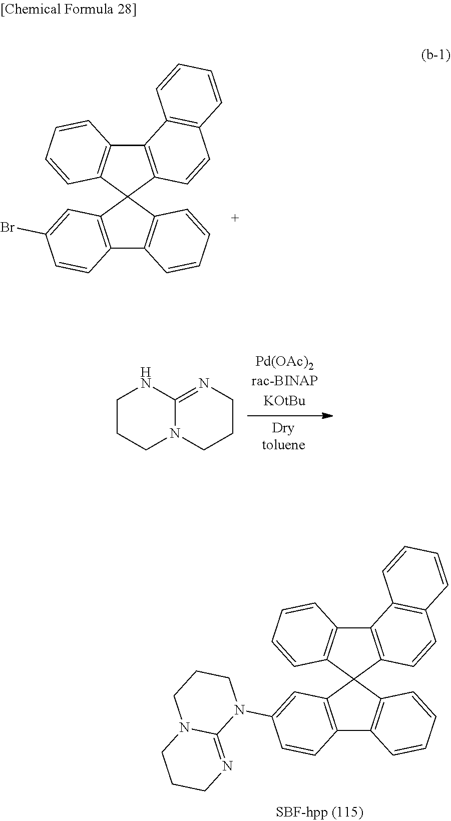 [SMILES: Brc1ccc2c(c1)C1(c3ccccc3-2)c2ccccc2-c2c1ccc1ccccc21 ; C1CN=C2NCCCN2C1 ; c1ccc2c(c1)-c1ccc(N3CCCN4CCCN=C43)cc1C21c2ccccc2-c2c1ccc1ccccc21]

By a train sublimation method, 1.9 g of the obtained pale yellow solid was purified. In the purification by sublimation, heating was performed for 48 hours at an argon flow rate of 10 mL/min, a pressure of 5.0 Pa, and a heating temperature of 235° C. As a result, 1.4 g of a target pale yellow solid was obtained at a collection rate of 74%.

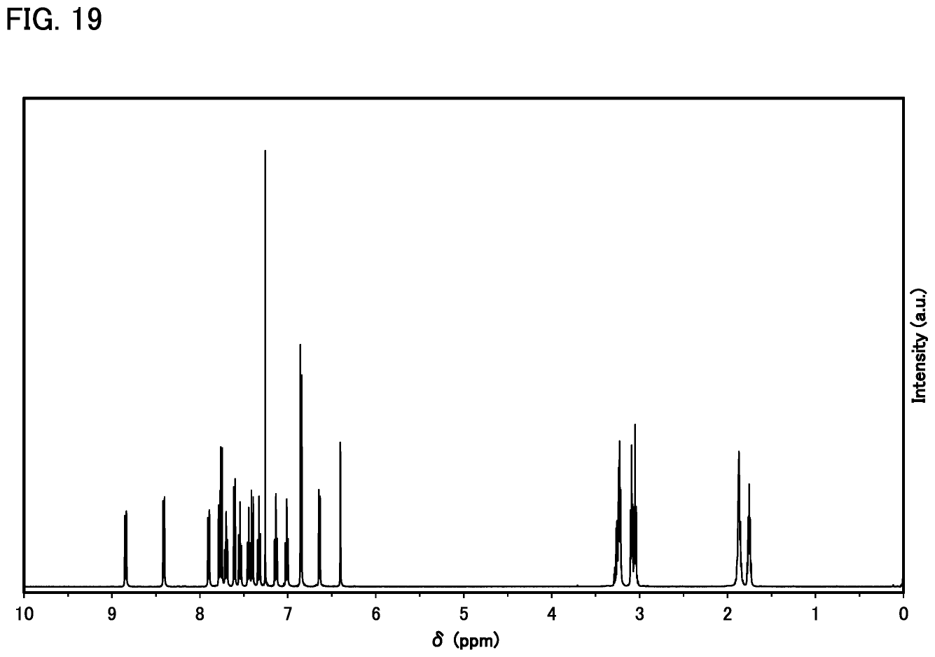

FIG. 19 shows a ¹H NMR spectrum of SBF-hpp after the purification by sublimation. Results of ¹H NMR measurement are shown below. The results show that SBF-hpp was obtained.

¹H NMR (CDCl₃, 500 MHz): δ=8.84 (d, J=8.5 Hz, 1H), 8.41 (d, J=8.0 Hz, 1H), 7.90 (d, J=8.0 Hz, 1H), 7.79-7.75 (m, 2H), 7.72-7.69 (m, 1H), 7.61 (d, J=8.5 Hz, 1H), 7.56-7.53 (m, 1H), 7.45 (td, J=7.8 Hz, 1.0 Hz, 1H), 7.40 (dd, J=8.5 Hz, 2.5 Hz, 1H), 7.34-7.31 (m, 1H), 7.14 (td, J=7.5 Hz, 1.5 Hz, 1H), 7.01 (t, J=7.5 Hz, 1H), 6.85 (d, J=9.0 Hz, 2H), 6.64 (d, J=7.5 Hz, 1H), 6.40 (sd, J=2.5 Hz, 1H), 3.29-3.21 (m, 4H), 3.10-3.02 (m, 4H), 1.89-1.84 (m, 2H), 1.78-1.73 (m, 2H).

Tg of SBF-hpp was measured. Note that Tg was measured with a differential scanning calorimeter (DSC8500, manufactured by PerkinElmer Japan Co., Ltd.) in a state where a powder was put on an aluminum cell and the temperature was increased at a rate of 40° C./min. As a result, Tg of SBF-hpp was 124° C.

### Example 3

### Synthesis Example 3

This example describes a synthesis method of 1-[3-(9,9′-spirobi[9H-fluoren]-2-yl)phenyl]-1,3,4,6,7,8-hexahydro-2H-pyrimido[1,2-a]pyrimidine (abbreviation: mSFP-hpp) (Structural Formula (133)), which is an organic compound of one embodiment of the present invention. The structure of mSFP-hpp is shown below.

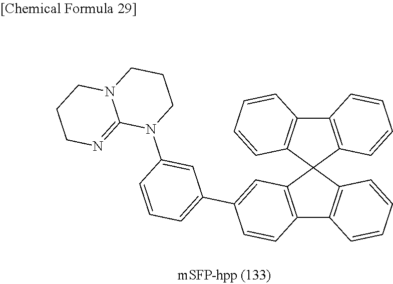 [SMILES: c1cc(-c2ccc3c(c2)C2(c4ccccc4-c4ccccc42)c2ccccc2-3)cc(N2CCCN3CCCN=C32)c1]

### Step 1: Synthesis of 2-(3-bromophenyl)-9,9′-spirobi[9H-fluorene]

In a 500-mL three-neck flask were put 5.4 g (15 mmol) of 9,9′-spirobi[9H-fluoren]-2-ylboronic acid, 7.6 g (27 mmol) of 3-bromoiodobenzene, 6.2 g (45 mmol) of potassium carbonate, 113 mL of toluene, 37 mL of ethanol, and 22 mL of water, and the mixture was degassed by stirring under reduced pressure. To this mixture was added 0.34 g (0.29 mmol) of tetrakis(triphenylphosphine)palladium(0), and the mixture was stirred at 90° C. under a nitrogen stream for 30 minutes. After the stirring, the mixture was cooled down to room temperature, followed by extraction with toluene. Then, the extracted solution was concentrated to give an oily substance. The oily substance was purified by silica gel column chromatography (developing solvent: toluene). The obtained fraction was concentrated to give an oily substance. The oily substance was purified by high performance liquid column chromatography (developing solvent: chloroform). The obtained fraction was concentrated to give 4.9 g of a target colorless transparent oily substance in a yield of 70%. The synthesis scheme of Step 1 is shown in Formula (c-1) below.

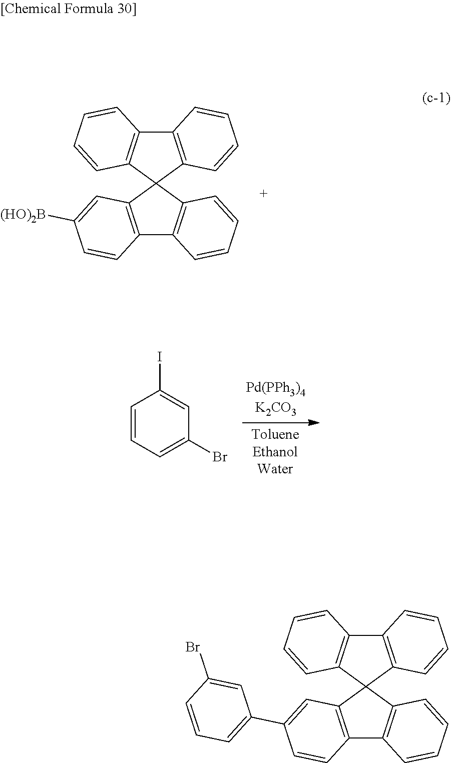 [SMILES: OB(O)c1ccc2c(c1)C1(c3ccccc3-c3ccccc31)c1ccccc1-2 ; Brc1cccc(I)c1 ; Brc1cccc(-c2ccc3c(c2)C2(c4ccccc4-c4ccccc42)c2ccccc2-3)c1]

### Step 2: Synthesis of mSFP-hpp

In a 300-mL three-neck flask were put 4.9 g (10 mmol) of 2-(3-bromophenyl)-9,9′-spirobi[9H-fluorene] synthesized in Step 1, 1.8 g (13 mmol) of 1,3,4,6,7,8-hexahydro-2H-pyrimido[1,2-a]pyrimidine, 0.39 g (0.63 mmol) of (±)-2,2′-bis(diphenylphosphino)-1,1′-binaphthyl (abbreviation: rac-BINAP), 3.2 g (29 mmol) of potassium tert-butoxide, and 130 mL of dehydrated toluene, and the mixture was degassed by stirring under reduced pressure. To this mixture was added 92 mg (0.41 mmol) of palladium(II) acetate, and the mixture was stirred at 90° C. under a nitrogen stream for 12 hours. After the stirring, the mixture was cooled down to room temperature. An insoluble matter was separated from the mixture by suction filtration, and the obtained filtrate was subjected to extraction with toluene. The extracted solution was concentrated to give an oily substance. A small amount of toluene and saturated saline were added to the oily substance, irradiation with ultrasonic waves was performed, and an organic layer was subjected to extraction with toluene. The extracted solution was concentrated to give an oily substance. A small amount of toluene and a small amount of ethyl acetate were added to the oily substance, irradiation with ultrasonic waves was performed, and a precipitated solid was collected by suction filtration. A sodium hydroxide solution and toluene were added to the solid, stirring was performed at room temperature overnight, and extraction with toluene was performed. The extracted solution was concentrated to give an oily substance. A small amount of toluene and hexane were added to the oily substance, irradiation with ultrasonic waves was performed, and a precipitated solid was collected by suction filtration, whereby 0.98 g of a target pale yellow solid was obtained in a yield of 18%. The synthesis scheme of mSFP-hpp is shown in Formula (c-2) below.

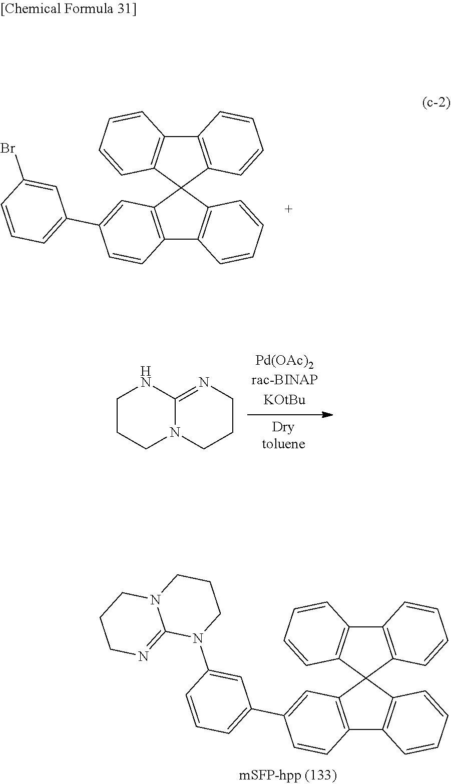 [SMILES: Brc1cccc(-c2ccc3c(c2)C2(c4ccccc4-c4ccccc42)c2ccccc2-3)c1 ; C1CN=C2NCCCN2C1 ; c1cc(-c2ccc3c(c2)C2(c4ccccc4-c4ccccc42)c2ccccc2-3)cc(N2CCCN3CCCN=C32)c1]

By a train sublimation method, 0.98 g of the obtained pale yellow solid was purified. In the purification by sublimation, heating was performed for 40 hours at an argon flow rate of 10 mL/min, a pressure of 6.2 Pa, and a heating temperature of 235° C. As a result, 0.57 g of a target pale yellow solid was obtained at a collection rate of 58%.

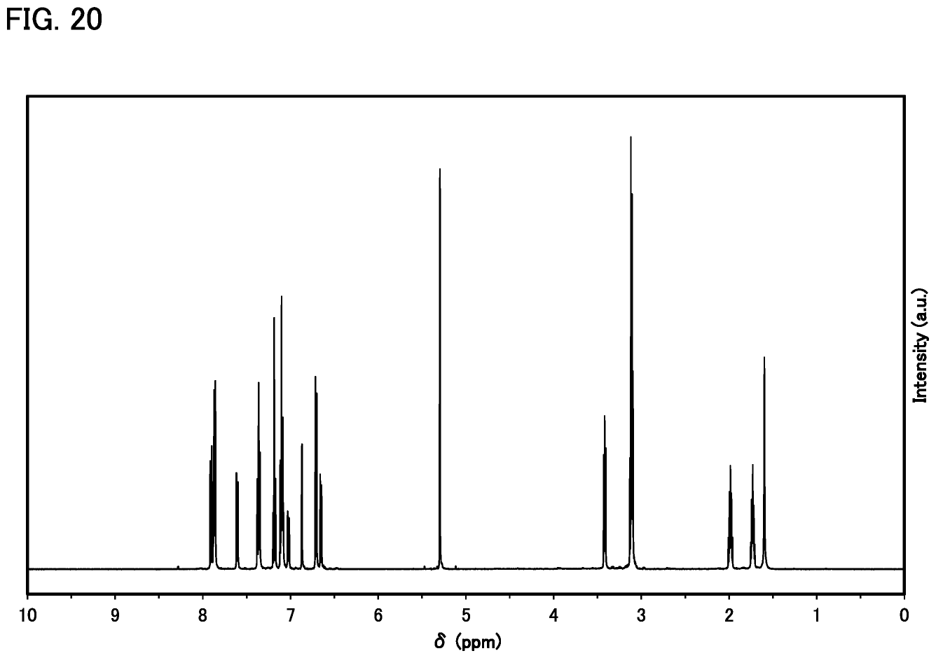

FIG. 20 shows a ¹H NMR spectrum of mSFP-hpp after the purification by sublimation. Results of ¹H NMR measurement are shown below. The results show that mSFP-hpp was obtained.

¹H NMR (CD₂Cl₂, 300 MHz): δ=7.92-7.86 (m, 4H), 7.61 (dd, J=8.0 Hz, 2.0 Hz, 1H), 7.38-7.35 (m, 3H), 7.20-7.17 (m, 2H), 7.12-7.08 (m, 4H), 7.04-7.01 (m, 1H), 6.87 (sd, J 1.5 Hz, 1H), 6.71 (d, J=7.5 Hz, 2H), 6.65 (d, J=7.5 Hz, 1H), 3.43-3.41 (m, 2H), 3.13-3.10 (m, 6H), 2.01-1.96 (m, 2H), 1.75-1.71 (m, 2H).

Tg of mSFP-hpp was measured. Note that Tg was measured with a differential scanning calorimeter (DSC8500, manufactured by PerkinElmer Japan Co., Ltd.) in a state where a powder was put on an aluminum cell and the temperature was increased at a rate of 40° C./min. As a result, Tg of mSFP-hpp was 113° C.

### Example 4

This example describes the light-emitting device 1 fabricated using Fha-hpp (Structural Formula (100)), which is the organic compound of one embodiment of the present invention. Note that the light-emitting device 1 is a light-emitting device where the organic compound layer is processed by a photolithography method. Structural formulae of organic compounds used for the light-emitting device 1 are shown below.

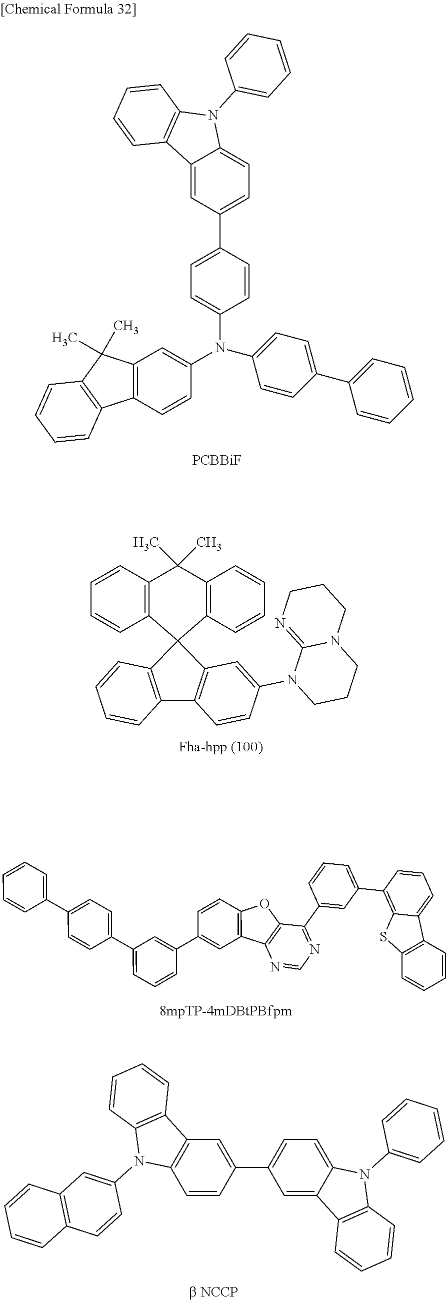

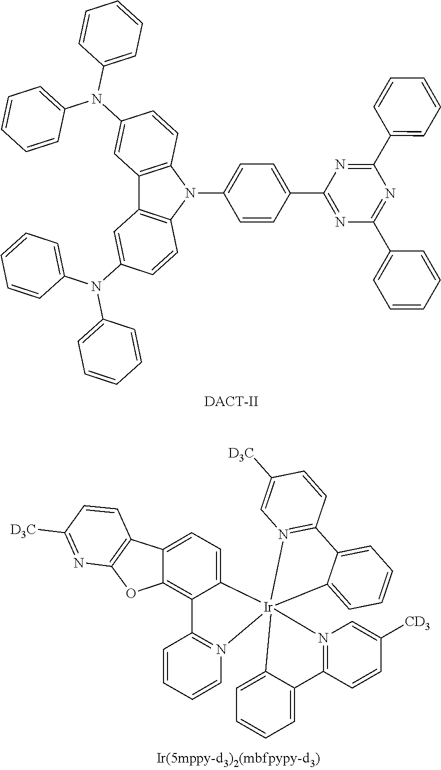

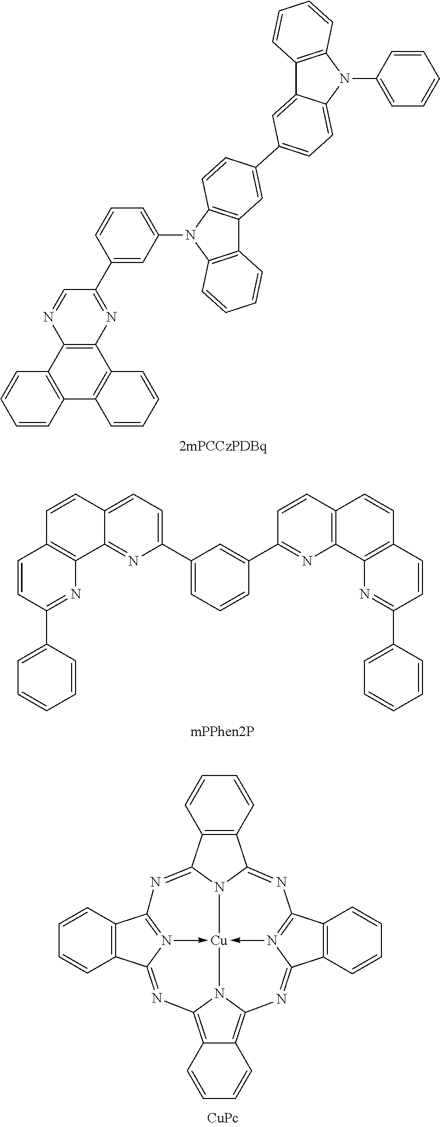

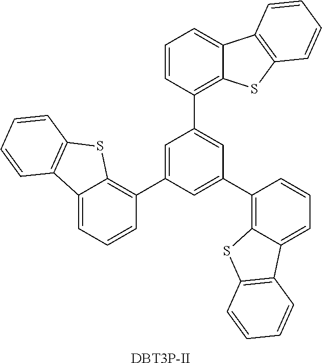 [SMILES: c1ccc2c(c1)sc1c(-c3cc(-c4cccc5c4sc4ccccc45)cc(-c4cccc5c4sc4ccccc45)c3)cccc12 ; "DBT3P-II"]

### (Fabrication Method of Light-Emitting Device 1)

First, as a reflective electrode, an alloy containing silver (Ag), palladium (Pd), and copper (Cu) (abbreviation: APC), was formed over a glass substrate to a thickness of 100 nm by a sputtering method, and then, as a transparent electrode, indium tin oxide containing silicon oxide (ITSO) was formed to a thickness of 100 nm by a sputtering method, whereby the first electrode 101 was formed. The electrode area was set to 4 mm² (2 mm×2 mm). Note that the transparent electrode functions as the anode, and the transparent electrode and the reflective electrode can be collectively regarded as the first electrode.

Next, in pretreatment for forming the light-emitting device over a substrate, the surface of the substrate was washed with water, baking was performed at 200° C. for one hour, and then UV ozone treatment was performed for 370 seconds.

After that, the substrate was transferred into a vacuum evaporation apparatus where the pressure was reduced to approximately 1×10⁻⁴ Pa, and was subjected to heat treatment at 170° C. for 30 minutes in a heating chamber of the vacuum evaporation apparatus, and then the substrate was cooled down for approximately 30 minutes.

Then, the substrate provided with the first electrode was fixed to a substrate holder provided in the vacuum evaporation apparatus such that the surface on which the first electrode was formed faced downward. Over the first electrode, N-(biphenyl-4-yl)-N-[4-(9-phenyl-9H-carbazol-3-yl)phenyl]-9,9-dimethyl-9H-fluoren-2-amine (abbreviation: PCBBiF) and a fluorine-containing electron acceptor material with a molecular weight of 672 (OCHD-003) were deposited by co-evaporation to a thickness of 10 nm such that the weight ratio of PCBBiF to OCHD-003 was 1:0.03, whereby a hole-injection layer was formed.

Over the hole-injection layer, PCBBiF was deposited by evaporation to a thickness of 60 nm, whereby a first hole-transport layer was formed.

Then, over the first hole-transport layer, 8-(1,1′:4′,1″-terphenyl-3-yl)-4-[3-(dibenzothiophen-4-yl)phenyl]-[1]benzofuro[3,2-d]pyrimidine (abbreviation: 8mpTP-4mDBtPBfpm), 9-(2-naphthyl)-9′-phenyl-9H,9′H-3,3′-bicarbazole (abbreviation: βNCCP), and [2-d₃-methyl-8-(2-pyridinyl-κN)benzofuro[2,3-b]pyridine-κC]bis[2-(5-d₃-methyl-2-pyridinyl-κN2)phenyl-κC]iridium(III) (abbreviation: Ir(5mppy-d₃)₂(mbfpypy-d₃)) were deposited by co-evaporation to a thickness of 40 nm such that the weight ratio of 8mpTP-4mDBtPBfpm, βNCCP, and Ir(5mppy-d₃)₂(mbfpypy-d₃) was 0.5:0.5:0.1, whereby a first light-emitting layer was formed.

Next, 3,6-bis(diphenylamino)-9-[4-(4,6-diphenyl-1,3,5-triazin-2-yl)phenyl]-9H-carbazole (abbreviation: DACT-II) was deposited by evaporation to a thickness of 10 nm, whereby a first electron-transport layer was formed.

After formation of the first electron-transport layer, 2,2′-(1,3-phenylene)bis(9-phenyl-1,10-phenanthroline) (abbreviation: mPPhen2P) and Fha-hpp (Structural Formula (100)), which is the organic compound of one embodiment of the present invention, were deposited by co-evaporation to a thickness of 5 nm such that the weight ratio of mPPhen2P to Fha-hpp was 6.6:3.3, whereby a first layer was formed. Then, copper phthalocyanine (abbreviation: CuPc) was deposited to a thickness of 2 nm, whereby a second layer was formed. Furthermore, PCBBiF and OCHD-003 were deposited by co-evaporation to a thickness of 10 nm such that the weight ratio of PCBBiF to OCHD-003 was 1:0.15 to form a third layer, whereby an intermediate layer including the first to third layers was formed.

Next, over the intermediate layer, PCBBiF was deposited by evaporation to a thickness of 55 nm, whereby a second hole-transport layer was formed.

Over the second hole-transport layer, 8mpTP-4mDBtPBfpm, βNCCP, and Ir(5mppy-d₃)₂(mbfpypy-d₃) were deposited by co-evaporation to a thickness of 40 nm such that the weight ratio of 8mpTP-4mDBtPBfpm, βNCCP, and Ir(5mppy-d₃)₂(mbfpypy-d₃) was 0.5:0.5:0.1, whereby a second light-emitting layer was formed.

After that, 2-{3-[3-(N-phenyl-9H-carbazol-3-yl)-9H-carbazol-9-yl]phenyl}dibenzo[f,h]quinoxaline (abbreviation: 2mPCCzPDBq) was deposited by evaporation to a thickness of 20 nm and mPPhen2P was further deposited by evaporation to a thickness of 20 nm, whereby a second electron-transport layer was formed.

After formation of the second electron-transport layer, processing by a photolithography method and heat treatment were performed.

### <<Processing by Photolithography Method and Heat Treatment>>

Here, the processing by a photolithography method and the heat treatment are described. First, the substrate was taken out from the vacuum evaporation apparatus and exposed to the air, and then aluminum oxide was deposited to a thickness of 30 nm by an ALD method using trimethylaluminum (abbreviation: TMA) as a precursor and water vapor as an oxidizer to form a first sacrificial layer.

Next, over the first sacrificial layer, a composite oxide containing indium, gallium, zinc, and oxygen (abbreviation: IGZO) was deposited to a thickness of 50 nm by a sputtering method to form a second sacrificial layer.

A resist was formed using a photoresist over the second sacrificial layer, and processing was performed by a photolithography method to form a slit having a width of 3 μm in a position 3.5 μm away from an end portion of the first electrode.

Specifically, the second sacrificial layer was processed using a chemical solution containing a phosphoric acid solution with the use of a resist as a mask, and then the first sacrificial layer was processed using an etching gas containing fluoroform (CHF₃) and helium (He) at a flow rate ratio of CHF₃:He=1:9. Then, the second electron-transport layer, the second light-emitting layer, the second hole-transport layer, the intermediate layer, the first electron-transport layer, the first light-emitting layer, the first hole-transport layer, and the hole-injection layer were processed using an etching gas containing oxygen (O₂).

After the processing by a photolithography method, the first and second sacrificial layers were removed using a basic chemical solution containing water as a solvent, so that the top surface of the second electron-transport layer was exposed. Then, the substrate was transferred into a vacuum evaporation apparatus where the pressure was reduced to approximately 1×10⁻⁴ Pa, and heat treatment was performed at 110° C. for 1 hour in a heating chamber of the vacuum evaporation apparatus.

The above is the description of the processing by a photolithography method and the heat treatment. As described above, in the processing by a photolithography method and the heat treatment, treatment using water or a chemical solution containing water as a solvent is performed.

After the processing by a photolithography method and the heat treatment, over the second electron-transport layer, lithium fluoride (LiF) and ytterbium (Yb) were deposited by co-evaporation to a thickness of 1.5 nm such that the volume ratio of LiF to Yb was 2:1 to form the electron-injection layer, and lastly silver (Ag) and magnesium (Mg) were deposited by co-evaporation to a thickness of 15 nm such that the volume ratio of Ag to Mg was 1:0.1 to form the second electrode, whereby the light-emitting device 1 was fabricated.

The second electrode is a semi-transmissive and semi-reflective electrode having a function of reflecting light and a function of transmitting light; thus, the light-emitting device of this example is a top-emission tandem device in which light is extracted through the second electrode. Over the second electrode, 4,4′,4″-(benzene-1,3,5-triyl)tri(dibenzothiophene) (abbreviation: DBT3P-II) was deposited by evaporation to a thickness of 70 nm as a cap layer to improve light extraction efficiency.

The structure of the light-emitting device 1 is shown in the following table.

TABLE 1

| | | Film | | |
|---|---|---|---|---|
| | | thickness | | Light-emitting device 1 |
| Cap layer | | 70 | nm | DBT3P-II |
| Second electrode | | 15 | nm | Ag:Mg (1:0.1) |
| Electron-injection layer | | 1.5 | nm | LiF:Yb (2:1) |
| | | | | Processing was performed by photolithography method |
| Second | 2 | 20 | nm | mPPhen2P |
| electron- | 1 | 20 | nm | 2mPCCzPDBq |
| transport layer | | | | |
| Second light-emitting layer | | 40 | nm | 8mpTP-4mDBtPBfpm:βNCCP:Ir(5mppy-d₃)₂(mbfpypy-d₃) |
| | | | | (0.5:0.5:0.1) |
| Second hole-transport layer | | 55 | nm | PCBBiF |
| Intermediate | Third layer | 10 | nm | PCBBiF:OCHD-003 (1:0.15) |
| layer | Second layer | 2 | nm | CuPc |
| | First layer | 5 | nm | mPPhen2P:Fha-hpp (6.6:3.3) |
| First electron-transport layer | | 10 | nm | DACT-II |
| First light-emitting layer | | 40 | nm | 8mpTP-4mDBtPBfpm:βNCCP:Ir(5mppy-d₃)₂(mbfpypy-d₃) |
| | | | | (0.5:0.5:0.1) |
| First hole-transport layer | | 60 | nm | PCBBiF |
| Hole-injection layer | | 10 | nm | PCBBiF:OCHD-003 (1:0.03) |
| First electrode | 2 | 100 | nm | ITSO |
| | 1 | 100 | nm | APC |

The light-emitting device 1 was sealed using a glass substrate in a glove box containing a nitrogen atmosphere so as not to be exposed to the air. Specifically, a UV curable sealing material was applied to surround the device, only the sealing material was irradiated with UV while the light-emitting device was not irradiated with the UV, and heat treatment was performed at 80° C. under an atmospheric pressure for one hour. Then, the initial characteristics of the light-emitting device were measured.

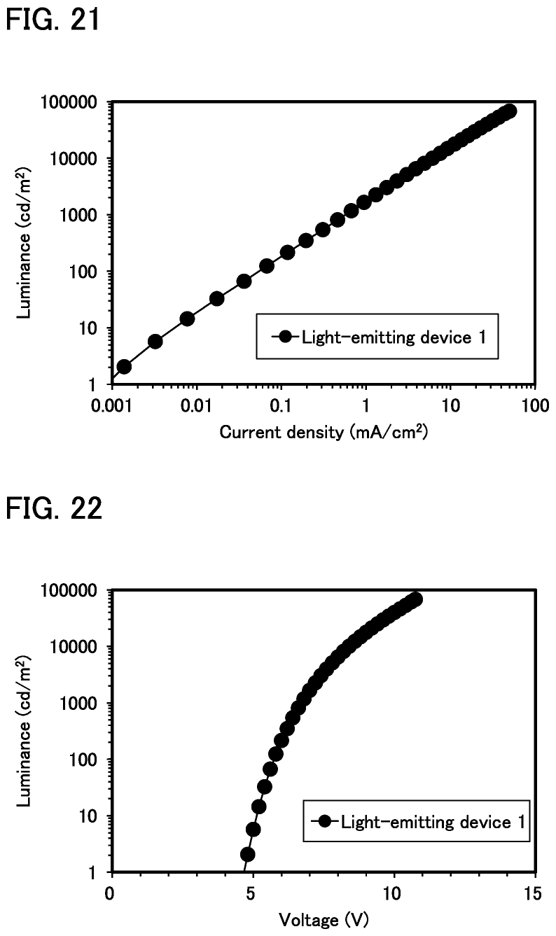

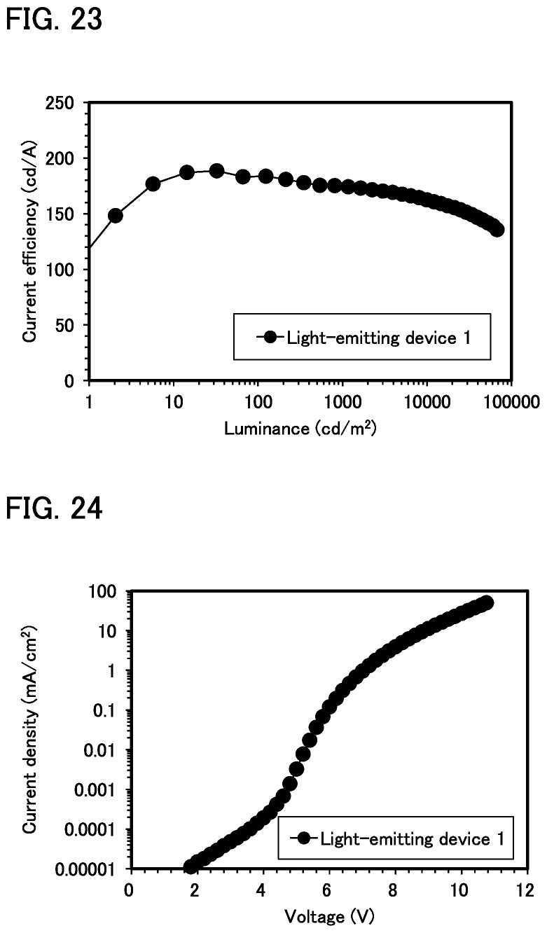

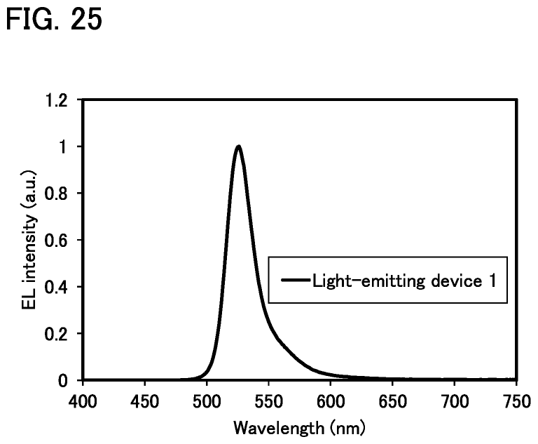

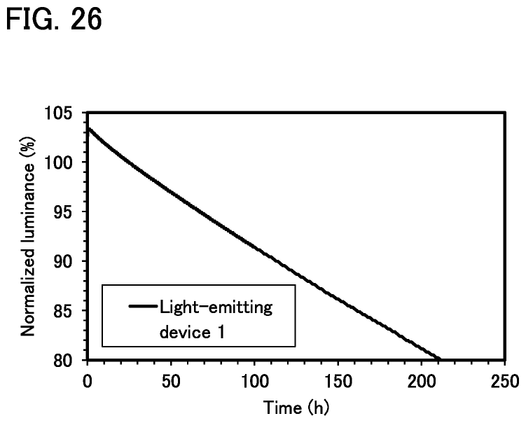

FIG. 21 shows the luminance-current density characteristics of the light-emitting device 1, FIG. 22 shows the luminance-voltage characteristics thereof, FIG. 23 shows the current efficiency-luminance characteristics thereof, FIG. 24 shows the current density-voltage characteristics thereof, and FIG. 25 shows the electroluminescence spectrum thereof. FIG. 26 shows a luminance change over driving time when the light-emitting device 1 was driven at a constant current of 2 mA (50 mA/cm²).

The following table shows the main characteristics of the light-emitting device 1 at a luminance of approximately 1000 cd/m². The luminance, CIE chromaticity, and electroluminescence spectrum were measured at normal temperature with a spectroradiometer (SR-UL1R manufactured by TOPCON TECHNOHOUSE CORPORATION).

TABLE 2

| | | | Current | | | | Current |
|---|---|---|---|---|---|---|---|
| | Voltage | Current | density | | | Luminance | efficiency |
| | (V) | (mA) | (mA/cm²) | Chromaticity x | Chromaticity y | (cd/m²) | (cd/A) |
| Light-emitting | 6.8 | 0.027 | 0.67 | 0.221 | 0.731 | 1172 | 174.0 |
| device 1 | | | | | | | |

FIG. 21 to FIG. 26 and the above table reveal that the light-emitting device 1 exhibits green light emission derived from Ir(5mppy-d₃)₂(mbfpypy-d₃) and has favorable light-emitting characteristics. In addition, the light-emitting device 1 has high current efficiency and is driven as a tandem light-emitting device. This indicates that the use of the organic compound of one embodiment of the present invention for an intermediate layer enables a light-emitting device to have favorable characteristics.

Since the organic compound of one embodiment of the present invention has low water solubility, problems such as dissolution of a layer including the organic compound and permeation of a chemical solution into the layer can be prevented even when a step using water is included in a manufacturing process of the light-emitting device including the organic compound of one embodiment of the present invention. Moreover, the organic compound of one embodiment of the present invention has a glass transition temperature higher than or equal to 100° C., thereby preventing abnormality in film quality in a heating step. Furthermore, as shown in this example, it is found that the use of the organic compound of one embodiment of the present invention for an intermediate layer of a light-emitting device enables the light-emitting device to have favorable characteristics. Thus, in manufacture of a light-emitting device whose manufacturing process includes a step using water, the use of the organic compound of one embodiment of the present invention for an intermediate layer enables the light-emitting device to have favorable characteristics while preventing degraded characteristics or a shape defect of the light-emitting device, for example, as compared to the case of using an organic compound having high water solubility and the case of using an organic compound having a low glass transition temperature.

### Example 5

This example describes the light-emitting device 2 fabricated using SBF-hpp (Structural Formula (115)), which is the organic compound of one embodiment of the present invention. Note that the light-emitting device 2 is a light-emitting device fabricated through what is called a continuous vacuum process, which does not include a step of processing the organic compound layer by a photolithography method. Structural formulae of organic compounds used for the light-emitting device 2 are shown below.

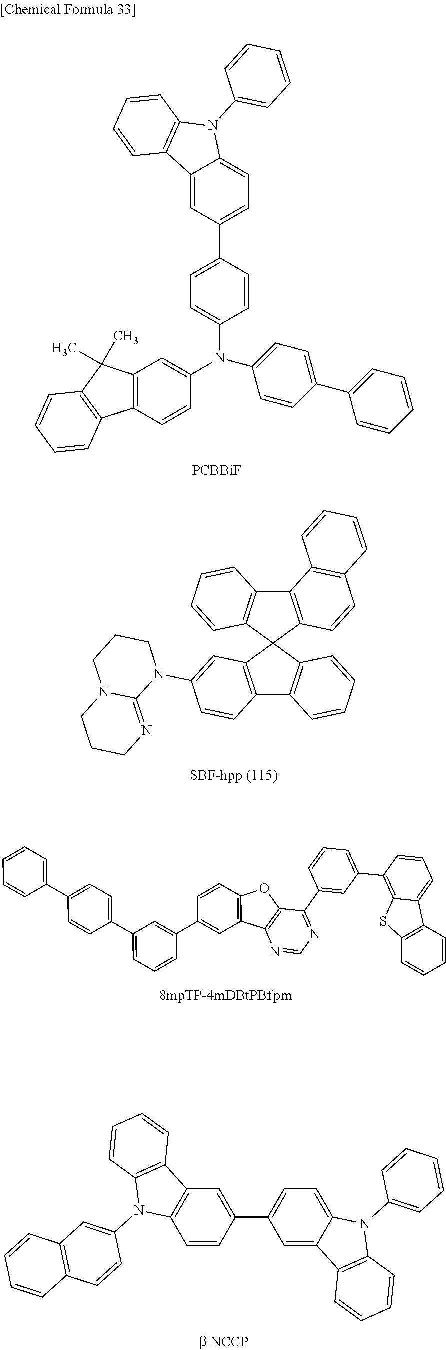

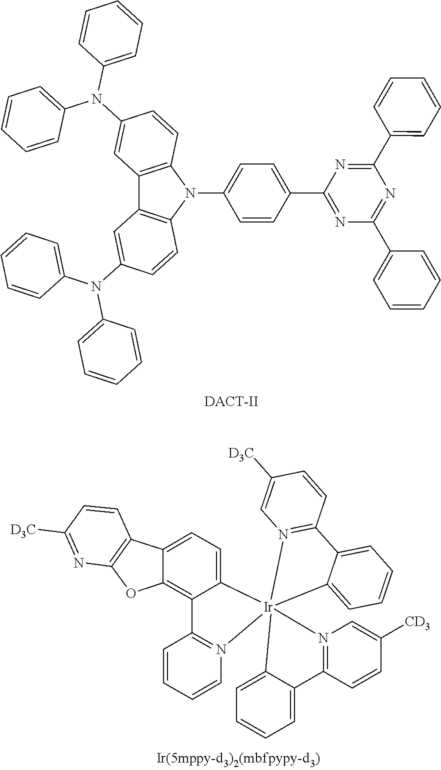

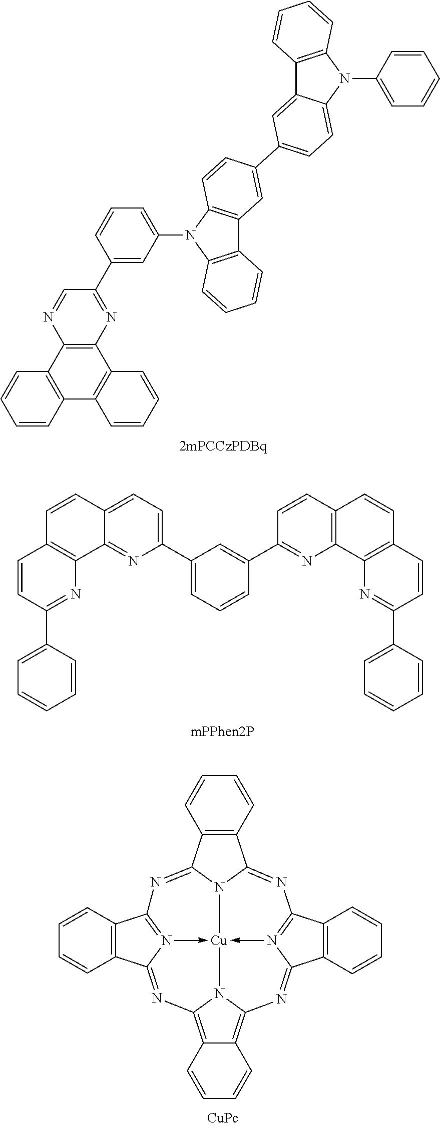

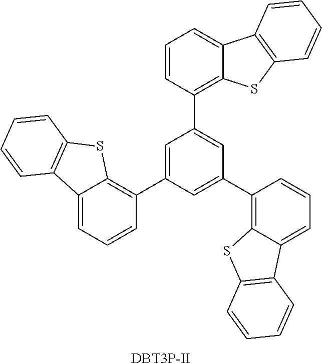 [SMILES: c1ccc2c(c1)sc1c(-c3cc(-c4cccc5c4sc4ccccc45)cc(-c4cccc5c4sc4ccccc45)c3)cccc12 ; "DBT3P-II"]

### (Fabrication Method of Light-Emitting Device 2)

The light-emitting device 2 is a light-emitting device fabricated through what is called a continuous vacuum process, which does not include a step of processing the organic compound layer by a photolithography method. That is, the fabrication method of the light-emitting device 2 is different from the fabrication method of the light-emitting device 1 in that, after formation of the second electron-transport layer, the electron-injection layer was successively formed without performing processing by a photolithography method and heat treatment.

The light-emitting device 2 is different from the light-emitting device 1 in the material and thickness of the first electrode, the thickness of the first hole-transport layer, the structure of the first layer of the intermediate layer, and the thickness of the second hole-transport layer. Specifically, in the first electrode, APC was replaced with Ag and the thickness of ITSO was set to 85 nm. The thickness of the first hole-transport layer was set to 80 nm. The first layer of the intermediate layer was formed by replacing Fha-hpp used for the light-emitting device 1 with SBF-hpp (Structural Formula (115)), which is the organic compound of another embodiment of the present invention, and depositing mPPhen2P and SBF-hpp by co-evaporation to a thickness of 5 nm such that the weight ratio of mPPhen2P to SBF-hpp was 0.4:0.6. The thickness of the second hole-transport layer was set to 50 nm.

The other components of the light-emitting device 2 were formed in a manner similar to that of the light-emitting device 1, and thus are not described here. The structure of the light-emitting device 2 is shown in the following table.

TABLE 3

| | | Film | | |
|---|---|---|---|---|
| | | thickness | | Light-emitting device 2 |
| Cap layer | | 70 | nm | DBT3P-II |
| Second electrode | | 15 | nm | Ag:Mg (1:0.1) |
| Electron-injection layer | | 1.5 | nm | LiF:Yb (2:1) |
| Second | 2 | 20 | nm | mPPhen2P |
| electron- | 1 | 20 | nm | 2mPCCzPDBq |
| transport layer | | | | |
| Second light-emitting layer | | 40 | nm | 8mpTP-4mDBtPBfpm:βNCCP:Ir(5mppy-d₃)₂(mbfpypy-d₃) |
| | | | | (0.5:0.5:0.1) |
| Second hole-transport layer | | 50 | nm | PCBBiF |
| Intermediate | Third layer | 10 | nm | PCBBiF:OCHD-003 (1:0.15) |
| layer | Second layer | 2 | nm | CuPc |
| | First layer | 5 | nm | mPPhen2P:SBF-hpp (0.4:0.6) |
| First electron-transport layer | | 10 | nm | DACT-II |
| First light-emitting layer | | 40 | nm | 8mpTP-4mDBtPBfpm:βNCCP:Ir(5mppy-d₃)₂(mbfpypy-d₃) |
| | | | | (0.5:0.5:0.1) |
| First hole-transport layer | | 80 | nm | PCBBiF |
| Hole-injection layer | | 10 | nm | PCBBiF:OCHD-003 (1:0.03) |
| First electrode | 2 | 85 | nm | ITSO |
| | 1 | 100 | nm | Ag |

The light-emitting device 2 was sealed using a glass substrate in a glove box containing a nitrogen atmosphere so as not to be exposed to the air. Specifically, a UV curable sealing material was applied to surround the device, only the sealing material was irradiated with UV while the light-emitting device was not irradiated with the UV, and heat treatment was performed at 80° C. under an atmospheric pressure for one hour. Then, the initial characteristics of the light-emitting device were measured.

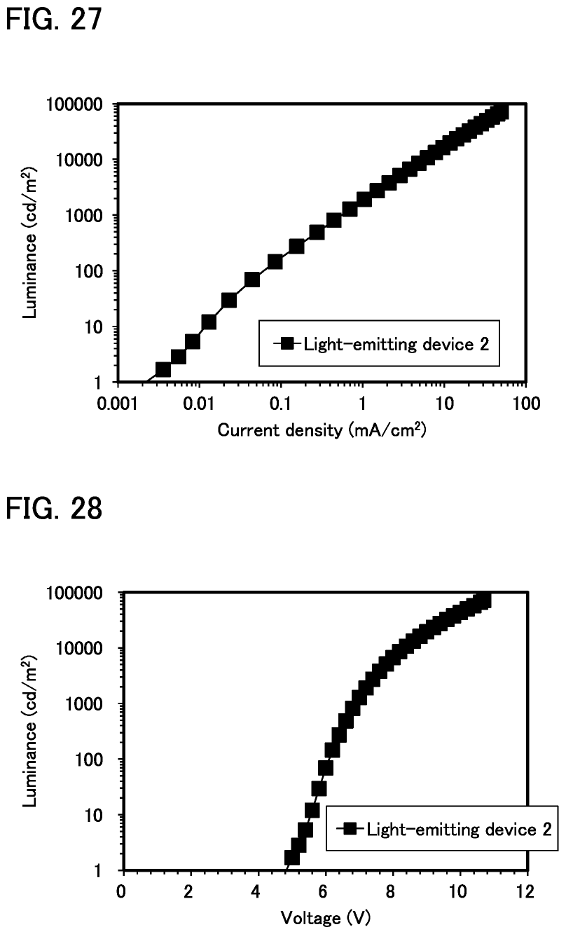

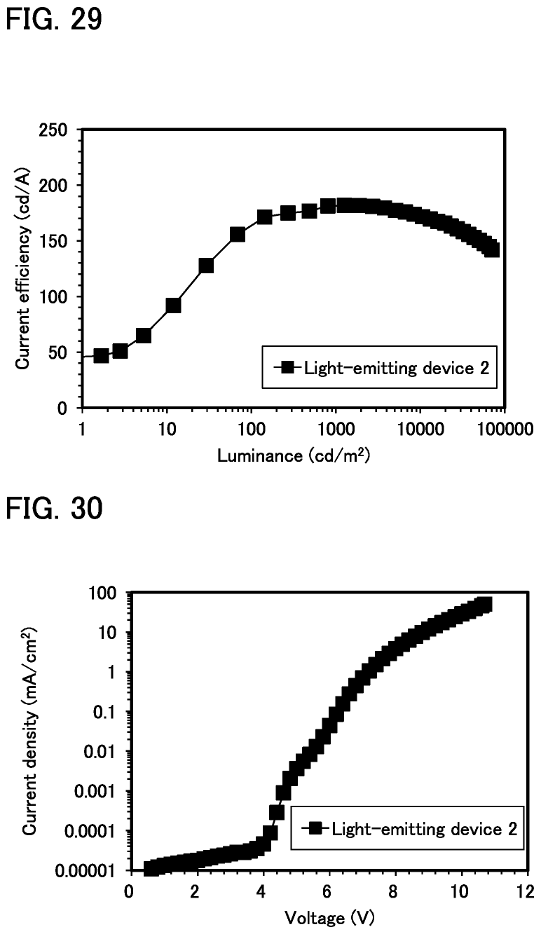

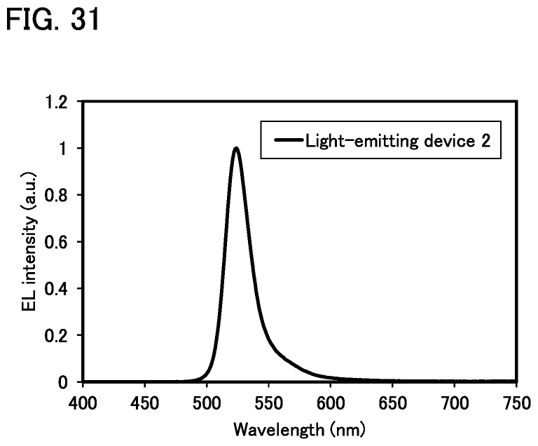

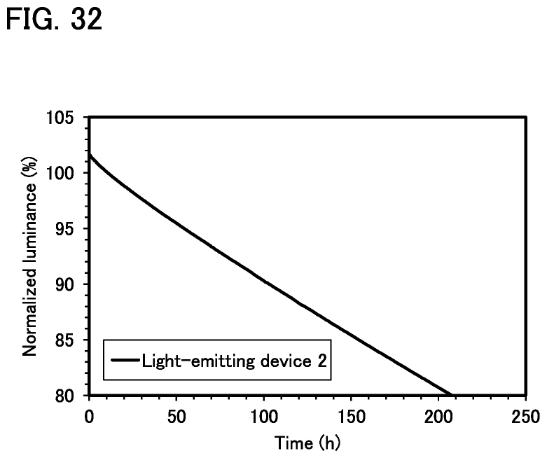

FIG. 27 shows the luminance-current density characteristics of the light-emitting device 2, FIG. 28 shows the luminance-voltage characteristics thereof, FIG. 29 shows the current efficiency-luminance characteristics thereof, FIG. 30 shows the current density-voltage characteristics thereof, FIG. 31 shows the electroluminescence spectrum thereof, and FIG. 32 shows a luminance change over driving time when the light-emitting device 2 was driven at a constant current of 2 mA (50 mA/cm²).

The following table shows the main characteristics of the light-emitting device 2 at a luminance of approximately 1000 cd/m². The luminance, CIE chromaticity, and electroluminescence spectrum were measured at normal temperature with a spectroradiometer (SR-UL1R manufactured by TOPCON TECHNOHOUSE CORPORATION).

TABLE 4

| | | | Current | | | | Current |
|---|---|---|---|---|---|---|---|
| | Voltage | Current | density | | | Luminance | efficiency |
| | (V) | (mA) | (mA/cm²) | Chromaticity x | Chromaticity y | (cd/m²) | (cd/A) |
| Light-emitting | 6.8 | 0.018 | 0.45 | 0.201 | 0.743 | 808 | 181 |
| device 2 | | | | | | | |

FIG. 27 to FIG. 32 and the above table reveal that the light-emitting device 2 exhibits green light emission derived from Ir(5mppy-d₃)₂(mbfpypy-d₃) and has favorable light-emitting characteristics. In addition, the light-emitting device 2 has high current efficiency and is driven as a tandem light-emitting device. This indicates that the use of the organic compound of one embodiment of the present invention for an intermediate layer enables a light-emitting device to have favorable characteristics.

### Example 6

This example describes the light-emitting device 3 fabricated using mSFP-hpp (Structural Formula (133)), which is the organic compound of one embodiment of the present invention. The light-emitting device 3 is a light-emitting device fabricated through what is called a continuous vacuum process, which does not include a step of processing the organic compound layer by a photolithography method. Structural formulae of organic compounds used for the light-emitting device 3 are shown below.

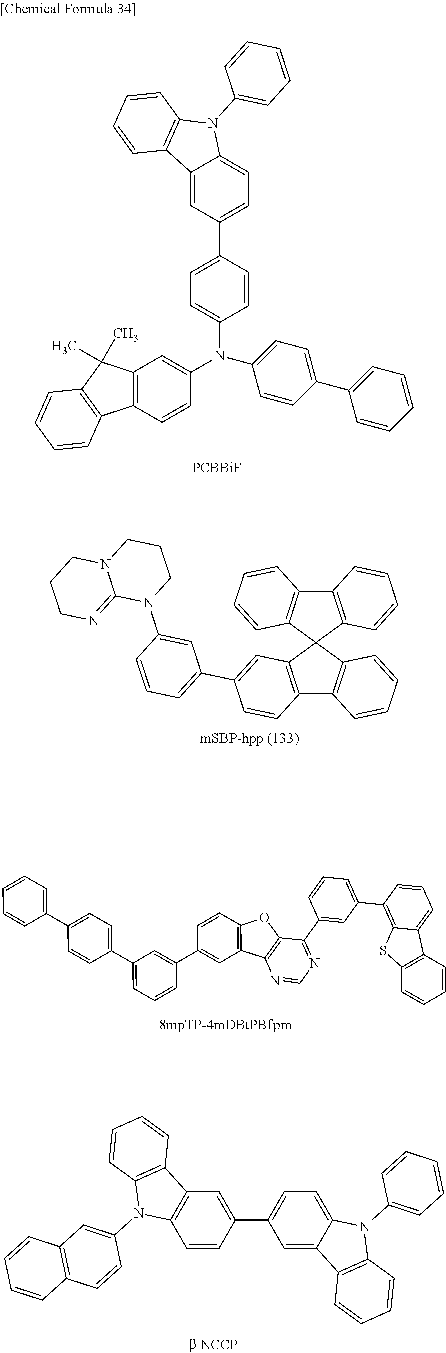

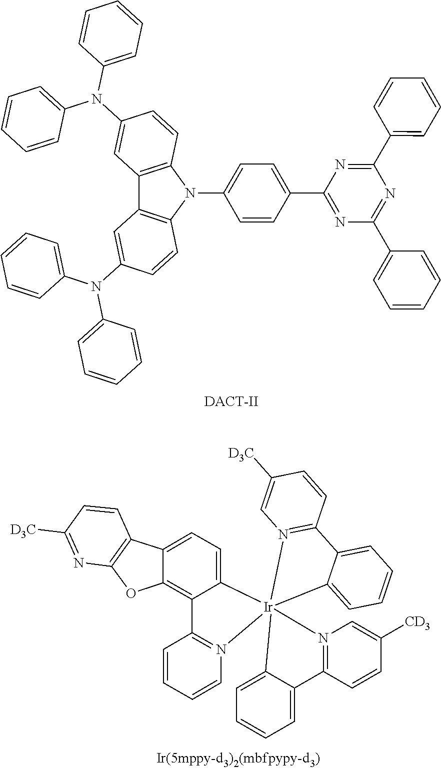

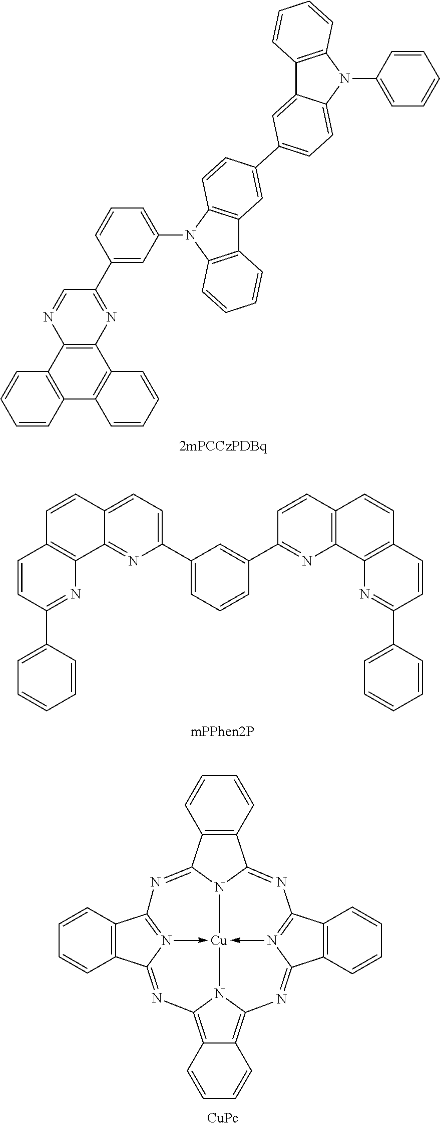

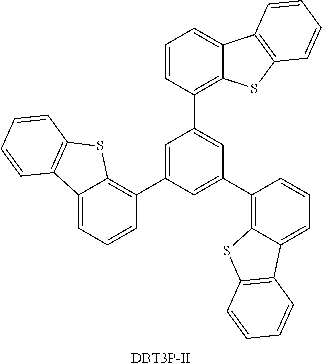 [SMILES: c1ccc2c(c1)sc1c(-c3cc(-c4cccc5c4sc4ccccc45)cc(-c4cccc5c4sc4ccccc45)c3)cccc12 ; "DBT3P-II"]

### (Fabrication Method of Light-Emitting Device 3)

Like the light-emitting device 2, the light-emitting device 3 is a light-emitting device fabricated through what is called a continuous vacuum process, which does not include a step of processing the organic compound layer by a photolithography method.

The light-emitting device 3 is different from the light-emitting device 2 in the thickness of the first electron-transport layer and the structure of the first layer of the intermediate layer. Specifically, the thickness of the first electron-transport layer was set to 20 nm. The first layer of the intermediate layer was formed by replacing SBF-hpp used for the light-emitting device 2 with mSFP-hpp (Structural Formula (133)), which is the organic compound of another embodiment of the present invention, and depositing mPPhen2P and mSFP-hpp by co-evaporation to a thickness of 5 nm such that the weight ratio of mPPhen2P to mSFP-hpp was 0.5:0.5.

The other components of the light-emitting device 3 were formed in a manner similar to that of the light-emitting device 2, and thus are not described here. The structure of the light-emitting device 3 is shown in the following table.

TABLE 5

| | | Film | | |
|---|---|---|---|---|
| | | thickness | | Light-emitting device 3 |
| Cap layer | | 70 | nm | DBT3P-II |
| Second electrode | | 15 | nm | Ag:Mg (1:0.1) |
| Electron-injection layer | | 1.5 | nm | LiF:Yb (2:1) |
| Second | 2 | 20 | nm | mPPhen2P |
| electron- | 1 | 20 | nm | 2mPCCzPDBq |
| transport layer | | | | |
| Second light-emitting layer | | 40 | nm | 8mpTP-4mDBtPBfpm:βNCCP:Ir(5mppy-d₃)₂(mbfpypy-d₃ |
| | | | | (0.5:0.5:0.1) |
| Second hole-transport layer | | 50 | nm | PCBBiF |
| Intermediate | Third layer | 10 | nm | PCBBiF:OCHD-003 (1:0.15) |
| layer | Second layer | 2 | nm | CuPc |
| | First layer | 5 | nm | mPPhen2P:mSFP-hpp (0.5:0.5) |
| First electron-transport layer | | 20 | nm | DACT-II |
| First light-emitting layer | | 40 | nm | 8mpTP-4mDBtPBfpm:βNCCP:Ir(5mppy-d₃)₂(mbfpypy-d₃) |
| | | | | (0.5:0.5:0.1) |
| First hole-transport layer | | 80 | nm | PCBBiF |
| Hole-injection layer | | 10 | nm | PCBBiF:OCHD-003 (1:0.03) |
| First electrode | 2 | 85 | nm | ITSO |
| | 1 | 100 | nm | Ag |

The light-emitting device 3 was sealed using a glass substrate in a glove box containing a nitrogen atmosphere so as not to be exposed to the air. Specifically, a UV curable sealing material was applied to surround the device, only the sealing material was irradiated with UV while the light-emitting device was not irradiated with the UV, and heat treatment was performed at 80° C. under an atmospheric pressure for one hour. Then, the initial characteristics of the light-emitting device were measured.

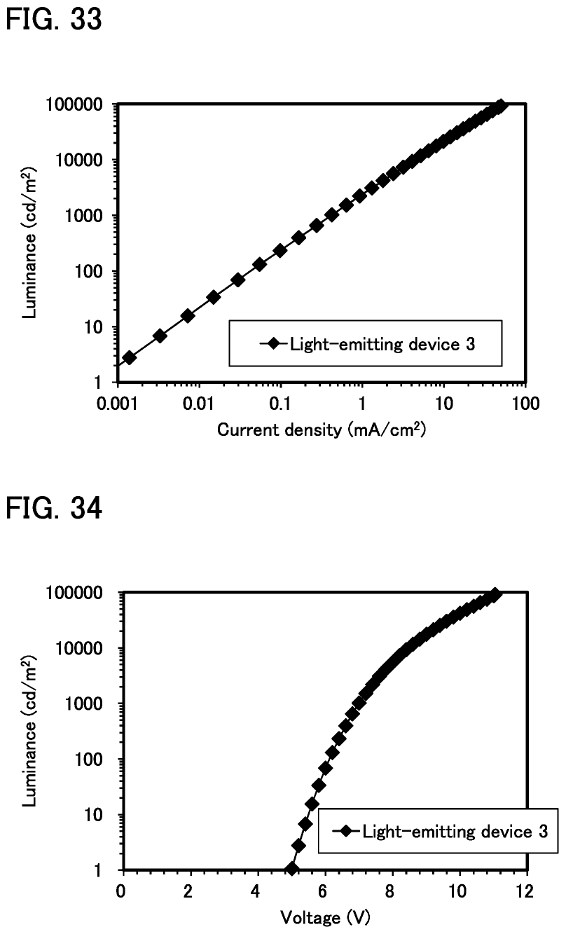

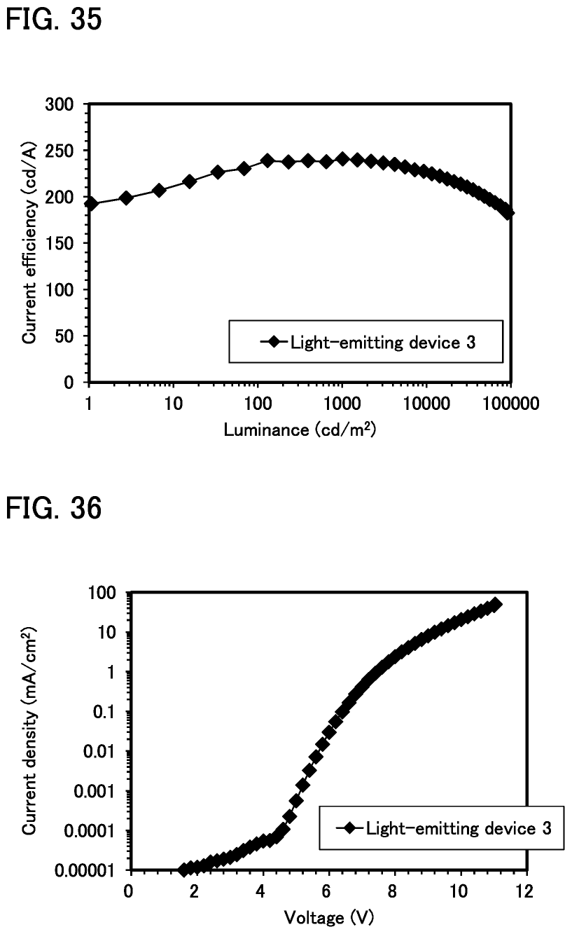

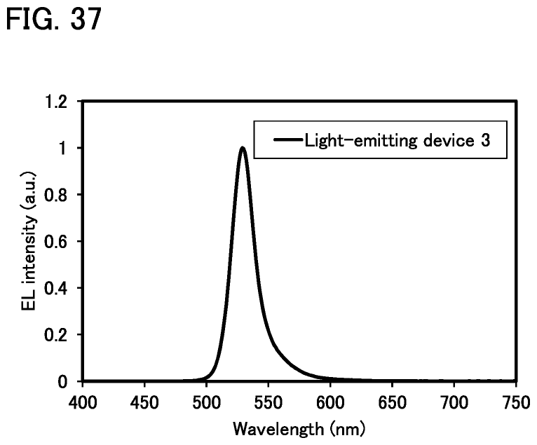

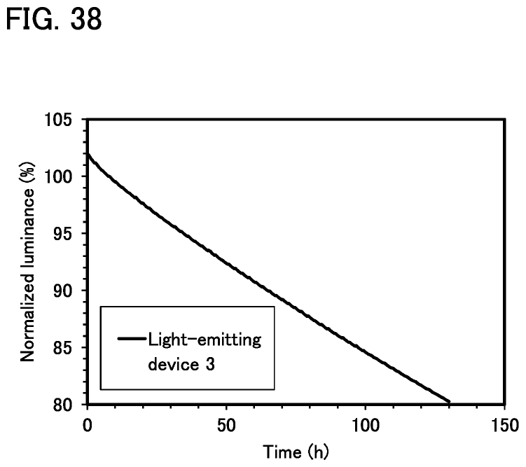

FIG. 33 shows the luminance—current density characteristics of the light-emitting device 3, FIG. 34 shows the luminance—voltage characteristics thereof, FIG. 35 shows the current efficiency—luminance characteristics thereof, FIG. 36 shows the current density—voltage characteristics thereof, FIG. 37 shows the electroluminescence spectrum thereof, and FIG. 38 shows a luminance change over driving time when the light-emitting device 3 was driven at a constant current of 2 mA (50 mA/cm²).

The following table shows the main characteristics of the light-emitting device 3 at a luminance of approximately 1000 cd/m². The luminance, CIE chromaticity, and electroluminescence spectrum were measured at normal temperature with a spectroradiometer (SR-UL1R manufactured by TOPCON TECHNOHOUSE CORPORATION).

TABLE 6

| | | | Current | | | | Current |
|---|---|---|---|---|---|---|---|
| | Voltage | Current | density | | | Luminance | efficiency |
| | (V) | (mA) | (mA/cm²) | Chromaticity x | Chromaticity y | (cd/m²) | (cd/A) |
| Light-emitting | 7.0 | 0.017 | 0.42 | 0.211 | 0.747 | 1015 | 241 |
| device 3 | | | | | | | |

FIG. 33 to FIG. 38 and the above table reveal that the light-emitting device 3 exhibits green light emission derived from Ir(5mppy-d₃)₂(mbfpypy-d₃) and has favorable light-emitting characteristics. In addition, the light-emitting device 3 has high current efficiency and is driven as a tandem light-emitting device. This indicates that the use of the organic compound of one embodiment of the present invention for an intermediate layer enables a light-emitting device to have favorable characteristics.

### Example 7

### Synthesis Example 4

This example describes a synthesis method of 1-[6-(9,9′-spirobi[9H-fluoren]-2-yl)pyridin-2-yl]-1,3,4,6,7,8-hexahydro-2H-pyrimido[1,2-a]pyrimidine (abbreviation: SFPy-hpp) (Structural Formula (137)), which is an organic compound of one embodiment of the present invention. The structure of SFPy-hpp is shown below.

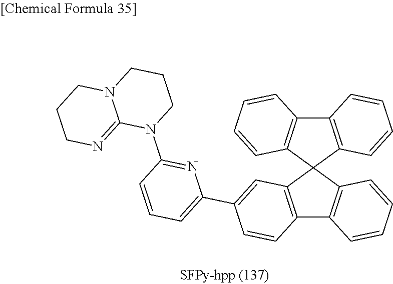 [SMILES: c1cc(-c2ccc3c(c2)C2(c4ccccc4-c4ccccc42)c2ccccc2-3)nc(N2CCCN3CCCN=C32)c1]

### Step 1: Synthesis of 2-bromo-6-(9,9′-spirobi[9H-fluoren]-2-yl)pyridine

In a 300-mL three-neck flask were put 3.8 g (11 mmol) of 9,9′-spirobi[9H-fluoren]-2-ylboronic acid, 4.5 g (19 mmol) of 2,6-dibromopyridine, 4.4 g (32 mmol) of potassium carbonate, 80 mL of toluene, 26 mL of ethanol, and 16 mL of water, and the mixture was degassed by stirring under reduced pressure. To this mixture was added 0.24 g (0.21 mmol) of tetrakis(triphenylphosphine)palladium(0), and the mixture was stirred at 90° C. under a nitrogen stream for 30 minutes. After the stirring, the mixture was cooled down to room temperature, and extraction with toluene was performed. Then, the extracted solution was concentrated to give an oily substance. The oily substance was purified by silica gel column chromatography (developing solvent: toluene). The obtained fraction was concentrated to give a solid. The solid was purified by high performance liquid column chromatography (developing solvent: chloroform). The obtained fraction was concentrated to give 2.9 g of a target white solid in a yield of 58%. The synthesis scheme of Step 1 is shown in Formula (d-1) below.

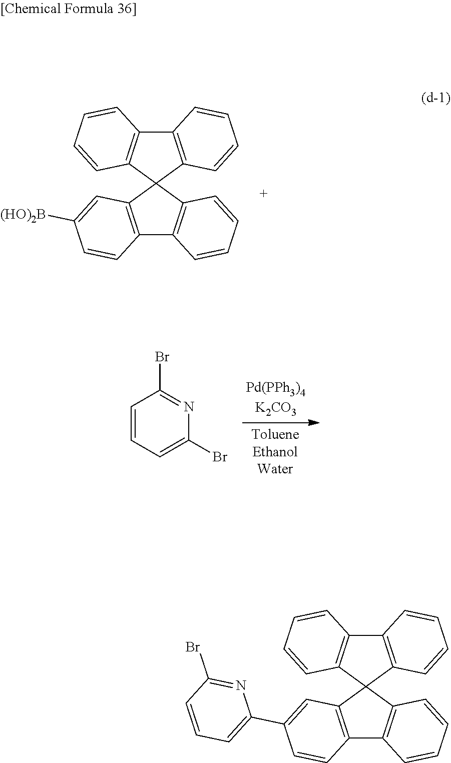 [SMILES: OB(O)c1ccc2c(c1)C1(c3ccccc3-c3ccccc31)c1ccccc1-2 ; Brc1cccc(Br)n1 ; Brc1cccc(-c2ccc3c(c2)C2(c4ccccc4-c4ccccc42)c2ccccc2-3)n1]

### Step 2: Synthesis of SFPy-hpp

In a 200-mL three-neck flask were put 2.9 g (6.1 mmol) of 2-bromo-6-(9,9′-spirobi[9H-fluoren]-2-yl)pyridine synthesized in Step 1, 1.1 g (7.9 mmol) of 1,3,4,6,7,8-hexahydro-2H-pyrimido[1,2-a]pyrimidine, 0.23 g (0.37 mmol) of (±)-2,2′-bis(diphenylphosphino)-1,1′-binaphthyl (abbreviation: rac-BINAP), 1.7 g (15 mmol) of potassium tert-butoxide, and 61 mL of dehydrated toluene, and the mixture was degassed by stirring under reduced pressure. To this mixture, 55 mg (0.24 mmol) of palladium(II) acetate was added and stirring was performed at 90° C. under a nitrogen stream for 7 hours. After the stirring, the mixture was cooled down to room temperature. An insoluble matter was separated from the mixture by suction filtration, and the obtained filtrate was subjected to extraction with toluene. The extracted solution was concentrated to give a solid. Toluene was added to the solid to dissolve the solid, saturated saline was added, irradiation with ultrasonic waves was performed, and then a precipitated solid was collected by suction filtration. A sodium hydroxide solution and toluene were added to the solid, stirring was performed at room temperature overnight, and extraction with toluene was performed. The extracted solution was concentrated to give an oily substance. A small amount of toluene and hexane were added to the oily substance, irradiation with ultrasonic waves was performed, and a precipitated solid was collected by suction filtration, whereby 1.3 g of a target pale yellow solid was obtained in a yield of 39%. The synthesis scheme of SFPy-hpp is shown in Formula (d-2) below.

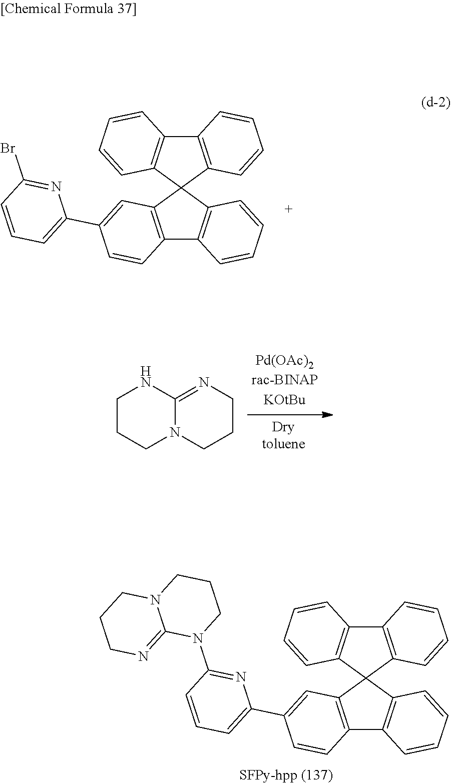 [SMILES: Brc1cccc(-c2ccc3c(c2)C2(c4ccccc4-c4ccccc42)c2ccccc2-3)n1 ; C1CN=C2NCCCN2C1 ; c1cc(-c2ccc3c(c2)C2(c4ccccc4-c4ccccc42)c2ccccc2-3)nc(N2CCCN3CCCN=C32)c1]

By a train sublimation method, 1.3 g of the obtained pale yellow solid was purified by sublimation. In the purification by sublimation, heating was performed for 42 hours at an argon flow rate of 10 mL/min, a pressure of 6.1 Pa, and a heating temperature of 240° C. As a result, 0.81 g of a target pale yellow solid was collected at a collection rate of 62%.

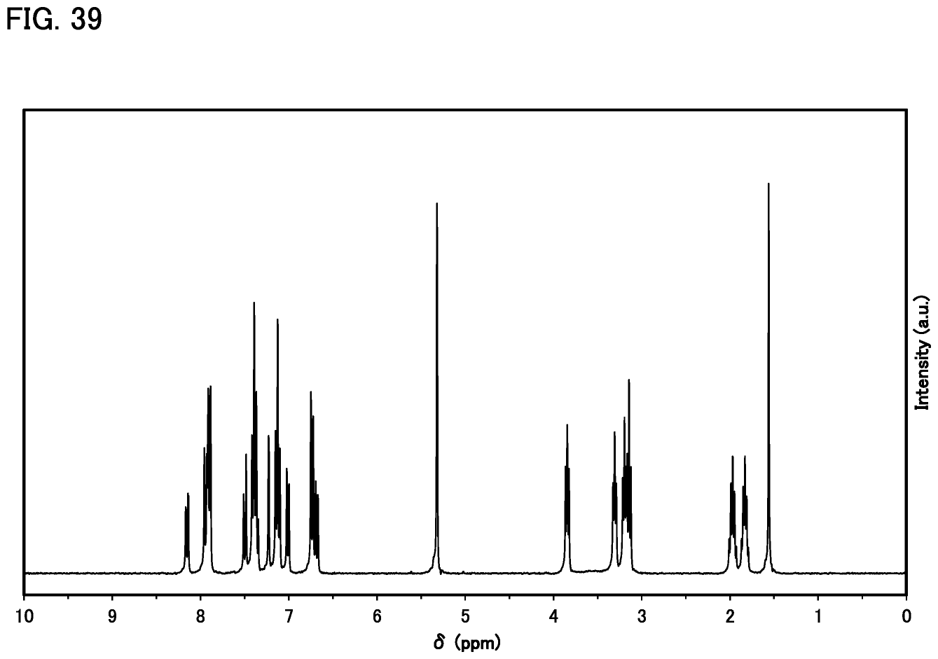

FIG. 39 shows a ¹H NMR spectrum of SFPy-hpp after the purification by sublimation. Results of ¹H NMR measurement are shown below. The results show that SFPy-hpp was obtained.

¹H NMR (CD₂Cl₂, 300 MHz): δ=8.16 (dd, J=7.8 Hz, 1.2 Hz, 1H), 7.96-7.89 (m, 4H), 7.51-7.35 (m, 5H), 7.23 (s, 1H), 7.13 (t, J=7.4 Hz, 3H), 7.01 (d, J=7.8 Hz, 1H), 6.75-6.67 (m, 3H), 3.84 (t, J=6.0 Hz, 2H), 3.31 (t, J=5.7 Hz, 2H), 3.22-3.12 (m, 4H), 2.01-1.93 (m, 2H), 1.87-1.79 (m, 2H).

Tg of SFPy-hpp was measured. Note that Tg was measured with a differential scanning calorimeter (DSC8500, manufactured by PerkinElmer Japan Co., Ltd.) in a state where a powder was put on an aluminum cell and the temperature was increased at a rate of 40° C./min. As a result, Tg of SFPy-hpp was 115° C.

### <Solubility Test of SFPy-hpp by LC-MS Analysis>

In the LC-MS analysis, liquid chromatography (LC) separation was carried out with ACQUITY UPLC manufactured by Waters Corporation, and MS analysis (mass spectrometry) was carried out with Xevo G2 Tof MS manufactured by Waters Corporation. Acquity UPLC BEH C8 (2.1×100 mm, 1.7 μm) was used as a column for the LC separation. Acetonitrile was used for Mobile Phase A and a 0.1% aqueous solution of formic acid was used for Mobile Phase B. The injection amount of the sample was 5.0 μL. Note that in the analysis, the wavelength of a photodiode array detector was set to 330 nm±1 nm.

In a 5-mL sample bottle, 1 mg of SFPy-hpp was put and 2 mL of N,N-dimethylformamide was added thereto. This mixture was irradiated with ultrasonic waves for five minutes. After it was confirmed that the solid was completely dissolved, this solution was diluted by 2.5 times with acetonitrile, whereby the concentration of the solution was adjusted to 200 mg/L. This solution was diluted with acetonitrile, whereby a solution with a concentration of 4 mg/L, a solution with a concentration of 2 mg/L, and a solution with a concentration of 0.2 mg/L were prepared. The prepared solutions were subjected to LC-MS analysis, and the peak area values derived from SFPy-hpp, which were obtained at the respective solution concentrations, were used to form calibration curves.

Next, the water solubility of SFPy-hpp was measured.

In a 5-mL sample bottle, 1 mg of SFPy-hpp was put and 1 mL of water was added thereto. This mixture was irradiated with ultrasonic waves for five minutes. This mixture was filtered through a membrane filter to remove the solid. The obtained solution was subjected to LC-MS analysis.

From the calibration curve and the signal intensity obtained by the LC-MS analysis, it is found that the solubility of SFPy-hpp in 1 mL of water is 0.00029 mg. The weight fraction of the water solubility of SFPy-hpp is therefore 2.9×10⁻⁷ g/mL.

The above results reveal that SFPy-hpp is an organic compound with extremely low water solubility.

It is thus found that the organic compound of one embodiment of the present invention can be favorably used for a light-emitting device whose fabrication process includes processing using water or a chemical solution containing water as a solvent (i.e., a light-emitting device involving processing by a lithography method).

### Example 8

### Synthesis Example 5

This example describes a synthesis method of 1-[6-(2′,7′-di-tert-butyl-9,9′-spirobi[9H-fluoren]-2-yl)pyridin-2-yl]-1,3,4,6,7,8-hexahydro-2H-pyrimido[1,2-a]pyrimidine (abbreviation: tBuSFPy-hpp) (Structural Formula (153)), which is an organic compound of one embodiment of the present invention. The structure of tBuSFPy-hpp is shown below.

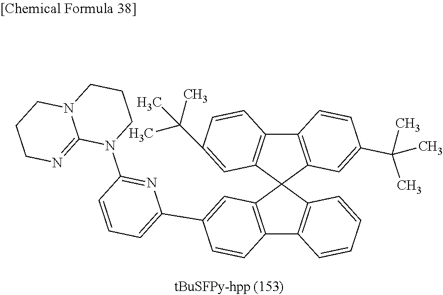 [SMILES: CC(C)(C)c1ccc2c(c1)C1(c3ccccc3-c3ccc(-c4cccc(N5CCCN6CCCN=C65)n4)cc31)c1cc(C(C)(C)C)ccc1-2]

### Step 1: Synthesis of 2-bromo-6-(2′,7′-di-tert-butyl-9,9′-spirobi[9H-fluoren]-2-yl)pyridine

In a 300-mL three-neck flask were put 4.9 g (10 mmol) of 2′,7′-di-tert-butyl-9,9′-spirobi[9H-fluoren]-2-ylboronic acid, 4.4 g (19 mmol) of 2,6-dibromo pyridine, 4.3 g (31 mmol) of potassium carbonate, 78 mL of toluene, 26 mL of ethanol, and 16 mL of water, and the mixture was degassed by stirring under reduced pressure. To this mixture was added 0.24 g (0.21 mmol) of tetrakis(triphenylphosphine)palladium(0), and the mixture was stirred at 90° C. under a nitrogen stream for 30 minutes. After the stirring, the mixture was cooled down to room temperature, followed by extraction with toluene. Then, the extracted solution was concentrated to give an oily substance. The oily substance was purified by silica gel column chromatography (developing solvent: toluene). The obtained fraction was concentrated to give an oily substance. The oily substance was purified by high performance liquid column chromatography (developing solvent: chloroform). The obtained fraction was concentrated to give 5.7 g of a target colorless transparent oily substance in a yield of 93%. The synthesis scheme of Step 1 is shown in (e-1) below.

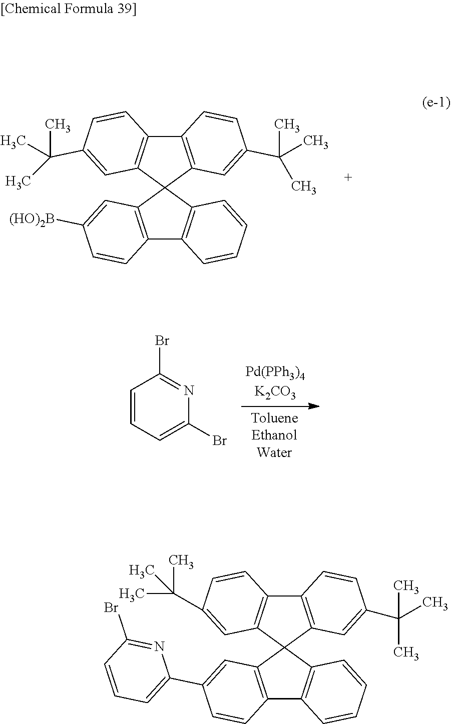 [SMILES: CC(C)(C)c1ccc2c(c1)C1(c3ccccc3-c3ccc(B(O)O)cc31)c1cc(C(C)(C)C)ccc1-2 ; Brc1cccc(Br)n1 ; CC(C)(C)c1ccc2c(c1)C1(c3ccccc3-c3ccc(-c4cccc(Br)n4)cc31)c1cc(C(C)(C)C)ccc1-2]

### Step 2: Synthesis of tBuSFPy-hpp

In a 300-mL three-neck flask were put 5.7 g (9.8 mmol) of 2-bromo-6-(2′,7′-di-tert-butyl-9,9′-spirobi[9H-fluoren]-2-yl)pyridine synthesized in Step 1, 1.7 g (12 mmol) of 1,3,4,6,7,8-hexahydro-2H-pyrimido[1,2-a]pyrimidine, 0.36 g (0.58 mmol) of (±)-2,2′-bis(diphenylphosphino)-1,1′-binaphthyl (abbreviation: rac-BINAP), 2.7 g (24 mmol) of potassium tert-butoxide, and 97 mL of dehydrated toluene, and the mixture was degassed by stirring under reduced pressure. To this mixture was added 87 mg (0.39 mmol) of palladium(II) acetate, and the mixture was stirred at 90° C. under a nitrogen stream for 7 hours. After the stirring, the mixture was cooled down to room temperature. An insoluble matter was separated from the mixture by suction filtration, and the obtained filtrate was subjected to extraction with toluene. The extracted solution was concentrated to give an oily substance. Toluene was added to the solid to dissolve the solid, saturated saline was added, irradiation with ultrasonic waves was performed, and then a precipitated solid was collected by suction filtration. Toluene and a sodium hydroxide solution were added to the solid, stirring was performed at room temperature overnight, and extraction with toluene was performed. The extracted solution was concentrated to give an oily substance. A small amount of toluene and hexane were added to the oily substance, irradiation with ultrasonic waves was performed, and a precipitated solid was collected by suction filtration, whereby 2.7 g of a target yellow solid was obtained in a yield of 43%. The synthesis scheme of tBuSFPy-hpp is shown in Formula (e-2) below.

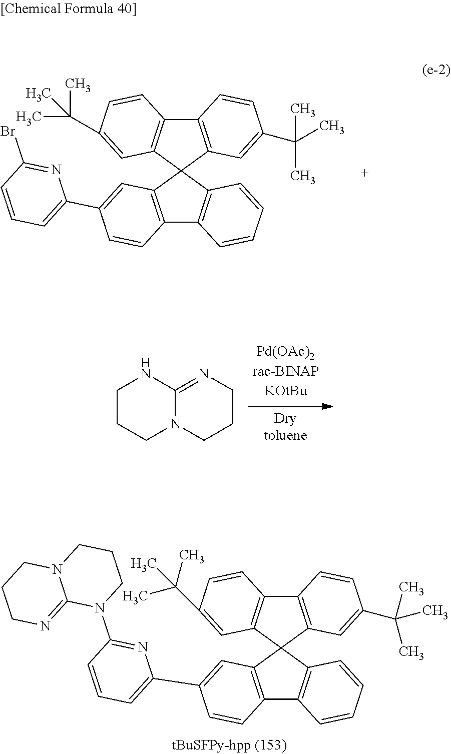 [SMILES: CC(C)(C)c1ccc2c(c1)C1(c3ccccc3-c3ccc(-c4cccc(Br)n4)cc31)c1cc(C(C)(C)C)ccc1-2 ; C1CN=C2NCCCN2C1 ; CC(C)(C)c1ccc2c(c1)C1(c3ccccc3-c3ccc(-c4cccc(N5CCCN6CCCN=C65)n4)cc31)c1cc(C(C)(C)C)ccc1-2]

By a train sublimation method, 2.7 g of the obtained pale yellow solid was purified. In the purification by sublimation, heating was performed for 42 hours at an argon flow rate of 18 mL/min, a pressure of 3.3 Pa, and a heating temperature of 250° C. As a result, 1.1 g of a target pale yellow solid was obtained at a collection rate of 41%.

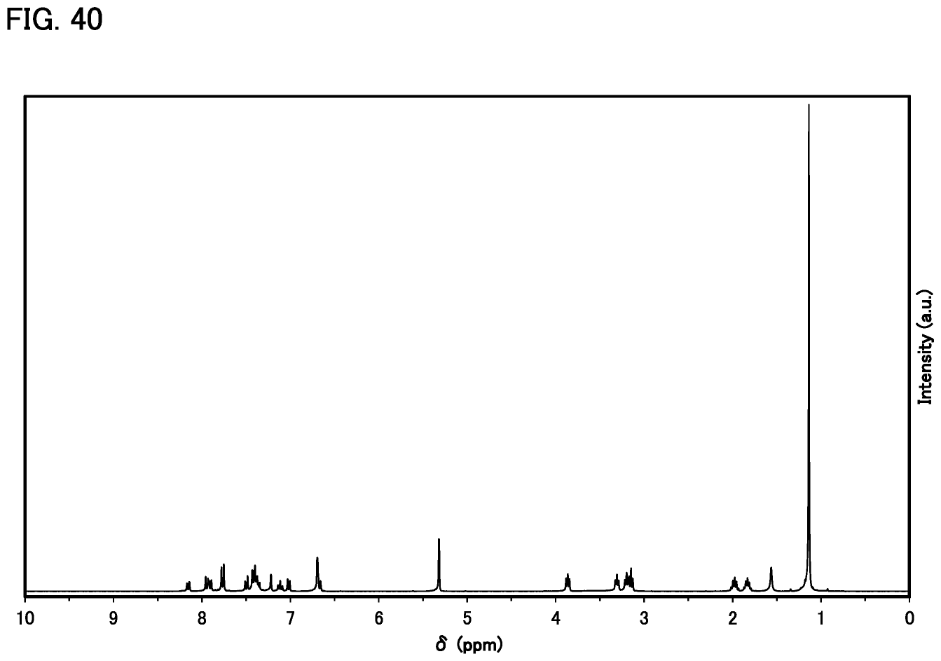

FIG. 40 shows a ¹H NMR spectrum of tBuSFPy-hpp after the purification by sublimation. Results of ¹H NMR measurement are shown below. The results show that tBuSFPy-hpp was obtained.

¹H NMR (CD₂Cl₂, 300 MHz): δ=8.15 (dd, J=8.1 Hz, 1.5 Hz, 1H), 7.96-7.89 (m, 2H), 7.77 (d, J=7.8 Hz, 2H), 7.51-7.35 (m, 5H), 7.22 (s, 1H), 7.11 (t, J=7.4 Hz, 1H), 7.02 (d, J=7.2 Hz, 1H), 6.70-6.66 (m, 3H), 3.86 (t, J=6.0 Hz, 2H), 3.31 (t, J=5.7 Hz, 2H), 3.22-3.13 (m, 4H), 2.02-1.93 (m, 2H), 1.87-1.79 (m, 2H), 1.14 (s, 18H).

Tg of tBuSFPy-hpp was measured. Note that Tg was measured with a differential scanning calorimeter (DSC8500, manufactured by PerkinElmer Japan Co., Ltd.) in a state where a powder was put on an aluminum cell and the temperature was increased at a rate of 40° C./min. As a result, tBuSFPy-hpp was 133° C.

### Example 9

This example describes the light-emitting device 4 fabricated using SFPy-hpp (Structural Formula (137)), which is the organic compound of one embodiment of the present invention, and the light-emitting device 5 fabricated using tBuSFPy-hpp (Structural Formula (153)), which is the organic compound of one embodiment of the present invention. The light-emitting devices 4 and 5 are light-emitting devices fabricated through what is called a continuous vacuum process, which does not include a step of processing the organic compound layer by a photolithography method. Structural formulae of organic compounds used for the light-emitting devices 4 and 5 are shown below.

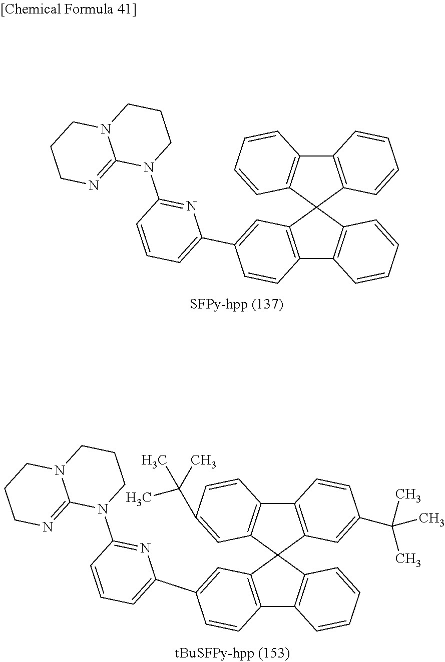 [SMILES: c1cc(-c2ccc3c(c2)C2(c4ccccc4-c4ccccc42)c2ccccc2-3)nc(N2CCCN3CCCN=C32)c1 ; CC(C)(C)c1ccc2c(c1)C1(c3ccccc3-c3ccc(-c4cccc(N5CCCN6CCCN=C65)n4)cc31)c1cc(C(C)(C)C)ccc1-2]

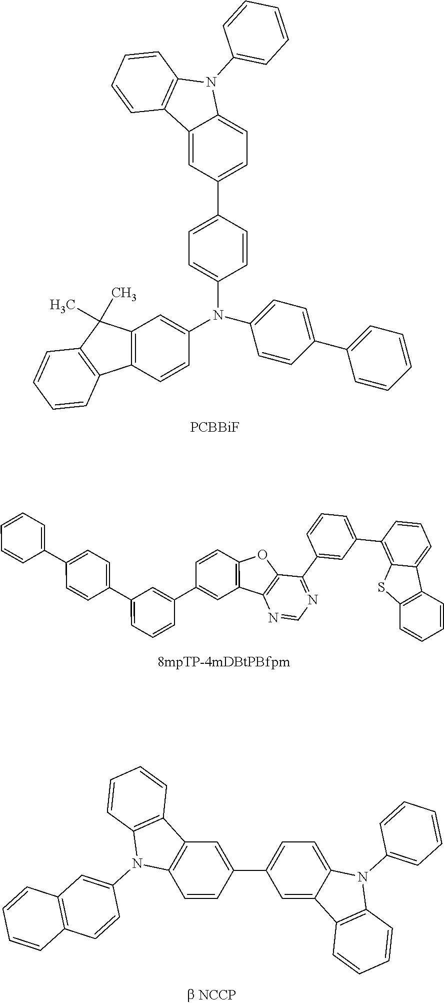

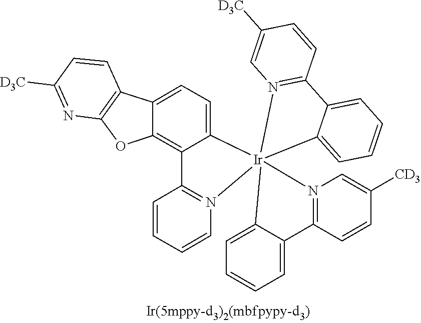

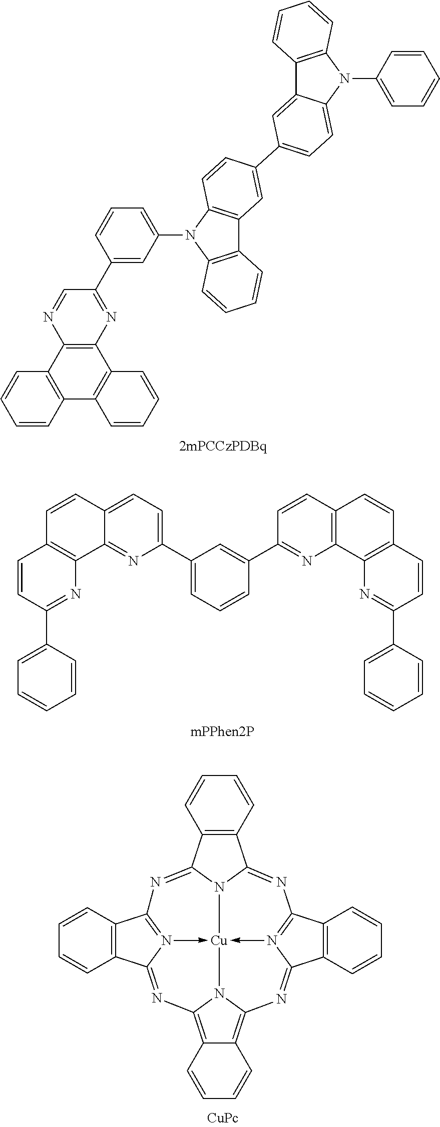

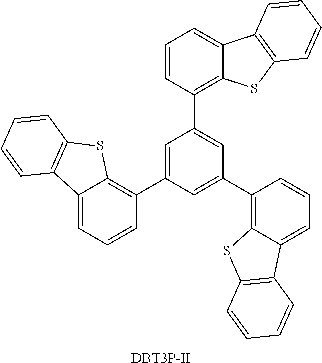 [SMILES: c1ccc2c(c1)sc1c(-c3cc(-c4cccc5c4sc4ccccc45)cc(-c4cccc5c4sc4ccccc45)c3)cccc12 ; "DBT3P-II"]

### (Fabrication Methods of Light-Emitting Devices 4 and 5)

The light-emitting devices 4 and 5 are light-emitting devices fabricated through what is called a continuous vacuum process, which does not include a step of processing the organic compound layer by a photolithography method.

The light-emitting device 4 is different from the light-emitting device 2 in the thickness of the first hole-transport layer, the structure of the first electron-transport layer, the structure of the first layer of the intermediate layer, and the thickness of the second hole-transport layer. Specifically, the thickness of the first hole-transport layer was set to 90 nm. The first electron-transport layer was formed by evaporation of 2mPCCzPDBq to a thickness of 10 nm. The first layer of the intermediate layer was formed by replacing SBF-hpp used for the light-emitting device 2 with SFPy-hpp, which is the organic compound of another embodiment of the present invention, and depositing mPPhen2P and SFPy-hpp by co-evaporation to a thickness of 5 nm such that the weight ratio of mPPhen2P to SFPy-hpp was 0.5:0.5. The thickness of the second hole-transport layer was set to 55 nm.

The light-emitting device 5 was obtained by replacing SFPy-hpp used for the first layer of the intermediate layer in the fabrication method of the light-emitting device 4 with tBuSFPy-hpp. That is, the first layer of the intermediate layer was formed by depositing mPPhen2P and tBuSFPy-hpp by co-evaporation to a thickness of 5 nm such that the weight ratio of mPPhen2P to tBuSFPy-hpp was 0.5:0.5.

The other components of the light-emitting devices 4 and 5 were formed in a manner similar to that of the light-emitting device 2, and thus are not described here. The structures of the light-emitting devices 4 and 5 are shown in the following table.

TABLE 7

| | | Film | | | |
|---|---|---|---|---|---|
| | | thickness | | Light-emitting device 4 | Light-emitting device 5 |
| Cap layer | | 70 | nm | DBT3P-II | |
| Second electrode | | 15 | nm | Ag:Mg (1:0.1) | |
| Electron-injection layer | | 1.5 | nm | LiF:Yb (2:1) | |
| Second | 2 | 20 | nm | mPPhen2P | |
| electron- | 1 | 20 | nm | 2mPCCzPDBq | |
| transport layer | | | | | |
| Second light-emitting layer | | 40 | nm | 8mpTP-4mDBtPBfpm:βNCCP:Ir(5mppy-d₃)₂(mbfpypy-d₃) | |
| | | | | (0.5:0.5:0.1) | |
| Second hole-transport layer | | 55 | nm | PCBBiF | |
| Intermediate | Third layer | 10 | nm | PCBBiF:OCHD-003 (1:0.15) | |
| layer | Second layer | 2 | nm | CuPc | |
| | First layer | 5 | nm | mPPhen2P:SFPy-hpp | mPPhen2P:tBuSFPy-hpp |
| | | | | (0.5:0.5) | (0.5:0.5) |

| First electron-transport layer | | 10 | nm | 2mPCCzPDBq |
|---|---|---|---|---|
| First light-emitting layer | | 40 | nm | 8mpTP-4mDBtPBfpm:βNCCP:Ir(5mppy-d₃)₂(mbfpypy-d₃) |
| | | | | (0.5:0.5:0.1) |
| First hole-transport layer | | 90 | nm | PCBBiF |
| Hole-injection layer | | 10 | nm | PCBBiF:OCHD-003 (1:0.03) |
| First electrode | 2 | 85 | nm | ITSO |
| | 1 | 100 | nm | Ag |

The light-emitting devices 4 and 5 were sealed using a glass substrate in a glove box containing a nitrogen atmosphere so as not to be exposed to the air. Specifically, a UV curable sealing material was applied to surround the devices, only the sealing material was irradiated with UV while the light-emitting devices were not irradiated with the UV, and heat treatment was performed at 80° C. under an atmospheric pressure for one hour. Then, the initial characteristics of the light-emitting devices were measured.

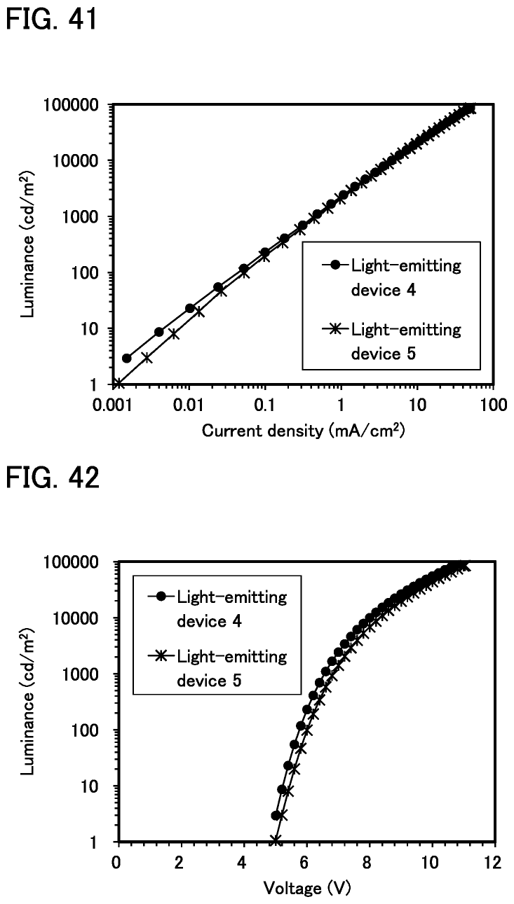

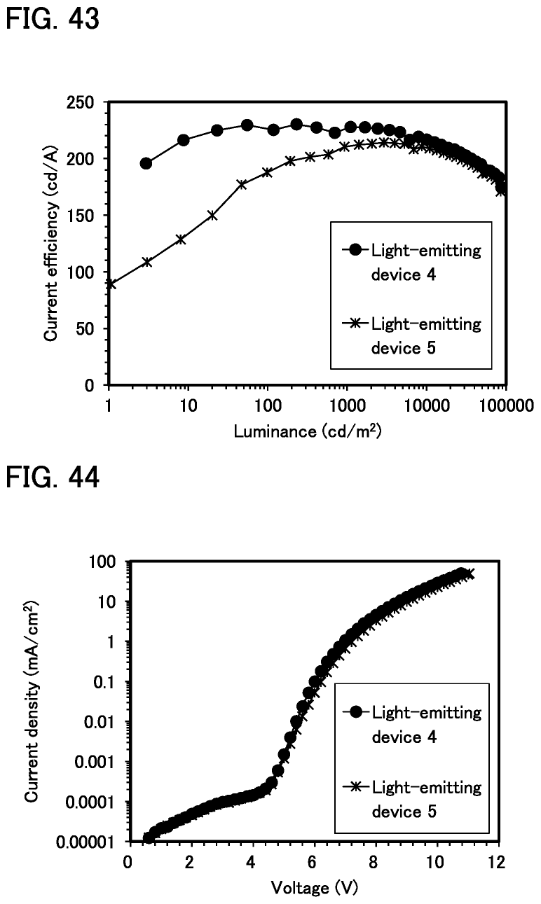

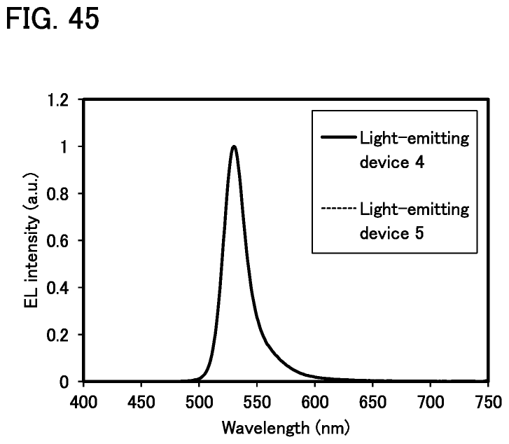

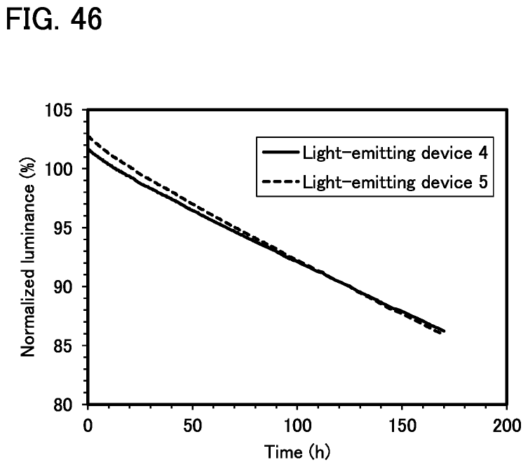

FIG. 41 shows the luminance-current density characteristics of the light-emitting devices 4 and 5, FIG. 42 shows the luminance-voltage characteristics thereof, FIG. 43 shows the current efficiency-luminance characteristics thereof, FIG. 44 shows the current density-voltage characteristics thereof, FIG. 45 shows the electroluminescence spectra thereof, and FIG. 46 shows luminance changes over driving time when the light-emitting devices 4 and 5 are driven at a constant current of 2 mA (50 mA/cm²).

The following table shows the main characteristics of the light-emitting devices 4 and 5 at a luminance of approximately 1000 cd/m². The luminance, CIE chromaticity, and electroluminescence spectrum were measured at normal temperature with a spectroradiometer (SR-UL1R manufactured by TOPCON TECHNOHOUSE CORPORATION).

TABLE 8

| | | | Current | | | | Current |
|---|---|---|---|---|---|---|---|
| | Voltage | Current | density | | | Luminance | efficiency |
| | (V) | (mA) | (mA/cm²) | Chromaticity x | Chromaticity y | (cd/m²) | (cd/A) |
| Light-emitting | 6.6 | 0.019 | 0.48 | 0.233 | 0.730 | 1103 | 228 |
| device 4 | | | | | | | |
| Light-emitting | 6.8 | 0.018 | 0.44 | 0.233 | 0.731 | 926 | 210 |
| device 5 | | | | | | | |

FIG. 41 to FIG. 46 and the above table reveal that the light-emitting devices 4 and 5 exhibit green light emission derived from Ir(5mppy-d₃)₂(mbfpypy-d₃) and have favorable light-emitting characteristics. In addition, the light-emitting devices 4 and 5 have high current efficiency and are driven as tandem light-emitting devices. This indicates that the use of the organic compound of one embodiment of the present invention for an intermediate layer enables a light-emitting device to have favorable characteristics.

This application is based on Japanese Patent Application Serial No. 2023-015356 filed with Japan Patent Office on Feb. 3, 2023, the entire contents of which are hereby incorporated by reference.

What is claimed is:

1. An organic compound represented by General Formula (G1), 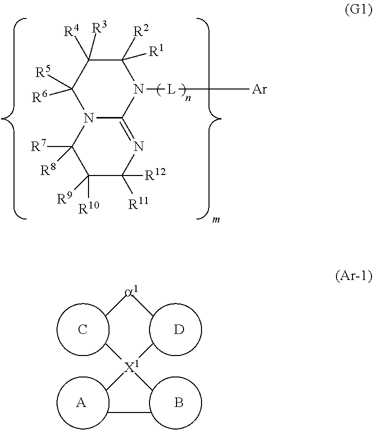
wherein in General Formula (G1):
Ar is an aromatic skeleton represented by General Formula (Ar-1);
L represents one of an alkylene group comprising 1 to 10 carbon atoms, a substituted or unsubstituted arylene group comprising 6 to 30 carbon atoms, and a substituted or unsubstituted heteroarylene group comprising 1 to 11 carbon atoms;
n represents an integer of 0 to 3;
m represents an integer of 1 to 4;
each of R¹ to R¹² independently represents one of hydrogen and an alkyl group comprising 1 to 10 carbon atoms;
in the case where n is 2 or more, Ls are the same or different from each other; and
in the case where m is 2 or more, Ls are the same or different from each other, n's are the same or different from each other, and each of R¹s to R¹²s are independently the same or different from each other, and
wherein in General Formula (Ar-1):
each of a ring A, a ring B, a ring C, and a ring D independently represents one of a substituted or unsubstituted benzene ring, a substituted or unsubstituted naphthalene ring, and a substituted or unsubstituted phenanthrene ring;
any m carbon atoms on the ring A, the ring B, the ring C, and the ring D each comprise a bond in General Formula (G1);
X¹ represents carbon, silicon, or germanium; and
α¹ represents one of a single bond, oxygen, sulfur, carbon comprising a substituent, silicon comprising a substituent, and germanium comprising a substituent.

2. The organic compound according to claim 1, wherein α¹ is represented by any of General Formulae (α-1) to (α-3), 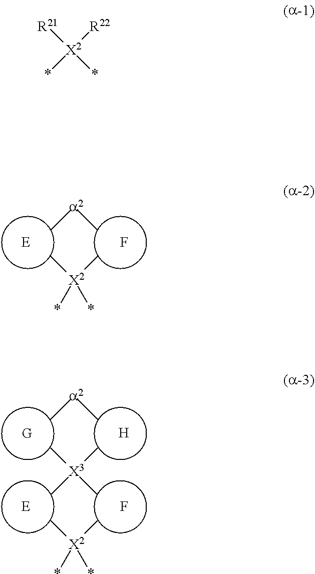
wherein in General Formulae (α-1) to (α-3):
each of X² and X³ independently represents one of carbon, silicon, and germanium;
α² represents one of a single bond, oxygen, sulfur, carbon comprising a substituent, silicon comprising a substituent, and germanium comprising a substituent;
each of a ring E, a ring F, a ring G, and a ring H independently represents one of a substituted or unsubstituted benzene ring, a substituted or unsubstituted naphthalene ring, and a substituted or unsubstituted phenanthrene ring;
each of R²¹ and R²² independently represents one of hydrogen, an alkyl group comprising 1 to 10 carbon atoms, a cycloalkyl group comprising 3 to 10 carbon atoms, a substituted or unsubstituted aryl group comprising 6 to 30 carbon atoms, and a substituted or unsubstituted heteroaryl group comprising 1 to 30 carbon atoms; and
* represents bonding positions of the ring C and the ring D in General Formula (Ar-1).

3. A light-emitting device comprising the organic compound according to claim 1.

4. A display apparatus comprising:
the light-emitting device according to claim 3; and
at least one of a transistor and a substrate.

5. An electronic device comprising:
the display apparatus according to claim 4; and
at least one of a sensor unit, an input unit, and a communication unit.

6. An organic compound represented by General Formula (G1), 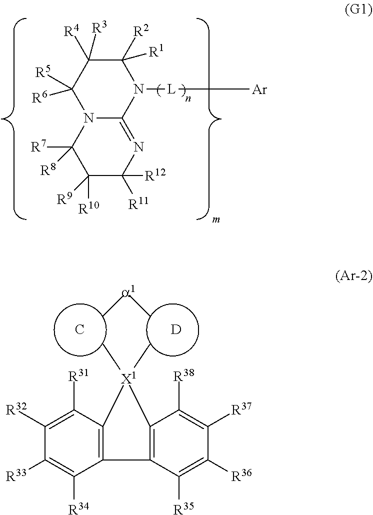
wherein in General Formula (G1):
Ar is an aromatic skeleton represented by General Formula (Ar-2);
L represents one of an alkylene group comprising 1 to 10 carbon atoms, a substituted or unsubstituted arylene group comprising 6 to 30 carbon atoms, and a substituted or unsubstituted heteroarylene group comprising 1 to 11 carbon atoms;
n represents an integer of 0 to 3;
m represents an integer of 1 to 4;
each of R¹ to R¹² independently represents one of hydrogen and an alkyl group comprising 1 to 10 carbon atoms;
in the case where n is 2 or more, Ls are the same or different from each other; and
in the case where m is 2 or more, Ls are the same or different from each other, n's are the same or different from each other, and each of R¹s to R¹²s are independently the same or different from each other, and
wherein in General Formula (Ar-2):
each of a ring C and a ring D independently represents one of a substituted or unsubstituted benzene ring, a substituted or unsubstituted naphthalene ring, and a substituted or unsubstituted phenanthrene ring;
X¹ represents one of carbon, silicon, and germanium;
α¹ represents one of a single bond, oxygen, sulfur, carbon comprising a substituent, silicon comprising a substituent, and germanium comprising a substituent;
any m groups of R³¹ to R³⁸ represent bonding positions in General Formula (G1); and
each of the other groups of R³¹ to R³⁸ independently represents one of hydrogen, an alkyl group comprising 1 to 10 carbon atoms, a cycloalkyl group comprising 3 to 10 carbon atoms, a substituted or unsubstituted aryl group comprising 6 to 30 carbon atoms, and a substituted or unsubstituted heteroaryl group comprising 1 to 30 carbon atoms.

7. The organic compound according to claim 6, wherein α¹ is represented by any of General Formulae (α-4) to (α-6), 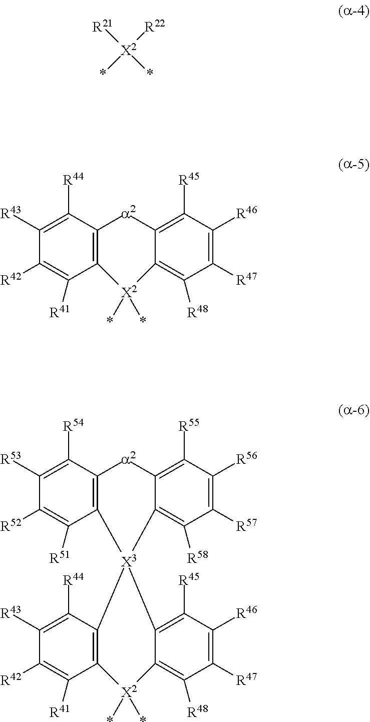
wherein in General Formulae (α-4) to (α-6):
each of X² and X³ independently represents one of carbon, silicon, and germanium;
α² represents one of a single bond, oxygen, sulfur, carbon comprising a substituent, silicon comprising a substituent, and germanium comprising a substituent;
each of R²¹, R²², R⁴¹ to R⁴⁸, and R⁵¹ to R⁵⁸ independently represents one of hydrogen, an alkyl group comprising 1 to 10 carbon atoms, a cycloalkyl group comprising 3 to 10 carbon atoms, a substituted or unsubstituted aryl group comprising 6 to 30 carbon atoms, and a substituted or unsubstituted heteroaryl group comprising 1 to 30 carbon atoms; and
* represents bonding positions of the ring C and the ring D in General Formula (Ar-2).

8. A light-emitting device comprising the organic compound according to claim 6.

9. A display apparatus comprising:
the light-emitting device according to claim 8; and
at least one of a transistor and a substrate.

10. An electronic device comprising:
the display apparatus according to claim 9; and
at least one of a sensor unit, an input unit, and a communication unit.

11. An organic compound represented by General Formula (G1), 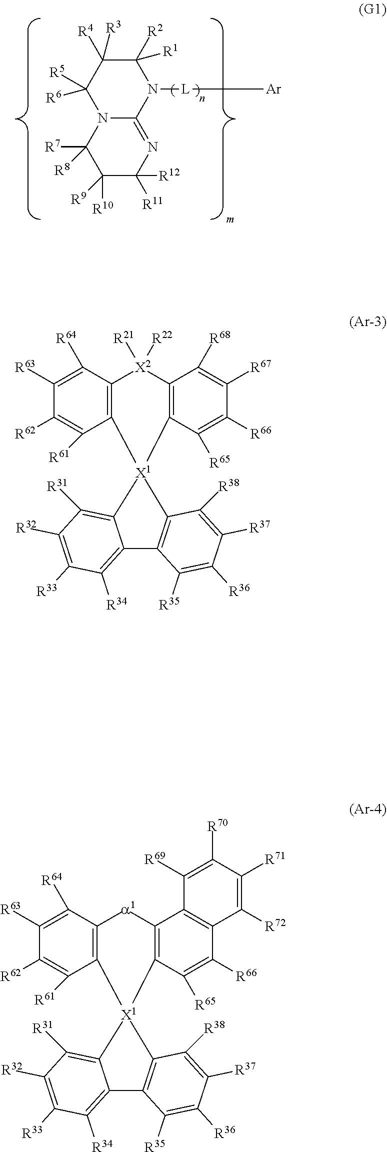
wherein in General Formula (G1):
Ar represents an aromatic skeleton represented by one of General Formula (Ar-3) and (Ar-4);
L represents one of an alkylene group comprising 1 to 10 carbon atoms, a substituted or unsubstituted arylene group comprising 6 to 30 carbon atoms, and a substituted or unsubstituted heteroarylene group comprising 1 to 11 carbon atoms;
n represents an integer of 0 to 3;
m represents an integer of 1 to 4;
each of R¹ to R¹² independently represents one of hydrogen and an alkyl group comprising 1 to 10 carbon atoms;
in the case where n is 2 or more, Ls are the same or different from each other;
in the case where m is 2 or more, Ls are the same or different from each other, n's are the same or different from each other, and each of R¹s to R¹²s are independently the same or different from each other, and
wherein in General Formulae (Ar-3) and (Ar-4):
each of X¹ and X² independently represents one of carbon, silicon, and germanium;
α¹ represents one of a single bond, oxygen, sulfur, carbon comprising a substituent, silicon comprising a substituent, and germanium comprising a substituent;
any m groups of R³¹ to R³⁸ and R⁶¹ to R⁶⁶ represent bonding positions in General Formula (G1); and
each of the other groups of R³¹ to R³⁸ and R⁶¹ to R⁶⁶, and R⁶⁷ to R⁷², R²¹, and R²² independently represents one of hydrogen, an alkyl group comprising 1 to 10 carbon atoms, a cycloalkyl group comprising 3 to 10 carbon atoms, a substituted or unsubstituted aryl group comprising 6 to 30 carbon atoms, and a substituted or unsubstituted heteroaryl group comprising 1 to 30 carbon atoms.

12. A light-emitting device comprising the organic compound according to claim 11.

13. A display apparatus comprising:
the light-emitting device according to claim 12; and
at least one of a transistor and a substrate.

14. An electronic device comprising:
the display apparatus according to claim 13; and
at least one of a sensor unit, an input unit, and a communication unit.
